(19) United States
(12) Patent Application Publication
(10) Pub. No.: US 2026/0265717 A1
(43) Pub. Date: Sep. 10, 2026

(54) SYSTEMS AND METHODS FOR TRANSPOSING CARGO NUCLEOTIDE SEQUENCES

(71) Applicant: Metagenomi, Inc., Emeryville, CA (US)

(72) Inventors: Brian C. THOMAS, Berkeley, CA (US); Lisa ALEXANDER, Albany, CA (US); Christopher BROWN, Albany, CA (US); Cristina Noel BUTTERFIELD, Oakland, CA (US); Gregory J. COST, Piedmont, CA (US); Daniela S.A. GOLTSMAN, Oakland, CA (US); Khak Khak KHAYI, Oakland, CA (US); Jason LIU, Berkeley, CA (US); Christine ROMANO, Emeryville, CA (US); Zhiqing WANG, Emeryville, CA (US); Drew DUNHAM, Emeryville, CA (US); Jennifer HONG, San Lorenzo, CA (US)

(73) Assignee: Metagenomi, Inc., Emeryville, CA (US)

(21) Appl. No.: 19/163,801

(22) PCT Filed: May 10, 2024
(86) PCT No.: PCT/US2024/028988

§ 371 Date: Sep. 9, 2025

Related U.S. Application Data
(60) Provisional application No. 63/501,227, filed on May 10, 2023.
(60) Provisional application No. 63/514,768, filed on Jul. 20, 2023.

(51) Int. Cl. C12N 9/22; C12N 9/12; C12N 15/11; C12N 15/90

(52) U.S. Cl. CPC C12N 9/226; C12N 9/1241; C12N 15/11; C12N 15/907; C12N 2310/20

(57) ABSTRACT

The present disclosure provides systems and methods for transposing a cargo nucleotide sequence into a target nucleic acid site in a target nucleic acid. These systems and methods may comprise a double-stranded nucleic acid comprising the cargo nucleotide sequence, wherein the cargo nucleotide sequence interacts with a recombinase complex, an effector complex comprising an effector and at least one engineered guide polynucleotide that hybridizes to the target nucleic acid, and the recombinase complex wherein the recombinase complex recruits the cargo nucleotide to the target nucleic acid site.

### CROSS-REFERENCE

This application claims the benefit of and priority to U.S. Provisional Patent Application No. 63/501,227 filed May 10, 2023, and U.S. Provisional Patent Application No. 63/514,768 filed Jul. 20, 2023, each of which is incorporated by reference in its entirety herein.

### SEQUENCE LISTING

The instant application contains a Sequence Listing which has been submitted electronically in XML format and is hereby incorporated by reference in its entirety. Said XML copy, created on May 3, 2024, is named MTG-026WO_SL.xml and is 1.536.000 bytes in size.

### BACKGROUND

Cas enzymes along with their associated Clustered Regularly Interspaced Short Palindromic Repeats (CRISPR) guide ribonucleic acids (RNAs) appear to be a pervasive (~45% of bacteria, ~84% of archaea) component of prokaryotic immune systems, serving to protect such microorganisms against non-self nucleic acids, such as infectious viruses and plasmids by CRISPR-RNA guided nucleic acid cleavage. While the deoxyribonucleic acid (DNA) elements encoding CRISPR RNA elements may be relatively conserved in structure and length, their CRISPR-associated (Cas) proteins are highly diverse, containing a wide variety of nucleic acid-interacting domains. While CRISPR DNA elements have been observed as early as 1987, the programmable endonuclease cleavage ability of CRISPR/Cas complexes has only been recognized relatively recently, leading to the use of recombinant CRISPR/Cas systems in diverse DNA manipulation and gene editing applications.

### SUMMARY

In some aspects, the present disclosure provides for a system for transposing a cargo nucleotide sequence into a target nucleic acid site comprising: a first double-stranded nucleic acid comprising a cargo nucleotide sequence configured to interact with a Tn7 type transposase complex; a Cas effector complex comprising a class 2, type V Cas effector and an engineered guide polynucleotide configured to hybridize to said target nucleotide sequence; and a Tn7 type transposase complex configured to bind said Cas effector complex, wherein said Tn7 type transposase complex comprises a TnsB subunit. In some embodiments, said cargo nucleotide sequence is flanked by a left-hand transposase recognition sequence and a right-hand transposase recognition sequence. In some embodiments, the system further comprises a second double-stranded nucleic acid comprising said target nucleic acid site. In some embodiments, the system further comprises a PAM sequence compatible with said Cas effector complex adjacent to said target nucleic acid site. In some embodiments, said PAM sequence is located 3′ of said target nucleic acid site.

In some aspects, the present disclosure provides a system for transposing a cargo nucleotide sequence into a target nucleic acid site in a target nucleic acid comprising a Cas effector complex comprising a class 2, type V Cas effector, a small prokaryotic ribosomal protein subunit S15, and an engineered guide polynucleotide that hybridizes to the target nucleic acid site; a Tn7 type transposase complex that binds the Cas effector complex and comprises a TnsB, TnsC, and TniQ component and an accessory protein comprising a sequence having at least 70% sequence identity to any one of SEQ ID NOs: 228-230 and 235-249; and a double-stranded nucleic acid that interacts with the Tn7 type transposase complex and comprises the cargo nucleotide sequence.

In some embodiments, the Cas effector complex binds non-covalently to the Tn7 type transposase complex. In some embodiments, the Cas effector complex is covalently linked to the Tn7 type transposase complex. In some embodiments, the Cas effector complex is fused to the Tn7 type transposase complex.

In some embodiments, the cargo nucleotide sequence is flanked by a left-hand transposase recognition sequence and a right-hand transposase recognition sequence recognized by the Tn7 type transposase complex. In some embodiments, the left-hand transposase recognition sequence comprises a sequence having at least 80% identity to any one of SEQ ID NOs: 9, 11, 36-38, 76, and 78. In some embodiments, the right-hand transposase recognition sequence comprises a sequence having at least 80% identity to any one of SEQ ID NOs: 8, 10, 39-44, 77, 79, and 93.

In some embodiments, the target nucleic acid comprises a PAM sequence compatible with the Cas effector complex. In some embodiments, the PAM sequence comprises SEQ ID NO: 31. In some embodiments, the PAM sequence is located about 50 to about 70 base pairs from the target nucleic acid site. In some embodiments, the PAM sequence is located 3′ of the target nucleic acid site. In some embodiments, the PAM sequence is located 5′ of the target nucleic acid site.

In some embodiments, the class 2, type V Cas effector is a Cas12k effector. In some embodiments, the class 2, type V Cas effector comprises a polypeptide comprising a sequence having at least 80% identity to any one of SEQ ID NOs: 1, 12, 16, 20-30, 64, 80-85, and 220.

In some embodiments, the class 2, type V Cas effector comprises a polypeptide comprising a sequence having at least 90% identity to any one of SEQ ID NOs: 1, 12, 16, 20-30, 64, 80-85, and 220. In some embodiments, the class 2, type V Cas effector comprises a polypeptide comprising a sequence of any one of SEQ ID NOs: 1, 12, 16, 20-30, 64, 80-85, and 220.

In some embodiments, the TnsB component comprises a polypeptide having a sequence having at least 80% identity to any one of SEQ ID NOs: 2, 13, 17, and 65. In some embodiments, the TnsB component comprises a polypeptide having a sequence having at least 90% identity to any one of SEQ ID NOs: 2, 13, 17, and 65. In some embodiments, the TnsB component comprises a polypeptide having a sequence of any one of SEQ ID NOs: 2, 13, 17, and 65.

In some embodiments, the Tn7 type transposase complex comprises at least a first polypeptide and a second polypeptide each independently comprising a sequence having at least 80% identity to any one of SEQ ID NOs: 3-4, 14-15, 18-19, 66-67, and 109-111. In some embodiments, the Tn7 type transposase complex comprises at least a first polypeptide and a second polypeptide each independently comprising a sequence having at least 90% identity to any one of SEQ ID NOs: 3-4, 14-15, 18-19, 66-67, and 109-111. In some embodiments, the Tn7 type transposase complex comprises at least a first polypeptide and a second polypeptide each independently comprising a sequence of any one of SEQ ID NOs: 3-4, 14-15, 18-19, 66-67, and 109-111.

In some embodiments, the engineered guide polynucleotide comprises a sequence comprising at least about 46-80 consecutive nucleotides having at least 80% identity to any one of SEQ ID NOs: 5-6, 32-33, 94-95, 104-105, 119-122, and 222. In some embodiments, the engineered guide polynucleotide comprises a sequence having at least 80% sequence identity to any one of SEQ ID NOs: 106, 107, 108, 5, 45-63, 68-75, 96-103, 123-140, and 754-944.

In some embodiments, the small prokaryotic ribosomal protein subunit S15 comprises a sequence having at least 80% sequence identity to any one of SEQ ID NOs: 187-189. In some embodiments, the small prokaryotic ribosomal protein subunit S15 is encoded by a sequence having at least 80% sequence identity to any one of SEQ ID NOs: 181-183.

In some embodiments, the class 2, type V Cas effector and the Tn7 type transposase complex are encoded by polynucleotide sequences comprising fewer than about 10 kilobases.

In some embodiments, the accessory protein is ClpX comprising a sequence having at least 80% sequence identity to any one of SEQ ID NOs: 235-249.

In another aspect, the present disclosure provides system for transposing a cargo nucleotide sequence into a target nucleic acid site in a target nucleic acid comprising: a Cas effector complex comprising a class 2, type V Cas effector, a small prokaryotic ribosomal protein subunit S15, and an engineered guide polynucleotide that hybridizes to the target nucleic acid site; a Tn7 type transposase complex that binds the Cas effector complex and comprises a functional domain (FD)-TniQ fusion, and an accessory protein; and a double-stranded nucleic acid that interacts with the Tn7 type transposase complex and comprises the cargo nucleotide sequence.

In some embodiments, the Cas effector complex binds non-covalently to the Tn7 type transposase complex. In some embodiments, the Cas effector complex is covalently linked to the Tn7 type transposase complex. In some embodiments, the Cas effector complex is fused to the Tn7 type transposase complex.

In some aspects, the present disclosure provides the functional domain (FD) comprises a sequence having at least 70% identity to any one of SEQ ID NOs: 257-307 and 1138-1242.

In some embodiments, the cargo nucleotide sequence is flanked by a left-hand transposase recognition sequence and a right-hand transposase recognition sequence recognized by the Tn7 type transposase complex. In some embodiments, the left-hand transposase recognition sequence comprises a sequence having at least 80% identity to any one of SEQ ID NOs: 9, 11, 36-38, 76, and 78. In some embodiments, the right-hand transposase recognition sequence comprises a sequence having at least 80% identity to any one of SEQ ID NOs: 8, 10, 39-44, 77, 79, and 93.

In some embodiments, the target nucleic acid comprises a PAM sequence compatible with the Cas effector complex. In some embodiments, the PAM sequence comprises SEQ ID NO: 31. In some embodiments, the PAM sequence is located about 50 to about 70 base pairs from the target nucleic acid site. In some embodiments, the PAM sequence is located 3′ of the target nucleic acid site. In some embodiments, the PAM sequence is located 5′ of the target nucleic acid site.

In some embodiments, the class 2, type V Cas effector is a Cas12k effector. In some embodiments, the class 2, type V Cas effector comprises a polypeptide comprising a sequence having at least 90% identity to any one of SEQ ID NOs: 1, 12, 16, 20-30, 64, 80-85, and 220. In some embodiments, the class 2, type V Cas effector comprises a polypeptide comprising a sequence of any one of SEQ ID NOs: 1, 12, 16, 20-30, 64, 80-85, and 220.

In some embodiments, the engineered guide polynucleotide comprises a sequence comprising at least about 46-80 consecutive nucleotides having at least 80% identity to any one of SEQ ID NOs: 5-6, 32-33, 94-95, 104-105, 119-122, and 222. In some embodiments, the engineered guide polynucleotide comprises a sequence having at least 80% sequence identity to any one of SEQ ID NOs: 106, 107, 108, 5, 45-63, 68-75, 96-103, 123-140, and 754-944.

In some embodiments, the small prokaryotic ribosomal protein subunit S15 comprises a sequence having at least 80% sequence identity to any one of SEQ ID NOs: 187-189. In some embodiments, the small prokaryotic ribosomal protein subunit S15 is encoded by a sequence having at least 80% sequence identity to any one of SEQ ID NOs: 181-183. In some embodiments, the class 2, type V Cas effector and the Tn7 type transposase complex are encoded by polynucleotide sequences comprising fewer than about 10 kilobases.

In some embodiments, the accessory protein comprises a sequence having at least 70% sequence identity to any one of SEQ ID NOs: 228-230 and 235-249. In some embodiments, the accessory protein is ClpX comprising a sequence having at least 80% sequence identity to any one of SEQ ID NOs: 235-249.

In another aspect, the present disclosure provides a system for transposing a cargo nucleotide sequence into a target nucleic acid site in a target nucleic acid comprising: a Cas effector complex comprising a class 2, type V Cas effector and an engineered guide polynucleotide that hybridizes to the target nucleic acid site, wherein the Cas effector complex comprises a polypeptide comprising a sequence having at least 80% sequence identity to any one of SEQ ID NOs: 1, 12, 16, 20-30, 64, 80-85, and 220: a Tn7 type transposase complex that binds the Cas effector complex and comprises a TnsB, TnsC, and TniQ component, the TnsB, TnsC, or TniQ component comprising a sequence having at least 80% sequence identity to any one of SEQ ID NOs: 2-4, 13-15, 17-19, 65-67, and 109-111, and an accessory protein comprising a sequence having at least 80% sequence identity to any one of SEQ ID NOs: 228-230 and 235-249; and a double-stranded nucleic acid that interacts with the Tn7 type transposase complex and comprises the cargo nucleotide sequence.

In some embodiments, the Cas effector complex binds non-covalently to the Tn7 type transposase complex. In some embodiments, the Cas effector complex is covalently linked to the Tn7 type transposase complex. In some embodiments, the Cas effector complex is fused to the Tn7 type transposase complex.

In some embodiments, the cargo nucleotide sequence is flanked by a left-hand transposase recognition sequence and a right-hand transposase recognition sequence recognized by the Tn7 type transposase complex. In some embodiments, the left-hand transposase recognition sequence comprises a sequence having at least 80% identity to any one of SEQ ID NOs: 9, 11, 36-38, 76, and 78. In some embodiments, the right-hand transposase recognition sequence comprises a sequence having at least 80% identity to any one of SEQ ID NOs: 8, 10, 39-44, 77, 79, and 93.

In some embodiments, the target nucleic acid comprises a PAM sequence compatible with the Cas effector complex. In some embodiments, the PAM sequence comprises SEQ ID NO: 31. In some embodiments, the PAM sequence is located about 50 to about 70 base pairs from the target nucleic acid site. In some embodiments, the PAM sequence is located 3′ of the target nucleic acid site. In some embodiments, the PAM sequence is located 5′ of the target nucleic acid site.

In some embodiments, the class 2, type V Cas effector is a Cas12k effector. In some embodiments, the class 2, type V Cas effector comprises a polypeptide comprising a sequence having at least 90% identity to any one of SEQ ID NOs: 1, 12, 16, 20-30, 64, 80-85, and 220.

In some embodiments, the class 2, type V Cas effector comprises a polypeptide comprising a sequence of any one of SEQ ID NOs: 1, 12, 16, 20-30, 64, 80-85, and 220.

In some embodiments, the TnsB, TnsC, or TniQ component comprises a sequence having at least 90% sequence identity to any one of SEQ ID NOs: 2-4, 13-15, 17-19, 65-67, and 109-111. In some embodiments, the TnsB, TnsC, or TniQ component comprises a sequence of any one of SEQ ID NOs: 2-4, 13-15, 17-19, 65-67, and 109-111.

In some embodiments, the engineered guide polynucleotide comprises a sequence comprising at least about 46-80 consecutive nucleotides having at least 80% identity to any one of SEQ ID NOs: 5-6, 32-33, 94-95, 104-105, 119-122, and 222. In some embodiments, the engineered guide polynucleotide comprises a sequence having at least 80% sequence identity to any one of SEQ ID NOs: 106, 107, 108, 5, 45-63, 68-75, 96-103, 123-140, and 754-944.

In some embodiments, the small prokaryotic ribosomal protein subunit S15 comprises a sequence having at least 80% sequence identity to any one of SEQ ID NOs: 187-189. In some embodiments, the small prokaryotic ribosomal protein subunit S15 is encoded by a sequence having at least 80% sequence identity to any one of SEQ ID NOs: 181-183.

In some embodiments, the class 2, type V Cas effector and the Tn7 type transposase complex are encoded by polynucleotide sequences comprising fewer than about 10 kilobases.

In some embodiments, the accessory protein is ClpX comprising a sequence having at least 90% sequence identity to any one of SEQ ID NOs: 235-249.

In some aspects, the present disclosure provides a system for transposing a cargo nucleotide sequence into a target nucleic acid site in a target nucleic acid comprising a Cas effector complex that hybridizes to the target nucleic acid site and comprising: i) a class 2, type V Cas effector comprising a sequence having at least 80% sequence identity to any one of SEQ ID NOs: 1, 81, 82, 83, and 85; and ii) an engineered guide polynucleotide having at least 80% identity to any one of SEQ ID NOs: 5, 6, 45-63, 68-75, 96-103, 123-140, and 754-944; a Tn7 type transposase complex that binds the Cas effector complex and comprises a TnsB, TnsC, and TniQ component, the TnsB, TnsC, or TniQ component comprising a sequence having at least 80% identity to any one of SEQ ID NOs: 2-4, and an accessory protein comprising a sequence having at least 80% sequence identity to any one of SEQ ID NOs: 228-230 and 235-249; and a double-stranded nucleic acid that interacts with the Tn7 type transposase complex and comprising in 5′ to 3′ order: i) a left-hand transposase recognition sequence comprising a sequence having at least 80% sequence identity to any one of SEQ ID NOs: 9, 11, 36, 37, and 38; ii) the cargo nucleotide sequence; and ii) a right-hand transposase recognition sequence comprising a sequence having at least 80% identity to any one of SEQ ID NOs: 8, 39-44, and 93.

In some aspects, the present disclosure provides a system for transposing a cargo nucleotide sequence into a target nucleic acid site in a target nucleic acid comprising: a Cas effector complex that hybridizes to the target nucleic acid site and comprising: i) a class 2, type V Cas effector comprising a sequence having at least 80% sequence identity to SEQ ID NOs: 12; and iii) an engineered guide polynucleotide having at least 80% identity to any one of SEQ ID NOs: 32, 102, 104, and 107; a Tn7 type transposase complex that binds the Cas effector complex and comprising a TnsB, TnsC, and TniQ component, the TnsB, TnsC, or TniQ component comprising a sequence having at least 80% identity to any one of SEQ ID NOs: 13-15, and an accessory protein comprising a sequence having at least 80% sequence identity to any one of SEQ ID NOs: 228-230 and 235-249; and a double-stranded nucleic acid that interacts with the Tn7 type transposase complex and comprising in 5′ to 3′ order: i) a left-hand transposase recognition sequence comprising a sequence having at least 80% sequence identity to SEQ ID NO: 76; ii) the cargo nucleotide sequence; and iii) a right-hand transposase recognition sequence comprising a sequence having at least 80% identity to SEQ ID NO: 77.

In some aspects, the present disclosure provides a system for transposing a cargo nucleotide sequence into a target nucleic acid site in a target nucleic acid comprising: a Cas effector complex that hybridizes to the target nucleic acid site and comprising: i) a class 2, type V Cas effector comprising a sequence having at least 80% sequence identity to SEQ ID NOs: 16; and ii) an engineered guide polynucleotide having at least 80% identity to any one of SEQ ID NOs: 33, 103, 105, and 108; a Tn7 type transposase complex that binds the Cas effector complex and comprises a TnsB, TnsC, and TniQ component, the TnsB, TnsC, or TniQ component comprising a sequence having at least 80% identity to any one of SEQ ID NOs: 17-19, an accessory protein comprising a sequence having at least 80% sequence identity to any one of SEQ ID NOs: 228-230 and 235-249; and a double-stranded nucleic acid that interacts with the Tn7 type transposase complex and comprising in 5′ to 3′ order: i) a left-hand transposase recognition sequence comprising a sequence having at least 80% sequence identity to SEQ ID NO: 78; ii) the cargo nucleotide sequence; and iii) a right-hand transposase recognition sequence comprising a sequence having at least 80% identity to SEQ ID NO: 79.

In some embodiments, the system further comprises a PAM sequence compatible with the Cas effector complex. In some embodiments, the PAM sequence comprises SEQ ID NO: 31.

In some embodiments, the PAM sequence is located about 50 to about 70 base pairs from the target nucleic acid site. In some embodiments, the PAM sequence is located 3′ of the target nucleic acid site. In some embodiments, the PAM sequence is located 5′ of the target nucleic acid site.

In some embodiments, the Cas effector complex further comprises a small prokaryotic ribosomal protein subunit S15. In some embodiments, the small prokaryotic ribosomal protein subunit S15 comprises a sequence having at least 80% sequence identity to any one of SEQ ID NOs: 187-189. In some embodiments, the accessory protein is ClpX comprising a sequence having at least 80% sequence identity to any one of SEQ ID NOs: 235-249.

In some aspects, the present disclosure provides an engineered nuclease system comprising: an endonuclease comprising a RuvC domain, the endonuclease being derived from an uncultivated microorganism and is a Class 2, type V-K Cas effector comprising at least 80% identity to any one of SEQ ID NOs: 1, 12, 16, 20-30, 64, 80-85, and 220; and an engineered guide RNA that forms a complex with the endonuclease and comprising a spacer sequence that hybridizes to a target nucleic acid sequence wherein the engineered guide polynucleotide comprises a sequence comprising at least 80% identity to any one of SEQ ID NOs: 754-944.

In some aspects, the present disclosure provides a method for transposing a cargo nucleotide sequence into a target nucleic acid site comprising introducing the system of any one of disclosed herein to a cell. In some aspects, the present disclosure provides a cell comprising the system of any one of the systems disclosed herein. In some embodiments, the cell is a eukaryotic cell.

In some embodiments, the cell is a mammalian cell. In some embodiments, the cell is an immortalized cell. In some embodiments, the cell is an insect cell. In some embodiments, the cell is a yeast cell. In some embodiments, the cell is a plant cell. In some embodiments, the cell is a fungal cell. In some embodiments, the cell is a prokaryotic cell. In some embodiments, the cell is an A549, HEK-293, HEK-293T, BHK, CHO, HeLa, MRC5, Sf9, Cos-1, Cos-7, Vero, BSC 1, BSC 40, BMT 10, W138, HeLa, Saos, C2C12, L cell, HT1080, HepG2, Huh7, K562, primary cell, or a derivative thereof. In some embodiments, the cell is an engineered cell. In some embodiments, the cell is a stable cell.

Additional aspects and advantages of the present disclosure will become readily apparent to those skilled in this art from the following detailed description, wherein only illustrative embodiments of the present disclosure are shown and described. As will be realized, the present disclosure is capable of other and different embodiments, and its several details are capable of modifications in various obvious respects, all without departing from the disclosure. Accordingly, the drawings and description are to be regarded as illustrative in nature, and not as restrictive.

### BRIEF DESCRIPTION OF THE DRAWINGS

The novel features of the disclosure are set forth with particularity in the appended claims. A better understanding of the features and advantages of the present disclosure will be obtained by reference to the following detailed description that sets forth illustrative embodiments, in which the principles of the disclosure are utilized, and the accompanying drawings (also “Figure” and “FIG.” herein), of which:

FIG. 1 depicts example organizations of CRISPR/Cas loci of different classes and types.

FIG. 2 depicts the architecture of a natural Class 2 Type II crRNA/tracrRNA pair shown e.g., for Cas9, compared to a hybrid sgRNA wherein the crRNA and tracrRNA are joined.

FIG. 3 depicts the two pathways found in Tn7 and Tn7-like elements.

FIGS. 4A-4B depict the genomic context of a Type V Tn7 CAST of the family MG64. FIG. 4A depicts that the MG64-1 CAST system comprises a CRISPR array (CRISPR repeats), a Type V nuclease, and three predicted transposase protein sequences. A tracrRNA was predicted in the intergenic region between the CAST effector and CRISPR array. Bottom: Multiple sequence alignment of the catalytic domain of transposase TnsB. The catalytic residues are indicated by boxes. FIG. 4B depicts that the two transposon ends were predicted for the MG64-1 CAST system.

FIG. 5 depicts depict predicted structures of corresponding sgRNAs of CAST systems described herein. Panel A of FIG. 5 (left) shows the predicted MG64-1 tracrRNA and crRNA duplex complexes at the repeat-antirepeat stem. Loop was truncated and a tetraloop of GAAA was added to the stem loop structure to produce the designed sgRNA shown in panel B of FIG. 5 (right).

FIG. 6 depicts the results of a transposition reaction targeted to a plasmid Library consisting of NNNNNNNN at the 5′ of the target spacer sequence. Reaction #1 indicates the presence of the target Library, #2 shows presence of Donor fragments in both transposition reactions, #3-5 shows sg specific PCR band that corresponds to proper transposition reactions.

FIGS. 7A-7D depict the results of Sanger sequencing. FIG. 7A shows Sanger sequencing of the donor target junction on the transposon Left End (LE) in LE-closer-to-PAM transposition reactions. Expected sequence is at the top of the panel, with a predicted transposition event 61 bp away from the PAM. Top chromatogram is sequencing result that begins from within the donor fragment. Clear signal is seen on the right end up until the donor/target junction (dotted line). This denotes a mix of transposition products. The bottom chromatogram of the panel is sequencing from the target to the donor/target junction. The signal from the left is clear signal until the point of junction. FIG. 7B shows Sanger sequencing of the donor target junction on the transposon Right End (RE) in LE-closer-to-PAM products. Expected sequence is at the top of the panel, with a predicted transposition event 61 bp away from the PAM. Top chromatogram is sequencing result than begin from within the donor fragment. Clear signal is seen on the left end up until the donor/target junction (dotted line). FIG. 7C is a close up of the PAM library. FIG. 7D is the SeqLogo analysis on NGS of the LE-closer-to-PAM events which indicates a very strong preference for NGTN in the PAM motif.

FIG. 8 depicts a phylogenetic gene tree of Cas12k effector sequences. The tree was inferred from a multiple sequence alignment of 64 Cas12k sequences recovered here (orange and black branches) and 229 reference Cas12k sequences from public databases (grey branches). Orange branches indicate Cas12k effectors with confirmed association with CAST transposon components.

FIG. 9 shows MG64 family CRISPR repeat alignment. Cas12k CAST CRISPR repeats contain a conserved motif 5′-GNNGGNNTGAAAG-3′. In MG64-1, short repeat-antirepeats (RAR) within the CRISPR repeat motif align with the tracrRNA. MG64 RAR motifs appear to define the start and end of the tracrRNA (5′ end: RAR1 (TTTC); 3′ end: RAR2 (CCNNC)).

FIG. 10A and FIG. 10B depicts secondary structure predicted from folding the CRISPR repeat+tracrRNA for MG64 systems.

FIG. 11A depicts the MG64-3 CRISPR locus. The tracrRNA is encoded upstream from the CRISPR array, while the transposon end is encoded downstream (inner black box). A sequence corresponding to a partial 3′ CRISPR repeat and a partial spacer are encoded within the transposon (outer box). The self-matching spacer is encoded outside of the transposon end.

FIG. 11B depicts tracrRNA sequence alignment for various CASTs provided herein. Alignment of tracrRNA sequences shows regions of conservation. In particular, the sequence “TGCTTTC” at sequence position 92-98 (top box) may be important for sgRNA tertiary structure and for a non-continuous repeat-anti-repeat pairing with the crRNA. The hairpin “CYCC(n6)GGRG” at positions 265-278 (bottom box) may be important for function, such as by positioning the downstream sequence for crRNA pairing.

FIG. 12A depicts the predicted structure of MG64-1 sgRNA.

FIG. 12B depicts the predicted structure of MG64-3 sgRNA.

FIG. 12C depicts the predicted structure of MG64-5 sgRNA.

FIGS. 13A-13C depicts PCR data which demonstrate that MG64-1 is active with sgRNA v2-1. Using the protocol described for in vitro targeted integrase activity, the effector protein and its TnsB. TnsC, and TniQ proteins were expressed in an in vitro transcription/translation system. After translation, the target DNA, cargo DNA, and sgRNA were added in reaction buffer. Integration was assayed by PCR across the target/donor junctions. FIG. 13A depicts a diagram illustrating the potential orientation of integrated donor DNA. PCR reactions 3, 4, 5, and 6 represent each integration ligation product depending on the orientation in which the donor was integrated at the target site. FIG. 13B depicts a gel image of PCR 4 (detecting the RE junction to the donor) of transposition showing: lane 1) apo (no sgRNA), lane 2) with sgRNA 1, and lane 3) with sgRNA v2-1. FIG. 13C depicts a gel image of PCR 5 (detecting the LE junction to the donor) of transposition showing: lane 1) apo (no sgRNA), lane 2) with sgRNA 1, and lane 3) with sgRNA v2-1.

FIG. 14 depicts PCR reaction 5 (LE proximal to PAM, top half of plot) and PCR reaction 4 (RE distal to PAM, bottom half of plot) plotted on the sequence and distance from the PAM for MG64-1. Analysis of the integration window indicates that 95% of the integrations that occur at the spacer PAM site are within a 10 bp window between 58 and 68 nucleotides away from the PAM. Differences in the integration distance between the distal and the proximal frequencies reflects the integration site duplication—a 3-5 base pair duplication as a result of staggered nuclease activity of the transposase upon integration.

FIG. 15 depicts the results of a colony PCR screen of Transposition Efficiency. After incubation. 18 colony forming units (CFUs) were visible on the plates; 8 on plate A (no IPTG, lanes labeled as A) and 10 on plate B (with 100 μM IPTG in recovery, lanes labeled as B). All 18 were analyzed by colony PCR, which gave a product band indicative of a successful transposition reaction (arrows).

FIG. 16 depicts sequencing results of select colony PCR products which confirm that they represent transposition events, as they span the junction between the LE and the PAM at the engineered target site, which is in the lacZ gene. The minimal LE sequence is indicated in blue at the top of the screen (min LE), while the target and PAM are indicated in grey. Some sequence variation is observed in the PCR products, but this variation is expected given that insertion can occur at variable distances upstream of the PAM.

FIG. 17 depicts the results of testing of engineered single guides for 64-1 transposition activity. Black boxes are lanes not pertaining to this experiment. Panel A of FIG. 17 depicts a gel image of PCR 4 (detecting the RE junction to the donor) of transposition: lane 1=apo (no sgRNA), lane 2=holo (+sgRNA), lane 3=sgRNA v1-1, lane 4=sgRNA v1-2, lane 5=sgRNA v1-3. Panel B of FIG. 17 depicts a gel image of PCR 5 (detecting the LE junction to the donor) of transposition: lane 1=apo (no sgRNA), lane 2=holo (+sgRNA), lane 3=sgRNA v1-1, lane 4=sgRNA v1-2, lane 5=sgRNA v1-3. Panel C of FIG. 17 depicts a gel image of PCR 4 (detecting the RE junction to the donor) of transposition: lane 1=apo (no sgRNA), lane 2=holo (+sgRNA), lane 3=sgRNA v1-4, lane 4=sgRNA v1-6, lane 5=sgRNA v1-7, lane 6=sgRNA v1-8, lane 7=sgRNA v1-9. Panel D of FIG. 17 depicts a gel image of PCR 5 (detecting the LE junction to the donor) of transposition: lane 1=apo (no sgRNA), lane 2=holo (+sgRNA), lane 3=sgRNA v1-4, lane 4=sgRNA v1-6, lane 5=sgRNA v1-7, lane 6=sgRNA v1-8, lane 7=sgRNA v1-9. Panel E of FIG. 17 depicts a gel image of PCR 4 (detecting the RE junction to the donor) of transposition: lane 1=apo (no sgRNA), lane 2=holo (+sgRNA), lane 3=sgRNA v1-5, lane 4=skip, lane 5=sgRNA v1-10. Panel F of FIG. 17 depicts a gel image of PCR 5 (detecting the LE junction to the donor) of transposition: lane 1=apo (no sgRNA), lane 2=holo (+sgRNA), lane 3=sgRNA v1-5, lane 4=skip, lane 5=sgRNA v1-10. Panel G of FIG. 17 depicts a gel image of PCR 4 (detecting the RE junction to the donor) of transposition: lane 1=apo (no sgRNA), lane 2=holo (+sgRNA), lane 3=sgRNAv1-17, lane 4=sgRNA v1-18, lane 5=skip, lane 6=sgRNA v1-19, lane 7=skip, lane 8=sgRNA v1-20. Panel H of FIG. 17 depicts a gel image of PCR 5 (detecting the LE junction to the donor) of transposition: lane 1=apo (no sgRNA), lane 2=holo (+sgRNA), lane 3=sgRNAv1-17, lane 4=sgRNA v1-18, lane 5=skip, lane 6=sgRNA v1-19, lane 7=skip, lane 8=sgRNA v1-20

FIG. 18 depicts the results of testing of engineered LE and RE for 64-1 transposition activity. Black boxes are lanes not pertaining to this experiment. Panel A of FIG. 18 depicts a gel image of PCR 4 (detecting the RE junction to the donor) of transposition: lane 1=apo (no sgRNA), lane 2=holo (+sgRNA), lane 3=LE 86 bp, lane 4=LE 105 bp, lane 5=RE 196 bp, lane 6=RE 242 bp, lane 7=RE Internal deletion 50, lane 8=RE internal deletion 81. Panel B of FIG. 18 depicts a gel image of PCR 5 (detecting the LE junction to the donor) of transposition: lane 1=apo (no sgRNA), lane 2=holo (+sgRNA), lane 3=LE 86 bp, lane 4=LE 105 bp, lane 5=RE 196 bp, lane 6=RE 242 bp, lane 7=RE Internal deletion 50, lane 8=RE internal deletion 81. Panel C of FIG. 18 depicts a gel image of PCR 4 (detecting the RE junction to the donor) of transposition: lane 1=apo (no sgRNA), lane 2=holo (+sgRNA), lane 3=RE internal deletion 81 and 178 bp, lane 4=skip, lane 5=RE internal deletion 81 and 196 bp, lane 6=skip, lane 7=RE internal deletion 81 and 212 bp, lane 8=skip. Panel D of FIG. 18 depicts a gel image of PCR 5 (detecting the LE junction to the donor) of transposition: lane 1=apo (no sgRNA), lane 2=holo (+sgRNA), lane 3=RE internal deletion 81 and 178 bp, lane 4=skip, lane 5=RE internal deletion 81 and 196 bp, lane 6=skip, lane 7=RE internal deletion 81 and 212 bp, lane 8=skip. Panel E of FIG. 18 depicts a gel image of PCR 4 (detecting the RE junction to the donor) of transposition: lane 1=apo (no sgRNA), lane 2=holo (+sgRNA), lane 3=RE internal deletion 81 and 178 bp+LE 68 bp, lane 4=RE internal deletion 81 and 178 bp+LE 86 bp, lane 5=skip, lane 6=RE internal deletion 81 and 178 bp+LE 105 bp, lane 7=skip. Panel F of FIG. 18 depicts a gel image of PCR 5 (detecting the LE junction to the donor) of transposition: lane 1=apo (no sgRNA), lane 2=holo (+sgRNA), lane 3=RE internal deletion 81 and 178 bp+LE 68 bp, lane 4=RE internal deletion 81 and 178 bp+LE 86 bp, lane 5=skip, lane 6=RE internal deletion 81 and 178 bp+LE 105 bp, lane 7=skip. Panel G of FIG. 18 depicts a gel image of PCR 6 (detecting the RE junction to the donor) of transposition: lane 1=apo (no sgRNA), lane 2=holo (+sgRNA), lane 3=0 bp overhang, lane 4=1 bp overhang, lane 5=2 bp overhang, lane 6=3 bp overhang, lane 7=5 bp overhang, lane 8=10 bp overhang.

FIG. 19 depicts the results of testing of engineered CAST components with an NLS for transposition activity. Black boxes are lanes not pertaining to this experiment. Panel A of FIG. 19 depicts a gel image of PCR 4 (detecting the RE junction to the donor) of transposition: lane 1=apo (no sgRNA), lane 2=holo (+sgRNA), lane 3=skip, lane 4=skip, lane 5=skip, lane 6=NLS-TnsB, lane 7=skip, lane 8=TnsB-NLS. Panel B of FIG. 19 depicts a gel image of PCR 5 (detecting the LE junction to the donor) of transposition: lane 1=apo (no sgRNA), lane 2=holo (+sgRNA), lane 3=skip, lane 4=skip, lane 5=skip, lane 6=NLS-TnsB, lane 7=skip, lane 8=TnsB-NLS. Panel C of FIG. 19 depicts a gel image of PCR 4 (detecting the RE junction to the donor) of transposition: lane 1=apo (no sgRNA), lane 2=holo (+sgRNA), lane 3=skip, lane 4=skip, lane 5=skip, lane 6=NLS-TniQ, lane 7=skip, lane 8=TniQ-NLS. Panel D of FIG. 19 depicts a gel image of PCR 5 (detecting the LE junction to the donor) of transposition: lane 1=apo (no sgRNA), lane 2=holo (+sgRNA), lane 3=skip, lane 4=skip, lane 5=skip, lane 6=NLS-TniQ, lane 7=skip, lane 8=TniQ-NLS. Panel E of FIG. 19 depicts a gel image of PCR 4 (detecting the RE junction to the donor) of transposition: lane 1=apo (no sgRNA), lane 2=holo (+sgRNA), lane 3=skip, lane 4=skip, lane 5=NLS-Cas12k, lane 6=Cas12k-NLS, lane 7=NLS-TnsC, lane 8=TnsC-NLS. Panel F of FIG. 19 depicts a gel image of PCR 5 (detecting the LE junction to the donor) of transposition: lane 1=apo (no sgRNA), lane 2=holo (+sgRNA), lane 3=skip, lane 4=skip, lane 5=NLS-Cas12k, lane 6=Cas12k-NLS, lane 7=NLS-TnsC, lane 8=TnsC-NLS. Panel G of FIG. 19 depicts a gel image of PCR 4 (detecting the RE junction to the donor) of transposition: lane 1=apo (no sgRNA), lane 2=holo (+sgRNA), lane 3=NLS-HA-TnsC, lane 4=NLS-TnsC-FLAG, lane 5=NLS-TnsC-HA, lane 6=NLS-TnsC-Myc, lane 7=NLS-FLAG-TnsC, lane 8=NLS-Myc-TnsC. Panel H of FIG. 19 depicts a gel image of PCR 5 (detecting the LE junction to the donor) of transposition: lane 1=apo (no sgRNA), lane 2=holo (+sgRNA), lane 3=NLS-HA-TnsC, lane 4=NLS-TnsC-FLAG, lane 5=NLS-TnsC-HA, lane 6=NLS-TnsC-Myc, lane 7=NLS-FLAG-TnsC, lane 8=NLS-Myc-TnsC. Panel I of FIG. 19 depicts a gel image of PCR 4 (detecting the RE junction to the donor) of transposition: lane 1=apo (no sgRNA), lane 2=holo (+sgRNA), lane 3=Cas 2×NLS apo (no sgRNA), lane 4=Cas 2×NLS holo (+sgRNA). Panel J of FIG. 19 depicts a gel image of PCR 5 (detecting the LE junction to the donor) of transposition: lane 1=apo (no sgRNA), lane 2=holo (+sgRNA), lane 3=Cas 2×NLS apo (no sgRNA), lane 4=Cas 2×NLS holo (+sgRNA)

FIG. 20 depicts engineered CAST-NLS acting as a single suite. All lanes have Cas12k-NLS and NLS-TniQ, TnsB, TnsC and sgRNA unless otherwise described. Panel A of FIG. 20 depicts gel image of PCR 4 (detecting the RE junction to the donor) of transposition: lane 1=apo (no sgRNA), lane 2=holo (+sgRNA), lane 3=NLS-TnsB, lane 4=TnsB-NLS, lane 5=NLS-TnsB and NLS-TnsC, lane 6=TnsB-NLS and NLS-TnsC. Panel B of FIG. 20 depicts gel image of PCR 5 (detecting the LE junction to the donor) of transposition: lane 1=apo (no sgRNA), lane 2=holo (+sgRNA), lane 3=NLS-TnsB, lane 4=TnsB-NLS, lane 5=NLS-TnsB and NLS-TnsC, lane 6=TnsB-NLS and NLS-TnsC.

FIG. 21 depicts the results of testing of Cas Effector and TniQ protein fusion for transposition activity. Panel A of FIG. 21 depicts a gel image of PCR 4 (detecting the RE junction to the donor) of transposition: lane 1=apo (no sgRNA) with Cas-TniQ fusion, lane 2=holo (+sgRNA) with Cas-TniQ fusion, lane 3=apo (no sgRNA) with TniQ-Cas fusion, lane 4=holo (+sgRNA) with TniQ-Cas fusion. Panel B of FIG. 21 depicts a gel image of PCR 5 (detecting the LE junction to the donor) of transposition: lane 1=apo (no sgRNA) with Cas-TniQ fusion, lane 2=holo (+sgRNA) with Cas-TniQ fusion, lane 3=apo (no sgRNA) with TniQ-Cas fusion, lane 4=holo (+sgRNA) with TniQ-Cas fusion. Panel C of FIG. 21 depicts a gel image of PCR 4 (detecting the RE junction to the donor) of transposition: lane 1=apo (no sgRNA) with TniQ-Cas fusion, lane 2=holo (+sgRNA) with TniQ-Cas fusion, lane 3=holo Cas alone, lane 4=apo (no sgRNA) with TniQ-48 Linker-Cas fusion, lane 5=holo (+sgRNA) with TniQ-48 Linker-Cas fusion, lane 6=apo (no sgRNA) with TniQ-68 Linker-Cas fusion, lane 7=holo (+sgRNA) with TniQ-68 Linker-Cas fusion, lane 8=holo (+sgRNA) with TniQ-72 Linker-Cas fusion. Panel D of FIG. 21 depicts a gel image of PCR 5 (detecting the LE junction to the donor) of transposition: lane 1=apo (no sgRNA) with TniQ-Cas fusion, lane 2=holo (+sgRNA) with TniQ-Cas fusion, lane 3=holo Cas alone, lane 4=apo (no sgRNA) with TniQ-48 Linker-Cas fusion, lane 5=holo (+sgRNA) with TniQ-48 Linker-Cas fusion, lane 6=apo (no sgRNA) with TniQ-68 Linker-Cas fusion, lane 7=holo (+sgRNA) with TniQ-68 Linker-Cas fusion, lane 8=holo (+sgRNA) with TniQ-72 Linker-Cas fusion. Panel E of FIG. 21 depicts a gel image of PCR 4 (detecting the RE junction to the donor) of transposition: lane 1=apo (no sgRNA), lane 2=holo (+sgRNA), lane 3=apo (no sgRNA) with NLS-TniQ-Cas-NLS fusion, lane 4=holo (+sgRNA) with NLS-TniQ-Cas-NLS fusion, lane 5=apo (no sgRNA) with NLS-TniQ-77 Linker-Cas-NLS fusion, lane 6=holo (+sgRNA) with NLS-TniQ-77 Linker-Cas-NLS fusion. Panel F of FIG. 21 depicts a gel image of PCR 5 (detecting the LE junction to the donor) of transposition: lane 1=apo (no sgRNA), lane 2=holo (+sgRNA), lane 3=apo (no sgRNA) with NLS-TniQ-Cas-NLS fusion, lane 4=holo (+sgRNA) with NLS-TniQ-Cas-NLS fusion, lane 5=apo (no sgRNA) with NLS-TniQ-77 Linker-Cas-NLS fusion, lane 6=holo (+sgRNA) with NLS-TniQ-77 Linker-Cas-NLS fusion. Panel G of FIG. 21 depicts a gel image of PCR 4 (detecting the RE junction to the donor) of transposition: lane 1=apo (no sgRNA), lane 2=holo (+sgRNA), lane 3=NLS-TniQ-Cas-NLS apo (no sgRNA), lane 4=NLS-TniQ-Cas-NLS holo (+sgRNA), lane 5=Cas-NLS-P2A-NLS-TniQ apo (no sgRNA), lane 6=Cas-NLS-P2A-NLS-TniQ holo (+sgRNA). Panel H of FIG. 21 depicts a gel image of PCR 5 (detecting the LE junction to the donor) of transposition: lane 1=apo (no sgRNA), lane 2=holo (+sgRNA), lane 3=NLS-TniQ-Cas-NLS apo (no sgRNA), lane 4=NLS-TniQ-Cas-NLS holo (+sgRNA), lane 5=Cas-NLS-P2A-NLS-TniQ apo (no sgRNA), lane 6=Cas-NLS-P2A-NLS-TniQ holo (+sgRNA).

FIG. 22 depicts the results of expression of TnsB and TnsC in human cells, followed by cell fractionation and in vitro transposition reactions. Panel A of FIG. 22 depicts a gel image of PCR 4 (detecting the RE junction to the donor) of transposition: lane 1=apo (no sgRNA), lane 2=holo (+sgRNA), lane 3=holo (+sgRNA) with Untreated (no TnsB) cytoplasm, lane 4=holo (+sgRNA) with untreated nucleoplasm, lane 5=holo (+sgRNA) with NLS-TnsB cell cytoplasm, lane 6=holo (+sgRNA) with NLS-TnsB cell nucleoplasm, lane 7=holo (+sgRNA) with TnsB-NLS cell cytoplasm, lane 8=holo (+sgRNA) with TnsB-NLS cell nucleoplasm, lane 9=holo (+sgRNA) with NLS-TniQ cell cytoplasm, lane 10=holo (+sgRNA) with NLS-TniQ cell nucleoplasm. Panel B of FIG. 22 depicts a gel image of PCR 5 (detecting the LE junction to the donor) of transposition: lane 1=apo (no sgRNA), lane 2=holo (+sgRNA), lane 3=holo (+sgRNA) with Untreated (no TnsB) cytoplasm, lane 4=holo (+sgRNA) with untreated nucleoplasm, lane 5=holo (+sgRNA) with NLS-TnsB cell cytoplasm, lane 6=holo (+sgRNA) with NLS-TnsB cell nucleoplasm, lane 7=holo (+sgRNA) with TnsB-NLS cell cytoplasm, lane 8=holo (+sgRNA) with TnsB-NLS cell nucleoplasm, lane 9=holo (+sgRNA) with NLS-TniQ cell cytoplasm, lane 10=holo (+sgRNA) with NLS-TniQ cell nucleoplasm. Panel C of FIG. 22 depicts a gel image of PCR 4 (detecting the RE junction to the donor) of transposition: lane 1=apo (no sgRNA), lane 2=holo (+sgRNA), lane 3=holo (+sgRNA) without TnsC, lane 4=holo (+sgRNA) with Untreated (no TnsC) cytoplasm, lane 5=holo (+sgRNA) with untreated nucleoplasm, lane 6=holo (+sgRNA) with NLS-HA-TnsC cell cytoplasm, lane 7=holo (+sgRNA) with NLS-HA-TnsC cell nucleoplasm, lane 8=holo (+sgRNA) with TnsC-NLS cell cytoplasm, lane 9=holo (+sgRNA) with TnsC-NLS cell nucleoplasm. Panel D of FIG. 22 depicts a gel image of PCR 5 (detecting the LE junction to the donor) of transposition: lane 1=apo (no sgRNA), lane 2=holo (+sgRNA), lane 3=holo (+sgRNA) without TnsC, lane 4=holo (+sgRNA) with Untreated (no TnsC) cytoplasm, lane 5=holo (+sgRNA) with untreated nucleoplasm, lane 6=holo (+sgRNA) with NLS-HA-TnsC cell cytoplasm, lane 7=holo (+sgRNA) with NLS-HA-TnsC cell nucleoplasm, lane 8=holo (+sgRNA) with TnsC-NLS cell cytoplasm, lane 9=holo (+sgRNA) with TnsC-NLS cell nucleoplasm. Panel E of FIG. 22 depicts a gel image of PCR 4 (detecting the RE junction to the donor) of transposition: lane 1=apo (no sgRNA), lane 2=holo (+sgRNA), lane 3=apo (no sgRNA) NLS-TnsB-IRES-NLS-TnsC cytoplasm, lane 4=holo (+sgRNA) NLS-TnsB-IRES-NLS-TnsC cytoplasm, lane 5=apo (no sgRNA) NLS-TnsB-IRES-NLS-TnsC nucleoplasm, lane 6=holo (+sgRNA) NLS-TnsB-IRES-NLS-TnsC nucleoplasm, lane 7=apo (no sgRNA) TnsB-NLS-IRES-NLS-TnsC cytoplasm, lane 8=holo (+sgRNA) TnsB-NLS-IRES-NLS-TnsC cytoplasm, lane 9=apo (no sgRNA) TnsB-NLS-IRES-NLS-TnsC nucleoplasm, lane 10=holo (+sgRNA) TnsB-NLS-IRES-NLS-TnsC nucleoplasm. Panel F of FIG. 22 depicts a gel image of PCR 5 (detecting the LE junction to the donor) of transposition: lane 1=apo (no sgRNA), lane 2=holo (+sgRNA), lane 3=apo (no sgRNA) NLS-TnsB-IRES-NLS-TnsC cytoplasm, lane 4=holo (+sgRNA) NLS-TnsB-IRES-NLS-TnsC cytoplasm, lane 5=apo (no sgRNA) NLS-TnsB-IRES-NLS-TnsC nucleoplasm, lane 6=holo (+sgRNA) NLS-TnsB-IRES-NLS-TnsC nucleoplasm, lane 7=apo (no sgRNA) TnsB-NLS-IRES-NLS-TnsC cytoplasm, lane 8=holo (+sgRNA) TnsB-NLS-IRES-NLS-TnsC cytoplasm, lane 9=apo (no sgRNA) TnsB-NLS-IRES-NLS-TnsC nucleoplasm, lane 10=holo (+sgRNA) TnsB-NLS-IRES-NLS-TnsC nucleoplasm.

FIG. 23 depicts the results of expression of Cas12k and TniQ linked constructs in human cells, followed by in vitro transposition testing. Panel A of FIG. 23 depicts a gel image of PCR 5 (detecting the LE junction to the donor) of transposition: lane 1=apo (no sgRNA), lane 2=holo (+sgRNA), lane 3=Cas-NLS holo (+sgRNA) cytoplasm, lane 4=Cas-NLS holo (+sgRNA) nucleoplasm, lane 5=Cas-NLS holo (+sgRNA) nucleoplasm+additional sgRNA, lane 6=Cas-NLS-P2A-NLS-TniQ holo (+sgRNA) cytoplasm, lane 7=Cas-NLS-P2A-NLS-TniQ holo (+sgRNA) nucleoplasm, lane 8=Cas-NLS-P2A-NLS-TniQ holo (+sgRNA) nucleoplasm+additional sgRNA. Panel B of FIG. 23 depicts a gel image of PCR 4 (detecting the RE junction to the donor) of transposition: lane 1=apo (no sgRNA), lane 2=holo (+sgRNA), lane 3=apo (no sgRNA) Cas-NLS-P2A-NLS-TniQ cytoplasm, lane 4=holo (+sgRNA) Cas-NLS-P2A-NLS-TniQ cytoplasm, lane 5=apo (no sgRNA) Cas-NLS-P2A-NLS-TniQ nucleoplasm, lane 6=holo (+sgRNA) Cas-NLS-P2A-NLS-TniQ nucleoplasm, lane 7=holo (+sgRNA) Cas-NLS-P2A-NLS-TniQ nucleoplasm+additional holo Cas-NLS, lane 8=holo (+sgRNA) Cas-NLS-P2A-NLS-TniQ nucleoplasm+NLS-TniQ. Panel C of FIG. 23 depicts a gel image of PCR 5 (detecting the LE junction to the donor) of transposition: lane 1=apo (no sgRNA), lane 2=holo (+sgRNA), lane 3=apo (no sgRNA) Cas-NLS-P2A-NLS-TniQ cytoplasm, lane 4=holo (+sgRNA) Cas-NLS-P2A-NLS-TniQ cytoplasm, lane 5=apo (no sgRNA) Cas-NLS-P2A-NLS-TniQ nucleoplasm, lane 6=holo (+sgRNA) Cas-NLS-P2A-NLS-TniQ nucleoplasm, lane 7=holo (+sgRNA) Cas-NLS-P2A-NLS-TniQ nucleoplasm+additional holo Cas-NLS, lane 8=holo (+sgRNA) Cas-NLS-P2A-NLS-TniQ nucleoplasm+NLS-TniQ. Panel D of FIG. 23 depicts a gel image of PCR 4 (detecting the RE junction to the donor) of transposition: lane 1=apo (no sgRNA), lane 2=holo (+sgRNA), lane 3=apo (no sgRNA) NLS-TniQ-Cas-NLS cytoplasm, lane 4=holo (+sgRNA) NLS-TniQ-Cas-NLS cytoplasm, lane 5=apo (no sgRNA) NLS-TniQ-Cas-NLS nucleoplasm, lane 6=holo (+sgRNA) NLS-TniQ-Cas-NLS nucleoplasm, lane 7=holo (+sgRNA) NLS-TniQ-Cas-NLS nucleoplasm+additional holo Cas-NLS, lane 8=holo (+sgRNA) NLS-TniQ-Cas-NLS nucleoplasm+NLS-TniQ. Panel E of FIG. 23 depicts a gel image of PCR 5 (detecting the LE junction to the donor) of transposition: lane 1=apo (no sgRNA), lane 2=holo (+sgRNA), lane 3=apo (no sgRNA) NLS-TniQ-Cas-NLS cytoplasm, lane 4=holo (+sgRNA) NLS-TniQ-Cas-NLS cytoplasm, lane 5=apo (no sgRNA) NLS-TniQ-Cas-NLS nucleoplasm, lane 6=holo (+sgRNA) NLS-TniQ-Cas-NLS nucleoplasm, lane 7=holo (+sgRNA) NLS-TniQ-Cas-NLS nucleoplasm+additional holo Cas-NLS, lane 8=holo (+sgRNA) NLS-TniQ-Cas-NLS nucleoplasm+NLS-TniQ. Panel F of FIG. 23 depicts a gel image of PCR 4 (detecting the RE junction to the donor) of transposition: lane 1=apo (no sgRNA), lane 2=holo (+sgRNA), lane 3=apo (no sgRNA) Cas-NLS-IRES-NLS-TniQ cytoplasm, lane 4=holo (+sgRNA) Cas-NLS-IRES-NLS-TniQ cytoplasm, lane 5=apo (no sgRNA) Cas-NLS-IRES-NLS-TniQ nucleoplasm, lane 6=apo (no sgRNA) Cas-NLS-IRES-NLS-TniQ nucleoplasm+additional PURExpress, lane 7=apo (no sgRNA) Cas-NLS-IRES-NLS-TniQ nucleoplasm+additional Cas-NLS, lane 8=apo (no sgRNA) Cas-NLS-IRES-NLS-TniQ nucleoplasm+NLS-TniQ, lane 9=holo (+sgRNA) Cas-NLS-IRES-NLS-TniQ nucleoplasm, lane 10=holo (+sgRNA) Cas-NLS-IRES-NLS-TniQ nucleoplasm+additional PURExpress, lane 11=holo (+sgRNA) Cas-NLS-IRES-NLS-TniQ nucleoplasm+additional Cas-NLS, lane 12=holo (+sgRNA) Cas-NLS-IRES-NLS-TniQ nucleoplasm+NLS-TniQ. Panel G of FIG. 23 depicts a gel image of PCR 5 (detecting the LE junction to the donor) of transposition: lane 1=apo (no sgRNA), lane 2=holo (+sgRNA), lane 3=apo (no sgRNA) Cas-NLS-IRES-NLS-TniQ cytoplasm, lane 4=holo (+sgRNA) Cas-NLS-IRES-NLS-TniQ cytoplasm, lane 5=apo (no sgRNA) Cas-NLS-IRES-NLS-TniQ nucleoplasm, lane 6=apo (no sgRNA) Cas-NLS-IRES-NLS-TniQ nucleoplasm+additional PURExpress, lane 7=apo (no sgRNA) Cas-NLS-IRES-NLS-TniQ nucleoplasm+additional Cas-NLS, lane 8=apo (no sgRNA) Cas-NLS-IRES-NLS-TniQ nucleoplasm+NLS-TniQ, lane 9=holo (+sgRNA) Cas-NLS-IRES-NLS-TniQ nucleoplasm, lane 10=holo (+sgRNA) Cas-NLS-IRES-NLS-TniQ nucleoplasm+additional PURExpress, lane 11=holo (+sgRNA) Cas-NLS-IRES-NLS-TniQ nucleoplasm+additional Cas-NLS, lane 12=holo (+sgRNA) Cas-NLS-IRES-NLS-TniQ nucleoplasm+NLS-TniQ.

FIG. 24 depicts electrophoretic mobility shift assay (EMSA) results of the 64-1 TnsB and its LE DNA sequence. The EMSA results confirm binding and TnsB recognition. The TnsB protein was expressed in an in vitro transcription/translation system, incubated with FAM-labeled DNA containing the LE sequence, and then separated on a native 5% TBE gel. Binding is observed as a shift upwards in the labeled band. Multiple TnsB binding sites leads to multiple shifts in the EMSA. Lane 1: FAM-labeled DNA only. Lane 2: FAM DNA plus the in vitro transcription/translation system (no TnsB protein). Lane 3: FAM DNA plus TnsB.

FIGS. 25A-25B depict Cas12k effector diversity. FIG. 25A depicts Cas12k CAST genomic context. The transposon is characterized by terminal inverted repeats (TIR, light orange bars), Tn7-like transposon genes (colored arrows), the dead effector Cas12k (orange arrow), a tracrRNA (pink half arrow), and CRISPR array. A “TAAA” target site duplication (TDS) was observed flanking the TIRs. Middle panel: MG64-1 non-coding region inset showing the tracrRNA, a pseudo repeat and self-targeting spacer, the CRISPR array and transposon left end TIR. Bottom panel: multiple alignment of the pseudo repeat and self-targeting spacer in a group of CAST homologs. FIG. 25B depicts unrooted phylogenetic tree of Cas12k effectors. Cas12k effectors recovered in this study are shown as orange (confirmed transposon in the genome) and black branches, while reference Cas12k sequences are shown in grey. Reference sequences ShCas12k and AcCas12k are shown with red arrows.

FIGS. 26A-26B depict multiple sequence alignment of CAST right (FIG. 26A) and left (FIG. 26B) ends. Transposon ends inverted motif “TGTNNA” is highlighted with a box.

FIG. 27 depicts alignment of Cas12k CAST tracrRNA sequences, showing regions of sequence and structural conservation. In particular, the sequence “TGCTTTC” at sequence position 88-92 may be important for sgRNA tertiary structure and for a non-continuous repeat-anti-repeat pairing with the crRNA. The hairpin “CYCC(n6)GGRG” at positions 279-294 may be important for function, possibly positioning the downstream sequence for crRNA pairing.

FIG. 28 depicts single guide RNA folding of active MG64-1. MG64-2, and MG64-6 CAST systems. An active, engineered sgRNA for MG64-1 is also shown.

FIGS. 29A-29B depict in vitro screening of CAST transposition with a PAM library.

FIG. 29A depicts the screening setup of in vitro PAM determination. FIG. 29B depicts a schematic of junction PCR for the detection of transposition products.

FIG. 30A depicts transposition junctions of MG64-1 CAST (left lane) and MG64-6 CAST (right lane) amplified by PCR.

FIG. 30B depicts SeqLogo representation of detected PAMs for MG64-1 (top).

FIG. 30C depicts integration frequency plotted by distance on proximal and distal distances of MG64-1.

FIG. 31 depicts single guide RNA engineering of 64-1. Deletion of the region between 130 bp and 190 bp (green and teal section of the structure) generated an sgRNA that directed strong transposition reactions (green bars on the heatmap).

FIG. 32 depicts MG64-2 sgRNA cross reactivity with MG64-1 and the PAM for the combination of the MG64-2 sgRNA plus the MG64-1 effector.

FIG. 33 depicts single guide RNA truncations in the coding DNA for the MG64-2 sgRNA in a sequence view and a secondary structure prediction model. Deleted regions and truncations in the sequence view are shown as bars (del1, del2, del3, del4, del5, and del6). Deleted regions in ovals in the secondary structure prediction model indicate the tested truncations (deletions 1, 2, 3, 4 across pseudoknot, 5, and 6).

FIG. 34 depicts data demonstrating that engineered MG64-2 sgRNAs are active with the MG64-1 CAST system. PCR reactions represent each possible integration junction or negative controls (Panel B of FIG. 29). Successful integration products are highlighted by arrows. Boxed lanes are not relevant for this experiment.

FIG. 35 depicts MG64-2 sgRNA split guide designs. sgRNAs fragments were synthesized separately then re-annealed before testing in transposition experiments.

FIG. 36 depicts data demonstrating that split MG64-2 sgRNAs are active with the MG64-1 CAST system. PCR reactions represent each possible integration junction or negative controls (Panel B of FIG. 29). Successful integration products are highlighted by arrows. Boxed lanes are not relevant for this experiment.

FIG. 37 depicts data demonstrating that LE and RE minimization maintained the transposition activity of the system.

FIGS. 38A-38C depict the results of E. coli integration with MG64-1. FIG. 38A depicts a schematic representation of introduction of a CAST system into E. coli. FIG. 38B depicts NGS data showing greater than 80% editing efficiency. FIG. 38C depicts off-target analysis showing that off-target integration greater than 1% of all the summed transposition events was not detected.

FIG. 39 depicts local insertion rates for various endogenous loci of the E. coli genome.

FIGS. 40A-40B depict the results of multi locus targeting. FIG. 40A depicts the respective local insertion frequencies at the endogenous and engineered loci. FIG. 40B depicts the relative insertion frequencies for on-target insertion at the endogenous locus, on-target insertion at the engineered locus, and off-target insertion. Integration at both loci combined accounted for greater than 95% of all integrations that occurred on the genome.

FIG. 41 depicts Sanger sequencing data of the integration PCR product which demonstrates that MG64-1 is active in vitro. The reaction is of the RE donor-target product and the point where the sequencing stops matching the donor DNA is when junction occurs (dark bars underneath sequencing peaks).

FIG. 42A shows a schematic representation of serial dilution of target DNA for in vitro transposition experiments. The CAST components are expressed and added to the reaction with in vitro transcribed sgRNA and donor plasmid. Target plasmid DNA is added at decreasing concentrations and tested for transposition experiments. When the minimum amount of target DNA is determined, transposition reactions are assayed by adding increasing amounts of human genomic DNA.

FIG. 42B shows an illustration of PCR amplification of transposition reactions. An 8N PAM plasmid library (8N-Target, Rxn #1) is targeted with the CAST system to integrate donor DNA (Rxn #2). Upon successful integration, junction PCR reactions are performed with primers to amplify the four putative integration reactions, based on the orientation of cargo integration (Rxn #3, #4, #5, and #6).

FIG. 42C illustrates PCR reaction products from in vitro transposition assays with serial dilutions of target plasmid DNA. Target, donor, and reactions #3, #4, #5, and #6 correspond to PCR integration products as shown in FIG. 42B.

FIG. 42D shows PCR reaction products from in vitro transposition assays with a fixed amount of target plasmid DNA (0.5 ng) while adding increasing amounts of human genomic DNA to increase the search space. Target, donor, and reactions #3, #4, #5, and #6 correspond to PCR integration products as shown in FIG. 42B.

FIG. 43A shows a schematic of transposition reactions across a high copy element. The target PCR product spans the wild-type target element when assayed with CAST proteins and sgRNA targeting one of the multiple arrayed targets. Integration can occur in either the forward orientation, the reverse orientation, or both. The forward transposition product is assayed by junction PCR that amplifies the region encompassing the LE of the donor DNA to the 5′ end of the target site (Fwd PCR). The reverse junction reaction assays the region encompassing the LE of the donor DNA to the 3′ end of the target element (Rev PCR).

FIG. 43B shows PCR reaction products from in vitro transposition assays at 15 target sites (guide) in LINE1 3′ elements in human genomic DNA. Target and reactions Fwd PCR and Rev PCR correspond to PCR integration products as shown in FIG. 43A.

FIG. 43C shows PCR reaction products from in vitro transposition assays at 15 target sites (guide) in SVA elements in human genomic DNA. Target and reactions Fwd PCR and Rev PCR correspond to PCR integration products as shown in FIG. 43A. Bands highlighted with an arrow indicate successful targeted integration.

FIG. 43D shows PCR reaction products from in vitro transposition assays at 15 target sites (guide) in HERV elements in human genomic DNA. Target and reactions Fwd PCR and Rev PCR correspond to PCR integration products as shown in FIG. 43A. Bands highlighted with an arrow indicate successful targeted integration.

FIG. 43E shows Sanger sequencing of the Fwd PCR integration product at multiple target sites of the LINE1 3′ elements. The point at which the sequencing trace stops matching the donor DNA (grey vertical bar) is where integration occurs.

FIG. 43F shows Sanger sequencing of the Rev PCR integration product at multiple target sites of the LINE1 3′ elements. The point at which the sequencing trace stops matching the target DNA (grey vertical bar) is where integration occurs.

FIG. 43G shows Sanger sequencing of the Fwd PCR integration product at SVA target site 3. The point at which the sequencing trace stops matching the donor DNA (grey vertical bar) is where integration occurs.

FIG. 43H shows Sanger sequencing of the Fwd PCR product at HERV target site 5. The point at which the sequencing trace stops matching the donor DNA (grey vertical bar) is where integration occurs.

FIG. 44 shows PCR reaction products from in vitro transposition assays at LINE1 target sites 12 and 15 in human genomic DNA with functional domains. Target and reactions Fwd PCR and Rev PCR correspond to PCR integration products as shown in FIG. 42A. Bands highlighted with an arrow indicate successful targeted integration.

FIGS. 45A-45B illustrate in vitro transposition experiments with CAST, S15, NLS-S15, and S15-NLS expressed from Eukaryotic transcription/translation reactions. FIG. 45A shows in vitro transposition reactions with MG64-1 CAST and S55. Wheat Germ Extract-expressed CAST components promote transposition without addition of S15, albeit at a low rate (faint bands highlighted with arrows). Addition of PURExpress reagent (Spent PUREx) increases transposition efficiency, as shown by the strength of the band at Rxn #5 (PURExpress reagent contains S15). Independent addition of S15 and S15-NLS translated from Wheat Germ Extract reactions increases transposition efficiency by MG64-1 in vitro compared with the other conditions tested (strong bands highlighted with arrows). FIG. 45B shows in vitro reactions of transposition with the NLS-S15 configuration. PURExpress reagent addition increases in vitro transposition (Lane 3) compared with CAST-components only conditions (Lane 2). The NLS-S15 configuration did not improve transposition (Lanes 4-5). Boxed Rxn #5 represents an expected band if transposition activity is detected.

FIGS. 46A-46H show a schematic of fusion plasmids for in cell transposition. FIG. 46A: two targeting complex plasmids and one donor plasmid are assembled for high copy elements Line1, targets 8, 12, and 15, and SVA target 3. FIG. 46B shows in cell transposition to high copy elements with H1core-TniQ or HMGN1-TniQ at LINE1 targets 8, 12, 15, and SVA target 3. Arrows indicate amplified transposition junction reactions in either forward (Fwd PCR) or reverse (Rev PCR) orientation of transposition. Mock control represents a reaction without targeting or donor plasmids. FIG. 46C shows Sanger sequencing of the PCR integration product Fwd PCR at LINE1 3′ target site 8. Integration was mediated by MG64-1 with the NLS-H1core-TniQ fusion. The point at which the sequencing trace stops matching the donor DNA (grey vertical bar) is where integration occurs. FIG. 46D shows Sanger sequencing of the PCR integration product Fwd PCR at LINE1 3′ target site 8. Integration was mediated by MG64-1 with the NLS-HMGN1-TniQ fusion. The point at which the sequencing trace stops matching the donor DNA (grey vertical bar) is where integration occurs. FIG. 46E shows Sanger sequencing of the PCR integration product Rev PCR at LINE1 3′ target site 12. Integration was mediated by MG64-1 with the NLS-H1core-TniQ fusion. The point at which the sequencing trace stops matching the donor DNA (grey vertical bar) is where integration occurs. FIG. 46F shows Sanger sequencing of the PCR integration product Rev PCR at LINE1 3′ target site 12. Integration was mediated by MG64-1 with the NLS-HMGN1-TniQ fusion. The point at which the sequencing trace stops matching the donor DNA (grey vertical bar) is where integration occurs. FIG. 46G shows Sanger sequencing of the PCR integration product Fwd PCR at LINE1 3′ target site 15. Integration was mediated by MG64-1 with the NLS-H1core-TniQ fusion. The point at which the sequencing trace stops matching the donor DNA (grey vertical bar) is where integration occurs. FIG. 46H shows Sanger sequencing of the PCR integration product Fwd PCR at LINE1 3′ target site 15. Integration was mediated by MG64-1 with the NLS-HMGN1-TniQ fusion. The point at which the sequencing trace stops matching the donor DNA (grey vertical bar) is where integration occurs.

FIGS. 47A-47D depict immunofluorescence staining for localization of Cas12k CAST components in human cells. FIG. 47A: Top row: detection of TnsB localization: mid-row: detection of Cas12k localization: bottom row: detection of TnsC localization. Images indicated that MG64-1 Cas12k and TnsB localize in the nucleus of mammalian cells, while TnsC localizes in the cytoplasm. Cas12k CAST proteins were tagged with an HA tag. Anti-HA antibody was used for protein detection. DAPI was used to stain DNA (nucleus). FIG. 47B: Top and bottom rows: detection of TniQ localization. Images indicated that MG64-1 TniQ localizes in the nucleus of mammalian cells. CAST proteins were tagged with an HA tag. Anti-HA antibody was used for protein detection. DAPI was used to stain DNA (nucleus). FIG. 47C: All rows: detection of TnsC co-localization with TniQ. Images indicated that, while some TnsC may stay in the cytoplasm, it now co-localizes in the nucleus with TniQ. CAST proteins were tagged with an HA tag. Anti-HA antibody was used for protein detection. DAPI was used to stain DNA (nucleus). FIG. 47D: Both rows: Cas12k, TnsB, TnsC, and TniQ co-delivered to HEK293T cells localize in the nucleus. CAST proteins were tagged with an HA tag. Anti-HA antibody was used for protein detection. DAPI was used to stain DNA (nucleus).

FIGS. 48A-48B depict in vitro screening of MG64-1 Cas12k CAST transposition. FIG. 48A: Diagram of the construct used for MG64-1 holocomplex purification. FIG. 48B: Schematic of junction PCR for the detection of transposition products. A target substrate with a 5′ PAM followed by the protospacer (Target, Rxn #1) is targeted with the CAST system to integrate cargo DNA (Rxn #2). Upon successful integration, junction PCR reactions are performed with primers to amplify the four putative integration reactions, based on the orientation of cargo integration.

FIGS. 48C-48D depict MG64-1 protein purification. FIG. 48C: Fractions collected during 2 L-scale purification of MG64-1 holocomplex run on stain free denaturing PAGE gel. FIG. 48D: Chromatogram of Size Exclusion Chromatography (SEC) performed on MG64-1 holo complex. The peak centered at 29.3 mL (peak 1) was used for in vitro activity assays.

FIG. 49A depicts in vitro transposition with Peak1-recovered holocomplex supplemented with TnT expressed components. Lane L) Ladder; Lane 1) TnT expressed CAST components apo condition (−sgRNA); Lane 2) TnT expressed CAST components holo condition (+sgRNA); Lane 3) Purified Peak1 complemented with TnT CAST components without additional supplementation of Cas12k (-TnT Cas12k); Lane 4) Purified Peak1 complemented with TnT CAST components without additional supplementation of TnsC (-TnT TnsC); Lane 5) Peak1 complemented with TnT CAST components without additional supplementation of TniQ (-TnT TniQ); Lane 6) Peak1 complemented with TnT CAST components without additional supplementation of S15 (-TnT S15).

FIG. 49B depicts Sanger sequencing of Lane 3, Lane 4. Lane 5, and Lane 6 from both pDonor and Target directions of the amplified LE to PAM target-donor junction. Vertical line delineates the transposition junction predicted for MG64-1 in the reference sequence. Degradation of signal from either direction results from a multitude of signals reflected in the PCR amplification.

FIG. 50 depicts the identification of ribosomal protein S15 homologs in Cyanobacterial genomic fragments. Candidate sequences from the same sample from where MG64-1 was recovered are highlighted by dark closed circles. The reference S15 from E. coli is indicated with an arrow.

FIGS. 51A and 51B depict a schematic of dual transcript (FIG. 51A) vs. all-in-one transposition components (FIG. 51B). For the dual transcript system, on one plasmid, one transcript is under control of the CMV-BetaGlobin promoter. Cas12k-sso7d-NLS is linked to S15 via a 2A self-cleaving peptide and an IRES element separates the second ORF, NLS-Functional Domain-TniQ, where here the Functional Domain is an H1-core or an HMGN1. The plasmid also contains either an untargeted (null) MG64-1 single guide or a targeted MG64-1 single guide. On the second plasmid, a CMV-BetaGlobin promoter is driving transcription of an NLS-TnsB and NLS-TnsC separated by an IRES element, and TIR (LE and RE) are flanking the bacterial replication origin and antibiotic resistance marker. In the single transcript condition (FIG. 51B), a single CMV-BetaGlobin promoter controls expression of an “all-in-one” transcript where Cas12k-sso7d-NLS, S15-NLS, NLS-Functional Domain-TniQ, NLS-TnsB and NLS-TnsC separated with 2A and IRES elements. This single helper plasmid (pHelper) also contains either an untargeted or targeted MG64-1 single guide under the control of a pU6 promoter. The second plasmid is a pDonor with LE and RE flanking either a reporter gene, a therapeutic transcript, or a selection marker.

FIG. 52 depicts testing of the all-in-one pHelper H1core plasmid in human cells. All transpositions are indicated as junction bands in the Forward LE image. Lane 1: testing the dual transcript system (FIG. 51A) with an untargeted single guide. Lane 2: testing the dual transcript system with a targeted single guide encoding for integration to the Line 1 3′ target 8. Lanes 3-5: testing the single transcript pHelper system with a pDonor that is expressing mNeon, a fluorescent protein. Lane 3: the all-in-one pHelper and mNeon pdonor without a targeted single guide. Lane 4: the all-in-one pHelper and mNeon pDonor with a guide targeting Line1 3′ target 8 at the ratio of 6 μg pHelper to 12 μg pDonor. Lane 5 is the all-in-one pHelper and mNeon pDonor with a guide targeting Line 1 3′ target 8 at the ration of 12 μg pHelper to 6 μg pDonor.

FIG. 53 depicts testing of a non-replicative donor in comparison to a replicative donor. Non-replicating donor has been truncated for the SV40 origin element that allows for the plasmid to propagate in human cells. Integration is performed with all-in-one vectors. Lane 1: Mock transfection controls. Lane 2: All-in-one pHelper targeting Line1 3′ target 8 with a replicative donor as a positive control. Lanes 3-5: All-in-one pHelper with no targeting single guide with replicative mNeon donor. Lanes 6-8: All-in-one pHelper with Line1 3′ target 8 single guide with replicative mNeon donor. Lanes 9-11: All-in-one pHelper with no targeting single guide with non-replicative mNeon donor. Lanes 12-14: All-in-one pHelper with Line1 3′ target 8 single guide with non-replicative mNeon donor.

FIG. 54 depicts addition of Clpx to transposition in cells. Lane 1: All-in-one pHelper with non-targeting single guide with a replicative mNeon pDonor. Lane 2: All-in-one targeting Line1 3′ target 8 with a replicative mNeon donor as a positive control. Lanes 3-6: Same conditions as Lane 2 but with (0.25 μg, 0.5 μg, 1 μg, and 2 μg) amounts of ClpX-NLS plasmid added to the transposition plasmid mix. Lanes 7-10: Same conditions as Lane 2 but with (0.25 μg, 0.5 μg, 1 μg, and 2 μg) amounts of NLS-ClpX plasmid added to the transposition plasmid mix.

FIG. 55 depicts a schematic of a three-plasmid system transfection targeting single copy loci. The pHelper plasmid contains a single guide targeting the single copy locus of interest. Replicative plasmids are used for all plasmids, pHelper, pDonor, and pClpX. The pDonor has TIR flanking a pCMV-BG driving expression of an mNeon fluorescent protein. pClpx is a pCMV-BetaGlobin promoter driving the E. coli ClpX-NLS sequence. All three plasmids are transfected into HEK293T cells at the ratio of 12 μg pHelper:6 μg pDonor:1 μg pClpX: 54 μL Mirus-LT1 (transfection reagent). Cells are then incubated for 72 hrs at 37° C. and harvested for their gDNA.

FIG. 56 depicts transposition of pDonor into the single copy target, AAVS1. AAVS1 is a safe harbor locus that allows for non-deleterious expression of exogenous genes in the human cell when cassettes are integrated into the human genome. AAVS1 is represented only a single time in the human genome on Chromosome 19. By assaying junction PCR of the genomic DNA harvested from cells that were transfected with pHelper targeting both AAVS1 target 5 and AAVS1 target 6, it was possible to visualize the transposition of the pDonor into the intended target is the Forward LE direction.

FIG. 57 depicts Sanger sequencing of transposed AAVS1 target 5 and AAVS1 target 6 in Forward LE direction. Transposition for AAVS5 has a primary transposition 63 bp away from the PAM and AAVS1 target 6 has a primary transposition event 61 bp away from the PAM.

FIGS. 58A and 58B depict NGS sequencing of transposition at AAVS targets 5 (FIG. 58A) and target 6 (FIG. 58B).

FIG. 59 depicts a schematic of NGS quantification. Hypothetical transposition is modeled above the un-integrated target sequence. The two primers should be as close as possible.

FIG. 60 depicts transposition at AAVS1 target 5. 6 gDNA preps of AAVS1 target 5 pHelper was amplified for transposition specific primers (top panel) with transposition bands reflecting the forward LE junction reaction from the genomic target to the donor cargo. No target pHelper are cultures where no target was specified for the spacer sequence. No transposition band is visible for the no target pHelper conditions. Bottom Panel: 25× cycle PCR amplification shows equal loading for the reactions for indexing steps for NGS sequencing for both AAVS1 target 5 pHelper treated and No target pHelper treated samples.

FIGS. 61A-61C depict NGS efficiency and allele visualization of non-edited alleles. FIG. 61A is a graph showing the measurement of NGS efficiency of AAVS1 target 5 compared to control. FIG. 61B depicts allele mapping of non-edited alleles in the no target NGS reads, showing a small fraction of reads that are able to map to the reference with SNPs. FIG. 61C depicts allele mapping of non-edited alleles in the AAVS1 target 5 reads, showing a similar fraction of reads that indicate SNPs have occurred at the genomic locus that are not due to the AAVS1 target 5 incorporation on the pHelper.

FIG. 62 depicts a schematic of plasmids used for the experiments in this disclosure.

FIGS. 63A-63B depict integration of linearized donor in HEK293T cells. FIG. 63A depicts NGS integration of linear CAST pDonor without a single guide (pHelper Null) or with an AAVS1-5 target (pHelper AIO). FIG. 63B depicts ddPCR integration of linear CAST pDonor without a single guide (pHelper Null) or with an AAVS1-5 target (pHelper AIO).

FIG. 64 depicts integration of plasmid donor in K562 cells. NGS integration of CAST pDonor with a single guide expression cassette targeting AAVS1-5 without a single guide containing helper plasmid (pHelper Null) or with AAVS1-5 target (pHelper AIO) in nucleofections that contain either 2×10⁵ (left) or 5×10⁵ (right) K562 cells.

FIGS. 65A-65B depict Hep3B transpositions using Lipofectamine 2000 and Lipofectamine 3000 showing NGS quantification of pDonor containing an sg cassette.

FIG. 66 depicts NGS detection of transpositions in human albumin (ALB) in HEK293T. Guide targets 1077-1138 were predicted sites at Albumin intron 1. 1139 and 1140 were guides constructed for AAVS1.

FIGS. 67A-67C depict NGS read alignments of modeled transposition targets at 60 bp away from the PAM for targets 1093 (FIG. 67A (SEQ ID NOS 1264-1274, respectively, in order of appearance)), 1101 (FIG. 67B (SEQ ID NOS 1275-1279, respectively, in order of appearance)), 1115 (FIG. 67C (SEQ ID NOS 1280-1281, respectively, in order of appearance)).

FIG. 68 depicts NGS detection of transpositions in mouse albumin (mALB) in Hepa1-6. Guide targets 1141-1182 were predicted sites at mouse Albumin intron 1.

FIGS. 69A-69C depict NGS read alignments of modeled transposition targets at 60 bp away from the PAM for targets 1148 (FIG. 69A (SEQ ID NOS 1282-1285, respectively, in order of appearance)), 1161 (FIG. 69B (SEQ ID NOS 1286-1288, respectively, in order of appearance)), and 1162 (FIG. 69C (SEQ ID NOS 1289-1297, respectively, in order of appearance)).

FIG. 70 depicts NGS detection of transpositions in mouse albumin (mROSA26) in Hepa1-6. Guide targets 1183-1167 were predicted sites at mRosa26.

FIGS. 71A-71E depict NGS read alignments of modeled transposition targets at 60 bp away from the PAM for targets (FIG. 71A (SEQ ID NOS 1298-1304, respectively, in order of appearance)), 1201 (FIG. 71B (SEQ ID NOS 1305-1313, respectively, in order of appearance)). 1205 (FIG. 71C (SEQ ID NOS 1314-1323, respectively, in order of appearance)), 1219 (FIG. 71D (SEQ ID NOS 1324-1327, respectively, in order of appearance)), and 1257 (FIG. 71E (SEQ ID NOS 1328-1330, respectively, in order of appearance)).

FIG. 72 depicts NGS readout of conditions with increasing copies of single guide. Control condition reflects a pHelper with all components expressed on the plasmid (Cas12k, S15, TniQ, TnsB, TnsC and single guide targeting AAVS1 5) with a donor plasmid containing a fluorescent marker. For the rest of the conditions, a FIX cargo was used to quantify integration. The 0×sg condition did not have a single guide on the pHelper plasmid with the FIX pDonor. The 1×sg condition had a single guide on the pHelper plasmid with the pDonor FIX. The 2×sg condition contained no single guide on the pHelper with 2 single guide cassettes targeting AAVS1-5 on the pDonor FIX plasmid. The 3×sg condition contained the pDonor FIX containing 2 single guide cassettes for AAVS1-5, with the pHelper plasmid containing an additional single guide cassette targeting AAVS1 5.

FIG. 73 depicts ddPCR detection of both LE and RE junctions of promoter driven Factor IX (FIX) delivery under unguided (pHelper Null) and AAVS1 guided (pHelper AAVS5) conditions.

FIG. 74 depicts MG161 family members are distant homologs of sso7d. The tree was inferred from a multiple sequence alignment of full-length protein sequences containing a PFam PF02294 domain hit. Reference sso7d sequences are highlighted with a triangle. The distance between tips is estimated as 0.5 substitutions per site (horizontal bar).

FIGS. 75A-75C depict MG161 functional domain encoded as tandem repeats. FIG. 75A shows the genomic context of a protein encoding multiple functional domains (FD). FD corresponds to tandem imperfect repeats (arrows labeled 161-12 through 161-18). FIG. 75B shows multiple sequence alignment (SEQ ID NOS 1331-1339, respectively, in order of appearance) of tandem repeat FD vs. a reference sso7d sequence from S. solfataricus. MG161-13 is 20% AAI to the reference sequence, while other FD have lower sequence identity. FIG. 75C shows 3D structure prediction of the ORF encoding repeated domains indicate that each domain forms a 4-beta sheet structure linked by flexible linkers.

FIG. 76 depicts that MG162 family members are distant homologs of HMGN1. The tree was inferred from a multiple sequence alignment of full-length protein sequences (SEQ ID NOS 1340-1353, respectively, in order of appearance) containing a PFam PF01101 domain hit. Reference HMGN1 sequences are highlighted with a triangle. The distance between tips is estimated as 0.7 substitutions per site (horizontal bar).

FIG. 77 depicts multiple sequence alignment of MG162 functional domain proteins vs. reference human and mouse HMGN1 sequences. The average pairwise percent identity of the alignments is 40.4%. The conserved RXSXRLS motif (SEQ ID NO: 1255) is highlighted with a black box.

FIGS. 78A-78B depict TniQ testing of fused Functional Domains. FIG. 78A shows a library of functional domains that was introduced into a TniQ expression vector and expressed separately with a pHelper vector containing other components needed for transposition (Cas12k. S15, TnsB. TnsC and a single guide targeting AAVS1-5). The Split vector control of the wild type H1 core TniQ fusion and pHelper was not detectable. FIG. 78B shows the top 14 most active functional domains from split vector testing retested in the single expression construct. Of all the constructs tested, 13 of 14 functional domains in single expression contexts resulted in greater efficiency of transposition over the WT Single Expression Control.

### BRIEF DESCRIPTION OF THE SEQUENCE LISTING

The Sequence Listing filed herewith provides exemplary polynucleotide and polypeptide sequences for use in methods, compositions, and systems according to the disclosure. Below are exemplary descriptions of sequences therein.

### MG64

SEQ ID NOs: 1, 12, 16, 20-30, 64, 80-85, and 220 show the full-length peptide sequences of MG64 Cas effectors.

SEQ ID NOs: 2-4, 13-15, 17-19, 65-67, and 109-111 show the peptide sequences of MG64 transposition proteins that may comprise a recombinase/transposase recognition complex associated with the MG64 Cas effector.

SEQ ID NOs: 5-6, 32-33, 94-95, 104-105, 119-122, and 222 show nucleotide sequences of MG64 tracrRNAs derived from the same loci as a MG64 Cas effector.

SEQ ID NOs: 7 and 34-35 show nucleotide sequences of MG64 target CRISPR repeats.

SEQ ID NOs: 106-108, 112-118, and 221 show nucleotide sequences of MG64 crRNAs.

SEQ ID NOs: 8, 10, 39-44, 77, 79, and 93 show nucleotide sequences of right-hand transposase recognition sequences associated with a MG64 system.

SEQ ID NOs: 9, 11, 36-38, 76, and 78 show nucleotide sequences of left-hand transposase recognition sequences associated with a MG64 system.

SEQ ID NOs: 45-63, 68-75, 96-103, and 123-140 show nucleotide sequences of single guide RNAs engineered to function with MG64 Cas effectors.

SEQ ID NO: 208 shows the nucleotide sequence of an MG64 expression construct.

SEQ ID NO: 223 shows the nucleotide sequence of an MG64 active donor.

SEQ ID NOs: 228-230 show the full-length peptide sequences of MG64 accessory proteins.

SEQ ID NOs: 233-234 show the nucleotide sequences of MG64 target sites.

SEQ ID NOs: 369-371 show the nucleotide sequences of MG64 active target sequences.

### MG190

SEQ ID NOs: 209-219 show the full-length peptide sequences of MG190 ribosomal protein S15 homologs.

### Other Sequences

SEQ ID NOs: 86-87, 192-207, and 1354-1383 show peptide sequences of nuclear localizing signals.

SEQ ID NOs: 88-89 show peptide sequences of linkers.

SEQ ID NOs: 90-92 show peptide sequences of epitope tags.

SEQ ID NOs: 141-143 show genomic target sequences.

SEQ ID NOs: 144-180 show target guide sequences.

SEQ ID NOs: 181-183 show nucleic acid sequences of the S15 fusion proteins.

SEQ ID NO: 184 shows a donor construct.

SEQ ID NO: 185 shows an MG64-1 sgRNA sequence.

SEQ ID NO: 186 shows a linker sequence.

SEQ ID NOs: 187-189 show amino acid sequences of the S15 fusion proteins.

SEQ ID NOs: 190-191 show promoter sequences.

SEQ ID NOs: 224-226 and 231-232 show the nucleotide sequences of primers.

SEQ ID NO: 227 shows the nucleotide sequence of a plasmid element.

SEQ ID NOs: 235-249 show the peptide sequences of ClpX accessory proteins.

SEQ ID NOs: 250-251 show the nucleotide sequences of primers.

SEQ ID NO: 252 shows the nucleotide sequence of a plasmid element.

SEQ ID NO: 253 shows the nucleotide sequence of a primer.

SEQ ID NOs: 254-256 show the nucleotide sequences of MG64 active target sequences.

SEQ ID NOs: 257-282 and 1138-1241 show the protein sequences of MG161 functional domains.

SEQ ID NOs: 283-307 and 1242-1254 show the protein sequences of MG162 functional domains.

SEQ ID NOs: 308-359 show the protein sequences of H1 core library.

SEQ ID NOs: 360-368 and 372-753 show the nucleotide sequences of primers and probes.

SEQ ID NOs: 754-944 show the nucleotide sequences of single guide targets.

SEQ ID NOs: 945-1135 show the nucleotide sequences of primer binding sequences.

SEQ ID NO: 1136 shows a nucleotide sequence of a promoter.

SEQ ID NO: 1137 shows a protein sequence of an expression construct.

### DETAILED DESCRIPTION

While various embodiments of the disclosure have been shown and described herein, it will be obvious to those skilled in the art that such embodiments are provided by way of example only. Numerous variations, changes, and substitutions may occur to those skilled in the art without departing from the disclosure. It should be understood that various alternatives to the embodiments of the disclosure described herein may be employed.

The practice of some methods disclosed herein employ, unless otherwise indicated, techniques of immunology, biochemistry, chemistry, molecular biology, microbiology, cell biology, genomics, and recombinant DNA. See for example Sambrook and Green, Molecular Cloning: A Laboratory Manual, 4th Edition (2012): the series Current Protocols in Molecular Biology (F. M. Ausubel, et al. eds.); the series Methods In Enzymology (Academic Press, Inc.), PCR 2: A Practical Approach (M. J. MacPherson, B. D. Hames and G. R. Taylor eds. (1995)), Harlow and Lane, eds. (1988) Antibodies, A Laboratory Manual, and Culture of Animal Cells: A Manual of Basic Technique and Specialized Applications, 6th Edition (R. I. Freshney, ed. (2010)).

As used herein, the singular forms “a”, “an” and “the” are intended to include the plural forms as well, unless the context clearly indicates otherwise. Furthermore, to the extent that the terms “including”, “includes”, “having”, “has”, “with”, or variants thereof are used in either the detailed description and/or the claims, such terms are intended to be inclusive in a manner similar to the term “comprising.”

The term “about” or “approximately” means within an acceptable error range for the particular value as determined by one of ordinary skill in the art, which will depend in part on how the value is measured or determined, i.e., the limitations of the measurement system. For example, “about” can mean within one or more than one standard deviation, per the practice in the art. Alternatively, “about” can mean a range of up to 20%, up to 15%, up to 10%, up to 5%, or up to 1% of a given value.

The term “nucleotide,” as used herein, refers to a base-sugar-phosphate combination. Contemplated nucleotides include naturally occurring nucleotides and synthetic nucleotides. Nucleotides are monomeric units of a nucleic acid sequence (e.g., deoxyribonucleic acid (DNA) and ribonucleic acid (RNA)). The term nucleotide includes ribonucleoside triphosphates adenosine triphosphate (ATP), uridine triphosphate (UTP), cytosine triphosphate (CTP), guanosine triphosphate (GTP) and deoxyribonucleoside triphosphates such as dATP, dCTP, dITP, dUTP, dGTP, dTTP, or derivatives thereof. Such derivatives include, for example, [αS]dATP, 7-deaza-dGTP and 7-deaza-dATP, and nucleotide derivatives that confer nuclease resistance on the nucleic acid molecule containing them. The term nucleotide as used herein encompasses dideoxyribonucleoside triphosphates (ddNTPs) and their derivatives. Illustrative examples of ddNTPs include, but are not limited to, ddATP, ddCTP, ddGTP, ddITP, and ddTTP. A nucleotide may be unlabeled or detectably labeled, such as using moieties comprising optically detectable moieties (e.g., fluorophores) or quantum dots. Detectable labels include, for example, radioactive isotopes, fluorescent labels, chemiluminescent labels, bioluminescent labels, and enzyme labels. Fluorescent labels of nucleotides include but are not limited fluorescein, 5-carboxyfluorescein (FAM), 2′7′-dimethoxy-4′5-dichloro-6-carboxyfluorescein (JOE), rhodamine, 6-carboxyrhodamine (R6G), N,N,N′,N′-tetramethyl-6-carboxyrhodamine (TAMRA). 6-carboxy-X-rhodamine (ROX), 4-(4′dimethylaminophenylazo) benzoic acid (DABCYL), Cascade Blue, Oregon Green, Texas Red, Cyanine and 5-(2′-aminoethyl)aminonaphthalene-1-sulfonic acid (EDANS). Specific examples of fluorescently labeled nucleotides include [R6G]dUTP, [TAMRA]dUTP, [R110]dCTP, [R6G]dCTP, [TAMRA]dCTP. [JOE]ddATP, [R6G]ddATP, [FAM]ddCTP, [R110]ddCTP, [TAMRA]ddGTP, [ROX]ddTTP, [dR6G]ddATP. [dR110]ddCTP, [dTAMRA]ddGTP, and [dROX]ddTTP available from Perkin Elmer, Foster City, Calif; FluoroLink DeoxyNucleotides, FluoroLink Cy3-dCTP, FluoroLink Cy5-dCTP, FluoroLink Fluor X-dCTP, FluoroLink Cy3-dUTP, and FluoroLink Cy5-dUTP available from Amersham, Arlington Heights, IL: Fluorescein-15-dATP, Fluorescein-12-dUTP, Tetramethyl-rodamine-6-dUTP, IR770-9-dATP. Fluorescein-12-ddUTP, Fluorescein-12-UTP, and Fluorescein-15-2′-dATP available from Boehringer Mannheim, Indianapolis, Ind.; and Chromosome Labeled Nucleotides, BODIPY-FL-14-UTP, BODIPY-FL-4-UTP, BODIPY-TMR-14-UTP, BODIPY-TMR-14-dUTP, BODIPY-TR-14-UTP, BODIPY-TR-14-dUTP, Cascade Blue-7-UTP. Cascade Blue-7-dUTP, fluorescein-12-UTP, fluorescein-12-dUTP, Oregon Green 488-5-dUTP, Rhodamine Green-5-UTP, Rhodamine Green-5-dUTP, tetramethylrhodamine-6-UTP, tetramethylrhodamine-6-dUTP, Texas Red-5-UTP, Texas Red-5-dUTP, and Texas Red-12-dUTP available from Molecular Probes, Eugene, Oreg. The term nucleotide encompasses chemically modified nucleotides. An exemplary chemically-modified nucleotide is biotin-dNTP. Non-limiting examples of biotinylated dNTPs include, biotin-dATP (e.g., bio-N6-ddATP, biotin-14-dATP), biotin-dCTP (e.g., biotin-1 l-dCTP, biotin-14-dCTP), and biotin-dUTP (e.g., biotin-11-dUTP, biotin-16-dUTP, biotin-20-dUTP).

The terms “polynucleotide,” “oligonucleotide,” and “nucleic acid” are used interchangeably to refer to a polymeric form of nucleotides of any length, either deoxyribonucleotides or ribonucleotides, or analogs thereof, either in single-, double-, or multi-stranded form. Contemplated polynucleotides include a gene or fragment thereof. Exemplary polynucleotides include, but are not limited to, DNA, RNA, coding or non-coding regions of a gene or gene fragment, loci (locus) defined from linkage analysis, exons, introns, messenger RNA (mRNA), transfer RNA (tRNA), ribosomal RNA (rRNA), short interfering RNA (siRNA), short-hairpin RNA (shRNA), micro-RNA (miRNA), ribozymes, cDNA, recombinant polynucleotides, branched polynucleotides, plasmids, vectors, cell-free polynucleotides including cell-free DNA (cfDNA) and cell-free RNA (cfRNA), nucleic acid probes, and primers. In a polynucleotide when referring to a T, a T means U (Uracil) in RNA and T (Thymine) in DNA. A polynucleotide can be exogenous or endogenous to a cell and/or exist in a cell-free environment. The term polynucleotide encompasses modified polynucleotides (e.g., altered backbone, sugar, or nucleobase). If present, modifications to the nucleotide structure are imparted before or after assembly of the polymer. Non-limiting examples of modifications include: 5-bromouracil, peptide nucleic acid, xeno nucleic acid, morpholinos, locked nucleic acids, glycol nucleic acids, threose nucleic acids, dideoxynucleotides, cordycepin, 7-deaza-GTP, fluorophores (e.g., rhodamine or fluorescein linked to the sugar), thiol-containing nucleotides, biotin-linked nucleotides, fluorescent base analogs, CpG islands, methyl-7-guanosine, methylated nucleotides, inosine, thiouridine, pseudouridine, dihydrouridine, queuosine, and wyosine. The sequence of nucleotides may be interrupted by non-nucleotide components.

The terms “peptide,” “polypeptide,” and “protein” are used interchangeably herein to refer to a polymer of at least two amino acid residues joined by peptide bond(s). This term does not connote a specific length of polymer, nor is it intended to imply or distinguish whether the peptide is produced using recombinant techniques, chemical or enzymatic synthesis, or is naturally occurring. The terms apply to naturally occurring amino acid polymers as well as amino acid polymers comprising at least one modified amino acid. In some cases, the polymer is interrupted by non-amino acids. The terms include amino acid chains of any length, including full length proteins, and proteins with or without secondary or tertiary structure (e.g., domains). The terms also encompass an amino acid polymer that has been modified, for example, by disulfide bond formation, glycosylation, lipidation, acetylation, phosphorylation, oxidation, and any other manipulation such as conjugation with a labeling component. The terms “amino acid” and “amino acids,” as used herein, refer to natural and non-natural amino acids, including, but not limited to, modified amino acids. Modified amino acids include amino acids that have been chemically modified to include a group or a chemical moiety not naturally present on the amino acid. The term “amino acid” includes both D-amino acids and L-amino acids.

As used herein, the “non-native” refers to a nucleic acid or polypeptide sequence that is non-naturally occurring. Non-native refers to a non-naturally occurring nucleic acid or polypeptide sequence that comprises modifications such as mutations, insertions, or deletions. The term non-native encompasses fusion nucleic acids or polypeptides that encodes or exhibits an activity (e.g., enzymatic activity, methyltransferase activity, acetyltransferase activity, kinase activity, ubiquitinating activity, etc.) of the nucleic acid or polypeptide sequence to which the non-native sequence is fused. A non-native nucleic acid or polypeptide sequence includes those linked to a naturally-occurring nucleic acid or polypeptide sequence (or a variant thereof) by genetic engineering to generate a chimeric nucleic acid or polypeptide sequence encoding a chimeric nucleic acid or polypeptide.

As used herein, “operably linked”, “operable linkage”, “operatively linked”, or grammatical equivalents thereof refer to an arrangement of genetic elements, e.g., a promoter, an enhancer, a polyadenylation sequence, etc., wherein an operation (e.g., movement or activation) of a first genetic element has some effect on the second genetic element. The effect on the second genetic element can be, but need not be, of the same type as operation of the first genetic element. For example, two genetic elements are operably linked if movement of the first element causes an activation of the second element. For instance, a regulatory element, which may comprise promoter and/or enhancer sequences, is operatively linked to a coding region if the regulatory element helps initiate transcription of the coding sequence. There may be intervening residues between the regulatory element and coding region so long as this functional relationship is maintained.

A “functional fragment” of a DNA or protein sequence refers to a fragment that retains a biological activity (either functional or structural) that is substantially similar to a biological activity of the full-length DNA or protein sequence. A biological activity of a DNA sequence includes its ability to influence expression in a manner attributed to the full-length sequence.

The terms “engineered,” “synthetic,” and “artificial” are used interchangeably herein to refer to an object that has been modified by human intervention. For example, the terms refer to a polynucleotide or polypeptide that is non-naturally occurring. An engineered peptide has, but does not require, low sequence identity (e.g., less than 50% sequence identity, less than 25% sequence identity, less than 10% sequence identity, less than 5% sequence identity, less than 1% sequence identity) to a naturally occurring human protein. For example, VPR and VP64 domains are synthetic transactivation domains. Non-limiting examples include the following: a nucleic acid modified by changing its sequence to a sequence that does not occur in nature; a nucleic acid modified by ligating it to a nucleic acid that it does not associate with in nature such that the ligated product possesses a function not present in the original nucleic acid: an engineered nucleic acid synthesized in vitro with a sequence that does not exist in nature; a protein modified by changing its amino acid sequence to a sequence that does not exist in nature; an engineered protein acquiring a new function or property. An “engineered” system comprises at least one engineered component.

The term “tracrRNA” or “tracr sequence” means trans-activating CRISPR RNA. tracrRNA interacts with the CRISPR (cr) RNA to form guide (g) RNA in type II and subtype V-B CRISPR-Cas systems. If the tracrRNA is engineered, it may have about 5%, 10%, 20%, 30%, 40%, 50%, 60%, 70%, 80%, 90%, 95%, or 100% sequence identity and/or sequence similarity to a wild type exemplary tracrRNA sequence (e.g., a tracrRNA from S. pyogenes, S. aureus). tracrRNA may refer to a modified form of a tracrRNA that can comprise a nucleotide change such as a deletion, insertion, or substitution, variant, mutation, or chimera. The term tracrRNA encompasses a nucleic acid that can be at least about 60% identical to a wild type exemplary tracrRNA (e.g., a tracrRNA from S. pyogenes, S. aureus, etc) sequence over a stretch of at least 6 contiguous nucleotides. For example, a tracrRNA sequence has at least about 60% identical, at least about 65% identical, at least about 70% identical, at least about 75% identical, at least about 80% identical, at least about 85% identical, at least about 90% identical, at least about 95% identical, at least about 98% identical, at least about 99% identical, or 100% identical to a wild type exemplary tracrRNA (e.g., a tracrRNA from S. pyogenes, S. aureus, etc) sequence over a stretch of at least 6 contiguous nucleotides. Type II tracrRNA sequences can be predicted on a genome sequence by identifying regions with complementarity to part of the repeat sequence in an adjacent CRISPR array.

As used herein, a “guide nucleic acid” or “guide polynucleotide” refers to a nucleic acid that may hybridize to a target nucleic acid and thereby directs an associated nuclease to the target nucleic acid. A guide nucleic acid is, but is not limited to, RNA (guide RNA or gRNA), DNA, or a mixture of RNA and DNA. A guide nucleic acid can include a crRNA or a tracrRNA or a combination of both. The term guide nucleic acid encompasses an engineered guide nucleic acid and a programmable guide nucleic acid to specifically bind to the target nucleic acid. A portion of the target nucleic acid may be complementary to a portion of the guide nucleic acid. The strand of a double-stranded target polynucleotide that is complementary to and hybridizes with the guide nucleic acid is the complementary strand. The strand of the double-stranded target polynucleotide that is complementary to the complementary strand, and therefore is not complementary to the guide nucleic acid is called noncomplementary strand. A guide nucleic acid having a polynucleotide chain is a “single guide nucleic acid.” A guide nucleic acid having two polynucleotide chains is a “double guide nucleic acid.” If not otherwise specified, the term “guide nucleic acid” is inclusive, referring to both single guide nucleic acids and double guide nucleic acids. A guide nucleic acid may comprise a segment referred to as a “nucleic acid-targeting segment” or a “nucleic acid-targeting sequence.” or a “spacer.” A nucleic acid-targeting segment can include a sub-segment referred to as a “protein binding segment” or “protein binding sequence” or “Cas protein binding segment.”

The term “sequence identity” or “percent identity” in the context of two or more nucleic acids or polypeptide sequences, refers to two (e.g., in a pairwise alignment) or more (e.g., in a multiple sequence alignment) sequences that are the same or have a specified percentage of amino acid residues or nucleotides that are the same, when compared and aligned for maximum correspondence over a local or global comparison window, as measured using a sequence comparison algorithm. Suitable sequence comparison algorithms for polypeptide sequences include, e.g., BLASTP using parameters of a wordlength (W) of 3, an expectation (E) of 10, and the BLOSUM62 scoring matrix setting gap costs at existence of 11, extension of 1, and using a conditional compositional score matrix adjustment for polypeptide sequences longer than 30 residues; BLASTP using parameters of a wordlength (W) of 2, an expectation (E) of 1000000, and the PAM30 scoring matrix setting gap costs at 9 to open gaps and 1 to extend gaps for sequences of less than 30 residues (these are the default parameters for BLASTP in the BLAST suite available at https://blast.ncbi.nlm.nih.gov): CLUSTALW with the Smith-Waterman homology search algorithm parameters with a match of 2, a mismatch of −1, and a gap of −1; MUSCLE with default parameters; MAFFT with parameters of a retree of 2 and max iterations of 1000; Novafold with default parameters; HMMER hmmalign with default parameters.

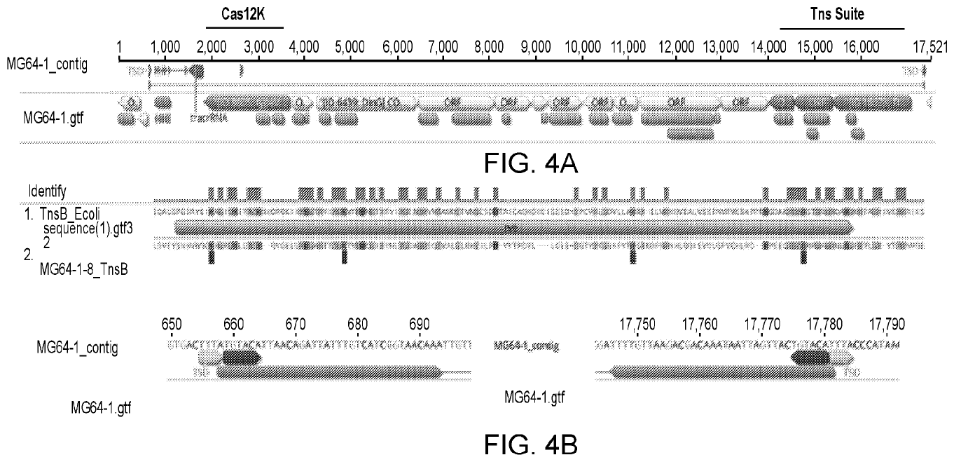

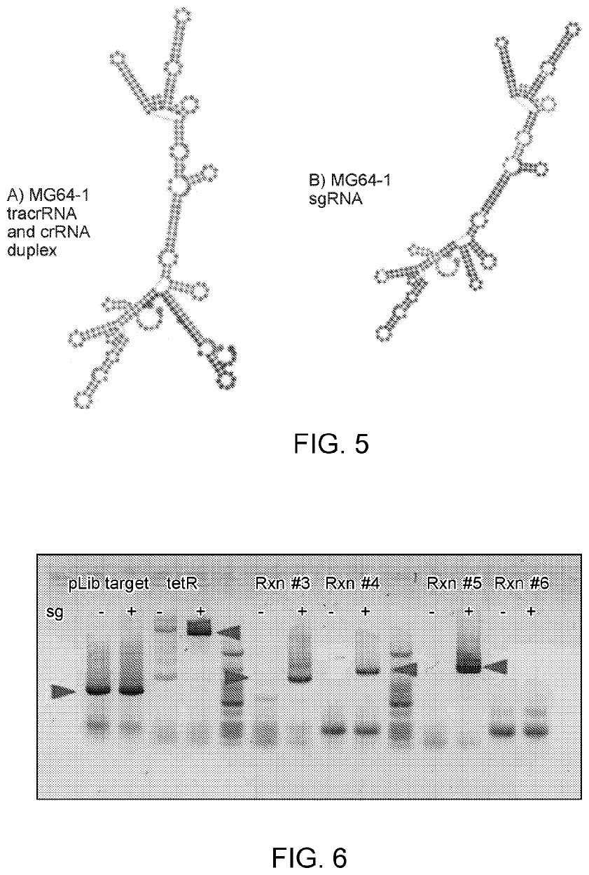

Included in the current disclosure are variants of any of the enzymes described herein with one or more conservative amino acid substitutions. Such conservative substitutions can be made in the amino acid sequence of a polypeptide without disrupting the three-dimensional structure or function of the polypeptide. Conservative substitutions can be accomplished by substituting amino acids with similar hydrophobicity, polarity, and R chain length for one another. Additionally or alternatively, by comparing aligned sequences of homologous proteins from different species, conservative substitutions can be identified by locating amino acid residues that have been mutated between species (e.g., non-conserved residues without altering the basic functions of the encoded proteins. Such conservatively substituted variants may include variants with at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, or at least about 99% identity any one of the systems described herein (e.g., MG64 systems described herein). In some embodiments, such conservatively substituted variants are functional variants. Such functional variants can encompass sequences with substitutions such that the activity of critical active site residues of the endonuclease are not disrupted. In some embodiments, a functional variant of any of the systems described herein lack substitution of at least one of the conserved or functional residues called out in FIGS. 4A, 4B and 5. In some embodiments, a functional variant of any of the systems described herein lacks substitution of all of the conserved or functional residues called out in FIGS. 4A, 4B and 5.

Also included in the current disclosure are variants of any of the enzymes described herein with substitution of one or more catalytic residues to decrease or eliminate activity of the enzyme (e.g. decreased-activity variants). In some embodiments, a decreased activity variant as a protein described herein comprises a disrupting substitution of at least one, at least two, or all three catalytic residues.

Conservative substitution tables providing functionally similar amino acids are available from a variety of references (see, for example, Creighton. Proteins: Structures and Molecular Properties (W H Freeman & Co.: ₂^{nd} Edition (December 1993))). The following eight groups each contain amino acids that are conservative substitutions for one another:
1) Alanine (A), Glycine (G);
2) Aspartic acid (D), Glutamic acid (E);
3) Asparagine (N), Glutamine (Q);
4) Arginine (R), Lysine (K):
5) Isoleucine (I), Leucine (L), Methionine (M), Valine (V);
6) Phenylalanine (F), Tyrosine (Y), Tryptophan (W):
7) Serine (S), Threonine (T); and
8) Cysteine (C), Methionine (M).

As used herein, the term “RuvC_III domain” refers to a third discontinuous segment of a RuvC endonuclease domain (the RuvC nuclease domain being comprised of three discontiguous segments, RuvC_I, RuvC_II, and RuvC_III). A RuvC domain or segments thereof can generally be identified by alignment to documented domain sequences, structural alignment to proteins with annotated domains, or by comparison to Hidden Markov Models (HMMs) built based on documented domain sequences (e.g., Pfam HMM PF18541 for RuvC_III).

As used herein, the term “HNH domain” refers to an endonuclease domain having characteristic histidine and asparagine residues. An HNH domain can generally be identified by alignment to documented domain sequences, structural alignment to proteins with annotated domains, or by comparison to Hidden Markov Models (HMMs) built based on documented domain sequences (e.g., Pfam HMM PF01844 for domain HNH).

As used herein, the term “recombinase” refers to an enzyme that mediates the recombination of DNA fragments located between recombinase recognition sequences, which results in the excision, insertion, inversion, exchange or translocation) of the DNA fragments located between the recombinase recognition sequences.

As used herein, the term “recombine,” or “recombination,” in the context of a nucleic acid modification (e.g., a genomic modification), refers to the process by which two or more nucleic acid molecules, or two or more regions of a single nucleic acid molecule, are modified by the action of a recombinase protein. Recombination can result in, inter alia, the excision, insertion, inversion, exchange, or translocation of a nucleic acid sequence, e.g., in or between one or more nucleic acid molecules.

As used herein, the term “transposon,” or “transposable element” refers to a nucleic acid sequence in a genome that is a mobile genetic element that can change its position in a genome. In some embodiments, the transposon transports additional “cargo DNA” excised from the genome. Transposons comprise, for example retrotransposons, DNA transposons, autonomous and non-autonomous transposons, and class III transposons. Transposon nucleic acid sequences comprise, for example genes coding for a cognate transposase, one or more recognition sequences for the transposase, or combinations thereof. In some embodiments, these transposons differ on the type of nucleic acid to transpose, the type of repeat at the ends of the transposon, the type of cargo to be carried or by the mode of transposition (i.e. self-repair or host-repair). As used herein, the term “transposase” or “transposases” refers to an enzyme that binds to the recognition sequences of a transposon and catalyzes its movement to another part of the genome. In some embodiments, the movement is by a cut and paste mechanism or a replicative transposition mechanism.

As used herein, the term “Tn7” or “Tn7-like transposase” refers to a family of transposases comprising three main components: a heteromeric transposase (TnsA and/or TnsB) alongside a regulator protein (TnsC). In addition to the TnsABC transposition proteins, Tn7 elements can encode dedicated target site-selection proteins, TnsD and TnsE. In conjunction with TnsABC, the sequence-specific DNA-binding protein TnsD directs transposition into a conserved site referred to as the “Tn7 attachment site.” attTn7. TnsD is a member of a large family of proteins that also includes TniQ. TniQ has been shown to target transposition into resolution sites of plasmids.

As used herein, the term “complex” refers to a joining of at least two components. The two components may each retain the properties/activities they had prior to forming the complex. The joining may be by covalent bonding, non-covalent bonding (i.e., hydrogen bonding, ionic interactions, Van der Waals interactions, and hydrophobic bond), use of a linker, fusion, or any other suitable method. In some embodiments, components in a complex are polynucleotides, polypeptides, or combinations thereof. For example, a complex may comprise a Cas protein and a guide nucleic acid.

In some embodiments, the CAST systems described herein comprise one or more Tn7 or Tn7 like transposases. In certain example embodiments, the Tn7 or Tn7 like transposase comprises a multimeric protein complex. In certain example embodiments, the multimeric protein complex comprises TnsA, TnsB, TnsC, or TniQ. In these combinations, the transposases (TnsA, TnsB, TnsC, TniQ) may form complexes or fusion proteins with each other.

In some embodiments, the CAST systems described herein comprise one or more Tn5053 or Tn5053-like transposases. In certain example embodiments, the Tn5053 or Tn5053-like transposase comprises a multimeric protein complex. In certain example embodiments, the multimeric protein complex comprises TnsA, TnsB, TnsC, or TniQ. In these combinations, the transposases (TnsA, TnsB, TnsC, TniQ) may form complexes or fusion proteins with each other.

As used herein, the term “Cas12k” (alternatively “class 2, type V-K”) refers to a subtype of Type V CRISPR systems that have been found to be defective in nuclease activity (e.g., they may comprise at least one defective RuvC domain that lacking at least one catalytic residue important for DNA cleavage). Such subtype of effectors have been generally associated with CAST systems.

In accordance with IUPAC conventions, the following abbreviations are used throughout the examples:
A=adenine
C=cytosine
G=guanine
T=thymine
R=adenine or guanine
Y=cytosine or thymine
S=guanine or cytosine
W=adenine or thymine
K=guanine or thymine
M=adenine or cytosine
B=C, G, or T
D=A, G, or T
H=A, C, or T
V=A, C, or G

### Overview

The discovery of new Cas enzymes with unique functionality and structure may offer the potential to further disrupt deoxyribonucleic acid (DNA) editing technologies, improving speed, specificity, functionality, and ease of use. Relative to the predicted prevalence of Clustered Regularly Interspaced Short Palindromic Repeats (CRISPR) systems in microbes and the sheer diversity of microbial species, relatively few functionally characterized CRISPR/Cas enzymes exist in the literature. This is partly because a huge number of microbial species may not be readily cultivated in laboratory conditions. Metagenomic sequencing from natural environmental niches that represent large numbers of microbial species may offer the potential to drastically increase the number of new CRISPR/Cas systems documented and speed the discovery of new oligonucleotide editing functionalities. A recent example of the fruitfulness of such an approach is demonstrated by the 2016 discovery of CasX/CasY CRISPR systems from metagenomic analysis of natural microbial communities.

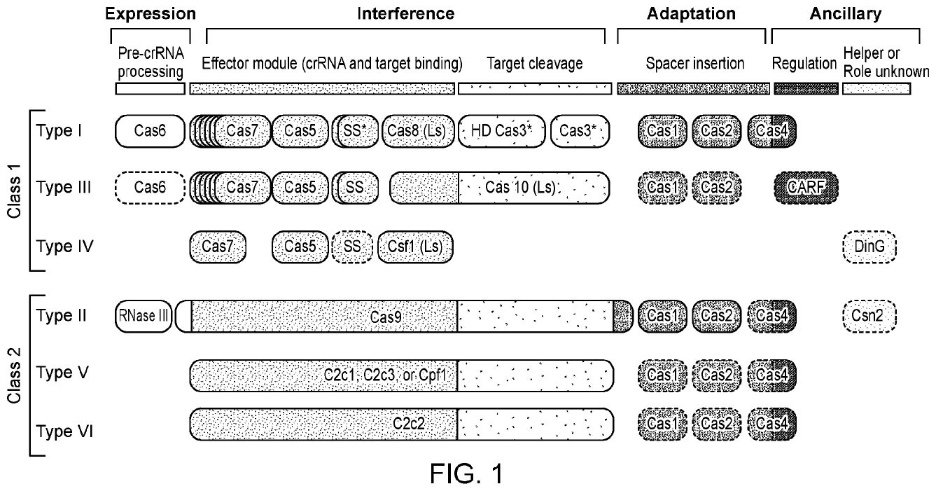

CRISPR/Cas systems are RNA-directed nuclease complexes that have been described to function as an adaptive immune system in microbes. In their natural context, CRISPR/Cas systems occur in CRISPR (clustered regularly interspaced short palindromic repeats) operons or loci, which generally comprise two parts: (i) an array of short repetitive sequences (30-40 bp) separated by equally short spacer sequences, which encode the RNA-based targeting element; and (ii) ORFs encoding the Cas encoding the nuclease polypeptide directed by the RNA-based targeting element alongside accessory proteins/enzymes. Efficient nuclease targeting of a particular target nucleic acid sequence generally requires both (i) complementary hybridization between the first 6-8 nucleic acids of the target (the target seed) and the crRNA guide; and (ii) the presence of a protospacer-adjacent motif (PAM) sequence within a defined vicinity of the target seed (the PAM usually being a sequence not commonly represented within the host genome). Depending on the exact function and organization of the system, CRISPR-Cas systems are commonly organized into 2 classes, 5 types and 16 subtypes based on shared functional characteristics and evolutionary similarity (see FIG. 1).

Class 1 CRISPR-Cas systems have large, multisubunit effector complexes, and comprise Types I, III, and IV.

Type I CRISPR-Cas systems are considered of moderate complexity in terms of components. In Type I CRISPR-Cas systems, the array of RNA-targeting elements is transcribed as a long precursor crRNA (pre-crRNA) that is processed at repeat elements to liberate short, mature crRNAs that direct the nuclease complex to nucleic acid targets when they are followed by a suitable short consensus sequence called a protospacer-adjacent motif (PAM). This processing occurs via an endoribonuclease subunit (Cas6) of a large endonuclease complex called Cascade, which also comprises a nuclease (Cas3) protein component of the crRNA-directed nuclease complex. Cas I nucleases function primarily as DNA nucleases.

Type III CRISPR systems may be characterized by the presence of a central nuclease, known as Cas10, alongside a repeat-associated mysterious protein (RAMP) that comprises Csm or Cmr protein subunits. Like in Type I systems, the mature crRNA is processed from a pre-crRNA using a Cas6-like enzyme. Unlike type I and II systems, type III systems appear to target and cleave DNA-RNA duplexes (such as DNA strands being used as templates for an RNA polymerase).

Type IV CRISPR-Cas systems possess an effector complex that comprises a highly reduced large subunit nuclease (csf1), two genes for RAMP proteins of the Cas5 (csf3) and Cas7 (csf2) groups, and, in some embodiments, a gene for a predicted small subunit; such systems are commonly found on endogenous plasmids.

Class 2 CRISPR-Cas systems generally have single-polypeptide multidomain nuclease effectors, and comprise Types II, V and VI.

Type II CRISPR-Cas systems are considered the simplest in terms of components. In Type II CRISPR-Cas systems, the processing of the CRISPR array into mature crRNAs does not require the presence of a special endonuclease subunit, but rather a small trans-encoded crRNA (tracrRNA) with a region complementary to the array repeat sequence: the tracrRNA interacts with both its corresponding effector nuclease (e.g., Cas9) and the repeat sequence to form a precursor dsRNA structure, which is cleaved by endogenous RNAse III to generate a mature effector enzyme loaded with both tracrRNA and crRNA. Type II nucleases are known as DNA nucleases. Type II effectors generally exhibit a structure consisting of a RuvC-like endonuclease domain that adopts the RNase H fold with an unrelated HNH nuclease domain inserted within the folds of the RuvC-like nuclease domain. The RuvC-like domain is responsible for the cleavage of the target (e.g., crRNA complementary) DNA strand, while the HNH domain is responsible for cleavage of the displaced DNA strand.

Type V CRISPR-Cas systems are characterized by a nuclease effector (e.g., Cas12) structure similar to that of Type II effectors, comprising a RuvC-like domain. Similar to Type II, most (but not all) Type V CRISPR systems use a tracrRNA to process pre-crRNAs into mature crRNAs; however, unlike Type II systems which requires RNAse III to cleave the pre-crRNA into multiple crRNAs, Type V systems are capable of using the effector nuclease itself to cleave pre-crRNAs. Like Type-II CRISPR-Cas systems, Type V CRISPR-Cas systems are again known as DNA nucleases. Unlike Type II CRISPR-Cas systems, some Type V enzymes (e.g., Cas12a) appear to have a robust single-stranded nonspecific deoxyribonuclease activity that is activated by the first crRNA directed cleavage of a double-stranded target sequence.

Type VI CRISPR-Cas systems have RNA-guided RNA endonucleases. Instead of RuvC-like domains, the single polypeptide effector of Type VI systems (e.g., Cas13) comprises two HEPN ribonuclease domains. Differing from both Type II and V systems, Type VI systems also appear to not need a tracrRNA for processing of pre-crRNA into crRNA. Similar to type V systems, however, some Type VI systems (e.g., C2C2) appear to possess robust single-stranded nonspecific nuclease (ribonuclease) activity activated by the first crRNA directed cleavage of a target RNA.

Because of their simpler architecture, Class 2 CRISPR-Cas have been most widely adopted for engineering and development as designer nuclease/genome editing applications.

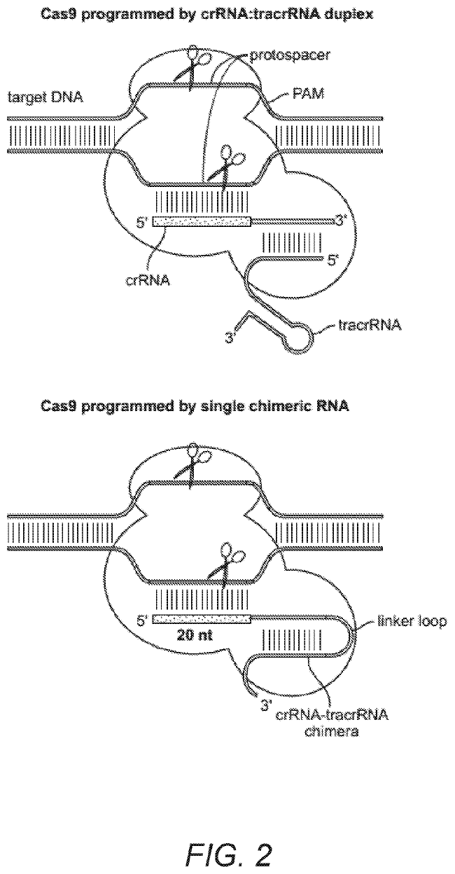

One of the early adaptations of such a system for in vitro use involved (i) recombinantly-expressed, purified full-length Cas9 (e.g., a Class 2, Type II Cas enzyme) isolated from S. pyogenes SF370. (ii) purified mature ~42 nt crRNA bearing a ~20 nt 5′ sequence complementary to the target DNA sequence desired to be cleaved followed by a 3′ tracr-binding sequence (the whole crRNA being in vitro transcribed from a synthetic DNA template carrying a T7 promoter sequence); (iii) purified tracrRNA in vitro transcribed from a synthetic DNA template carrying a T7 promoter sequence, and (iv) Mg²⁺. A later improved, engineered system involved the crRNA of (ii) joined to the 5′ end of (iii) by a linker (e.g., GAAA) to form a single fused synthetic guide RNA (sgRNA) capable of directing Cas9 to a target by itself (compare top and bottom panel of FIG. 2).

Such engineered systems can be adapted for use in mammalian cells by providing DNA vectors encoding (i) an ORF encoding codon-optimized Cas9 (e.g., a Class 2, Type II Cas enzyme) under a suitable mammalian promoter with a C-terminal nuclear localization sequence (e.g., SV40 NLS) and a suitable poly adenylation signal (e.g., TK pA signal); and (ii) an ORF encoding an sgRNA (having a 5′ sequence beginning with G followed by 20 nt of a complementary targeting nucleic acid sequence joined to a 3′ tracr-binding sequence, a linker, and the tracrRNA sequence) under a suitable Polymerase III promoter (e.g., the U6 promoter).

Transposons are mobile elements that can move between positions in a genome. Such transposons have evolved to limit the negative effects they exert on the host. A variety of regulatory mechanisms are used to maintain transposition at a low frequency and sometimes coordinate transposition with various cell processes. Some prokaryotic transposons also can mobilize functions that benefit the host or otherwise help maintain the element. Certain transposons may have also evolved mechanisms of tight control over target site selection, the most notable example being the Tn7 family.

Transposon Tn7 and similar elements may be reservoirs for antibiotic resistance and pathogenesis functions in clinical settings, as well as encoding other adaptive functions in natural environments. The Tn7 system, for example, has evolved mechanisms to almost completely avoid integrating into important host genes, but also maximize dispersal of the element by recognizing mobile plasmids and bacteriophage capable of moving Tn7 between host bacteria.

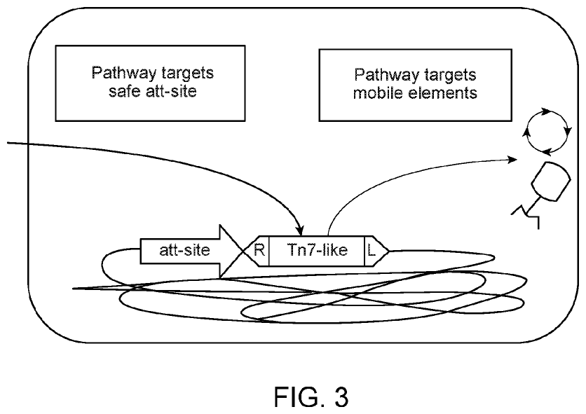

Tn7 and Tn7-like elements may control where and when they insert, possessing one pathway that directs insertion into a single conserved position in bacterial genomes and a second pathway that appears to be adapted to maximizing targeting into mobile plasmids capable of transporting the element between bacteria (see FIG. 3). The association between Tn7-like transposons and CRISPR-Cas systems suggests that the transposons might have hijacked CRISPR effectors to generate R-loops in target sites and facilitate the spread of transposons via plasmids and phages.

### MG64 Systems

Provided herein, in some embodiments, are MG64 systems for transposing a cargo nucleotide sequence into a target nucleic acid site. See FIGS. 4A-4B.

Described herein, in certain embodiments, are system for transposing a cargo nucleotide sequence into a target nucleic acid site in a target nucleic acid comprising: a) a Cas effector complex comprising a class 2, type V Cas effector, a small prokaryotic ribosomal protein subunit S15, and an engineered guide polynucleotide configured to hybridize to the target nucleic acid site: b) a Tn7 type transposase complex configured to bind the Cas effector complex and comprising a TnsB, TnsC, and TniQ component and an accessory protein; and c) a double-stranded nucleic acid configured to interact with the Tn7 type transposase complex and comprising the cargo nucleotide sequence.

In some aspects, the present disclosure provides a system for transposing a cargo nucleotide sequence into a target nucleic acid site in a target nucleic acid comprising a Cas effector complex comprising a class 2, type V Cas effector, a small prokaryotic ribosomal protein subunit S15, and an engineered guide polynucleotide that hybridizes to the target nucleic acid site; a Tn7 type transposase complex that binds the Cas effector complex and comprises a TnsB, TnsC, and TniQ component and an accessory protein comprising a sequence having at least 70% sequence identity to any one of SEQ ID NOs: 228-230 and 235-249; and a double-stranded nucleic acid that interacts with the Tn7 type transposase complex and comprises the cargo nucleotide sequence.

In another aspect, the present disclosure provides system for transposing a cargo nucleotide sequence into a target nucleic acid site in a target nucleic acid comprising: a Cas effector complex comprising a class 2, type V Cas effector, a small prokaryotic ribosomal protein subunit S15, and an engineered guide polynucleotide that hybridizes to the target nucleic acid site; a Tn7 type transposase complex that binds the Cas effector complex and comprises a functional domain (FD)-TniQ fusion, and an accessory protein; and a double-stranded nucleic acid that interacts with the Tn7 type transposase complex and comprises the cargo nucleotide sequence.

In another aspect, the present disclosure provides system for transposing a cargo nucleotide sequence into a target nucleic acid site in a target nucleic acid comprising: a Cas effector complex comprising a class 2, type V Cas effector, a small prokaryotic ribosomal protein subunit S15, and an engineered guide polynucleotide that hybridizes to the target nucleic acid site; a Tn7 type transposase complex that binds the Cas effector complex and comprises a functional domain (FD)-TniQ fusion, wherein the functional domain (FD) comprises a sequence having at least 80% identity to any one of SEQ ID NOs: 257-307 and 1138-1242, and an accessory protein; and a double-stranded nucleic acid that interacts with the Tn7 type transposase complex and comprises the cargo nucleotide sequence.

In some aspects, the present disclosure provides a system for transposing a cargo nucleotide sequence into a target nucleic acid site in a target nucleic acid comprising a Cas effector complex comprising a class 2, type V Cas effector, a small prokaryotic ribosomal protein subunit S15, and an engineered guide polynucleotide that hybridizes to the target nucleic acid site: a Tn7 type transposase complex that binds the Cas effector complex and comprises a TnsB, TnsC, and TniQ component and an accessory protein comprising a sequence having at least 70%, 75%, 80%, 85%, 90%, 95%, 98%, 99% or 100% sequence identity to any one of SEQ ID NOs: 228-230 and 235-249; and a double-stranded nucleic acid that interacts with the Tn7 type transposase complex and comprises the cargo nucleotide sequence.

In some embodiments, the system comprises a double-stranded nucleic acid comprising a cargo nucleotide sequence. In some embodiments, this cargo nucleotide sequence interacts with a Tn7 type or Tn5053 type transposase complex. In some embodiments, the system comprises a Cas effector complex. In some embodiments, the Cas effector complex comprises a class 2, type V Cas effector and an engineered guide polynucleotide configured to hybridize to the target nucleotide sequence. In some embodiments, the system comprises a Tn7 type or Tn5053 type transposase complex configured to bind the Cas effector complex, wherein the Tn7 type or Tn5053 type transposase complex comprises a TnsB subunit.

In some embodiments, the cargo nucleotide sequence is flanked by a left-hand transposase recognition sequence. In some embodiments, the cargo nucleotide sequence is flanked by a right-hand transposase recognition sequence. In some embodiments, the cargo nucleotide sequence is flanked by a left-hand transposase recognition sequence and a right-hand transposase recognition sequence.

In some embodiments, a target nucleic acid comprises the target nucleic acid site. In some embodiments, the target nucleic acid comprises a PAM sequence compatible with the Cas effector complex adjacent to the target nucleic acid site. In some embodiments, the PAM sequence is located 3′ of the target nucleic acid site. In some embodiments, the PAM sequence is located 5′ of the target nucleic acid site.

In some embodiments, the engineered guide polynucleotide is configured to bind the class 2, type V Cas effector. In some embodiments, the class 2, type V Cas effector is a class 2, type V-K effector. In some embodiments, the class 2, type V Cas effector comprises a polypeptide comprising a sequence having at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, or at least about 99% identity to SEQ ID NO: 1, 12, 16, 20-30, 64, 80-85, and 220. In some embodiments, the class 2, type V Cas effector comprises a polypeptide comprising a sequence having at least about 70% identity to SEQ ID NOs: 1, 12, 16, 20-30, 64, 80-85, and 220. In some embodiments, the class 2, type V Cas effector comprises a polypeptide comprising a sequence having at least about 75% identity to SEQ ID NOs: 1, 12, 16, 20-30, 64, 80-85, and 220. In some embodiments, the class 2, type V Cas effector comprises a polypeptide comprising a sequence having at least about 80% identity to SEQ ID NOs: 1, 12, 16, 20-30, 64, 80-85, and 220. In some embodiments, the class 2, type V Cas effector comprises a polypeptide comprising a sequence having at least about 85% identity to SEQ ID NOs: 1, 12, 16, 20-30, 64, 80-85, and 220. In some embodiments, the class 2, type V Cas effector comprises a polypeptide comprising a sequence having at least about 90% identity to SEQ ID NOs: 1, 12, 16, 20-30, 64, 80-85, and 220. In some embodiments, the class 2, type V Cas effector comprises a polypeptide comprising a sequence having at least about 91% identity to SEQ ID NOs: 1, 12, 16, 20-30, 64, 80-85, and 220. In some embodiments, the class 2, type V Cas effector comprises a polypeptide comprising a sequence having at least about 92% identity to SEQ ID NOs: 1, 12, 16, 20-30, 64, 80-85, and 220. In some embodiments, the class 2, type V Cas effector comprises a polypeptide comprising a sequence having at least about 93% identity to SEQ ID NOs: 1, 12, 16, 20-30, 64, 80-85, and 220. In some embodiments, the class 2, type V Cas effector comprises a polypeptide comprising a sequence having at least about 94% identity to SEQ ID NOs: 1, 12, 16, 20-30, 64, 80-85, and 220. In some embodiments, the class 2, type V Cas effector comprises a polypeptide comprising a sequence having at least about 95% identity to SEQ ID NOs: 1, 12, 16, 20-30, 64, 80-85, and 220. In some embodiments, the class 2, type V Cas effector comprises a polypeptide comprising a sequence having at least about 96% identity to SEQ ID NOs: 1, 12, 16, 20-30, 64, 80-85, and 220. In some embodiments, the class 2, type V Cas effector comprises a polypeptide comprising a sequence having at least about 97% identity to SEQ ID NOs: 1, 12, 16, 20-30, 64, 80-85, and 220. In some embodiments, the class 2, type V Cas effector comprises a polypeptide comprising a sequence having at least about 98% identity to SEQ ID NOs: 1, 12, 16, 20-30, 64, 80-85, and 220. In some embodiments, the class 2, type V Cas effector comprises a polypeptide comprising a sequence having at least about 99% identity to SEQ ID NOs: 1, 12, 16, 20-30, 64, 80-85, and 220. In some embodiments, the class 2, type V Cas effector comprises a polypeptide comprising a sequence having 100% identity to SEQ ID NOs: 1, 12, 16, 20-30, 64, 80-85, and 220.

In some embodiments, the TnsB subunit comprises a polypeptide having a sequence having at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, or at least about 99% identity to SEQ ID NO: 2, 13, 17, and 65. In some embodiments, the TnsB subunit comprises a polypeptide having a sequence identical to SEQ ID NO: 2, 13, 17, and 65. In some embodiments, the TnsB component comprises a polypeptide comprising a sequence having at least about 70% identity to SEQ ID NOs: 2, 13, 17, and 65. In some embodiments, the TnsB component comprises a polypeptide comprising a sequence having at least about 75% identity to SEQ ID NOs: 2, 13, 17, and 65. In some embodiments, the TnsB component comprises a polypeptide comprising a sequence having at least about 80% identity to SEQ ID NOs: 2, 13, 17, and 65. In some embodiments, the TnsB component comprises a polypeptide comprising a sequence having at least about 85% identity to SEQ ID NOs: 2, 13, 17, and 65. In some embodiments, the TnsB component comprises a polypeptide comprising a sequence having at least about 90% identity to SEQ ID NOs: 2, 13, 17, and 65. In some embodiments, the TnsB component comprises a polypeptide comprising a sequence having at least about 91% identity to SEQ ID NOs: 2, 13, 17, and 65. In some embodiments, the TnsB component comprises a polypeptide comprising a sequence having at least about 92% identity to SEQ ID NOs: 2, 13, 17, and 65. In some embodiments, the TnsB component comprises a polypeptide comprising a sequence having at least about 93% identity to SEQ ID NOs: 2, 13, 17, and 65. In some embodiments, the TnsB component comprises a polypeptide comprising a sequence having at least about 94% identity to SEQ ID NOs: 2, 13, 17, and 65. In some embodiments, the TnsB component comprises a polypeptide comprising a sequence having at least about 95% identity to SEQ ID NOs: 2, 13, 17, and 65. In some embodiments, the TnsB component comprises a polypeptide comprising a sequence having at least about 96% identity to SEQ ID NOs: 2, 13, 17, and 65. In some embodiments, the TnsB component comprises a polypeptide comprising a sequence having at least about 97% identity to SEQ ID NOs: 2, 13, 17, and 65. In some embodiments, the TnsB component comprises a polypeptide comprising a sequence having at least about 98% identity to SEQ ID NOs: 2, 13, 17, and 65. In some embodiments, the TnsB component comprises a polypeptide comprising a sequence having at least about 99% identity to SEQ ID NOs: 2, 13, 17, and 65. In some embodiments, the TnsB component comprises a polypeptide comprising a sequence having 100% identity to SEQ ID NOs: 2, 13, 17, and 65.

In some embodiments, the functional domain (FD)-TniQ fusion comprises a polypeptide having a sequence having at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, or at least about 99% identity to SEQ ID NO: 257-307 and 1138-1242. In some embodiments, the FD-TniQ fusion comprises a polypeptide having a sequence identical to SEQ ID NO: 257-307 and 1138-1242. In some embodiments, the FD-TniQ fusion comprises a polypeptide comprising a sequence having at least about 70% identity to SEQ ID NOs: 257-307 and 1138-1242. In some embodiments, the FD-TniQ fusion comprises a polypeptide comprising a sequence having at least about 75% identity to SEQ ID NOs: 257-307 and 1138-1242. In some embodiments, the FD-TniQ fusion comprises a polypeptide comprising a sequence having at least about 80% identity to SEQ ID NOs: 257-307 and 1138-1242. In some embodiments, the FD-TniQ fusion comprises a polypeptide comprising a sequence having at least about 85% identity to SEQ ID NOs: 257-307 and 1138-1242. In some embodiments, the FD-TniQ fusion comprises a polypeptide comprising a sequence having at least about 90% identity to SEQ ID NOs: 257-307 and 1138-1242. In some embodiments, the FD-TniQ fusion comprises a polypeptide comprising a sequence having at least about 91% identity to SEQ ID NOs: 257-307 and 1138-1242. In some embodiments, the FD-TniQ fusion comprises a polypeptide comprising a sequence having at least about 92% identity to SEQ ID NOs: 257-307 and 1138-1242. In some embodiments, the FD-TniQ fusion comprises a polypeptide comprising a sequence having at least about 93% identity to SEQ ID NOs: 257-307 and 1138-1242. In some embodiments, the FD-TniQ fusion comprises a polypeptide comprising a sequence having at least about 94% identity to SEQ ID NOs: 257-307 and 1138-1242. In some embodiments, the FD-TniQ fusion comprises a polypeptide comprising a sequence having at least about 95% identity to SEQ ID NOs: 257-307 and 1138-1242. In some embodiments, the FD-TniQ fusion comprises a polypeptide comprising a sequence having at least about 96% identity to SEQ ID NOs: 257-307 and 1138-1242. In some embodiments, the FD-TniQ fusion comprises a polypeptide comprising a sequence having at least about 97% identity to SEQ ID NOs: 257-307 and 1138-1242. In some embodiments, the FD-TniQ fusion comprises a polypeptide comprising a sequence having at least about 98% identity to SEQ ID NOs: 257-307 and 1138-1242. In some embodiments, the FD-TniQ fusion comprises a polypeptide comprising a sequence having at least about 99% identity to SEQ ID NOs: 257-307 and 1138-1242. In some embodiments, the FD-TniQ fusion comprises a polypeptide comprising a sequence having 100% identity to SEQ ID NOs: 257-307 and 1138-1242.

In some embodiments, the Tn7 type transposase complex comprises at least one polypeptide comprising a sequence having at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, or at least about 99% identity to any one of SEQ ID NOs: 3-4, 14-15, 18-19, 66-67, and 109-111. In some embodiments, the Tn7 type transposase complex comprises a polypeptide comprising a sequence having at least about 70% identity to SEQ ID NOs: 3-4, 14-15, 18-19, 66-67, and 109-111. In some embodiments, the Tn7 type transposase complex comprises a polypeptide comprising a sequence having at least about 75% identity to SEQ ID NOs: 3-4, 14-15, 18-19, 66-67, and 109-111. In some embodiments, the Tn7 type transposase complex comprises a polypeptide comprising a sequence having at least about 80% identity to SEQ ID NOs: 3-4, 14-15, 18-19, 66-67, and 109-111. In some embodiments, the Tn7 type transposase complex comprises a polypeptide comprising a sequence having at least about 85% identity to SEQ ID NOs: 3-4, 14-15, 18-19, 66-67, and 109-111. In some embodiments, the Tn7 type transposase complex comprises a polypeptide comprising a sequence having at least about 90% identity to SEQ ID NOs: 3-4, 14-15, 18-19, 66-67, and 109-111. In some embodiments, the Tn7 type transposase complex comprises a polypeptide comprising a sequence having at least about 91% identity to SEQ ID NOs: 3-4, 14-15, 18-19, 66-67, and 109-111. In some embodiments, the Tn7 type transposase complex comprises a polypeptide comprising a sequence having at least about 92% identity to SEQ ID NOs: 3-4, 14-15, 18-19, 66-67, and 109-111. In some embodiments, the Tn7 type transposase complex comprises a polypeptide comprising a sequence having at least about 93% identity to SEQ ID NOs: 3-4, 14-15, 18-19, 66-67, and 109-111. In some embodiments, the Tn7 type transposase complex comprises a polypeptide comprising a sequence having at least about 94% identity to SEQ ID NOs: 3-4, 14-15, 18-19, 66-67, and 109-111. In some embodiments, the Tn7 type transposase complex comprises a polypeptide comprising a sequence having at least about 95% identity to SEQ ID NOs: 3-4, 14-15, 18-19, 66-67, and 109-111. In some embodiments, the Tn7 type transposase complex comprises a polypeptide comprising a sequence having at least about 96% identity to SEQ ID NOs: 3-4, 14-15, 18-19, 66-67, and 109-111. In some embodiments, the Tn7 type transposase complex comprises a polypeptide comprising a sequence having at least about 97% identity to SEQ ID NOs: 3-4, 14-15, 18-19, 66-67, and 109-111. In some embodiments, the Tn7 type transposase complex comprises a polypeptide comprising a sequence having at least about 98% identity to SEQ ID NOs: 3-4, 14-15, 18-19, 66-67, and 109-111. In some embodiments, the Tn7 type transposase complex comprises a polypeptide comprising a sequence having at least about 99% identity to SEQ ID NOs: 3-4, 14-15, 18-19, 66-67, and 109-111. In some embodiments, the Tn7 type transposase complex comprises a polypeptide comprising a sequence having 100% identity to SEQ ID NOs: 3-4, 14-15, 18-19, 66-67, and 109-111.

In some embodiments, the Tn7 type transposase complex comprises at least a first polypeptide and a second polypeptide each independently comprising a sequence having at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, or at least about 99% identity to any one of SEQ ID NOs: 3-4, 14-15, 18-19, 66-67, and 109-111 In some embodiments, the Tn7 type transposase complex comprises at least a first polypeptide and a second polypeptide each independently comprising a sequence with at least 70% sequence identity to any one of SEQ ID NOs: 3-4, 14-15, 18-19, 66-67, and 109-111. In some embodiments, the Tn7 type transposase complex comprises at least a first polypeptide and a second polypeptide each independently comprising a sequence with at least 75% sequence identity to any one of SEQ ID NOs: 3-4, 14-15, 18-19, 66-67, and 109-111. In some embodiments, the Tn7 type transposase complex comprises at least a first polypeptide and a second polypeptide each independently comprising a sequence with at least 80% sequence identity to any one of SEQ ID NOs: 3-4, 14-15, 18-19, 66-67, and 109-111. In some embodiments, the Tn7 type transposase complex comprises at least a first polypeptide and a second polypeptide each independently having at least about 85% identity to SEQ ID NOs: 3-4, 14-15, 18-19, 66-67, and 109-111. In some embodiments, the Tn7 type transposase complex comprises at least a first polypeptide and a second polypeptide each independently having at least about 90% identity to SEQ ID NOs: 3-4, 14-15, 18-19, 66-67, and 109-111. In some embodiments, the Tn7 type transposase complex comprises at least a first polypeptide and a second polypeptide each independently having at least about 91% identity to SEQ ID NOs: 3-4, 14-15, 18-19, 66-67, and 109-111. In some embodiments, the Tn7 type transposase complex comprises at least a first polypeptide and a second polypeptide each independently having at least about 92% identity to SEQ ID NOs: 3-4, 14-15, 18-19, 66-67, and 109-111. In some embodiments, the Tn7 type transposase complex comprises at least a first polypeptide and a second polypeptide each independently having at least about 93% identity to SEQ ID NOs: 3-4, 14-15, 18-19, 66-67, and 109-111. In some embodiments, the Tn7 type transposase complex comprises at least a first polypeptide and a second polypeptide each independently having at least about 94% identity to SEQ ID NOs: 3-4, 14-15, 18-19, 66-67, and 109-111. In some embodiments, the Tn7 type transposase complex comprises at least a first polypeptide and a second polypeptide each independently having at least about 95% identity to SEQ ID NOs: 3-4, 14-15, 18-19, 66-67, and 109-111. In some embodiments, the Tn7 type transposase complex comprises at least a first polypeptide and a second polypeptide each independently having at least about 96% identity to SEQ ID NOs: 3-4, 14-15, 18-19, 66-67, and 109-111. In some embodiments, the Tn7 type transposase complex comprises at least a first polypeptide and a second polypeptide each independently having at least about 97% identity to SEQ ID NOs: 3-4, 14-15, 18-19, 66-67, and 109-111. In some embodiments, the Tn7 type transposase complex comprises at least a first polypeptide and a second polypeptide each independently having at least about 98% identity to SEQ ID NOs: 3-4, 14-15, 18-19, 66-67, and 109-111. In some embodiments, the Tn7 type transposase complex comprises at least a first polypeptide and a second polypeptide each independently having at least about 99% identity to SEQ ID NOs: 3-4, 14-15, 18-19, 66-67, and 109-111. In some embodiments, the Tn7 type transposase complex comprises at least a first polypeptide and a second polypeptide each independently having 100% identity to SEQ ID NOs: 3-4, 14-15, 18-19, 66-67, and 109-111.

In some embodiments, the Tn7 type transposase complex comprises an accessory protein. In some embodiments, the accessory protein is ClpX.

In some embodiments, the accessory protein comprises a sequence having at least 80% sequence identity to any one of SEQ ID NOs: 228-230 and 235-249. In some embodiments, the accessory protein comprises at least one polypeptide comprising a sequence having at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, or at least about 99% identity to any one of SEQ ID NOs: 228-230 and 235-249. In some embodiments, the accessory protein comprises a polypeptide comprising a sequence having at least about 70% identity to SEQ ID NOs: 228-230 and 235-249. In some embodiments, the accessory protein comprises a polypeptide comprising a sequence having at least about 75% identity to SEQ ID NOs: 228-230 and 235-249. In some embodiments, the accessory protein comprises a polypeptide comprising a sequence having at least about 80% identity to SEQ ID NOs: 228-230 and 235-249. In some embodiments, the accessory protein comprises a polypeptide comprising a sequence having at least about 85% identity to SEQ ID NOs: 228-230 and 235-249. In some embodiments, the accessory protein comprises a polypeptide comprising a sequence having at least about 90% identity to SEQ ID NOs: 228-230 and 235-249. In some embodiments, the accessory protein comprises a polypeptide comprising a sequence having at least about 91% identity to SEQ ID NOs: 228-230 and 235-249. In some embodiments, the accessory protein comprises a polypeptide comprising a sequence having at least about 92% identity to SEQ ID NOs: 228-230 and 235-249. In some embodiments, the accessory protein comprises a polypeptide comprising a sequence having at least about 93% identity to SEQ ID NOs: 228-230 and 235-249. In some embodiments, the accessory protein comprises a polypeptide comprising a sequence having at least about 94% identity to SEQ ID NOs: 228-230 and 235-249. In some embodiments, the accessory protein comprises a polypeptide comprising a sequence having at least about 95% identity to SEQ ID NOs: 228-230 and 235-249. In some embodiments, the accessory protein comprises a polypeptide comprising a sequence having at least about 96% identity to SEQ ID NOs: 228-230 and 235-249. In some embodiments, the accessory protein comprises a polypeptide comprising a sequence having at least about 97% identity to SEQ ID NOs: 228-230 and 235-249. In some embodiments, the accessory protein comprises a polypeptide comprising a sequence having at least about 98% identity to SEQ ID NOs: 228-230 and 235-249. In some embodiments, the accessory protein comprises a polypeptide comprising a sequence having at least about 99% identity to SEQ ID NOs: 228-230 and 235-249. In some embodiments, the accessory protein comprises a polypeptide comprising a sequence having 100% identity to SEQ ID NOs: 228-230 and 235-249.

In some embodiments, a system disclosed herein comprises at least one engineered guide polynucleotide, e.g., a gRNA.

In some embodiments, the engineered guide polynucleotide comprises a sequence comprising at least about 46-80 consecutive nucleotides having at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, or at least about 99% identity to any one of SEQ ID NOs: 5-6, 32-33, 94-95, 104-105, 119-122, and 222. In some embodiments, the engineered guide polynucleotide comprises a sequence comprising at least about 46-80 consecutive nucleotides having at least about 70% identity to SEQ ID NOs: 5-6, 32-33, 94-95, 104-105, 119-122, and 222. In some embodiments, the engineered guide polynucleotide comprises a sequence comprising at least about 46-80 consecutive nucleotides having at least about 75% identity to SEQ ID NOs: 5-6, 32-33, 94-95, 104-105, 119-122, and 222. In some embodiments, the engineered guide polynucleotide comprises a sequence comprising at least about 46-80 consecutive nucleotides having at least about 80% identity to SEQ ID NOs: 5-6, 32-33, 94-95, 104-105, 119-122, and 222. In some embodiments, the engineered guide polynucleotide comprises a sequence comprising at least about 46-80 consecutive nucleotides having at least about 85% identity to SEQ ID NOs: 5-6, 32-33, 94-95, 104-105, 119-122, and 222. In some embodiments, the engineered guide polynucleotide comprises a sequence comprising at least about 46-80 consecutive nucleotides having at least about 90% identity to SEQ ID NOs: 5-6, 32-33, 94-95, 104-105, 119-122, and 222. In some embodiments, the engineered guide polynucleotide comprises a sequence comprising at least about 46-80 consecutive nucleotides having at least about 91% identity to SEQ ID NOs: 5-6, 32-33, 94-95, 104-105, 119-122, and 222. In some embodiments, the engineered guide polynucleotide comprises a sequence comprising at least about 46-80 consecutive nucleotides having at least about 92% identity to SEQ ID NOs: 5-6, 32-33, 94-95, 104-105, 119-122, and 222. In some embodiments, the engineered guide polynucleotide comprises a sequence comprising at least about 46-80 consecutive nucleotides having at least about 93% identity to SEQ ID NOs: 5-6, 32-33, 94-95, 104-105, 119-122, and 222. In some embodiments, the engineered guide polynucleotide comprises a sequence comprising at least about 46-80 consecutive nucleotides having at least about 94% identity to SEQ ID NOs: 5-6, 32-33, 94-95, 104-105, 119-122, and 222. In some embodiments, the engineered guide polynucleotide comprises a sequence comprising at least about 46-80 consecutive nucleotides having at least about 95% identity to SEQ ID NOs: 5-6, 32-33, 94-95, 104-105, 119-122, and 222. In some embodiments, the engineered guide polynucleotide comprises a sequence comprising at least about 46-80 consecutive nucleotides having at least about 96% identity to SEQ ID NOs: 5-6, 32-33, 94-95, 104-105, 119-122, and 222. In some embodiments, the engineered guide polynucleotide comprises a sequence comprising at least about 46-80 consecutive nucleotides having at least about 97% identity to SEQ ID NOs: 5-6, 32-33, 94-95, 104-105, 119-122, and 222. In some embodiments, the engineered guide polynucleotide comprises a sequence comprising at least about 46-80 consecutive nucleotides having at least about 98% identity to SEQ ID NOs: 5-6, 32-33, 94-95, 104-105, 119-122, and 222. In some embodiments, the engineered guide polynucleotide comprises a sequence comprising at least about 46-80 consecutive nucleotides having at least about 99% identity to SEQ ID NOs: 5-6, 32-33, 94-95, 104-105, 119-122, and 222. In some embodiments, the engineered guide polynucleotide comprises a sequence comprising at least about 46-80 consecutive nucleotides having 100% identity to SEQ ID NOs: 5-6, 32-33, 94-95, 104-105, 119-122, and 222.

In some embodiments, the engineered guide polynucleotide is a guide RNA and comprises a sequence comprising at least about 46-80 consecutive nucleotides having at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, or at least about 99% identity to any one of SEQ ID NOs: 45-63, 68-75, 96-103, 123-140, and 185. In some embodiments, the engineered guide polynucleotide is a guide RNA and comprises a sequence comprising at least about 46-80 consecutive nucleotides identical to any one of SEQ ID NOs: 45-63, 68-75, 96-103, 123-140, and 185. In some embodiments, the engineered guide polynucleotide is a guide RNA and comprises a sequence comprising at least about 46-80 consecutive nucleotides having at least about 70% identity to SEQ ID NOs: 45-63, 68-75, 96-103, 123-140, and 185. In some embodiments, the engineered guide polynucleotide is a guide RNA and comprises a sequence comprising at least about 46-80 consecutive nucleotides having at least about 75% identity to SEQ ID NOs: 45-63, 68-75, 96-103, 123-140, and 185. In some embodiments, the engineered guide polynucleotide is a guide RNA and comprises a sequence comprising at least about 46-80 consecutive nucleotides having at least about 80% identity to SEQ ID NOs: 45-63, 68-75, 96-103, 123-140, and 185. In some embodiments, the engineered guide polynucleotide is a guide RNA and comprises a sequence comprising at least about 46-80 consecutive nucleotides having at least about 85% identity to SEQ ID NOs: 45-63, 68-75, 96-103, 123-140, and 185. In some embodiments, the engineered guide polynucleotide is a guide RNA and comprises a sequence comprising at least about 46-80 consecutive nucleotides having at least about 90% identity to SEQ ID NOs: 45-63, 68-75, 96-103, 123-140, and 185. In some embodiments, the engineered guide polynucleotide is a guide RNA and comprises a sequence comprising at least about 46-80 consecutive nucleotides having at least about 91% identity to SEQ ID NOs: 45-63, 68-75, 96-103, 123-140, and 185. In some embodiments, the engineered guide polynucleotide is a guide RNA and comprises a sequence comprising at least about 46-80 consecutive nucleotides having at least about 92% identity to SEQ ID NOs: 45-63, 68-75, 96-103, 123-140, and 185. In some embodiments, the engineered guide polynucleotide is a guide RNA and comprises a sequence comprising at least about 46-80 consecutive nucleotides having at least about 93% identity to SEQ ID NOs: 45-63, 68-75, 96-103, 123-140, and 185. In some embodiments, the engineered guide polynucleotide is a guide RNA and comprises a sequence comprising at least about 46-80 consecutive nucleotides having at least about 94% identity to SEQ ID NOs: 45-63, 68-75, 96-103, 123-140, and 185. In some embodiments, the engineered guide polynucleotide is a guide RNA and comprises a sequence comprising at least about 46-80 consecutive nucleotides having at least about 95% identity to SEQ ID NOs: 45-63, 68-75, 96-103, 123-140, and 185. In some embodiments, the engineered guide polynucleotide is a guide RNA and comprises a sequence comprising at least about 46-80 consecutive nucleotides having at least about 96% identity to SEQ ID NOs: 45-63, 68-75, 96-103, 123-140, and 185. In some embodiments, the engineered guide polynucleotide is a guide RNA and comprises a sequence comprising at least about 46-80 consecutive nucleotides having at least about 97% identity to SEQ ID NOs: 45-63, 68-75, 96-103, 123-140, and 185. In some embodiments, the engineered guide polynucleotide is a guide RNA and comprises a sequence comprising at least about 46-80 consecutive nucleotides having at least about 98% identity to SEQ ID NOs: 45-63, 68-75, 96-103, 123-140, and 185. In some embodiments, the engineered guide polynucleotide is a guide RNA and comprises a sequence comprising at least about 46-80 consecutive nucleotides having at least about 99% identity to SEQ ID NOs: 45-63, 68-75, 96-103, 123-140, and 185. In some embodiments, the engineered guide polynucleotide is a guide RNA and comprises a sequence comprising at least about 46-80 consecutive nucleotides having 100% identity to SEQ ID NOs: 45-63, 68-75, 96-103, 123-140, and 185.

In some embodiments, the engineered guide polynucleotide is a guide RNA and comprises a sequence comprising at least about 46-80 consecutive nucleotides having at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, or at least about 99% identity to any one of SEQ ID NOs: 106, 107, 108, 5, 45-63, 68-75, 96-103, 123-140, and 754-944. In some embodiments, the engineered guide polynucleotide is a guide RNA and comprises a sequence comprising at least about 46-80 consecutive nucleotides identical to any one of SEQ ID NOs: 106, 107, 108, 5, 45-63, 68-75, 96-103, 123-140, and 754-944. In some embodiments, the engineered guide polynucleotide is a guide RNA and comprises a sequence comprising at least about 46-80 consecutive nucleotides having at least about 70% identity to SEQ ID NOs: 106, 107, 108, 5, 45-63, 68-75, 96-103, 123-140, and 754-944. In some embodiments, the engineered guide polynucleotide is a guide RNA and comprises a sequence comprising at least about 46-80 consecutive nucleotides having at least about 75% identity to SEQ ID NOs: 106, 107, 108, 5, 45-63, 68-75, 96-103, 123-140, and 754-944. In some embodiments, the engineered guide polynucleotide is a guide RNA and comprises a sequence comprising at least about 46-80 consecutive nucleotides having at least about 80% identity to SEQ ID NOs: 106, 107, 108, 5, 45-63, 68-75, 96-103, 123-140, and 754-944. In some embodiments, the engineered guide polynucleotide is a guide RNA and comprises a sequence comprising at least about 46-80 consecutive nucleotides having at least about 85% identity to SEQ ID NOs: 106, 107, 108, 5, 45-63, 68-75, 96-103, 123-140, and 754-944. In some embodiments, the engineered guide polynucleotide is a guide RNA and comprises a sequence comprising at least about 46-80 consecutive nucleotides having at least about 90% identity to SEQ ID NOs: 106, 107, 108, 5, 45-63, 68-75, 96-103, 123-140, and 754-944. In some embodiments, the engineered guide polynucleotide is a guide RNA and comprises a sequence comprising at least about 46-80 consecutive nucleotides having at least about 91% identity to SEQ ID NOs: 106, 107, 108, 5, 45-63, 68-75, 96-103, 123-140, and 754-944. In some embodiments, the engineered guide polynucleotide is a guide RNA and comprises a sequence comprising at least about 46-80 consecutive nucleotides having at least about 92% identity to SEQ ID NOs: 106, 107, 108, 5, 45-63, 68-75, 96-103, 123-140, and 754-944. In some embodiments, the engineered guide polynucleotide is a guide RNA and comprises a sequence comprising at least about 46-80 consecutive nucleotides having at least about 93% identity to SEQ ID NOs: 106, 107, 108, 5, 45-63, 68-75, 96-103, 123-140, and 754-944. In some embodiments, the engineered guide polynucleotide is a guide RNA and comprises a sequence comprising at least about 46-80 consecutive nucleotides having at least about 94% identity to SEQ ID NOs: 106, 107, 108, 5, 45-63, 68-75, 96-103, 123-140, and 754-944. In some embodiments, the engineered guide polynucleotide is a guide RNA and comprises a sequence comprising at least about 46-80 consecutive nucleotides having at least about 95% identity to SEQ ID NOs: 106, 107, 108, 5, 45-63, 68-75, 96-103, 123-140, and 754-944. In some embodiments, the engineered guide polynucleotide is a guide RNA and comprises a sequence comprising at least about 46-80 consecutive nucleotides having at least about 96% identity to SEQ ID NOs: 106, 107, 108, 5, 45-63, 68-75, 96-103, 123-140, and 754-944. In some embodiments, the engineered guide polynucleotide is a guide RNA and comprises a sequence comprising at least about 46-80 consecutive nucleotides having at least about 97% identity to SEQ ID NOs: 106, 107, 108, 5, 45-63, 68-75, 96-103, 123-140, and 754-944. In some embodiments, the engineered guide polynucleotide is a guide RNA and comprises a sequence comprising at least about 46-80 consecutive nucleotides having at least about 98% identity to SEQ ID NOs: 106, 107, 108, 5, 45-63, 68-75, 96-103, 123-140, and 754-944. In some embodiments, the engineered guide polynucleotide is a guide RNA and comprises a sequence comprising at least about 46-80 consecutive nucleotides having at least about 99% identity to SEQ ID NOs: 106, 107, 108, 5, 45-63, 68-75, 96-103, 123-140, and 754-944. In some embodiments, the engineered guide polynucleotide is a guide RNA and comprises a sequence comprising at least about 46-80 consecutive nucleotides having 100% identity to SEQ ID NOs: 106, 107, 108, 5, 45-63, 68-75, 96-103, 123-140, and 754-944.

In some embodiments, the engineered guide polynucleotide is a guide RNA and comprises a sequence having at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, or at least about 99% identity to any one of SEQ ID NOs: 106, 107, 108, 5, 45-63, 68-75, 96-103, 123-140, and 754-944. In some embodiments, the engineered guide polynucleotide is a guide RNA comprises a sequence identical to any one of SEQ ID NOs: 106, 107, 108, 5, 45-63, 68-75, 96-103, 123-140, and 754-944. In some embodiments, the engineered guide polynucleotide is a guide RNA comprises a sequence having at least about 70% identity to SEQ ID NOs: 106, 107, 108, 5, 45-63, 68-75, 96-103, 123-140, and 754-944. In some embodiments, the engineered guide polynucleotide is a guide RNA comprises a sequence having at least about 75% identity to SEQ ID NOs: 106, 107, 108, 5, 45-63, 68-75, 96-103, 123-140, and 754-944. In some embodiments, the engineered guide polynucleotide is a guide RNA comprises a sequence having at least about 80% identity to SEQ ID NOs: 106, 107, 108, 5, 45-63, 68-75, 96-103, 123-140, and 754-944. In some embodiments, the engineered guide polynucleotide is a guide RNA comprises a sequence having at least about 85% identity to SEQ ID NOs: 106, 107, 108, 5, 45-63, 68-75, 96-103, 123-140, and 754-944. In some embodiments, the engineered guide polynucleotide is a guide RNA comprises a sequence having at least about 90% identity to SEQ ID NOs: 106, 107, 108, 5, 45-63, 68-75, 96-103, 123-140, and 754-944. In some embodiments, the engineered guide polynucleotide is a guide RNA comprises a sequence having at least about 91% identity to SEQ ID NOs: 106, 107, 108, 5, 45-63, 68-75, 96-103, 123-140, and 754-944. In some embodiments, the engineered guide polynucleotide is a guide RNA comprises a sequence having at least about 92% identity to SEQ ID NOs: 106, 107, 108, 5, 45-63, 68-75, 96-103, 123-140, and 754-944. In some embodiments, the engineered guide polynucleotide is a guide RNA comprises a sequence having at least about 93% identity to SEQ ID NOs: 106, 107, 108, 5, 45-63, 68-75, 96-103, 123-140, and 754-944. In some embodiments, the engineered guide polynucleotide is a guide RNA comprises a sequence having at least about 94% identity to SEQ ID NOs: 106, 107, 108, 5, 45-63, 68-75, 96-103, 123-140, and 754-944. In some embodiments, the engineered guide polynucleotide is a guide RNA comprises a sequence having at least about 95% identity to SEQ ID NOs: 106, 107, 108, 5, 45-63, 68-75, 96-103, 123-140, and 754-944. In some embodiments, the engineered guide polynucleotide is a guide RNA comprises a sequence having at least about 96% identity to SEQ ID NOs: 106, 107, 108, 5, 45-63, 68-75, 96-103, 123-140, and 754-944. In some embodiments, the engineered guide polynucleotide is a guide RNA comprises a sequence having at least about 97% identity to SEQ ID NOs: 106, 107, 108, 5, 45-63, 68-75, 96-103, 123-140, and 754-944. In some embodiments, the engineered guide polynucleotide is a guide RNA comprises a sequence having at least about 98% identity to SEQ ID NOs: 106, 107, 108, 5, 45-63, 68-75, 96-103, 123-140, and 754-944. In some embodiments, the engineered guide polynucleotide is a guide RNA comprises a sequence having at least about 99% identity to SEQ ID NOs: 106, 107, 108, 5, 45-63, 68-75, 96-103, 123-140, and 754-944. In some embodiments the engineered guide polynucleotide is a guide RNA comprises a sequence having 100% identity to SEQ ID NOs: 106, 107, 108, 5, 45-63, 68-75, 96-103, 123-140, and 754-944.

In some aspects, the present disclosure provides a system for transposing a cargo nucleotide sequence into a target nucleic acid site in a target nucleic acid comprising a Cas effector complex that hybridizes to the target nucleic acid site and comprising: i) a class 2, type V Cas effector comprising a sequence having at least 80% sequence identity to any one of SEQ ID NOs: 1, 81, 82, 83, and 85; and ii) an engineered guide polynucleotide having at least 80% identity to any one of SEQ ID NOs: 5, 6, 45-63, 68-75, 96-103, 123-140, and 754-944: a Tn7 type transposase complex that binds the Cas effector complex and comprises a TnsB, TnsC, and TniQ component, the TnsB, TnsC, or TniQ component comprising a sequence having at least 80% identity to any one of SEQ ID NOs: 2-4, and an accessory protein comprising a sequence having at least 80% sequence identity to any one of SEQ ID NOs: 228-230 and 235-249; and a double-stranded nucleic acid that interacts with the Tn7 type transposase complex and comprising in 5′ to 3′ order: i) a left-hand transposase recognition sequence comprising a sequence having at least 80% sequence identity to any one of SEQ ID NOs: 9, 11, 36, 37, and 38; ii) the cargo nucleotide sequence; and ii) a right-hand transposase recognition sequence comprising a sequence having at least 80% identity to any one of SEQ ID NOs: 8, 39-44, and 93.

In some aspects, the present disclosure provides a system for transposing a cargo nucleotide sequence into a target nucleic acid site in a target nucleic acid comprising: a Cas effector complex that hybridizes to the target nucleic acid site and comprising: i) a class 2, type V Cas effector comprising a sequence having at least 80% sequence identity to SEQ ID NOs: 12; and iii) an engineered guide polynucleotide having at least 80% identity to any one of SEQ ID NOs: 32, 102, 104, and 107; a Tn7 type transposase complex that binds the Cas effector complex and comprising a TnsB, TnsC, and TniQ component, the TnsB, TnsC, or TniQ component comprising a sequence having at least 80% identity to any one of SEQ ID NOs: 13-15, and an accessory protein comprising a sequence having at least 80% sequence identity to any one of SEQ ID NOs: 228-230 and 235-249; and a double-stranded nucleic acid that interacts with the Tn7 type transposase complex and comprising in 5′ to 3′ order: i) a left-hand transposase recognition sequence comprising a sequence having at least 80% sequence identity to SEQ ID NO: 76: ii) the cargo nucleotide sequence; and iii) a right-hand transposase recognition sequence comprising a sequence having at least 80% identity to SEQ ID NO: 77.

In some aspects, the present disclosure provides a system for transposing a cargo nucleotide sequence into a target nucleic acid site in a target nucleic acid comprising: a Cas effector complex that hybridizes to the target nucleic acid site and comprising: i) a class 2, type V Cas effector comprising a sequence having at least 80% sequence identity to SEQ ID NOs: 16; and ii) an engineered guide polynucleotide having at least 80% identity to any one of SEQ ID NOs: 33, 103, 105, and 108; a Tn7 type transposase complex that binds the Cas effector complex and comprises a TnsB, TnsC, and TniQ component, the TnsB, TnsC, or TniQ component comprising a sequence having at least 80% identity to any one of SEQ ID NOs: 17-19, an accessory protein comprising a sequence having at least 80% sequence identity to any one of SEQ ID NOs: 228-230 and 235-249; and a double-stranded nucleic acid that interacts with the Tn7 type transposase complex and comprising in 5′ to 3′ order: i) a left-hand transposase recognition sequence comprising a sequence having at least 80% sequence identity to SEQ ID NO: 78; ii) the cargo nucleotide sequence; and iii) a right-hand transposase recognition sequence comprising a sequence having at least 80% identity to SEQ ID NO: 79.

In some embodiments, the system further comprises a PAM sequence compatible with the Cas effector complex. In some embodiments, the PAM sequence comprises SEQ ID NO: 31.

In some embodiments, the PAM sequence is located about 50 to about 70 base pairs from the target nucleic acid site. In some embodiments, the PAM sequence is located 3′ of the target nucleic acid site. In some embodiments, the PAM sequence is located 5′ of the target nucleic acid site.

In some embodiments, the guide RNAs comprise various structural elements including but not limited to: a spacer sequence which binds to the protospacer sequence (target sequence), a crRNA, and an optional tracrRNA. In some embodiments, the guide RNA comprises a crRNA comprising a spacer sequence. In some embodiments, the guide RNA additionally comprises a tracrRNA or a modified tracrRNA.

In some embodiments, the systems provided herein comprise one or more guide RNAs. In some embodiments, the guide RNA comprises a sense sequence. In some embodiments, the guide RNA comprises an anti-sense sequence. In some embodiments, the guide RNA comprises nucleotide sequences other than the region complementary to or substantially complementary to a region of a target sequence. For example, a crRNA is part or considered part of a guide RNA, or is comprised in a guide RNA, e.g., a crRNA:tracrRNA chimera.

In some embodiments, the guide RNA comprises synthetic nucleotides or modified nucleotides. In some embodiments, the guide RNA comprises one or more inter-nucleoside linkers modified from the natural phosphodiester. In some embodiments, all of the inter-nucleoside linkers of the guide RNA, or contiguous nucleotide sequence thereof, are modified. For example, in some embodiments, the inter nucleoside linkage comprises Sulphur (S), such as a phosphorothioate inter-nucleoside linkage.

In some embodiments, the guide RNA comprises modifications to a ribose sugar or nucleobase. In some embodiments, the guide RNA comprises one or more nucleosides comprising a modified sugar moiety, wherein the modified sugar moiety is a modification of the sugar moiety when compared to the ribose sugar moiety found in deoxyribose nucleic acid (DNA) and RNA. In some embodiments, the modification is within the ribose ring structure. Exemplary modifications include, but are not limited to, replacement with a hexose ring (HNA), a bicyclic ring having a biradical bridge between the C2 and C4 carbons on the ribose ring (e.g., locked nucleic acids (LNA)), or an unlinked ribose ring which typically lacks a bond between the C2 and C3 carbons (e.g., UNA). In some embodiments, the sugar-modified nucleosides comprise bicyclohexose nucleic acids or tricyclic nucleic acids. In some embodiments, the modified nucleosides comprise nucleosides where the sugar moiety is replaced with a non-sugar moiety, for example peptide nucleic acids (PNA) or morpholino nucleic acids.

In some embodiments, the guide RNA comprises one or more modified sugars. In some embodiments, the sugar modifications comprise modifications made by altering the substituent groups on the ribose ring to groups other than hydrogen, or the 2′-OH group naturally found in DNA and RNA nucleosides. In some embodiments, substituents are introduced at the 2′, 3′, 4′ or 5′ positions, or combinations thereof. In some embodiments, nucleosides with modified sugar moieties comprise 2′ modified nucleosides, e.g., 2′ substituted nucleosides. A 2′ sugar modified nucleoside, in some embodiments, is a nucleoside that has a substituent other than —H or —OH at the 2′ position (2′ substituted nucleoside) or comprises a 2′ linked biradical, and comprises 2′ substituted nucleosides and LNA (2′-4′ biradical bridged) nucleosides. Examples of 2′-substituted modified nucleosides comprise, but are not limited to, 2′-O-alkyl-RNA, 2′-O-methyl-RNA, 2′-alkoxy-RNA, 2′-O-methoxyethyl-RNA (MOE), 2′-amino-DNA, 2′-Fluoro-RNA, and 2′-F-ANA nucleosides. In some embodiments, the modification in the ribose group comprises a modification at the 2′ position of the ribose group. In some embodiments, the modification at the 2′ position of the ribose group is selected from the group consisting of 2′-O-methyl, 2′-fluoro, 2′-deoxy, and 2′-O-(2-methoxyethyl).

In some embodiments, the guide RNA comprises one or more modified sugars. In some embodiments, the guide RNA comprises only modified sugars. In certain embodiments, the guide RNA comprises greater than about 10%, 25%, 50%, 75%, or 90% modified sugars. In some embodiments, the modified sugar is a bicyclic sugar. In some embodiments, the modified sugar comprises a 2′-O-methoxyethyl group. In some embodiments, the guide RNA comprises both inter-nucleoside linker modifications and nucleoside modifications.

In some embodiments, the guide RNA comprises a sequence complementary to a eukaryotic, fungal, plant, mammalian, or human genomic polynucleotide sequence. In some embodiments, the guide RNA comprises a sequence complementary to a eukaryotic genomic polynucleotide sequence. In some embodiments, the guide RNA comprises a sequence complementary to a fungal genomic polynucleotide sequence. In some embodiments, the guide RNA comprises a sequence complementary to a plant genomic polynucleotide sequence. In some embodiments, the guide RNA comprises a sequence complementary to a mammalian genomic polynucleotide sequence. In some embodiments, the guide RNA comprises a sequence complementary to a human genomic polynucleotide sequence.

In some embodiments, the guide RNA is 30-250 nucleotides in length. In some embodiments, the guide RNA is more than 90 nucleotides in length. In some embodiments, the guide RNA is less than 245 nucleotides in length. In some embodiments, the guide RNA is 30, 40, 50, 60, 70, 80, 90, 100, 120, 140, 160, 180, 200, 220, 240, or more than 240 nucleotides in length. In some embodiments, the guide RNA is about 30 to about 40, about 30 to about 50, about 30 to about 60, about 30 to about 70, about 30 to about 80, about 30 to about 90, about 30 to about 100, about 30 to about 120, about 30 to about 140, about 30 to about 160, about 30 to about 180, about 30 to about 200, about 30 to about 220, about 30 to about 240, about 50 to about 60, about 50 to about 70, about 50 to about 80, about 50 to about 90, about 50 to about 100, about 50 to about 120, about 50 to about 140, about 50 to about 160, about 50 to about 180, about 50 to about 200, about 50 to about 220, about 50 to about 240, about 100 to about 120, about 100 to about 140, about 100 to about 160, about 100 to about 180, about 100 to about 200, about 100 to about 220, about 100 to about 240, about 160 to about 180, about 160 to about 200, about 160 to about 220, or about 160 to about 240 nucleotides in length.

In some embodiments, the left-hand transposase recognition sequence comprises a sequence having at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, or at least about 99% identity to SEQ ID NO: 9, 11, 36-38, 76, and 78. In some embodiments, the left-hand transposase recognition sequence comprises a sequence having at least about 70% identity to SEQ ID NOs: 9, 11, 36-38, 76, and 78. In some embodiments, the left-hand transposase recognition sequence comprises a sequence having at least about 75% identity to SEQ ID NOs: 9, 11, 36-38, 76, and 78. In some embodiments, the left-hand transposase recognition sequence comprises a sequence having at least about 80% identity to SEQ ID NOs: 9, 11, 36-38, 76, and 78. In some embodiments, the left-hand transposase recognition sequence comprises a sequence having at least about 85% identity to SEQ ID NOs: 9, 11, 36-38, 76, and 78. In some embodiments, the left-hand transposase recognition sequence comprises a sequence having at least about 90% identity to SEQ ID NOs: 9, 11, 36-38, 76, and 78. In some embodiments, the left-hand transposase recognition sequence comprises a sequence having at least about 91% identity to SEQ ID NOs: 9. It. 36-38, 76, and 78. In some embodiments, the left-hand transposase recognition sequence comprises a sequence having at least about 92% identity to SEQ ID NOs: 9, 11, 36-38, 76, and 78. In some embodiments, the left-hand transposase recognition sequence comprises a sequence having at least about 93% identity to SEQ ID NOs: 9, 11, 36-38, 76, and 78. In some embodiments, the left-hand transposase recognition sequence comprises a sequence having at least about 94% identity to SEQ ID NOs: 9, 11, 36-38, 76, and 78. In some embodiments, the left-hand transposase recognition sequence comprises a sequence having at least about 95% identity to SEQ ID NOs: 9, 11, 36-38, 76, and 78. In some embodiments, the left-hand transposase recognition sequence comprises a sequence having at least about 96% identity to SEQ ID NOs: 9, 11, 36-38, 76, and 78. In some embodiments, the left-hand transposase recognition sequence comprises a sequence having at least about 97% identity to SEQ ID NOs: 9, 11, 36-38, 76, and 78. In some embodiments, the left-hand transposase recognition sequence comprises a sequence having at least about 98% identity to SEQ ID NOs: 9, 11, 36-38, 76, and 78. In some embodiments, the left-hand transposase recognition sequence comprises a sequence having at least about 99% identity to SEQ ID NOs: 9, 11, 36-38, 76, and 78. In some embodiments, the left-hand transposase recognition sequence comprises a sequence having 100% identity to SEQ ID NOs: 9, 11, 36-38, 76, and 78.

In some embodiments, the right-hand transposase recognition sequence comprises a sequence having at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, or at least about 99% identity to SEQ ID NO: 8, 10, 39-44, 77, 79, and 93 In some embodiments, the right-hand transposase recognition sequence comprises a sequence having at least about 70% identity to SEQ ID NOs: 8, 10, 39-44, 77, 79, and 93. In some embodiments, the right-hand transposase recognition sequence comprises a sequence having at least about 75% identity to SEQ ID NOs: 8, 10, 39-44, 77, 79, and 93. In some embodiments, the right-hand transposase recognition sequence comprises a sequence having at least about 80% identity to SEQ ID NOs: 8, 10, 39-44, 77, 79, and 93. In some embodiments, the right-hand transposase recognition sequence comprises a sequence having at least about 85% identity to SEQ ID NOs: 8, 10, 39-44, 77, 79, and 93. In some embodiments, the right-hand transposase recognition sequence comprises a sequence having at least about 90% identity to SEQ ID NOs: 8, 10, 39-44, 77, 79, and 93. In some embodiments, the right-hand transposase recognition sequence comprises a sequence having at least about 91% identity to SEQ ID NOs: 8, 10, 39-44, 77, 79, and 93. In some embodiments, the right-hand transposase recognition sequence comprises a sequence having at least about 92% identity to SEQ ID NOs: 8, 10, 39-44, 77, 79, and 93. In some embodiments, the right-hand transposase recognition sequence comprises a sequence having at least about 93% identity to SEQ ID NOs: 8, 10, 39-44, 77, 79, and 93. In some embodiments, the right-hand transposase recognition sequence comprises a sequence having at least about 94% identity to SEQ ID NOs: 8, 10, 39-44, 77, 79, and 93. In some embodiments, the right-hand transposase recognition sequence comprises a sequence having at least about 95% identity to SEQ ID NOs: 8, 10, 39-44, 77, 79, and 93. In some embodiments, the right-hand transposase recognition sequence comprises a sequence having at least about 96% identity to SEQ ID NOs: 8, 10, 39-44, 77, 79, and 93. In some embodiments, the right-hand transposase recognition sequence comprises a sequence having at least about 97% identity to SEQ ID NOs: 8, 10, 39-44, 77, 79, and 93. In some embodiments, the right-hand transposase recognition sequence comprises a sequence having at least about 98% identity to SEQ ID NOs: 8, 10, 39-44, 77, 79, and 93. In some embodiments, the right-hand transposase recognition sequence comprises a sequence having at least about 99% identity to SEQ ID NOs: 8, 10, 39-44, 77, 79, and 93. In some embodiments, the right-hand transposase recognition sequence comprises a sequence having 100% identity to SEQ ID NOs: 8, 10, 39-44, 77, 79, and 93.

In some embodiments, the class 2, type V Cas effector and the Tn7 type transposase complex are encoded by polynucleotide sequences comprising fewer than about 20 kilobases, fewer than about 15 kilobases, fewer than about 10 kilobases, or fewer than about 5 kilobases.

In some embodiments, the class 2, type V effector comprises a nuclear localization sequence (NLS). In some embodiments, the NLS is at an N-terminus of the class 2, type V effector. In some embodiments, the NLS is at a C-terminus of the class 2, type V effector. In some embodiments, the NLS is at an N-terminus and a C-terminus of the class 2, type V effector.

In some embodiments, the NLS comprises a sequence of any one of SEQ ID NOs: 192-207 and 1354-1383, or a sequence having at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, or at least about 99% identity to any one of SEQ ID NOs: 192-207 and 1354-1383. In some embodiments, the NLS comprises a sequence having at least about 80% identity to SEQ ID NOs: 192-207 and 1354-1383. In some embodiments, the NLS comprises a sequence having at least about 85% identity to SEQ ID NOs: 192-207 and 1354-1383. In some embodiments, the NLS comprises a sequence having at least about 90% identity to SEQ ID NOs: 192-207 and 1354-1383. In some embodiments, the NLS comprises a sequence having at least about 91% identity to SEQ ID NOs: 192-207 and 1354-1383. In some embodiments, the NLS comprises a sequence having at least about 92% identity to SEQ ID NOs: 192-207 and 1354-1383. In some embodiments, the NLS comprises a sequence having at least about 93% identity to SEQ ID NOs: 192-207 and 1354-1383. In some embodiments, the NLS comprises a sequence having at least about 94% identity to SEQ ID NOs: 192-207 and 1354-1383. In some embodiments, the NLS comprises a sequence having at least about 95% identity to SEQ ID NOs: 192-207 and 1354-1383. In some embodiments, the NLS comprises a sequence having at least about 96% identity to SEQ ID NOs: 192-207 and 1354-1383. In some embodiments, the NLS comprises a sequence having at least about 97% identity to SEQ ID NOs: 192-207 and 1354-1383. In some embodiments, the NLS comprises a sequence having at least about 98% identity to SEQ ID NOs: 192-207 and 1354-1383. In some embodiments, the NLS comprises a sequence having at least about 99% identity to SEQ ID NOs: 192-207 and 1354-1383. In some embodiments, the NLS comprises a sequence having 100% identity to SEQ ID NOs: 192-207 and 1354-1383.

TABLE 1
Exemplary NLS Sequences

| Source | NLS amino acid sequence | SEQ ID NO: |
|---|---|---|
| SV40 | PKKKRKV | 192 |
| nucleoplasmin | KRPAATKKAGQAKKKK | 193 |
| bipartite NLS | | |
| c-myc NLS | PAAKRVKLD | 194 |
| c-myc NLS | RQRRNELKRSP | 195 |
| hRNPA1 M9 NLS | NQSSNFGPMKGGNFGGRSSGPYGGGGQYFAKPRNQGGY | 196 |
| Importin-alpha | RMRIZFKNKGKDTAELRRRRVEVSVELRKAKKDEQILKRRNV | 197 |
| IBB domain | | |
| Myoma T protein | VSRKRPRP | 198 |
| Myoma T protein | PPKKARED | 199 |
| p53 | PQPKKKPL | 200 |
| mouse c-abl IV | SALIKKKKKMAP | 201 |
| influenza virus | DRLRR | 202 |
| NS1 | | |
| influenza virus | PKQKKRK | 203 |
| NS1 | | |
| Hepatitis virus | RKLKKKIKKL | 204 |
| delta antigen | | |
| mouse Mx1 | REKKKFLKRR | 205 |
| protein | | |
| human poly(ADP- | KRKGDEVDGVDEVAKKKSKK | 206 |
| ribose) polymerase | | |
| steroid hormone | RKCLQAGMNLEARKTKK | 207 |
| receptors (human) | | |
| glucocorticoid | | |
| 5′ SV40 NLS | MAPKKKRKVGGGGS | 1354 |
| 3′ SV40 1 NLS | SGGAPKKKRKV | 1355 |
| 3′ SV40 2 NLS | SGGAPKKKRKV | 1356 |
| 3′ SV40 3 NLS | SGGAPKKKRKVSGGAPKKKRKVSGGAPKKKRKV | 1357 |
| 5′ Nucleoplasmin | MKRPAATKKAGQAKKKKGGGGS | 1358 |
| NLS | | |
| 3′ Nucleoplasmin 1 | SGGKRPAATKKAGQAKKKK | 1359 |
| NLS | | |
| 3′ Nucleoplasmin 2 | SGGKRPAATKKAGQAKKKKSGGKRPAATKKAGQAKKKK | 1360 |
| NLS | | |
| 3′ Nucleoplasmin 3 | SGGKRPAATKKAGQAKKKKSGGKRPAATKKAGQAKKK | 1361 |
| NLS | KSGGKRPAATKKAGQAKKKK | |
| 5′ c-Myc NLS | MPAAKRVKLDGGGGS | 1362 |
| 3′ c-Myc 1 NLS | SGGPAAKRVKLD | 1363 |
| 3′ c-Myc 2 NLS | SGGPAAKRVKLDSGGPAAKRVKLD | 1364 |
| 3′ c-Myc 3 NLS | SGGPAAKRVKLDSGGPAAKRVKLDSGGPAAKRVKLD | 1365 |
| Synthetic 5′ Class | MQAAKRPRTTGGGGS | 1366 |
| 2-1 NLS | | |
| Synthetic 3′ Class | SGGQAAKRPRTT | 1367 |
| 2-1 1 NLS | | |
| Synthetic 3′ Class | SGGQAAKRPRTTSGGQAAKRPRTT | 1368 |
| 2-1 2 NLS | | |
| Synthetic 5′ Class | MRAAKRPRTTGGGGS | 1369 |
| 2-2 NLS | | |
| Synthetic 3′ Class | SGGRAAKRPRTT | 1370 |
| 2-2 1 NLS | | |
| Synthetic 3′ Class | SGGRAAKRPRTTSGGRAAKRPRTT | 1371 |
| 2-2 2 NLS | | |
| Synthetic 5′ Class | MRAAKRPRTTGGGGS | 1372 |
| 2-2 NLS | | |
| Synthetic 3′ Class | SGGRAAKRPRTT | 1373 |
| 2-2 1 NLS | | |
| Synthetic 3′ Class | SGGRAAKRPRTTSGGRAAKRPRTT | 1374 |
| 2-2 2 NLS | | |
| Synthetic 5′ Class | MAAAKRSWSMAFGGGGS | 1375 |
| 3 NLS | | |
| Synthetic 3′ Class | MSGGAAAKRSWSMAF | 1376 |
| 3 1 NLS | | |
| Synthetic 3′ Class | MSGGAAAKRSWSMAFMSGGAAAKRSWSMAF | 1377 |
| 3 2 NLS | | |
| Synthetic 5′ Class | MRAAKRKYFAVGGGGS | 1378 |
| 4 NLS | | |
| Synthetic 3′ Class | MSGGRAAKRKYFAV | 1379 |
| 41 NLS | | |
| Synthetic 3′ Class | MSGGRAAKRKYFAVMSGGRAAKRKYFAV | 1380 |
| 42 NLS | | |
| 5′ HCV NLS | MPKPQRKTKRGGGGS | 1381 |
| 3′ HCV NLS | SGGPPRKKRTVV | 1382 |
| 3′ hRNP A1 NLS | SGGFGNYNNQSSNFGPMKGGNFGGRSSGPY | 1383 |

In some embodiments, the Cas effector complex further comprises a small prokaryotic ribosomal protein subunit S15. In some embodiments, the S15 fusion protein is encoded by a sequence having at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, or at least about 99% identity to any one of SEQ ID NOs: 181-183. In some embodiments, the S15 is encoded by a sequence having at least about 70% identity to SEQ ID NOs: 181-183. In some embodiments, the S15 is encoded by a sequence having at least about 75% identity to SEQ ID NOs: 181-183. In some embodiments, the S15 is encoded by a sequence having at least about 80% identity to SEQ ID NOs: 181-183. In some embodiments, the S15 is encoded by a sequence having at least about 85% identity to SEQ ID NOs: 181-183. In some embodiments, the S15 is encoded by a sequence having at least about 90% identity to SEQ ID NOs: 181-183. In some embodiments, the S15 is encoded by a sequence having at least about 91% identity to SEQ ID NOs: 181-183. In some embodiments, the S15 is encoded by a sequence having at least about 92% identity to SEQ ID NOs: 181-183. In some embodiments, the S15 is encoded by a sequence having at least about 93% identity to SEQ ID NOs: 181-183. In some embodiments, the S15 is encoded by a sequence having at least about 94% identity to SEQ ID NOs: 181-183. In some embodiments, the S15 is encoded by a sequence having at least about 95% identity to SEQ ID NOs: 181-183. In some embodiments, the S15 is encoded by a sequence having at least about 96% identity to SEQ ID NOs: 181-183. In some embodiments, the S15 is encoded by a sequence having at least about 97% identity to SEQ ID NOs: 181-183. In some embodiments, the S15 is encoded by a sequence having at least about 98% identity to SEQ ID NOs: 181-183. In some embodiments, the S15 is encoded by a sequence having at least about 99% identity to SEQ ID NOs: 181-183. In some embodiments, the S15 is encoded by a sequence having 100% identity to SEQ ID NOs: 181-183.

In some embodiments, the S15 comprises a sequence having at least about 70% identity to SEQ ID NOs: 187-189. In some embodiments, the S15 comprises a sequence having at least about 75% identity to SEQ ID NOs: 187-189. In some embodiments, the S15 comprises a sequence having at least about 80% identity to SEQ ID NOs: 187-189. In some embodiments, the S15 comprises a sequence having at least about 85% identity to SEQ ID NOs: 187-189. In some embodiments, the S15 comprises a sequence having at least about 90% identity to SEQ ID NOs: 187-189. In some embodiments, the S15 comprises a sequence having at least about 91% identity to SEQ ID NOs: 187-189. In some embodiments, the S15 comprises a sequence having at least about 92% identity to SEQ ID NOs: 187-189. In some embodiments, the S15 comprises a sequence having at least about 93% identity to SEQ ID NOs: 187-189. In some embodiments, the S15 comprises a sequence having at least about 94% identity to SEQ ID NOs: 187-189. In some embodiments, the S15 comprises a sequence having at least about 95% identity to SEQ ID NOs: 187-189. In some embodiments, the S15 comprises a sequence having at least about 96% identity to SEQ ID NOs: 187-189. In some embodiments, the S15 comprises a sequence having at least about 97% identity to SEQ ID NOs: 187-189. In some embodiments, the S15 comprises a sequence having at least about 98% identity to SEQ ID NOs: 187-189. In some embodiments, the S15 comprises a sequence having at least about 99% identity to SEQ ID NOs: 187-189. In some embodiments, the S15 comprises a sequence having 100% identity to SEQ ID NOs: 187-189.

In some embodiments, the Cas effector complex comprises one or more linkers linking the class 2, type V effector, the small prokaryotic ribosomal protein subunit 515, the transposase, the gRNA, or combinations thereof. In some embodiments, the linker comprises at least about 10, 20, 30, 40, 50, 60, 70, 80, 90, 100, 200, 300, or 400 amino acids. In some embodiments, the linker comprises at least about 10, 20, 30, 40, 50, 60, 70, 80, 90, 100, 200, 300, 400, 500, 600, 700, 800, 900, or 1000 nucleotides. In some embodiments, the linker is encoded by a sequence of SEQ ID NO: 186, or a sequence having at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, or at least about 99% identity of SEQ ID NO: 186. In some embodiments, the linker is encoded by SEQ ID NO: 186.

In some aspects, the present disclosure provides an engineered nuclease system comprising: an endonuclease comprising a RuvC domain, the endonuclease being derived from an uncultivated microorganism and is a Class 2, type V-K Cas effector comprising at least 80% identity to any one of SEQ ID NOs: 1, 12, 16, 20-30, 64, 80-85, and 220; and an engineered guide RNA that forms a complex with the endonuclease and comprising a spacer sequence that hybridizes to a target nucleic acid sequence wherein the engineered guide polynucleotide comprises a sequence comprising at least 80% identity to any one of SEQ ID NOs: 754-944.

### Fusion Proteins

Described herein, in some embodiments, are systems for transposing a cargo nucleotide sequence into a target nucleic acid site comprising a fusion protein or a nucleic acid encoding the fusion protein. In some embodiments, the fusion protein or a nucleic acid encoding the fusion protein comprises a class 2, type V effector, a small prokaryotic ribosomal protein subunit S15, a transposase, a gRNA, or combinations thereof. In some embodiments, the fusion protein comprises one or more transposases.

In some embodiments, a nuclear localization sequence (NLS) is fused to the class 2, type V effector. In some embodiments, the NLS is fused at an N-terminus of the class 2, type V effector. In some embodiments, the NLS is fused at a C-terminus of the class 2, type V effector. In some embodiments, the NLS is fused at an N-terminus and a C-terminus of the class 2, type V effector.

In some embodiments, the NLS comprises a sequence of any one of SEQ ID NOs: 192-207 and 1354-1383, or a sequence having at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, or at least about 99% identity to any one of SEQ ID NOs: 192-207 and 1354-1383. In some embodiments, the NLS comprises a sequence having at least about 80% identity to SEQ ID NOs: 192-207 and 1354-1383. In some embodiments, the NLS comprises a sequence having at least about 85% identity to SEQ ID NOs: 192-207 and 1354-1383. In some embodiments, the NLS comprises a sequence having at least about 90% identity to SEQ ID NOs: 192-207 and 1354-1383. In some embodiments, the NLS comprises a sequence having at least about 91% identity to SEQ ID NOs: 192-207 and 1354-1383. In some embodiments, the NLS comprises a sequence having at least about 92% identity to SEQ ID NOs: 192-207 and 1354-1383. In some embodiments, the NLS comprises a sequence having at least about 93% identity to SEQ ID NOs: 192-207 and 1354-1383. In some embodiments, the NLS comprises a sequence having at least about 94% identity to SEQ ID NOs: 192-207 and 1354-1383. In some embodiments, the NLS comprises a sequence having at least about 95% identity to SEQ ID NOs: 192-207 and 1354-1383. In some embodiments, the NLS comprises a sequence having at least about 96% identity to SEQ ID NOs: 192-207 and 1354-1383. In some embodiments, the NLS comprises a sequence having at least about 97% identity to SEQ ID NOs: 192-207 and 1354-1383. In some embodiments, the NLS comprises a sequence having at least about 98% identity to SEQ ID NOs: 192-207 and 1354-1383. In some embodiments, the NLS comprises a sequence having at least about 99% identity to SEQ ID NOs: 192-207 and 1354-1383. In some embodiments, the NLS comprises a sequence having 100% identity to SEQ ID NOs: 192-207 and 1354-1383.

In some embodiments, the fusion protein or a nucleic acid encoding the fusion protein comprises a fusion of S15 and a nuclear localization sequence (NLS). In some embodiments, the NLS is fused at an N-terminus of S15. In some embodiments, the NLS is fused at a C-terminus of S15. In some embodiments, the NLS is fused at an N-terminus and a C-terminus of S15.

In some embodiments, the S15 fusion protein further comprises a cleavable peptide. In some embodiments, the peptide is a 2A peptide.

In some embodiments, the S15 fusion protein is encoded by a sequence with at least 80% sequence identity to any one of SEQ ID NOs: 181-183. In some embodiments, the S15 fusion protein is encoded by a sequence with at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, or at least about 99% identity to any one of SEQ ID NOs: 181-183. In some embodiments, the Cas effector complex further comprises a small prokaryotic ribosomal protein subunit S15. In some embodiments, the S15 fusion protein is encoded by a sequence with at least 80% sequence identity to any one of SEQ ID NOs: 181-183. In some embodiments, the S15 fusion protein is encoded by a sequence with at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, or at least about 99% identity to any one of SEQ ID NOs: 181-183. In some embodiments, the S15 is encoded by a sequence having at least about 70% identity to SEQ ID NOs: 181-183. In some embodiments, the S15 is encoded by a sequence having at least about 75% identity to SEQ ID NOs: 181-183. In some embodiments, the S15 is encoded by a sequence having at least about 80% identity to SEQ ID NOs: 181-183. In some embodiments, the S15 is encoded by a sequence having at least about 85% identity to SEQ ID NOs: 181-183. In some embodiments, the S15 is encoded by a sequence having at least about 90% identity to SEQ ID NOs: 181-183. In some embodiments, the S15 is encoded by a sequence having at least about 91% identity to SEQ ID NOs: 181-183. In some embodiments, the S15 is encoded by a sequence having at least about 92% identity to SEQ ID NOs: 181-183. In some embodiments, the S15 is encoded by a sequence having at least about 93% identity to SEQ ID NOs: 181-183. In some embodiments, the S15 is encoded by a sequence having at least about 94% identity to SEQ ID NOs: 181-183. In some embodiments, the S15 is encoded by a sequence having at least about 95% identity to SEQ ID NOs: 181-183. In some embodiments, the S15 is encoded by a sequence having at least about 96% identity to SEQ ID NOs: 181-183. In some embodiments, the S15 is encoded by a sequence having at least about 97% identity to SEQ ID NOs: 181-183. In some embodiments, the S15 is encoded by a sequence having at least about 98% identity to SEQ ID NOs: 181-183. In some embodiments, the S15 is encoded by a sequence having at least about 99% identity to SEQ ID NOs: 181-183. In some embodiments, the S15 is encoded by a sequence having 100% identity to SEQ ID NOs: 181-183.

In some embodiments, the S15 fusion protein comprises a sequence having at least about 70% sequence identity to any one of SEQ ID NOs: 187-189. In some embodiments, the S15 fusion protein has at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, or at least about 99% identity to any one of SEQ ID NOs: 187-189. In some embodiments, the S15 comprises a sequence having at least about 70% identity to SEQ ID NOs: 187-189. In some embodiments, the S15 comprises a sequence having at least about 75% identity to SEQ ID NOs: 187-189. In some embodiments, the S15 comprises a sequence having at least about 80% identity to SEQ ID NOs: 187-189. In some embodiments, the S15 comprises a sequence having at least about 85% identity to SEQ ID NOs: 187-189. In some embodiments, the S15 comprises a sequence having at least about 90% identity to SEQ ID NOs: 187-189. In some embodiments, the S15 comprises a sequence having at least about 91% identity to SEQ ID NOs: 187-189. In some embodiments, the S15 comprises a sequence having at least about 92% identity to SEQ ID NOs: 187-189. In some embodiments, the S15 comprises a sequence having at least about 93% identity to SEQ ID NOs: 187-189. In some embodiments, the S15 comprises a sequence having at least about 94% identity to SEQ ID NOs: 187-189. In some embodiments, the S15 comprises a sequence having at least about 95% identity to SEQ ID NOs: 187-189. In some embodiments, the S15 comprises a sequence having at least about 96% identity to SEQ ID NOs: 187-189. In some embodiments, the S15 comprises a sequence having at least about 97% identity to SEQ ID NOs: 187-189. In some embodiments, the S15 comprises a sequence having at least about 98% identity to SEQ ID NOs: 187-189. In some embodiments, the S15 comprises a sequence having at least about 99% identity to SEQ ID NOs: 187-189. In some embodiments, the S15 comprises a sequence having 100% identity to SEQ ID NOs: 187-189.

In some embodiments, an NLS is fused to the transposase. In some embodiments, the transposase is TnsB, TnsC, or TniQ. In some embodiments, the transposase is TnsB. In some embodiments, the transposase is TnsC. In some embodiments, the transposase is TniQ. In some embodiments, the NLS is fused at an N-terminus of the transposase. In some embodiments, the NLS is fused at a C-terminus of the transposase. In some embodiments, the NLS is fused at an N-terminus and a C-terminus of the transposase.

In some embodiments, the fusion protein or a nucleic acid encoding the fusion protein comprises a gRNA described herein (for example a dual gRNA or a single gRNA).

In some embodiments, the class 2, type V effector, the small prokaryotic ribosomal protein subunit S15, the transposase, the gRNA, or a fusion protein comprises a tag. In some embodiments, the tag is an affinity tag. In some embodiments the tag is a polypeptide or a polynucleotide. Exemplary affinity tags include, but are not limited to, a His-tag, a Flag tag, a Myc-tag, an MBP-tag, and a GST-tag.

In some embodiments, the class 2, type V effector, the small prokaryotic ribosomal protein subunit S15, the transposase, or a fusion protein, comprises a protease cleavage site. Exemplary protease cleavage sites include, but are not limited to, a TEV site, a C3 site, a Factor Xa site, and an Enterokinase site.

### Cells

Described herein, in certain embodiments, is a cell comprising the systems described herein.

In some embodiments, the cell is a eukaryotic cell (e.g., a plant cell, an animal cell, a protist cell, or a fungi cell), a mammalian cell (a Chinese hamster ovary (CHO) cell, baby hamster kidney (BHK), human embryo kidney (HEK), mouse myeloma (NS0), or human retinal cells), an immortalized cell (e.g., a HeLa cell, a COS cell, a HEK-293T cell, a MDCK cell, a 3T3 cell, a PC12 cell, a Huh7 cell, a HepG2 cell, a K562 cell, a N2a cell, or a SY5Y cell), an insect cell (e.g., a Spodoptera frugiperda cell, a Trichoplusia ni cell, a Drosophila melanogaster cell, a S2 cell, or a Heliothis virescens cell), a yeast cell (e.g., a Saccharomyces cerevisiae cell, a Cryptococcus cell, or a Candida cell), a plant cell (e.g., a parenchyma cell, a collenchyma cell, or a sclerenchyma cell), a fungal cell (e.g., a Saccharomyces cerevisiae cell, a Cryptococcus cell, or a Candida cell), or a prokaryotic cell (e.g., a E. coli cell, a streptococcus bacterium cell, a streptomyces soil bacteria cell, or an archaea cell). In some embodiments, the cell is a eukaryotic cell. In some embodiments, the cell is a mammalian cell. In some embodiments, the cell is an immortalized cell. In some embodiments, the cell is an insect cell. In some embodiments, the cell is a yeast cell. In some embodiments, the cell is a plant cell. In some embodiments, the cell is a fungal cell. In some embodiments, the cell is a prokaryotic cell.

In some embodiments, the cell is an A549, HEK-293, HEK-293T, BHK, CHO, HeLa, MRC5, Sf9, Cos-1, Cos-7, Vero, BSC 1, BSC 40, BMT 10, W138, HeLa, Saos, C2C12, L cell, HT1080, HepG2, Huh7, K562, a primary cell, or derivative thereof.

### Delivery and Vectors

Disclosed herein, in some embodiments, are nucleic acid sequences encoding a MG64 system comprising a class 2, type V effector, a small prokaryotic ribosomal protein subunit 515, a transposase, a gRNA, a fusion protein or a gene editing system disclosed herein.

In some embodiments, the nucleic acid encoding the MG64 system is a DNA, for example a linear DNA, a plasmid DNA, or a minicircle DNA. In some embodiments, the nucleic acid encoding the MG64 system is an RNA, for example a mRNA.

In some embodiments, the nucleic acid encoding the MG64 system is delivered by a nucleic acid-based vector. In some embodiments, the nucleic acid-based vector is a plasmid (e.g., circular DNA molecules that can autonomously replicate inside a cell), cosmid (e.g., pWE or sCos vectors), artificial chromosome, human artificial chromosome (HAC), yeast artificial chromosomes (YAC), bacterial artificial chromosome (BAC), P1-derived artificial chromosomes (PAC), phagemid, phage derivative, bacmid, or virus. In some embodiments, the nucleic acid-based vector is selected from the list consisting of pSF-CMV-NEO-NH2-PPT-3XFLAG, pSF-CMV-NEO-COOH-3XFLAG, pSF-CMV-PURO-NH2-GST-TEV, pSF-OXB20-COOH-TEV-FLAG(R)-6His, pCEP4 pDEST27, pSF-CMV-Ub-KrYFP, pSF-CMV-FMDV-daGFP, pEF1a-mCherry-N1 vector, pEF1a-tdTomato vector, pSF-CMV-FMDV-Hygro, pSF-CMV-PGK-Puro, pMCP-tag(m), pSF-CMV-PURO-NH2-CMYC, pSF-OXB20-BetaGal, pSF-OXB20-Fluc, pSF-OXB20, pSF-Tac, pRI 101-AN DNA, pCambia2301, pTYB21, pKLAC2, pAc5.1/V5-His A, and pDEST8.

In some embodiments, the nucleic acid-based vector comprises a promoter. In some embodiments, the promoter is selected from the group consisting of a mini promoter, an inducible promoter, a constitutive promoter, and derivatives thereof. In some embodiments, the promoter is selected from the group consisting of CMV, CBA, EF1a, CAG, PGK, TRE, U6, UAS, T7, Sp6, lac, araBad, trp, Ptac, p5, p19, p40, Synapsin, CaMKII, GRK1, and derivatives thereof. In some embodiments the promoter is a U6 promoter. In some embodiments, the promoter is a CAG promoter. In some embodiments, the promoter is encoded by a sequence of any one of SEQ ID NOs: 190-191, or a sequence having at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, or at least about 99% identity of any one of SEQ ID NOs: 190-191.

In some embodiments, the nucleic acid-based vector is a virus. In some embodiments, the virus is an alphavirus, a parvovirus, an adenovirus, an AAV, a baculovirus, a Dengue virus, a lentivirus, a herpesvirus, a poxvirus, an anellovirus, a bocavirus, a vaccinia virus, or a retrovirus. In some embodiments, the virus is an alphavirus. In some embodiments, the virus is a parvovirus. In some embodiments, the virus is an adenovirus. In some embodiments, the virus is an AAV. In some embodiments, the virus is a baculovirus. In some embodiments, the virus is a Dengue virus. In some embodiments, the virus is a lentivirus. In some embodiments, the virus is a herpesvirus. In some embodiments, the virus is a poxvirus. In some embodiments, the virus is an anellovirus. In some embodiments, the virus is a bocavirus. In some embodiments, the virus is a vaccinia virus. In some embodiments, the virus is or a retrovirus.

In some embodiments, the AAV is AAV1, AAV2, AAV3, AAV4, AAV5, AAV6, AAV7. AAV8. AAV9, AAV10, AAV11, AAV12, AAV13. AAV14, AAV15, AAV16, AAV-rh8, AAV-rh10, AAV-rh20, AAV-rh39. AAV-rh74, AAV-rhM4-1, AAV-hu37, AAV-Anc80, AAV-Anc80L65, AAV-7m8, AAV-PHP-B, AAV-PHP-EB, AAV-2.5, AAV-2tYF, AAV-3B, AAV-LK03, AAV-HSC1, AAV-HSC2, AAV-HSC3, AAV-HSC4, AAV-HSC5, AAV-HSC6, AAV-HSC7, AAV-HSC8, AAV-HSC9, AAV-HSC10, AAV-HSC11, AAV-HSC12, AAV-HSC13, AAV-HSC14. AAV-HSC15, AAV-TT, AAV-DJ/8, AAV-Myo, AAV-NP40, AAV-NP59, AAV-NP22, AAV-NP66, AAV-HSC16, or a derivative thereof. In some embodiments, the herpesvirus is HSV type 1, HSV-2, VZV, EBV, CMV, HHV-6, HHV-7, or HHV-8.

In some embodiments, the virus is AAV1 or a derivative thereof. In some embodiments, the virus is AAV2 or a derivative thereof. In some embodiments, the virus is AAV3 or a derivative thereof. In some embodiments, the virus is AAV4 or a derivative thereof. In some embodiments, the virus is AAV5 or a derivative thereof. In some embodiments, the virus is AAV6 or a derivative thereof. In some embodiments, the virus is AAV7 or a derivative thereof. In some embodiments, the virus is AAV8 or a derivative thereof. In some embodiments, the virus is AAV9 or a derivative thereof. In some embodiments, the virus is AAV10 or a derivative thereof. In some embodiments, the virus is AAV 11 or a derivative thereof. In some embodiments, the virus is AAV12 or a derivative thereof. In some embodiments, the virus is AAV13 or a derivative thereof. In some embodiments, the virus is AAV14 or a derivative thereof. In some embodiments, the virus is AAV15 or a derivative thereof. In some embodiments, the virus is AAV16 or a derivative thereof. In some embodiments, the virus is AAV-rh8 or a derivative thereof. In some embodiments, the virus is AAV-rh10 or a derivative thereof. In some embodiments, the virus is AAV-rh20 or a derivative thereof. In some embodiments, the virus is AAV-rh39 or a derivative thereof. In some embodiments, the virus is AAV-rh74 or a derivative thereof. In some embodiments, the virus is AAV-rhM4-1 or a derivative thereof. In some embodiments, the virus is AAV-hu37 or a derivative thereof. In some embodiments, the virus is AAV-Anc80 or a derivative thereof. In some embodiments, the virus is AAV-Anc80L65 or a derivative thereof. In some embodiments, the virus is AAV-7m8 or a derivative thereof. In some embodiments, the virus is AAV-PHP-B or a derivative thereof. In some embodiments, the virus is AAV-PHP-EB or a derivative thereof. In some embodiments, the virus is AAV-2.5 or a derivative thereof. In some embodiments, the virus is AAV-2tYF or a derivative thereof. In some embodiments, the virus is AAV-3B or a derivative thereof. In some embodiments, the virus is AAV-LK03 or a derivative thereof. In some embodiments, the virus is AAV-HSC1 or a derivative thereof. In some embodiments, the virus is AAV-HSC2 or a derivative thereof. In some embodiments, the virus is AAV-HSC3 or a derivative thereof. In some embodiments, the virus is AAV-HSC4 or a derivative thereof. In some embodiments, the virus is AAV-HSC5 or a derivative thereof. In some embodiments, the virus is AAV-HSC6 or a derivative thereof. In some embodiments, the virus is AAV-HSC7 or a derivative thereof. In some embodiments, the virus is AAV-HSC8 or a derivative thereof. In some embodiments, the virus is AAV-HSC9 or a derivative thereof. In some embodiments, the virus is AAV-HSC10 or a derivative thereof. In some embodiments, the virus is AAV-HSC11 or a derivative thereof. In some embodiments, the virus is AAV-HSC12 or a derivative thereof. In some embodiments, the virus is AAV-HSC13 or a derivative thereof. In some embodiments, the virus is AAV-HSC14 or a derivative thereof. In some embodiments, the virus is AAV-HSC15 or a derivative thereof. In some embodiments, the virus is AAV-TT or a derivative thereof. In some embodiments, the virus is AAV-DJ/8 or a derivative thereof. In some embodiments, the virus is AAV-Myo or a derivative thereof. In some embodiments, the virus is AAV-NP40 or a derivative thereof. In some embodiments, the virus is AAV-NP59 or a derivative thereof. In some embodiments, the virus is AAV-NP22 or a derivative thereof. In some embodiments, the virus is AAV-NP66 or a derivative thereof. In some embodiments, the virus is AAV-HSC16 or a derivative thereof.

In some embodiments, the virus is HSV-1 or a derivative thereof. In some embodiments, the virus is HSV-2 or a derivative thereof. In some embodiments, the virus is VZV or a derivative thereof. In some embodiments, the virus is EBV or a derivative thereof. In some embodiments, the virus is CMV or a derivative thereof. In some embodiments, the virus is HHV-6 or a derivative thereof. In some embodiments, the virus is HHV-7 or a derivative thereof. In some embodiments, the virus is HHV-8 or a derivative thereof.

In some embodiments, the nucleic acid encoding the MG64 system is delivered by a non-nucleic acid-based delivery system (e.g., a non-viral delivery system). In some embodiments, the non-viral delivery system is a liposome. In some embodiments, the nucleic acid is associated with a lipid. The nucleic acid associated with a lipid, in some embodiments, is encapsulated in the aqueous interior of a liposome, interspersed within the lipid bilayer of a liposome, attached to a liposome via a linking molecule that is associated with both the liposome and the nucleic acid, entrapped in a liposome, complexed with a liposome, dispersed in a solution containing a lipid, mixed with a lipid, combined with a lipid, contained as a suspension in a lipid, contained or complexed with a micelle, or otherwise associated with a lipid. In some embodiments, the nucleic acid is comprised in a lipid nanoparticle (LNP).

In some embodiments, the fusion protein or genome editing system is introduced into the cell in any suitable way, either stably or transiently. In some embodiments, a fusion protein or genome editing system is transfected into the cell. In some embodiments, the cell is transduced or transfected with a nucleic acid construct that encodes a fusion protein or genome editing system. For example, a cell is transduced (e.g., with a virus encoding a fusion protein or genome editing system), or transfected (e.g., with a plasmid encoding a fusion protein or genome editing system) with a nucleic acid that encodes a fusion protein or genome editing system, or the translated fusion protein or genome editing system. In some embodiments, the transduction is a stable or transient transduction. In some embodiments, cells expressing a fusion protein or genome editing system or containing a fusion protein or genome editing system are transduced or transfected with one or more gRNA molecules, for example, when the fusion protein or genome editing system comprises a CRISPR nuclease. In some embodiments, a plasmid expressing a fusion protein or genome editing system is introduced into cells through electroporation, transient (e.g., lipofection) and stable genome integration (e.g., piggybac) and viral transduction (for example lentivirus or AAV) or other methods known to those of skill in the art. In some embodiments, the gene editing system is introduced into the cell as one or more polypeptides. In some embodiments, delivery is achieved through the use of RNP complexes. Delivery methods to cells for polypeptides and/or RNPs are known in the art, for example by electroporation or by cell squeezing.

Exemplary methods of delivery of nucleic acids include lipofection, nucleofection, electroporation, stable genome integration (e.g., piggybac), microinjection, biolistics, virosomes, liposomes, immunoliposomes, polycation or lipid nucleic acid conjugates, naked DNA, artificial virions, and agent-enhanced uptake of DNA. Lipofection is described in e.g., U.S. Pat. Nos. 5,049,386; 4,946,787; and 4,897,355) and lipofection reagents are sold commercially (e.g., Transfectam™, Lipofectin™ and SF Cell Line 4D-Nucleofector X Kit™ (Lonza)). Cationic and neutral lipids that are suitable for efficient receptor-recognition lipofection of polynucleotides include those of WO 91/17424 and WO 91/16024. In some embodiments, the delivery is to cells (e.g., in vitro or ex vivo administration) or target tissues (e.g., in vivo administration). In some embodiments, the nucleic acid is comprised in a liposome or a nanoparticle that specifically targets a host cell.

Additional methods for the delivery of nucleic acids to cells are known to those skilled in the art. See, for example, US 2003/0087817.

In some embodiments, the present disclosure provides a cell comprising a vector or a nucleic acid described herein. In some embodiments, the cell expresses a gene editing system or parts thereof. In some embodiments, the cell is a human cell. In some embodiments, the cell is genome edited ex vivo. In some embodiments, the cell is genome edited in vivo.

### Methods for Transposition

The present disclosure provides methods for transposing a cargo nucleotide sequence into a target nucleic acid site. In some embodiments, the method comprises expressing a system described herein within a cell or introducing a system described herein to a cell. In some embodiments, the method comprises contacting a cell with a system described herein.

In some embodiments, the method comprises contacting a double-stranded nucleic acid comprising the cargo nucleotide sequence with a Cas effector complex comprising a class 2, type V Cas effector and at least one engineered guide polynucleotide configured to hybridize to the target nucleotide sequence. In some embodiments, the method comprises contacting the double-stranded nucleic acid comprising the cargo nucleotide sequence with a Tn7 type transposase complex configured to bind the Cas effector complex, wherein the Tn7 type transposase complex comprises a TnsB subunit. In some embodiments, the method comprises contacting the double-stranded nucleic acid comprising the cargo nucleotide sequence with a double-stranded target nucleic acid comprising the target nucleic acid site.

In some embodiments, the cargo nucleotide sequence is flanked by a left-hand transposase recognition sequence. In some embodiments, the cargo nucleotide sequence is flanked by a right-hand transposase recognition sequence. In some embodiments, the cargo nucleotide sequence is flanked by a left-hand transposase recognition sequence and a right-hand transposase recognition sequence. In some embodiments, the method further comprises a PAM sequence compatible with the Cas effector complex adjacent to the target nucleic acid site. In some embodiments, the PAM sequence is located 3′ of the target nucleic acid site.

In some embodiments, the engineered guide polynucleotide is configured to bind the class 2, type V Cas effector. In some embodiments, the class 2, type V Cas effector comprises a polypeptide comprising a sequence having at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, or at least about 99% identity to SEQ ID NO: 1, 12, 16, 20-30, 64, 80-85, and 220. In some embodiments, the class 2, type V Cas effector comprises a polypeptide comprising a sequence having at least about 70% identity to SEQ ID NOs: 1, 12, 16, 20-30, 64, 80-85, and 220. In some embodiments, the class 2, type V Cas effector comprises a polypeptide comprising a sequence having at least about 75% identity to SEQ ID NOs: 1, 12, 16, 20-30, 64, 80-85, and 220. In some embodiments, the class 2, type V Cas effector comprises a polypeptide comprising a sequence having at least about 80% identity to SEQ ID NOs: 1, 12, 16, 20-30, 64, 80-85, and 220. In some embodiments, the class 2, type V Cas effector comprises a polypeptide comprising a sequence having at least about 85% identity to SEQ ID NOs: 1, 12, 16, 20-30, 64, 80-85, and 220. In some embodiments, the class 2, type V Cas effector comprises a polypeptide comprising a sequence having at least about 90% identity to SEQ ID NOs: 1, 12, 16, 20-30, 64, 80-85, and 220. In some embodiments, the class 2, type V Cas effector comprises a polypeptide comprising a sequence having at least about 91% identity to SEQ ID NOs: 1, 12, 16, 20-30, 64, 80-85, and 220. In some embodiments, the class 2, type V Cas effector comprises a polypeptide comprising a sequence having at least about 92% identity to SEQ ID NOs: 1, 12, 16, 20-30, 64, 80-85, and 220. In some embodiments, the class 2, type V Cas effector comprises a polypeptide comprising a sequence having at least about 93% identity to SEQ ID NOs: 1, 12, 16, 20-30, 64, 80-85, and 220. In some embodiments, the class 2, type V Cas effector comprises a polypeptide comprising a sequence having at least about 94% identity to SEQ ID NOs: 1, 12, 16, 20-30, 64, 80-85, and 220. In some embodiments, the class 2, type V Cas effector comprises a polypeptide comprising a sequence having at least about 95% identity to SEQ ID NOs: 1, 12, 16, 20-30, 64, 80-85, and 220. In some embodiments, the class 2, type V Cas effector comprises a polypeptide comprising a sequence having at least about 96% identity to SEQ ID NOs: 1, 12, 16, 20-30, 64, 80-85, and 220. In some embodiments, the class 2, type V Cas effector comprises a polypeptide comprising a sequence having at least about 97% identity to SEQ ID NOs: 1, 12, 16, 20-30, 64, 80-85, and 220. In some embodiments, the class 2, type V Cas effector comprises a polypeptide comprising a sequence having at least about 98% identity to SEQ ID NOs: 1, 12, 16, 20-30, 64, 80-85, and 220. In some embodiments, the class 2, type V Cas effector comprises a polypeptide comprising a sequence having at least about 99% identity to SEQ ID NOs: 1, 12, 16, 20-30, 64, 80-85, and 220. In some embodiments, the class 2, type V Cas effector comprises a polypeptide comprising a sequence having 100% identity to SEQ ID NOs: 1, 12, 16, 20-30, 64, 80-85, and 220.

In some embodiments, the TnsB subunit comprises a polypeptide having a sequence having at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, or at least about 99% identity to SEQ ID NO: 2, 13, 17, and 65. In some embodiments, the TnsA subunit comprises a polypeptide having a sequence identical to SEQ ID NO: 2, 13, 17, and 65. In some embodiments, the TnsB component comprises a polypeptide comprising a sequence having at least about 70% identity to SEQ ID NOs: 2, 13, 17, and 65. In some embodiments, the TnsB component comprises a polypeptide comprising a sequence having at least about 75% identity to SEQ ID NOs: 2, 13, 17, and 65. In some embodiments, the TnsB component comprises a polypeptide comprising a sequence having at least about 80% identity to SEQ ID NOs: 2, 13, 17, and 65. In some embodiments, the TnsB component comprises a polypeptide comprising a sequence having at least about 85% identity to SEQ ID NOs: 2, 13, 17, and 65. In some embodiments, the TnsB component comprises a polypeptide comprising a sequence having at least about 90% identity to SEQ ID NOs: 2, 13, 17, and 65. In some embodiments, the TnsB component comprises a polypeptide comprising a sequence having at least about 91% identity to SEQ ID NOs: 2, 13, 17, and 65. In some embodiments, the TnsB component comprises a polypeptide comprising a sequence having at least about 92% identity to SEQ ID NOs: 2, 13, 17, and 65. In some embodiments, the TnsB component comprises a polypeptide comprising a sequence having at least about 93% identity to SEQ ID NOs: 2, 13, 17, and 65. In some embodiments, the TnsB component comprises a polypeptide comprising a sequence having at least about 94% identity to SEQ ID NOs: 2, 13, 17, and 65. In some embodiments, the TnsB component comprises a polypeptide comprising a sequence having at least about 95% identity to SEQ ID NOs: 2, 13, 17, and 65. In some embodiments, the TnsB component comprises a polypeptide comprising a sequence having at least about 96% identity to SEQ ID NOs: 2, 13, 17, and 65. In some embodiments, the TnsB component comprises a polypeptide comprising a sequence having at least about 97% identity to SEQ ID NOs: 2, 13, 17, and 65. In some embodiments, the TnsB component comprises a polypeptide comprising a sequence having at least about 98% identity to SEQ ID NOs: 2, 13, 17, and 65. In some embodiments, the TnsB component comprises a polypeptide comprising a sequence having at least about 99% identity to SEQ ID NOs: 2, 13, 17, and 65. In some embodiments, the TnsB component comprises a polypeptide comprising a sequence having 100% identity to SEQ ID NOs: 2, 13, 17, and 65.

In some embodiments, the Tn7 type transposase complex comprises at least one polypeptide (e.g., at least 1, 2, 3, 4, 5, 6, or more than 6 polypeptides) comprising a sequence having at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, or at least about 99% identity to any one of SEQ ID NOs: 3-4, 14-15, 18-19, 66-67, and 109-111. In some embodiments, the Tn7 type transposase complex comprises a polypeptide comprising a sequence having at least about 70% identity to SEQ ID NOs: 3-4, 14-15, 18-19, 66-67, and 109-111. In some embodiments, the Tn7 type transposase complex comprises a polypeptide comprising a sequence having at least about 75% identity to SEQ ID NOs: 3-4, 14-15, 18-19, 66-67, and 109-111. In some embodiments, the Tn7 type transposase complex comprises a polypeptide comprising a sequence having at least about 80% identity to SEQ ID NOs: 3-4, 14-15, 18-19, 66-67, and 109-111. In some embodiments, the Tn7 type transposase complex comprises a polypeptide comprising a sequence having at least about 85% identity to SEQ ID NOs: 3-4, 14-15, 18-19, 66-67, and 109-111. In some embodiments, the Tn7 type transposase complex comprises a polypeptide comprising a sequence having at least about 90% identity to SEQ ID NOs: 3-4, 14-15, 18-19, 66-67, and 109-111. In some embodiments, the Tn7 type transposase complex comprises a polypeptide comprising a sequence having at least about 91% identity to SEQ ID NOs: 3-4, 14-15, 18-19, 66-67, and 109-111. In some embodiments, the Tn7 type transposase complex comprises a polypeptide comprising a sequence having at least about 92% identity to SEQ ID NOs: 3-4, 14-15, 18-19, 66-67, and 109-111. In some embodiments, the Tn7 type transposase complex comprises a polypeptide comprising a sequence having at least about 93% identity to SEQ ID NOs: 3-4, 14-15, 18-19, 66-67, and 109-111. In some embodiments, the Tn7 type transposase complex comprises a polypeptide comprising a sequence having at least about 94% identity to SEQ ID NOs: 3-4, 14-15, 18-19, 66-67, and 109-111. In some embodiments, the Tn7 type transposase complex comprises a polypeptide comprising a sequence having at least about 95% identity to SEQ ID NOs: 3-4, 14-15, 18-19, 66-67, and 109-111. In some embodiments, the Tn7 type transposase complex comprises a polypeptide comprising a sequence having at least about 96% identity to SEQ ID NOs: 3-4, 14-15, 18-19, 66-67, and 109-111. In some embodiments, the Tn7 type transposase complex comprises a polypeptide comprising a sequence having at least about 97% identity to SEQ ID NOs: 3-4, 14-15, 18-19, 66-67, and 109-111. In some embodiments, the Tn7 type transposase complex comprises a polypeptide comprising a sequence having at least about 98% identity to SEQ ID NOs: 3-4, 14-15, 18-19, 66-67, and 109-111. In some embodiments, the Tn7 type transposase complex comprises a polypeptide comprising a sequence having at least about 99% identity to SEQ ID NOs: 3-4, 14-15, 18-19, 66-67, and 109-111. In some embodiments, the Tn7 type transposase complex comprises a polypeptide comprising a sequence having 100% identity to SEQ ID NOs: 3-4, 14-15, 18-19, 66-67, and 109-111.

In some embodiments, the Tn7 type transposase complex comprises at least a first polypeptide and a second polypeptide each independently comprising a sequence having at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, or at least about 99% identity to any one of SEQ ID NOs: 3-4, 14-15, 18-19, 66-67, and 109-111. In some embodiments, the Tn7 type transposase complex comprises at least a first polypeptide and a second polypeptide each independently comprising a sequence with at least 70% sequence identity to any one of SEQ ID NOs: 3-4, 14-15, 18-19, 66-67, and 109-111. In some embodiments, the Tn7 type transposase complex comprises at least a first polypeptide and a second polypeptide each independently comprising a sequence with at least 75% sequence identity to any one of SEQ ID NOs: 3-4, 14-15, 18-19, 66-67, and 109-111. In some embodiments, the Tn7 type transposase complex comprises at least a first polypeptide and a second polypeptide each independently comprising a sequence with at least 80% sequence identity to any one of SEQ ID NOs: 3-4, 14-15, 18-19, 66-67, and 109-111. In some embodiments, the Tn7 type transposase complex comprises at least a first polypeptide and a second polypeptide each independently having at least about 85% identity to SEQ ID NOs: 3-4, 14-15, 18-19, 66-67, and 109-111. In some embodiments, the Tn7 type transposase complex comprises at least a first polypeptide and a second polypeptide each independently having at least about 90% identity to SEQ ID NOs: 3-4, 14-15, 18-19, 66-67, and 109-111. In some embodiments, the Tn7 type transposase complex comprises at least a first polypeptide and a second polypeptide each independently having at least about 91% identity to SEQ ID NOs: 3-4, 14-15, 18-19, 66-67, and 109-111. In some embodiments, the Tn7 type transposase complex comprises at least a first polypeptide and a second polypeptide each independently having at least about 92% identity to SEQ ID NOs: 3-4, 14-15, 18-19, 66-67, and 109-111. In some embodiments, the Tn7 type transposase complex comprises at least a first polypeptide and a second polypeptide each independently having at least about 93% identity to SEQ ID NOs: 3-4, 14-15, 18-19, 66-67, and 109-111. In some embodiments, the Tn7 type transposase complex comprises at least a first polypeptide and a second polypeptide each independently having at least about 94% identity to SEQ ID NOs: 3-4, 14-15, 18-19, 66-67, and 109-111. In some embodiments, the Tn7 type transposase complex comprises at least a first polypeptide and a second polypeptide each independently having at least about 95% identity to SEQ ID NOs: 3-4, 14-15, 18-19, 66-67, and 109-111. In some embodiments, the Tn7 type transposase complex comprises at least a first polypeptide and a second polypeptide each independently having at least about 96% identity to SEQ ID NOs: 3-4, 14-15, 18-19, 66-67, and 109-111. In some embodiments, the Tn7 type transposase complex comprises at least a first polypeptide and a second polypeptide each independently having at least about 97% identity to SEQ ID NOs: 3-4, 14-15, 18-19, 66-67, and 109-111. In some embodiments, the Tn7 type transposase complex comprises at least a first polypeptide and a second polypeptide each independently having at least about 98% identity to SEQ ID NOs: 3-4, 14-15, 18-19, 66-67, and 109-111. In some embodiments, the Tn7 type transposase complex comprises at least a first polypeptide and a second polypeptide each independently having at least about 99% identity to SEQ ID NOs: 3-4, 14-15, 18-19, 66-67, and 109-111. In some embodiments, the Tn7 type transposase complex comprises at least a first polypeptide and a second polypeptide each independently having 100% identity to SEQ ID NOs: 3-4, 14-15, 18-19, 66-67, and 109-111.

In some embodiments, the engineered guide polynucleotide comprises a sequence comprising at least about 46-80 consecutive nucleotides having at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, or at least about 99% identity to any one of SEQ ID NOs: 5-6, 32-33, 94-95, 104-105, 119-122, and 222. In some embodiments, the engineered guide polynucleotide comprises a sequence comprising at least about 46-80 consecutive nucleotides having at least about 70% identity to SEQ ID NOs: 5-6, 32-33, 94-95, 104-105, 119-122, and 222. In some embodiments, the engineered guide polynucleotide comprises a sequence comprising at least about 46-80 consecutive nucleotides having at least about 75% identity to SEQ ID NOs: 5-6, 32-33, 94-95, 104-105, 119-122, and 222. In some embodiments, the engineered guide polynucleotide comprises a sequence comprising at least about 46-80 consecutive nucleotides having at least about 80% identity to SEQ ID NOs: 5-6, 32-33, 94-95, 104-105, 119-122, and 222. In some embodiments, the engineered guide polynucleotide comprises a sequence comprising at least about 46-80 consecutive nucleotides having at least about 85% identity to SEQ ID NOs: 5-6, 32-33, 94-95, 104-105, 119-122, and 222. In some embodiments, the engineered guide polynucleotide comprises a sequence comprising at least about 46-80 consecutive nucleotides having at least about 90% identity to SEQ ID NOs: 5-6, 32-33, 94-95, 104-105, 119-122, and 222. In some embodiments, the engineered guide polynucleotide comprises a sequence comprising at least about 46-80 consecutive nucleotides having at least about 91% identity to SEQ ID NOs: 5-6, 32-33, 94-95, 104-105, 119-122, and 222. In some embodiments, the engineered guide polynucleotide comprises a sequence comprising at least about 46-80 consecutive nucleotides having at least about 92% identity to SEQ ID NOs: 5-6, 32-33, 94-95, 104-105, 119-122, and 222. In some embodiments, the engineered guide polynucleotide comprises a sequence comprising at least about 46-80 consecutive nucleotides having at least about 93% identity to SEQ ID NOs: 5-6, 32-33, 94-95, 104-105, 119-122, and 222. In some embodiments, the engineered guide polynucleotide comprises a sequence comprising at least about 46-80 consecutive nucleotides having at least about 94% identity to SEQ ID NOs: 5-6, 32-33, 94-95, 104-105, 119-122, and 222. In some embodiments, the engineered guide polynucleotide comprises a sequence comprising at least about 46-80 consecutive nucleotides having at least about 95% identity to SEQ ID NOs: 5-6, 32-33, 94-95, 104-105, 119-122, and 222. In some embodiments, the engineered guide polynucleotide comprises a sequence comprising at least about 46-80 consecutive nucleotides having at least about 96% identity to SEQ ID NOs: 5-6, 32-33, 94-95, 104-105, 119-122, and 222. In some embodiments, the engineered guide polynucleotide comprises a sequence comprising at least about 46-80 consecutive nucleotides having at least about 97% identity to SEQ ID NOs: 5-6, 32-33, 94-95, 104-105, 119-122, and 222. In some embodiments, the engineered guide polynucleotide comprises a sequence comprising at least about 46-80 consecutive nucleotides having at least about 98% identity to SEQ ID NOs: 5-6, 32-33, 94-95, 104-105, 119-122, and 222. In some embodiments, the engineered guide polynucleotide comprises a sequence comprising at least about 46-80 consecutive nucleotides having at least about 99% identity to SEQ ID NOs: 5-6, 32-33, 94-95, 104-105, 119-122, and 222. In some embodiments, the engineered guide polynucleotide comprises a sequence comprising at least about 46-80 consecutive nucleotides having 100% identity to SEQ ID NOs: 5-6, 32-33, 94-95, 104-105, 119-122, and 222.

In some embodiments, the engineered guide polynucleotide is a guide RNA and comprises a sequence having at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, or at least about 99% identity to any one of SEQ ID NOs: 106, 107, 108, 5, 45-63, 68-75, 96-103, 123-140, and 754-944. In some embodiments, the engineered guide polynucleotide is a guide RNA comprises a sequence identical to any one of SEQ ID NOs: 106, 107, 108, 5, 45-63, 68-75, 96-103, 123-140, and 754-944. In some embodiments, the engineered guide polynucleotide is a guide RNA comprises a sequence having at least about 70% identity to SEQ ID NOs: 106, 107, 108, 5, 45-63, 68-75, 96-103, 123-140, and 754-944. In some embodiments, the engineered guide polynucleotide is a guide RNA comprises a sequence having at least about 75% identity to SEQ ID NOs: 106, 107, 108, 5, 45-63, 68-75, 96-103, 123-140, and 754-944. In some embodiments, the engineered guide polynucleotide is a guide RNA comprises a sequence having at least about 80% identity to SEQ ID NOs: 106, 107, 108, 5, 45-63, 68-75, 96-103, 123-140, and 754-944. In some embodiments, the engineered guide polynucleotide is a guide RNA comprises a sequence having at least about 85% identity to SEQ ID NOs: 106, 107, 108, 5, 45-63, 68-75, 96-103, 123-140, and 754-944. In some embodiments, the engineered guide polynucleotide is a guide RNA comprises a sequence having at least about 90% identity to SEQ ID NOs: 106, 107, 108, 5, 45-63, 68-75, 96-103, 123-140, and 754-944. In some embodiments, the engineered guide polynucleotide is a guide RNA comprises a sequence having at least about 91% identity to SEQ ID NOs: 106, 107, 108, 5, 45-63, 68-75, 96-103, 123-140, and 754-944. In some embodiments, the engineered guide polynucleotide is a guide RNA comprises a sequence having at least about 92% identity to SEQ ID NOs: 106, 107, 108, 5, 45-63, 68-75, 96-103, 123-140, and 754-944. In some embodiments, the engineered guide polynucleotide is a guide RNA comprises a sequence having at least about 93% identity to SEQ ID NOs: 106, 107, 108, 5, 45-63, 68-75, 96-103, 123-140, and 754-944. In some embodiments, the engineered guide polynucleotide is a guide RNA comprises a sequence having at least about 94% identity to SEQ ID NOs: 106, 107, 108, 5, 45-63, 68-75, 96-103, 123-140, and 754-944. In some embodiments, the engineered guide polynucleotide is a guide RNA comprises a sequence having at least about 95% identity to SEQ ID NOs: 106, 107, 108, 5, 45-63, 68-75, 96-103, 123-140, and 754-944. In some embodiments, the engineered guide polynucleotide is a guide RNA comprises a sequence having at least about 96% identity to SEQ ID NOs: 106, 107, 108, 5, 45-63, 68-75, 96-103, 123-140, and 754-944. In some embodiments, the engineered guide polynucleotide is a guide RNA comprises a sequence having at least about 97% identity to SEQ ID NOs: 106, 107, 108, 5, 45-63, 68-75, 96-103, 123-140, and 754-944. In some embodiments, the engineered guide polynucleotide is a guide RNA comprises a sequence having at least about 98% identity to SEQ ID NOs: 106, 107, 108, 5, 45-63, 68-75, 96-103, 123-140, and 754-944. In some embodiments, the engineered guide polynucleotide is a guide RNA comprises a sequence having at least about 99% identity to SEQ ID NOs: 106, 107, 108, 5, 45-63, 68-75, 96-103, 123-140, and 754-944. In some embodiments the engineered guide polynucleotide is a guide RNA comprises a sequence having 100% identity to SEQ ID NOs: 106, 107, 108, 5, 45-63, 68-75, 96-103, 123-140, and 754-944.

In some embodiments, the left-hand transposase recognition sequence comprises a sequence having at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, or at least about 99% identity to SEQ ID NO: 9, 11, 36-38, 76, and 78. In some embodiments, the left-hand transposase recognition sequence comprises a sequence having at least about 70% identity to SEQ ID NOs: 9, 11, 36-38, 76, and 78. In some embodiments, the left-hand transposase recognition sequence comprises a sequence having at least about 75% identity to SEQ ID NOs: 9, 11, 36-38, 76, and 78. In some embodiments, the left-hand transposase recognition sequence comprises a sequence having at least about 80% identity to SEQ ID NOs: 9, 11, 36-38, 76, and 78. In some embodiments, the left-hand transposase recognition sequence comprises a sequence having at least about 85% identity to SEQ ID NOs: 9, 11, 36-38, 76, and 78. In some embodiments, the left-hand transposase recognition sequence comprises a sequence having at least about 90% identity to SEQ ID NOs: 9, 11, 36-38, 76, and 78. In some embodiments, the left-hand transposase recognition sequence comprises a sequence having at least about 91% identity to SEQ ID NOs: 9, 11, 36-38, 76, and 78. In some embodiments, the left-hand transposase recognition sequence comprises a sequence having at least about 92% identity to SEQ ID NOs: 9, 11, 36-38, 76, and 78. In some embodiments, the left-hand transposase recognition sequence comprises a sequence having at least about 93% identity to SEQ ID NOs: 9, 11, 36-38, 76, and 78. In some embodiments, the left-hand transposase recognition sequence comprises a sequence having at least about 94% identity to SEQ ID NOs: 9, 11, 36-38, 76, and 78. In some embodiments, the left-hand transposase recognition sequence comprises a sequence having at least about 95% identity to SEQ ID NOs: 9, 11, 36-38, 76, and 78. In some embodiments, the left-hand transposase recognition sequence comprises a sequence having at least about 96% identity to SEQ ID NOs: 9, 11, 36-38, 76, and 78. In some embodiments, the left-hand transposase recognition sequence comprises a sequence having at least about 97% identity to SEQ ID NOs: 9, 11, 36-38, 76, and 78. In some embodiments, the left-hand transposase recognition sequence comprises a sequence having at least about 98% identity to SEQ ID NOs: 9, 11, 36-38, 76, and 78. In some embodiments, the left-hand transposase recognition sequence comprises a sequence having at least about 99% identity to SEQ ID NOs: 9, 11, 36-38, 76, and 78. In some embodiments, the left-hand transposase recognition sequence comprises a sequence having 100% identity to SEQ ID NOs: 9, 11, 36-38, 76, and 78.

In some embodiments, the right-hand transposase recognition sequence comprises a sequence having at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, or at least about 99% identity to SEQ ID NO: 8, 10, 39-44, 77, 79, and 93. In some embodiments, the right-hand transposase recognition sequence comprises a sequence having at least about 70% identity to SEQ ID NOs: 8, 10, 39-44, 77, 79, and 93. In some embodiments, the right-hand transposase recognition sequence comprises a sequence having at least about 75% identity to SEQ ID NOs: 8, 10, 39-44, 77, 79, and 93. In some embodiments, the right-hand transposase recognition sequence comprises a sequence having at least about 80% identity to SEQ ID NOs: 8, 10, 39-44, 77, 79, and 93. In some embodiments, the right-hand transposase recognition sequence comprises a sequence having at least about 85% identity to SEQ ID NOs: 8, 10, 39-44, 77, 79, and 93. In some embodiments, the right-hand transposase recognition sequence comprises a sequence having at least about 90% identity to SEQ ID NOs: 8, 10, 39-44, 77, 79, and 93. In some embodiments, the right-hand transposase recognition sequence comprises a sequence having at least about 91% identity to SEQ ID NOs: 8, 10, 39-44, 77, 79, and 93. In some embodiments, the right-hand transposase recognition sequence comprises a sequence having at least about 92% identity to SEQ ID NOs: 8, 10, 39-44, 77, 79, and 93. In some embodiments, the right-hand transposase recognition sequence comprises a sequence having at least about 93% identity to SEQ ID NOs: 8, 10, 39-44, 77, 79, and 93. In some embodiments, the right-hand transposase recognition sequence comprises a sequence having at least about 94% identity to SEQ ID NOs: 8, 10, 39-44, 77, 79, and 93. In some embodiments, the right-hand transposase recognition sequence comprises a sequence having at least about 95% identity to SEQ ID NOs: 8, 10, 39-44, 77, 79, and 93. In some embodiments, the right-hand transposase recognition sequence comprises a sequence having at least about 96% identity to SEQ ID NOs: 8, 10, 39-44, 77, 79, and 93. In some embodiments, the right-hand transposase recognition sequence comprises a sequence having at least about 97% identity to SEQ ID NOs: 8, 10, 39-44, 77, 79, and 93. In some embodiments, the right-hand transposase recognition sequence comprises a sequence having at least about 98% identity to SEQ ID NOs: 8, 10, 39-44, 77, 79, and 93. In some embodiments, the right-hand transposase recognition sequence comprises a sequence having at least about 99% identity to SEQ ID NOs: 8, 10, 39-44, 77, 79, and 93. In some embodiments, the right-hand transposase recognition sequence comprises a sequence having 100% identity to SEQ ID NOs: 8, 10, 39-44, 77, 79, and 93.

In some embodiments, the class 2, type V Cas effector and the Tn7 type transposase complex are encoded by polynucleotide sequences comprising fewer than about 20 kilobases, fewer than about 15 kilobases, fewer than about 10 kilobases, or fewer than about 5 kilobases.

### Uses

Systems of the present disclosure may be used for various applications, such as, for example, nucleic acid editing (e.g., gene editing) or binding to a nucleic acid molecule (e.g., sequence-specific binding). Such systems may be used, for example, for remediating (e.g., removing or replacing) a genetically inherited mutation that may cause a disease in a subject; inactivating a gene in order to ascertain its function in a cell; as a diagnostic tool to detect disease-causing genetic elements (e.g., via cleavage of reverse-transcribed viral RNA or an amplified DNA sequence encoding a disease-causing mutation); as deactivated enzymes in combination with a probe to target and detect a specific nucleotide sequence (e.g., sequence encoding antibiotic resistance int bacteria); to render viruses inactive or incapable of infecting host cells by targeting viral genomes; to add genes or amend metabolic pathways to engineer organisms to produce valuable small molecules, macromolecules, or secondary metabolites; to establish a gene drive element for evolutionary selection, and/or to detect cell perturbations by foreign small molecules and nucleotides as a biosensor.

### Kits

In some embodiments, this disclosure provides kits comprising one or more nucleic acid constructs encoding the various components of the fusion protein or genome editing system described herein. e.g., comprising a nucleotide sequence encoding the components of the fusion protein or genome editing system capable of modifying a target DNA sequence. In some embodiments, the nucleotide sequence comprises a heterologous promoter that drives expression of the RNA genome editing system components.

In some embodiments, the class 2, type V effector, the small prokaryotic ribosomal protein subunit S15, the transposase, the gRNA, or a fusion protein or gene editing system comprising any combination thereof disclosed herein is assembled into a pharmaceutical, diagnostic, or research kit to facilitate its use in therapeutic, diagnostic, or research applications. A kit may include one or more containers housing any of the vectors disclosed herein and instructions for use.

The kit may be designed to facilitate use of the methods described herein by researchers and can take many forms. Each of the compositions of the kit, where applicable, may be provided in liquid form (e.g., in solution), or in solid form, (e.g., a dry powder). In certain cases, some of the compositions may be constitutable or otherwise processable (e.g., to an active form), for example, by the addition of a suitable solvent or other species (for example, water or a cell culture medium), which may or may not be provided with the kit. As used herein, “instructions” can define a component of instruction and/or promotion, and typically involve written instructions on or associated with packaging of the disclosure. Instructions also can include any oral or electronic instructions provided in any manner such that a user will clearly recognize that the instructions are to be associated with the kit, for example, audiovisual (e.g., videotape, DVD, etc.), Internet, and/or web-based communications, etc. The written instructions, in some embodiments, are in a form prescribed by a governmental agency regulating the manufacture, use, or sale of pharmaceuticals or biological products, which instructions can also reflect approval by the agency of manufacture, use, or sale for animal administration.

### EXAMPLES

The following examples are given for the purpose of illustrating various embodiments of the disclosure and are not meant to limit the present disclosure in any fashion. The present examples, along with the methods described herein, are presently representative of preferred embodiments, are exemplary, and are not intended as limitations on the scope of the disclosure. Changes therein and other uses which are encompassed within the spirit of the disclosure as defined by the scope of the claims will occur to those skilled in the art.

### Example 1—(General Protocol) PAM Sequence Identification/Confirmation for Systems Described Herein

Putative endonucleases were expressed in an E. coli lysate-based expression system. PAM sequences were determined by sequencing plasmids containing randomly-generated potential PAM sequences that are able to be cleaved by the putative nucleases. In this system, an E. coli codon optimized nucleotide sequence encoding the putative nuclease was transcribed and translated in vitro from a PCR fragment under control of a T7 promoter. A second PCR fragment with a minimal CRISPR array composed of a T7 promoter followed by a repeat-spacer-repeat sequence was transcribed in the same reaction. Successful expression of the endonuclease and repeat-spacer-repeat sequence in the E. coli lysate-based expression system followed by CRISPR array processing provided active in vitro CRISPR nuclease complexes.

A library of target plasmids containing a spacer sequence matching that in the minimal array preceded by 8N mixed bases (potential PAM sequences) was incubated with the output of the expression system reaction. After 1-3 hr, the reaction was stopped and the DNA was recovered via a DNA clean-up kit. Adapter sequences were blunt-end ligated to DNA with active PAM sequences that were cleaved by the endonuclease, whereas DNA that was not cleaved was inaccessible for ligation. DNA segments comprising active PAM sequences were then amplified by PCR with primers specific to the library and the adapter sequence. The PCR amplification products were resolved on a gel to identify amplicons that correspond to cleavage events. The amplified segments of the cleavage reaction were also used as templates for preparation of an NGS library or as a substrate for Sanger sequencing. Sequencing this resulting library, which is a subset of the starting 8N library, revealed sequences with PAM activity compatible with the CRISPR complex. For PAM testing with a processed RNA construct, the same procedure was repeated except that an in vitro transcribed RNA was added along with the plasmid library and the minimal CRISPR array template was omitted.

Analysis of the intergenic regions surrounding the Cas effector and CRISPR array identified a potential anti-repeat sequence corresponding to the duplexing sequence of the tracrRNA. TracrRNA and crRNA repeat were folded and trimmed, adding a tetraloop sequence of GAAA to maintain the stem loop region of the crRNA-tracrRNA complex.

### Example 2A —In Vitro Targeted Integrase Activity

Integrase activity was assayed with a previously identified PAM but may be conducted with a PAM library substrate instead, with reduced efficiency. One arrangement of components for in vitro testing involved three plasmids other than that containing the donor sequence: (1) an expression plasmid with effector (or effectors) under a T7 promoter; (2) an expression plasmid with transposase genes under a T7 promoter: a sgRNA or crRNA and tracrRNA; (3) a target plasmid which contained the spacer site and appropriate PAM; and (4) a donor plasmid which contained the required left end (LE) and right end (RE) DNA sequences for transposition around a cargo gene (e.g., a selection marker such as a Tet resistance gene). Using an in vitro transcription/translation system (e.g., E. coli lysate- or reticulocyte lysate-based system), the effector and transposase genes were expressed. After expression, the RNA, target DNA, and donor DNA were added and incubated to allow for transposition to occur. Transposition was detected via PCR across the junction of the transposase site, with one primer on the target DNA and one primer on the donor DNA. The resulting PCR product was sequenced via NGS to determine the exact insertion topology relative to the sgRNA/crRNA targeted site. The primers were located downstream such that a variety of insertion sites were accommodated and detected. Primers were designed such that integration was detected in either orientation of cargo and on either side of the spacer, as the integration direction was also not previously documented.

Integration efficiency was measured via quantitative PCR (qPCR) measurements of the experimental output of target DNA with integrated cargo, normalized to the amount of unmodified target DNA also measured via qPCR.

This assay may be conducted with purified protein components rather than from lysate-based expression. In this case the proteins were expressed in an E coli protease deficient B strain under a T7 inducible promoter, the cells were lysed using sonication, and the His-tagged protein of interest was purified using Ni-NTA affinity chromatography on an FPLC system. Purity was determined using densitometry of the protein bands resolved on SDS-PAGE and Coomassie stained acrylamide gels. The protein was desalted in storage buffer composed of 50 mM Tris-HCl, 300 mM NaCl, 1 mM TCEP, 5% glycerol; pH 7.5 (or other buffers as determined for maximum stability) and stored at −80° C. After purification the effector(s) and transposase(s) were added to the sgRNA, target DNA, and donor DNA as described above in a reaction buffer, for example 26 mM HEPES pH 7.5, 4.2 mM TRIS pH 8, 50 μg/mL BSA, 2 mM ATP, 2.1 mM DTT, 0.05 mM EDTA, 0.2 mM MgCl₂, 28 mM NaCl, 21 mM KCl, 1.35% glycerol, (final pH 7.5) supplemented with 15 mM Mg(Oac)₂.

### Example 2B—In Vitro Activity

### Targeted Nuclease

In situ expression and protein sequence analyses indicated that some RNA guided effectors are active nucleases. They contained predicted endonuclease-associated domains (matching RuvC and HNH_endonuclease domains), and/or predicted HNH and RuvC catalytic residues.

Candidate activity was tested with engineered single guide RNA sequences using the E. coli lysate-based expression system and in vitro transcribed RNA. Active proteins that successfully cleaved the library yielded a band around 170 bp in the gel.

### DNA Integration and Transposition

Transposons are predicted to be active when the genomic sequences encoding them contain one or more protein sequences with transposase and/or integrase function within the left and right ends of the transposon. A Tn7 transposon, as defined here, may comprise a catalytic transposase TnsB, but may also contain TnsA, TnsC, TnsD. TnsE, TniQ, and/or other transposase or integrases. The transposon ends comprise predicted transposase binding sites, which contain direct and/or inverted repeats of 15 bp to 150 bp in length flanking the transposase proteins and other ‘cargo’ genes. Protein sequence analysis indicated that the transposases contain integrase domains, transposase domains and/or transposase catalytic residues, suggesting that they are active (e.g., FIG. 4A).

### Targeted DNA Integration

Putative CRISPR-associated transposons (CAST) contain a DNA and/or RNA targeting CRISPR nuclease or effector and proteins with predicted transposase function in the vicinity of a CRISPR array. In some systems, the nuclease is predicted to be active based on the presence of endonuclease-associated catalytic domains and/or catalytic residues.

In some systems, the effector is predicted to have homology with documented CRISPR effector proteins, but to be inactive based on the absence of endonuclease domains and/or catalytic residues. The transposases are predicted to be associated with the effector when the CRISPR loci (inactive CRISPR nuclease and array) and the transposase proteins are located within the predicted transposon left and right ends (FIG. 4A). In this case, the effector is predicted to direct DNA integration to specific genomic locations based on a guide RNA.

CAST activity was tested with five types of components (1) a Cas effector protein expressed by in vitro expression systems, (2) a target DNA fragment or plasmid containing the target sequence and PAM corresponding to the Cas enzyme, (3) a donor DNA fragments containing a marker or fragment of DNA flanked by the LE and RE of the transposase system in a DNA fragment or plasmid (4) any combination of transposase proteins expressed using in vitro expression systems, and (5) an engineered in vitro transcribed single guide RNA sequence. Active systems that successfully transposed the donor fragment were assayed by PCR amplification of the donor-target junction.

After performing the transposition reaction, PCR amplification of the junction showed that proper donor-target formation was made, and the transposition reaction was sg dependent. (FIG. 6). PCR amplification of reactions #3 and #4 indicated that both orientations of the donor relative to the target were made: one where the LE is closer to the PAM, and one where the RE is closer to the PAM. While both transposition orientations were made, there was a preference for donor integration in the target where the LE is closer to the PAM, represented by strong band present for reactions #4 and #5.

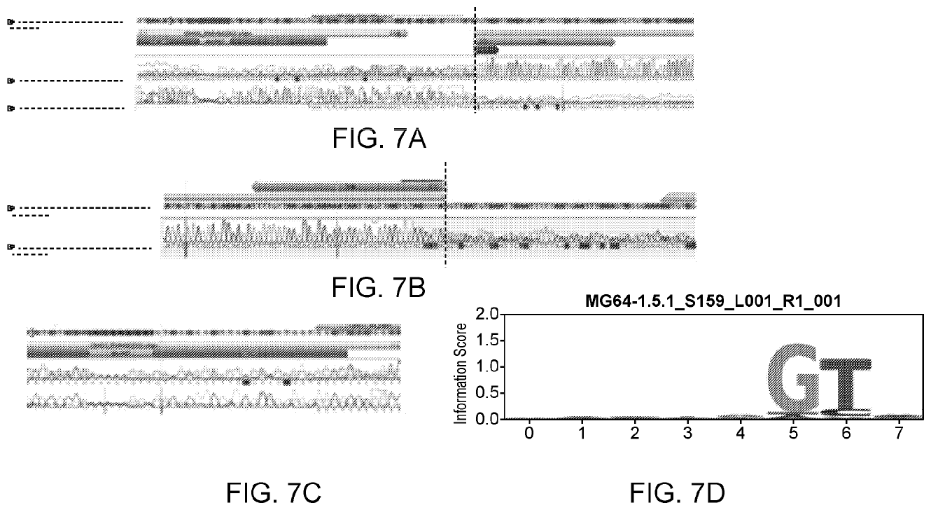

Sanger sequencing of the preferred orientation product was performed. Of the integrations that occurred with the LE closer to the PAM, there was a clear degradation of the sequencing chromatogram signal from either the forward or reverse direction over the target/donor junction. This indicated that, of the products that were oriented with the LE closer to the PAM, integration occurred in a range of nucleotides, with the primary product of LE-closer-to-PAM products as a 61 bp integration from the PAM (FIG. 7A). Sequencing that originated from the donor over the donor-target junction defined the composition of the essential outer bounds of the LE and RE sequences (FIGS. 7A-7B). Further investigation of the LE and RE domains may determine the inner limits of the LE and RE sequences that are essential for transposition. Sequencing of the RE on LE-closer-to-PAM products showed a 3 bp duplication downstream of the donor RE (FIG. 7B). This is in part due to the Tn7 transposase integration event that cleaved and ligated the donor fragment at a staggered cut site. A 3 bp duplication is smaller than the expected 5 bp of duplication from other Tn7 transposases.

Sanger sequencing of the PCR amplified product over the 8N library of the target plasmid also elucidated that the PAM preference of the MG64-1 effector as a nGTn/nGTt on the 5′ end of the spacer (FIG. 7C). NGS analysis of the PAM library target corroborated the nGTn motif specificity at the 5′ end.

### Example 3—Predicted RNA Folding

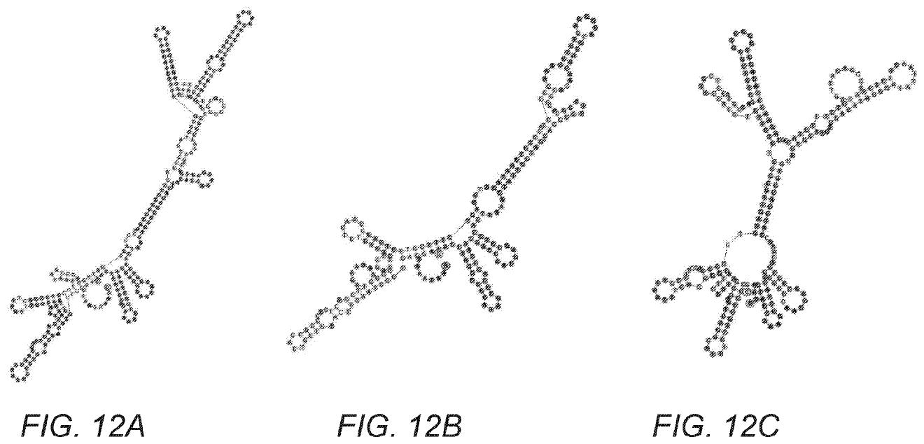

Predicted RNA folding of the active single RNA sequence was computed at 37° using the method of Andronescu 2007. All hairpin-loop secondary structures were singly deleted from the structure and iteratively compiled into a smaller single guide. In a second approach, the tracrRNA of MG64-1 was aligned to documented type Vk tracrRNA, and areas of unique insertions were mutated out of the single guide, and minimized by 57 bases. FIG. 12A depicts the predicted structure of MG64-1 sgRNA. FIG. 12B depicts the predicted structure of MG64-3 sgRNA. FIG. 12C depicts the predicted structure of MG64-5 sgRNA. The color of the bases corresponds to the probability of base pairing of that base, wherein red represents high probability and blue represents low probability.

### Example 4—Transposon End Verification Via Gel Shift

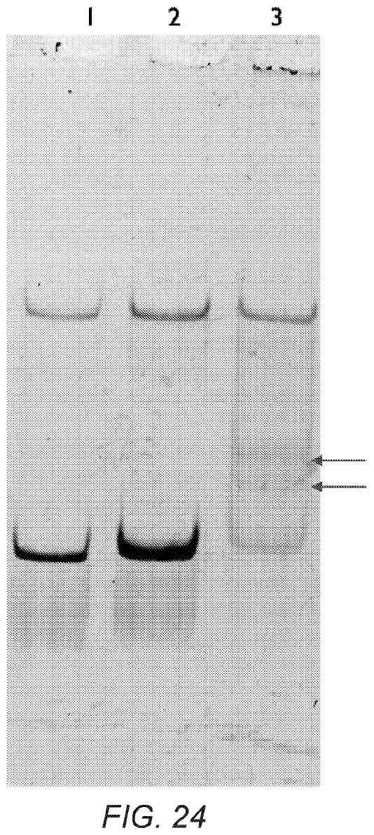

The transposon ends were tested for TnsB binding via an electrophoretic mobility shift assay (EMSA). In this case the potential LE or RE was synthesized as a DNA fragment (100-500 bp) and end-labeled with FAM via PCR with FAM-labeled primers. The TnsB protein was synthesized in an in vitro transcription/translation system. After synthesis, 1 μL of TnsB protein was added to 50 nM of the labeled RE or LE in a 10 μL reaction in binding buffer (20 mM HEPES pH 7.5, 2.5 mM Tris pH 7.5, 10 mM NaCl. 0.0625 mM EDTA, 5 mM TCEP, 0.005% BSA, 1 μg/mL poly(dI-dC), and 5% glycerol). The binding was incubated at 30° for 40 minutes, then 2 μL of 6× loading buffer (60 mM KCl, 10 mM Tris pH 7, 6, 50% glycerol) was added. The binding reaction was separated on a 5% TBE gel and visualized. Shifts of the LE or RE in the presence of TnsB were attributed to successful binding and were indicative of transposase activity (FIG. 24).

### Example 5—Integrase Activity in E. coli

As E. coli lacks the capacity to efficiently repair genomic double-stranded DNA breaks, transformation of E. coli by agents able to cause double-stranded breaks in the E. coli genome causes cell death. Exploiting this phenomenon, endonuclease or effector-assisted integrase activity was tested in E. coli by recombinantly expressing either the endonuclease or effector-assisted integrase and a guide RNA (determined e.g., as in Example 3) in a target strain with spacer/target and PAM sequences integrated into its genomic DNA.

Engineered strains were then transformed with a plasmid containing the nuclease or effector with single guide RNA, a plasmid expressing the integrase and accessory genes, and a plasmid containing a temperature sensitive origin of replication with a selectable marker flanked by left end (LE) and right end (RE) transposon motifs for integration. Transformants induced for expression of these genes were then screened for transfer of the marker to the genomic target by selection at restrictive temperature for plasmid replication and the marker integration in the genome was confirmed by PCR.

Off target integrations were screened using an unbiased approach. In brief, purified gDNA was fragmented with Tn5 transposase or shearing, and DNA of interest was then PCR amplified using primers specific to a ligated adaptor and the selectable marker. The amplicons were then prepared for NGS sequencing. Analysis of the resulting sequences were trimmed of the transposon sequences and flanking sequences were mapped to the genome to determine insertion position, and off target insertion rates were determined.

### Example 6—Colony PCR Screen of Transposase Activity

For testing of nuclease or effector assisted integrase activity in bacterial cells, strain MGB0032 was constructed from BL21(DE3) E. coli cells which were engineered to contain the target and corresponding PAM sequence specific to MG64_1. MGB0032 E. coli cells were then transformed with pJL56 (plasmid that expresses the MG64_1 effector and helper suite, ampicillin resistant) and pTCM 64_1 sg, a chloramphenicol-resistant plasmid that expresses the single guide RNA sequence for the engineered target of interest driven by a T7 promoter.

An MGB0032 culture containing both plasmids was then grown to a saturation, diluted at least 1:10 into growth culture with appropriate antibiotics, and incubated at 37° C. until OD of approximately 1. Cells from this growth stage were made electrocompetent and transformed with streamlined 64_1 pDonor, a plasmid bearing a tetracycline resistance marker flanked by left end (LE) and right end (RE) transposon motifs for integration. Electroporated cells were then recovered for 2 hours on LB medium in the presence or absence of IPTG at a final concentration of 100 μM before being plated on LB-agar-ampicillin-chloramphenicol-tetracycline and incubated 4 days at 37° C. Sterile toothpicks were used to sample each resultant CFU, which was mixed into water. To this solution was added Q5 High Fidelity PCR mastermix (New England Biolabs) and primers LA155 (5′-GCTCTTCCGATCTNNNNNGATGAGCGCATTGTTAGATTTCAT-3′ (SEQ ID NO: 1256)) and oJL50 (5′-AAACCGACATCGCAGGCTTC-3′ (SEQ ID NO: 1257)). These primers flank the predicted insertion junction. The predicted product size was 609 bp. DNA amplified PCR product was visualized on a 2% agarose gel. Sanger sequencing of PCR products confirmed the transposition event.

### Example 7—in Cell Expression/In Vitro Assay

To test the functionality of the NLS constructs in a physiologically relevant environment, constructs cloned with active NLS-tagged CAST components were integrated into K562 cells using lentiviral transduction. Briefly, constructs cloned into lentiviral transfer plasmids were transfected into 293T cells with envelope and packaging plasmids, and virus containing supernatant was harvested from the media after 72 hr incubation. Media containing virus was then incubated with K562 cell lines with 8 μg/mL of polybrene for 72 hrs, and transfected cells were then selected for integration in bulk using Puromycin at 1 μg/mL for 4 days. Cell lines undergoing selection were harvested at the end of 4 days, and differentially lysed for nuclear and cytoplasmic fractions. Subsequent fractions were then tested for transposition capability with a complementary set of in vitro expressed components.

10 million cells were centrifuged and washed once with 1×PBS pH 7.4. Supernatant wash was aspirated completely to the cell pellet, and flash frozen at −80° C. for 16 hrs. After thawing on ice, cell pellet size was measured by mass, and appropriate extraction volumes of cell fractionation and nuclear extraction reagent (NE-PER) was used to natively extract proteins in cell fractions. Briefly, cytoplasmic extraction reagent was used at 1:10 mass of cells to volume of extraction reagent. Cell suspension was mixed by vortexing and lysed with non-ionic detergent. Cells were then centrifuged at 16.000×g at 4° C. for 5 minutes. Cytoplasmic extraction supernatant was then decanted and saved for in vitro testing. Nuclear extraction reagent was then added 1:2 original cell mass to nuclear extraction reagent and incubated on ice for 1 hr on ice with intermittent vortexing. Nuclear suspension was then centrifuged at 16,000×g for 10 minutes at 4° C. and supernatant nuclear extract was decanted and tested for in vitro transposition activity. Using 4 μL of each cell and nuclear extract for each condition, the in vitro transposition reaction was performed with a complementary set of in vitro expressed proteins, donor DNA, pTarget, and buffer. Evidence of transposition activity was assayed by PCR amplification of donor-target junctions.

### Example 8—Activity in Mammalian Cells (Prophetic)

To show targeting and cleavage activity in mammalian cells, nuclear localization sequences are fused to the C terminus of each of the nuclease or effector proteins and integrase proteins and the fusion proteins are purified. A single guide RNA targeting a genomic locus of interest is synthesized and incubated with the nuclease/effector protein to form a ribonucleoprotein complex. Cells are transfected with a plasmid containing a selectable neomycin resistance marker (NeoR) or a fluorescent marker flanked by the left end (LE) and right end (RE) motifs, recovered for 4-6 hours, and subsequently electroporated with nuclease RNP and integrase proteins. Integration of a plasmid into the genome is quantified by counting G418-resistant colonies or fluorescence activated cell cytometry. Genomic DNA is extracted 72 hours after electroporation and used for the preparation of an NGS-library. Off target frequency is assayed by fragmenting the genome and preparing amplicons of the transposon marker and flanking DNA for NGS library preparation. At least 40 different target sites are chosen for testing each targeting system's activity.

### Example 9—Activity of Targeted Nuclease

In situ expression and protein sequence analyses suggested that some RNA guided effectors are active nucleases. They contain predicted endonuclease-associated domains (matching RuvC and HNH_endonuclease domains) and predicted HNH and RuvC catalytic residues (FIG. 4A).

Candidate activity was tested with engineered single guide RNA sequences using the in vitro expression system and in vitro transcribed RNA. Active proteins that successfully cleaved the library yielded a band around 170 bp in the gel.

### Example 10—Identification of Transposons

Transposons are predicted to be active when they contain one or more protein sequences with transposase and/or integrase function between the left and right ends of the transposon. A Tn7 transposon, as defined here, may comprise a catalytic transposase TnsB, but may also contain TnsA, TnsC, TnsD, TnsE, TniQ, and/or other transposases or integrases. The transposon ends comprise predicted transposase binding sites, which contain direct and/or inverted repeats of 15 bp to 150 bp in length flanking the transposase proteins and other ‘cargo’ genes. Protein sequence analysis indicated that the transposases contain integrase domains, transposase domains and/or transposase catalytic residues, suggesting that they are active (e.g., FIG. 4A and Panel A of FIG. 5).

### Example 11—Identification of CRISPR-Associated Transposons

Putative CRISPR-associated transposons (CAST) contain a DNA and/or RNA targeting CRISPR effector and proteins with predicted transposase function in the vicinity of a CRISPR array. In some systems, the effector is predicted to have nuclease activity based on the presence of endonuclease-associated catalytic domains and/or catalytic residues (e.g., FIG. 4A). The transposases were predicted to be associated with the active nucleases when the CRISPR loci (CRISPR nuclease and array) and the transposase proteins are located between the predicted transposon left and right ends (e.g., FIGS. 4B-4C). In this case, the effector was predicted to direct DNA integration to specific genomic locations based on a guide RNA.

In some systems, the effector was predicted to have homology with documented CRISPR effector proteins, but to be inactive based on the absence of endonuclease domains and/or catalytic residues (Panel A of FIG. 5). The transposases were predicted to be associated with the effector when the CRISPR loci (inactive CRISPR nuclease and array) and the transposase proteins were located within the predicted transposon left and right ends (Panels A and B of FIG. 5).

### Example 12—CAST Identification

CRISPR-associated transposons (CAST) are systems that comprise a transposon that has evolved to interact with a CRISPR system to promote targeted integration of DNA cargo.

CASTs are genomic sequences encoding one or more protein sequences involved in DNA transposition within the signature left and right ends of the transposon. A Tn7 transposon, as defined here, may comprise a catalytic transposase TnsB, but may also contain a catalytic transposase TnsA, a loader protein TnsC or TniB, and target recognition proteins TnsD, TnsE, TniQ, and/or other transposon-associated components. The transposon ends comprise predicted transposase binding sites, which contain direct and/or inverted repeats of 15 bp to 150 bp in length flanking the transposon machinery and other ‘cargo’ genes.

In addition. CASTs also encode a DNA and/or RNA targeting CRISPR nuclease or effector in the vicinity of a CRISPR array. In some systems, the effector was predicted to be an active nuclease based on the presence of endonuclease-associated catalytic domains and/or catalytic residues. In some systems, the effector was predicted to have sequence similarity with documented CRISPR effector proteins, but to be inactive based on the absence of endonuclease domains and/or catalytic residues. The transposons were predicted to be associated with the effector when the CRISPR locus and the transposon-associated proteins were located within the predicted transposon left and right ends. In this case, the effector was predicted to direct DNA integration to specific genomic locations based on a guide RNA.

### Example 13—Class 2 Cas12K CAST

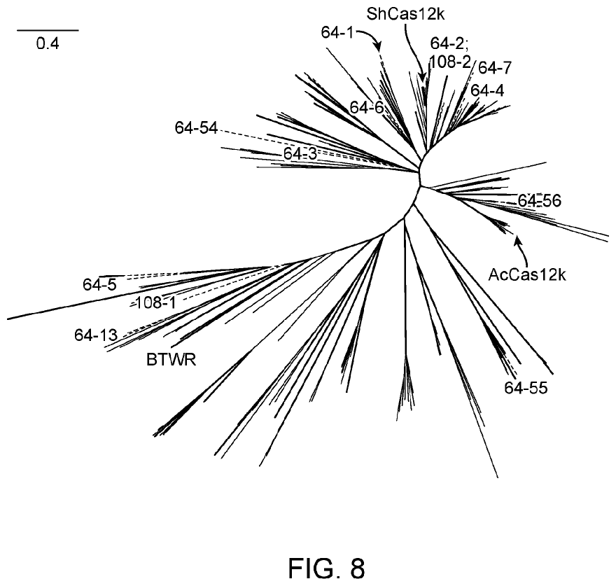

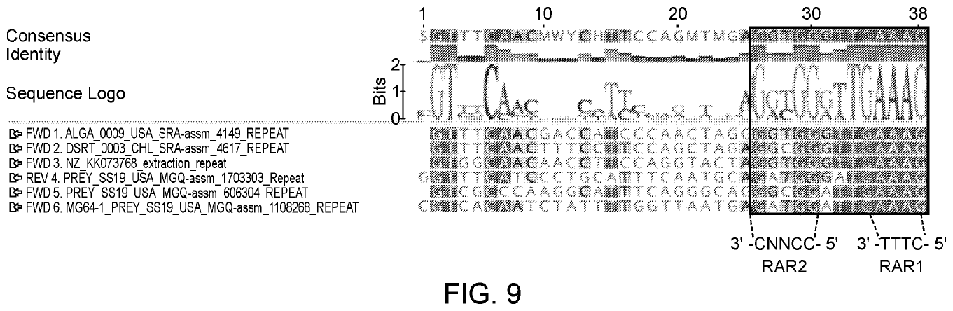

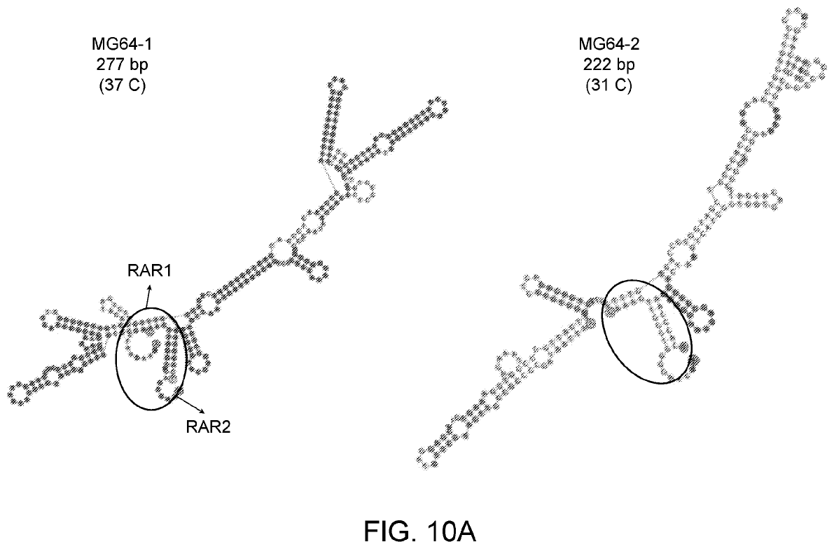

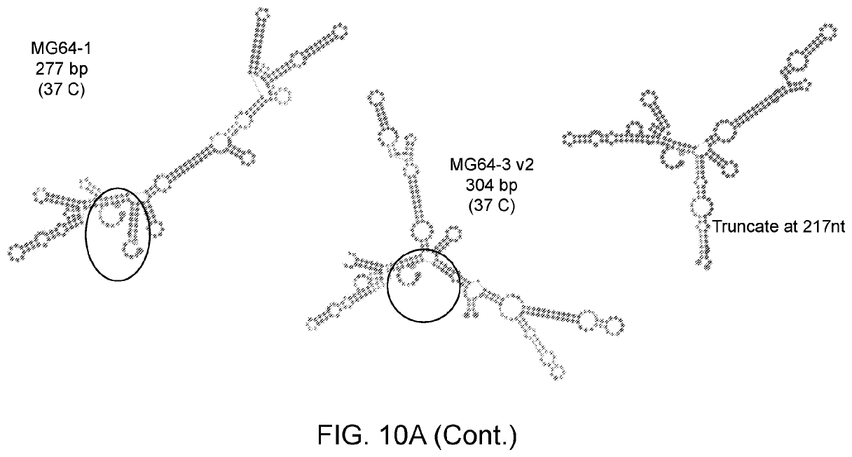

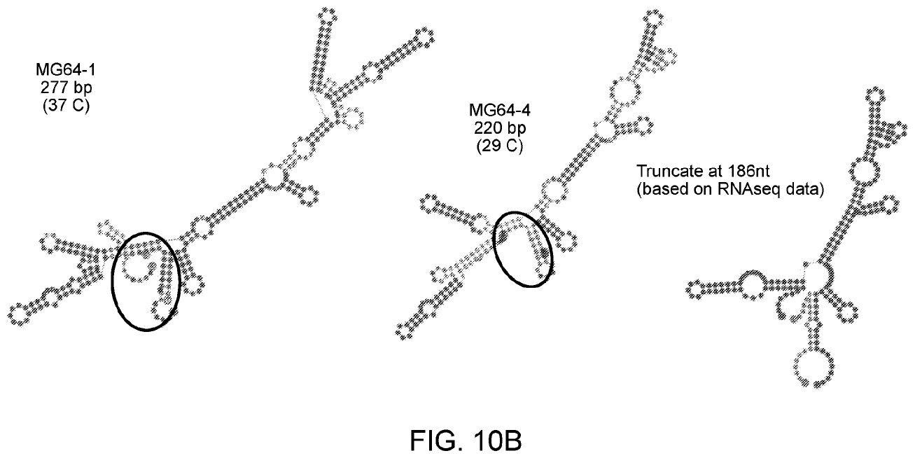

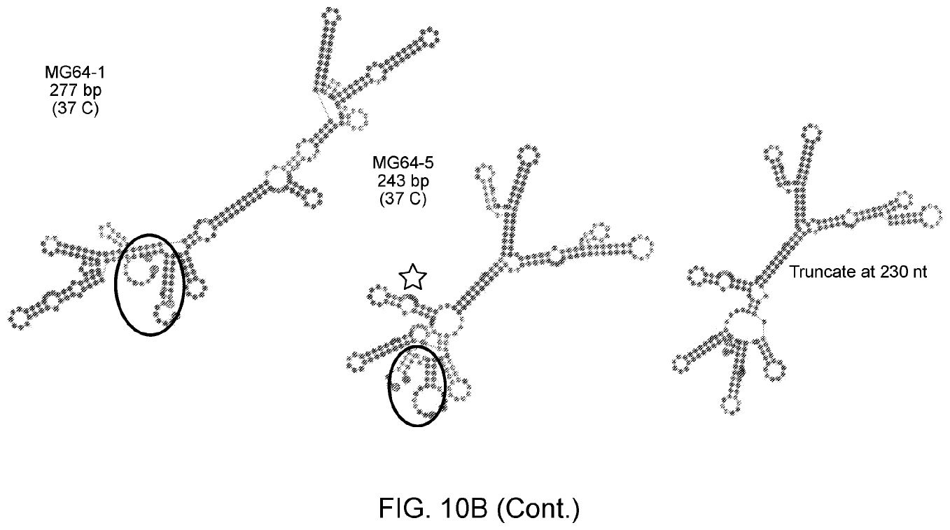

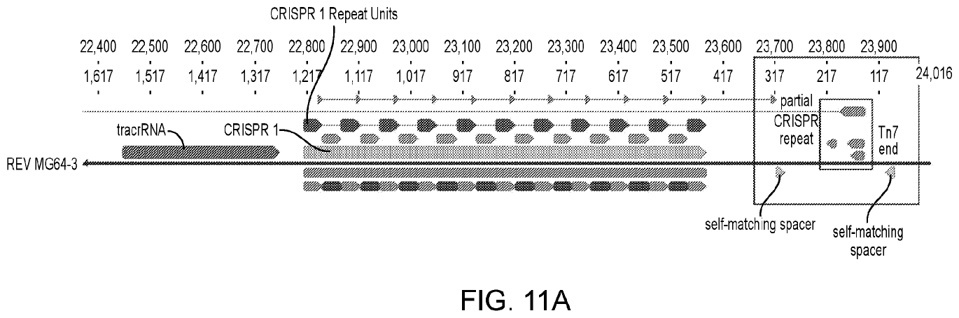

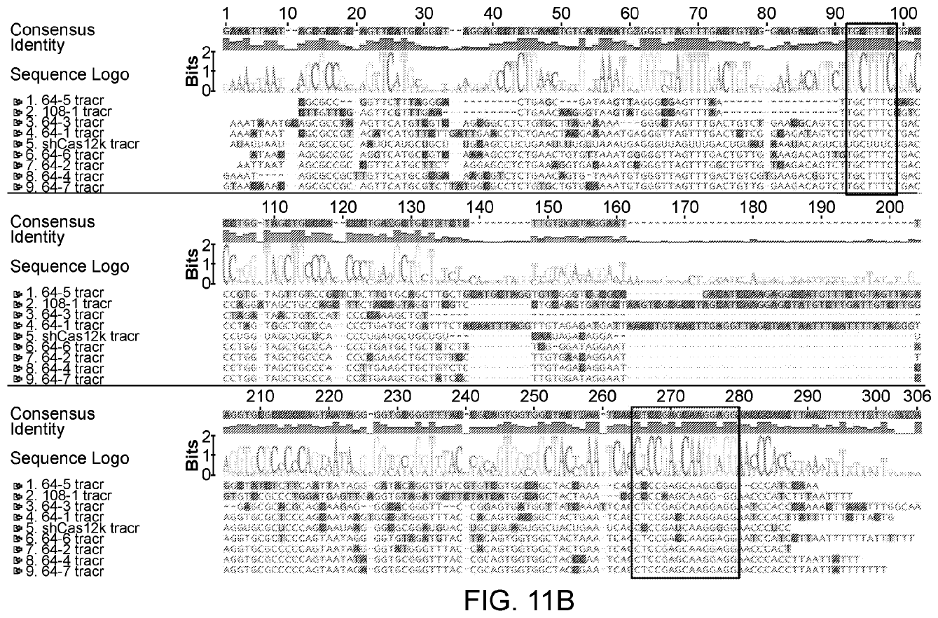

Cas12k CAST systems encode a nuclease-defective CRISPR Cas12k effector, a CRISPR array, a tracrRNA, and Tn7-like transposition proteins. Cas12k effectors are phylogenetically diverse and features that confirm their association with CASTs have been confirmed for several (FIG. 8). For example, the transposon left end was identified downstream from the MG64-3 CRISPR locus, as shown by terminal inverted repeats and self-matching spacer sequences (FIG. 11A). Cas12k CAST CRISPR repeats (crRNA) contain a conserved motif 5′-GNNGGNNTGAAAG-3′ (FIG. 9). Short repeat-antirepeats (RAR) within the crRNA motif aligned with different regions of the tracrRNA (FIG. 9 and FIG. 10), and RAR motifs appeared to define the start and end of the tracrRNA (for example, for MG64-1, the 5′ end of the tracrRNA contained RAR1 (TTTC) and the 3′ end contained RAR2 (CCNNC), (FIG. 10A).

### Example 14—Transposon End Prediction

Transposon ends were estimated from intergenic regions flanking the effector and the transposon machinery. For example, for Cas12k CAST, the intergenic region located directly upstream from TnsB and directly downstream from the CRISPR locus, were predicted as containing the Tn7 transposon left and right ends (LE and RE).

Direct and inverted repeats (DR/IR) of ~12 bp were predicted on the contig, with up to 2 mismatches. In addition, the Dotplot algorithm was used to find short (~10-20 bp) DR/IR flanking CAST transposons. Matching DR/IR located in intergenic regions flanking CAST effector and transposon genes are predicted to encode transposon binding sites. LE and RE extracted from intergenic regions, which encode putative transposon binding sites, were aligned to define the transposon ends boundaries. Putative transposon LE and RE ends are regions: a) located within 400 bp upstream and downstream from the first and last predicted transposon encoded genes: b) sharing multiple short inverted repeats; and c) sharing >65% nucleotide id.

### Example 15—Single Guide Design

Analysis of the intergenic regions surrounding the Cas effector and CRISPR array identified a potential anti-repeat sequence and a conserved “CYCC(n6)GGRG” stem loop structure neighboring the antirepeat corresponding to the duplexing sequence of the tracrRNA (FIG. 11B). TracrRNA and crRNA repeat were folded and trimmed, adding a tetraloop sequence of GAAA to maintain the stem loop region of the crRNA-tracrRNA complementary sequence.

### Example 16—In Vitro Integration Activity Using Targeted Nuclease

In situ expression and protein sequence analyses indicated that some RNA guided effectors are active nucleases. They contain predicted endonuclease-associated domains (matching RuvC and HNH_endonuclease domains), and/or predicted HNH and RuvC catalytic residues. Candidate activity was tested with engineered single guide RNA sequences using the in vitro expression system and in vitro transcribed RNA. Active proteins that successfully cleaved the library yielded a band around 170 bp in the gel.

### Example 17—Programmable DNA Integration

CAST activity was tested with five types of components (1) a Cas effector protein (SEQ ID NO: 1) expressed in vitro expression systems, (2) a target DNA fragment or plasmid containing the target sequence and PAM corresponding to the Cas enzyme (SEQ ID NO: 31), (3) a donor DNA fragment containing a marker or fragment of DNA flanked by the LE and RE of the transposase system in a DNA fragment or plasmid (SEQ ID NOs: 8-11) (4) any combination of transposase proteins expressed using in vitro expression systems (SEQ ID NO: 2-4), and (5) an engineered in vitro transcribed single guide RNA sequence (SEQ ID NO: 5). Active systems that successfully transposed the donor fragment were assayed by PCR amplification of the donor-target junction.

After performing the transposition reaction, PCR amplification of the junction showed that proper donor-target formation occurred and that the transposition reaction was sg dependent. (FIG. 9). PCR amplification of reactions #3 and #4 indicated that both orientations of the donor relative to the target were made: one where the LE is closer to the PAM, and one where the RE is closer to the PAM. While both transposition orientations occurred, there appeared to be a preference for donor integration in the target where the LE is closer to the PAM, represented by strong band present for reactions #4 and #5.

Sanger sequencing of the preferred orientation product was performed. Of the integrations that occurred with the LE closer to the PAM, there was a clear degradation of the sequencing chromatogram signal from either the forward or reverse direction over the target/donor junction. This indicated that, of the products that were oriented with the LE closer to the PAM, integration occurred in a range of nucleotides, with the primary product of LE-closer-to-PAM products as a 61 bp integration from the PAM (FIG. 10A). Sequencing that originated from the donor over the donor-target junction defined the composition of the essential outer bounds of the LE and RE sequences (FIG. 10A, FIG. 10B). Sequencing of the RE on LE-closer-to-PAM products showed a 3 bp duplication downstream of the donor RE (FIG. 10B). This is in part due to the Tn7 transposase integration event that cleaved and ligated the donor fragment at a staggered cut site. A 3 bp duplication is smaller than the expected 5 bp of duplication from other Tn7 transposases.

Sanger sequencing of the PCR amplified product over the 8N library of the target plasmid also indicated that the PAM preference of the MG64-1 effector as a nGTn/nGTt on the 5′ end of the spacer (FIG. 10C). NGS analysis of the PAM library target corroborated that the nGTn motif selectivity at the 5′ end.

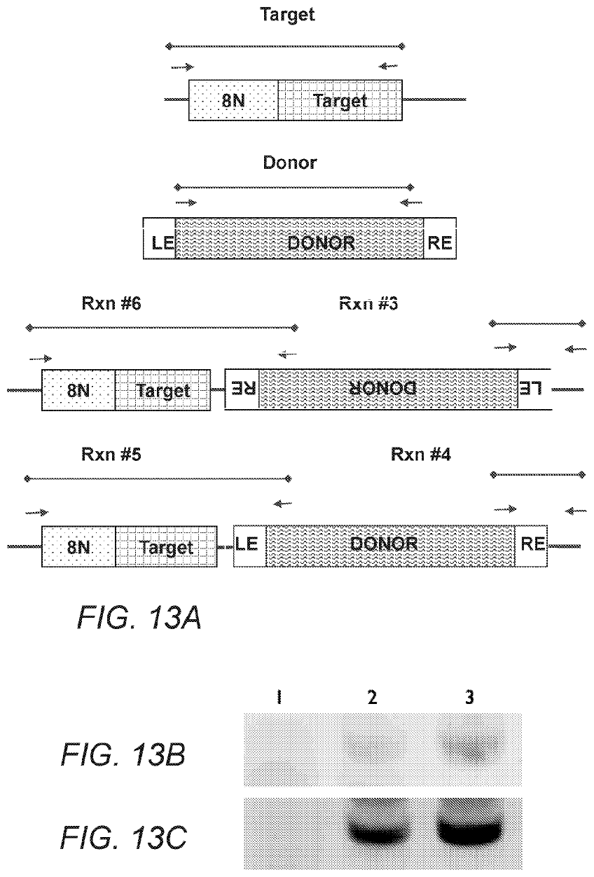

Further development of single guide testing confirmed activity of MG64-1 with a new sgRNA scaffold (FIG. 13).

### Example 18—Integration Window Determination

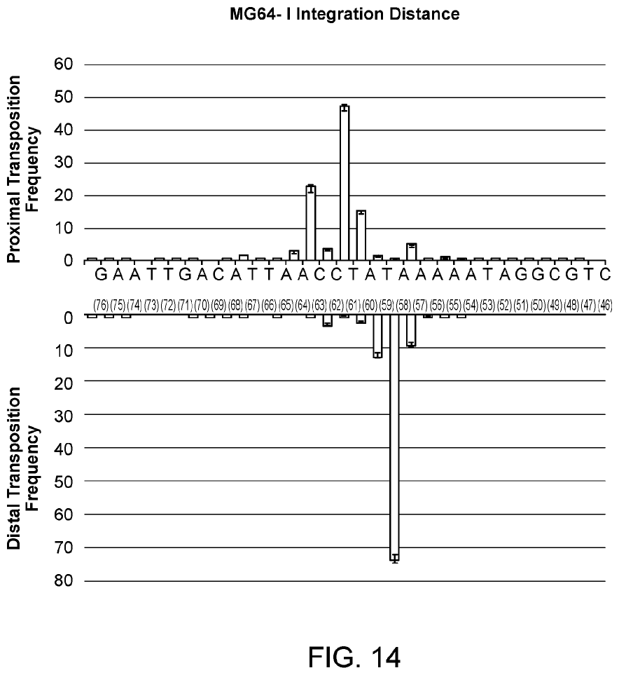

PCR junctions of the PAM that were amplified were indexed for NGS libraries and sequenced. Reads were mapped and quantified using an amplicon sequence of a putative transposition sequence with a 60 bp distance of integration from the PAM (guideseq=20 bp 3′ end of LE or RE, center of window=0, window size=20). Indel histogram was normalized to total indel reads detected, and frequencies were plotted relative to the 60 bp reference sequence (FIG. 14)

Both PCR reactions 5 (LE proximal to PAM, FIG. 14 top panel) and PCR 4 (RE distal to PAM, FIG. 14 bottom panel) were plotted on the sequence and distance from the PAM for MG64-1. Analysis of the integration window indicates that 95% of the integrations that occurred at the spacer PAM site were within a 10 bp window between 58 and 68 nucleotides away from the PAM. Differences in the integration distance between the distal and the proximal frequencies reflected the integration site duplication—a 3-5 base pair duplication as a result of staggered nuclease activity of the transposase upon integration.

### Example 19—Colony PCR Screen of Transposase Activity

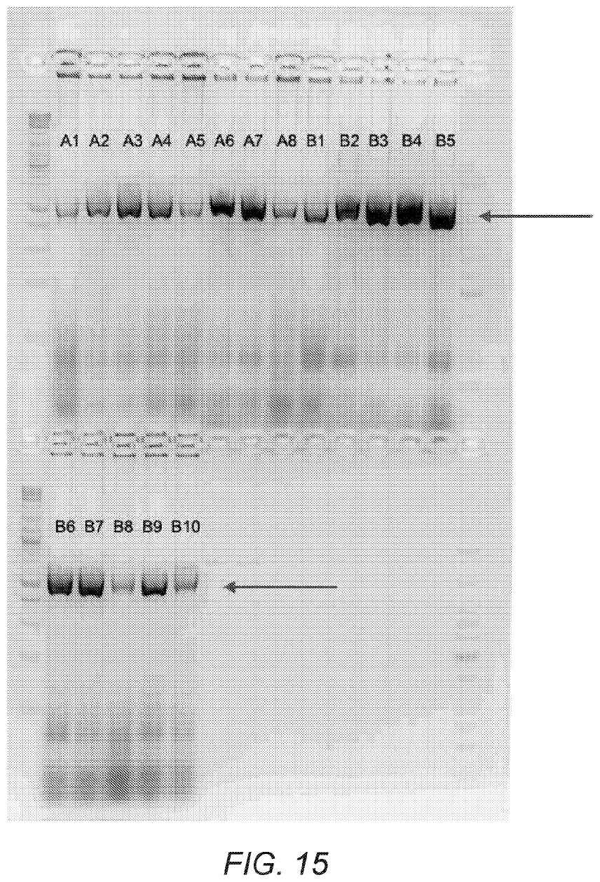

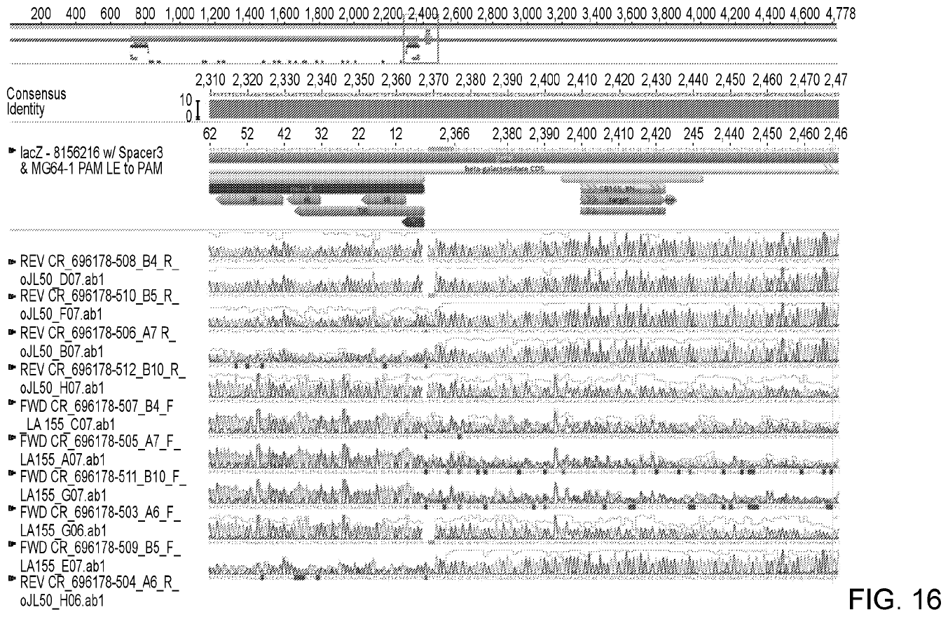

Transposition activity was assayed via a colony PCR screen. After transformation with the pDonor plasmids, E. coli were plated onto LB-agar containing ampicillin, chloramphenicol, and tetracycline. Select CFUs were added to a solution containing PCR reagents and primers that flank the selected insertion junction. PCR reactions of the integration products were visible on a gel (FIG. 15). Sequencing results of select colony PCR products confirmed that they represent transposition events, as they spanned the junction between the LE and the PAM at the engineered target site, which is in the lacZ gene (FIG. 16).

### Example 20—Single Guide Engineering

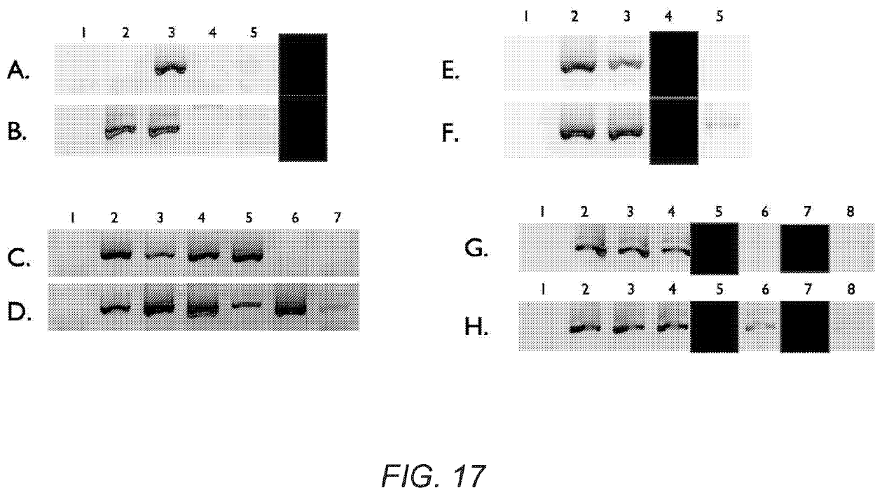

Predicted RNA folding of the active single RNA sequence was computed at 37° using the method of Andronescu 2007. All hairpin-loop secondary structures were single deleted from the construct and iteratively compiled into a smaller single guide. Engineered single guides (esg) 4, 6, 7, 8, 9 were active for donor transposition (Panels C and D of FIG. 17), with engineered sgRNAs 8 and 9 being weaker single guides and transposing with PCR 5 (Panel D of FIG. 17). Engineered guide 5 was able to transpose, however engineered sgRNA 10 weakly transposed with PCR 5 (Panels E and F of FIG. 17) Esg 17 is a combination of deletions in esg6 and esg7, and esg 18 is a combination of esg 4 and esg 5. Both were able to strongly transpose across both PCR 4 and 5 (Panels G and H of FIG. 17), However, combinatorial addition of esg 6 and esg 18 making esg 19, resulted in a weaker transposition in PCR5, and addition of esg 7 to esg 19, making esg 20 results in a very weak junction of transposition for PCR 5 (Panels G and H of FIG. 17). In a second approach, the tracrRNA of MG64-1 was aligned to documented type Vk tracrRNA, and areas of unique insertions were mutated out of the single guide. sgRNA was minimized by truncation of insertion sequences of the MG64-1 sgRNA (FIG. 14). 2 subsequent deletions, esg 2 and esg 3 were also tested (Panels A and B of FIG. 17) but neither esg 2 nor esg 3 resulted in appreciable transposition, thus the single guide was minimized by 57 bases.

### Example 21—LE-RE Minimization

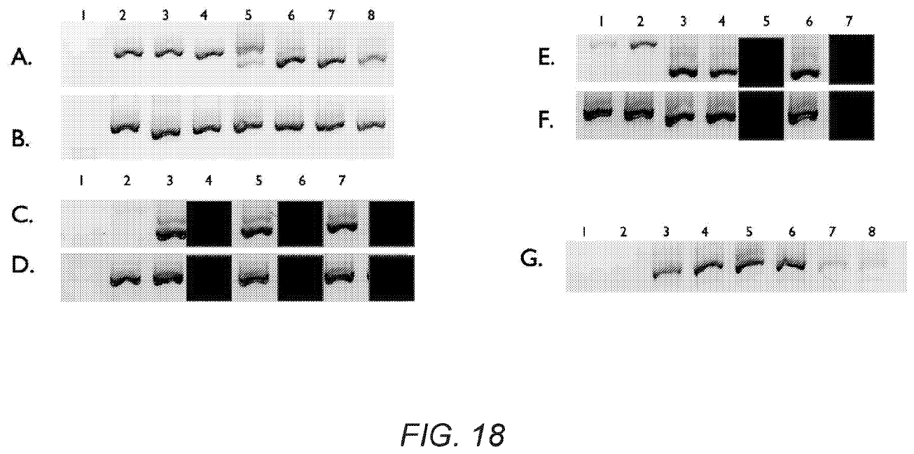

Sequencing of the target-transposition junction aided in identification of the terminal inverted repeats by identifying the outmost sequence from the donor plasmid that was incorporated into the target reaction. By performing repeat analysis of 14 bp with variability of 10%, short repeats contained within the terminal ends were identified and truncations of these minimal ends to preserve the repeats while deleting superfluous sequence were designed. Prediction and cloning was done in multiple iterations, with each interaction tested with in vitro transposition. Initial LE and RE deletions were singly designed and cloned to the 68 bp, 86 bp, and 105 bp for the LE, 178 bp, 196 bp and 242 bp for the RE. The RE of 64-1 also had a noticeable span of sequence without a repeat, so internal deletions of both 50 bp and 81 bp were designed and cloned. Transposition among all single deletions was robust for both PCR 4 and PCR 5 (Panels A and B of FIG. 18) and internal deletion of 81 bp was subsequently pursued with combinatorial deletions for the RE. Trimmed ends of the former 178, 196 and 212 bp were cloned on the 81 bp internal deletion and transposition was tested. Transposition was active for all constructs designed. In combination with LE of 68 bp, transposition proved active down to a LE region of 68 bp combined with a RE region of 96 bp (Panels E and F of FIG. 18).

### Example 22—Overhang Influence of Transposition

In order to test whether superfluous sequence outside of the TnsB binding motifs were necessary for transposition, oligos designed for the TGTACA motifs of both LE and RE were designed and synthesized with 0, 1, 2, 3, 5 and 10 bp extra base pairs. These synthesized oligos were used to generate donor PCR fragments with overhangs and tested for their ability to transpose into the target site. Most noticeably, PCR6 was rarely detected from the in vitro reactions, (Panel G of FIG. 18, lanes 1, 2) however with a small 0-3 bp overhang, efficient integration at PCR 6 was detected, reflecting a RE proximal to PAM orientation that is not detected with a larger flanking sequence.

### Example 23—CAST NLS Design

Eukaryotic genome editing for therapeutic purposes is largely dependent on the import of editing enzymes into the nucleus. Small polypeptide stretches of larger proteins signal to cellular components for protein import across the nuclear membrane. Placement of these tags is not trivial, as import function versus function of the protein to which it is fused are potential tradeoffs depending on the location of the NLS tag. In order to test functional orientations of the NLS to each of the components of the CAST complex, constructs were designed and synthesized which fused Nucleoplasmin NLS to the N-terminus and SV40 NLS to the C-terminus of each of the components of the MG CAST. Protein of these constructs were expressed in cell free in vitro transcription/translation reactions and tested for in vitro transposition activity with a complement set of untagged components. NLS-tagged constructs were assessed for maintenance of activity by PCR of the donor-target junction using PCR 4 (Assessing RE distal transpositions) and the cognate transposition event, PCR 5 (LE to proximal transposition).

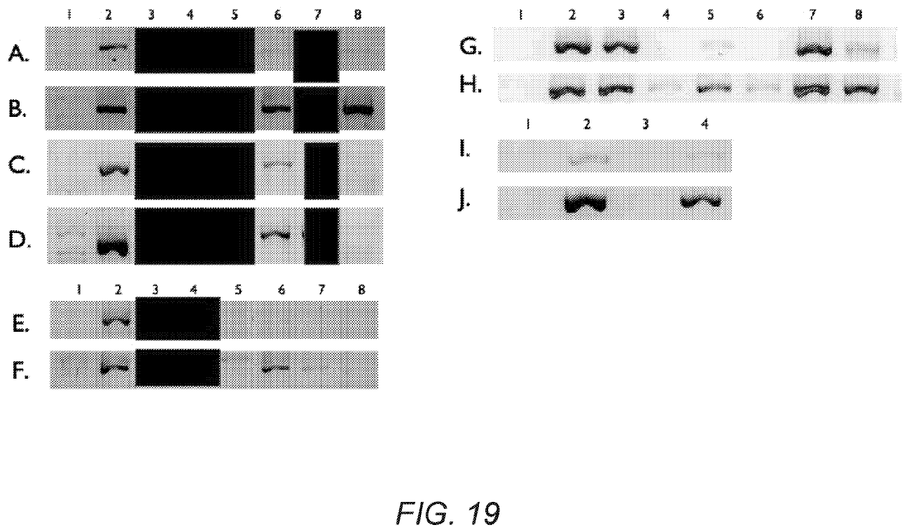

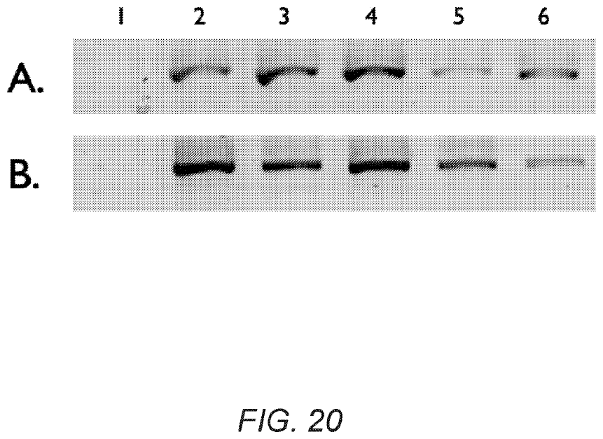

Most components resulted in a single NLS orientation that maintained activity. TnsB was the CAST component that was active with both N-terminal NLS and C terminal NLS by both PCR4 and PCR 5 (Panels A and B of FIG. 19). TniQ was active with N-terminal NLS tags (Panels C and D of FIG. 19). And Cas12k component was active with a C-terminal tagged NLS (Panels E and F of FIG. 19, lanes 5, 6). Further development of a Cas12k with both Nucleoplasmin and SV40 NLS tags were tested and found to be active (Panels I and J of FIG. 19, Lane 4). TnsC was weakly active with an N-terminal NLS (Panels E and F of FIG. 19, lane 7), but further exploration of the TnsC tagging identified new working NLS-HA-TnsC and NLS-FLAG-TnsC constructs (Panels G and H of FIG. 19, lanes 3 and 7, respectively). The end result was a completely NLS-tagged suite of components that were active in vitro with both orientations of NLS-TnsB and TnsB-NLS (Panels A and B of FIG. 20 lanes 5, 6).

### Example 24—Cas12k and TniQ Protein Fusion Construct Design and Testing

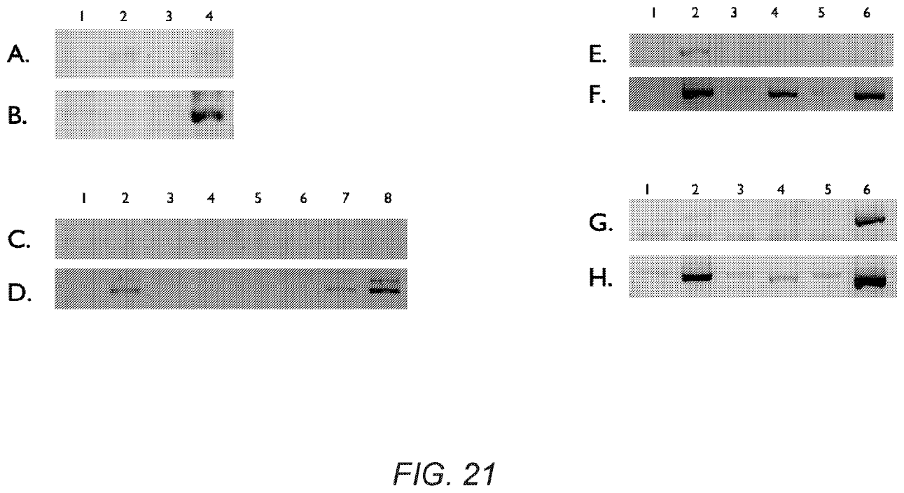

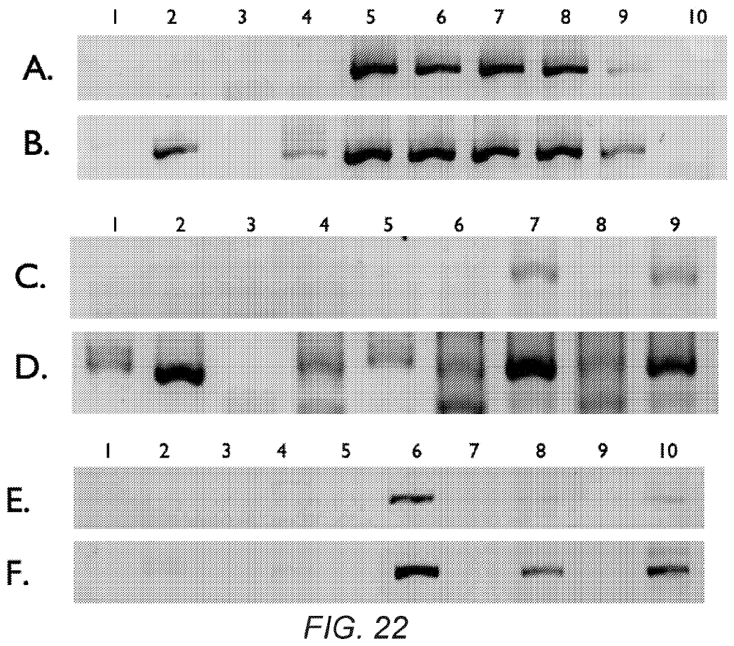

In an effort to simplify the expression of the protein components and minimize delivery of these components into cells, fusion constructs were designed, synthesized, and tested between the Cas12k effector and the TniQ protein. Both orientations of the TniQ fused to the Cas12k were designed and synthesized, a C-terminal fusion, Cas-TniQ, and an N terminal fusion, TniQ-Cas. While both constructs were weakly active for PCR4 (Panel A of FIG. 21), when expressed in vitro and assayed for transposition abilities, PCR5 junction was robustly formed by the TniQ-Cas fusion protein (Panel B of FIG. 21). Transpositions lengths were assayed with variable linker domains including the original (20 amino acid linker), 48, 68 72 and 77 (Panels C, D, E, and F of FIG. 21). NLS tags were then linked to the N terminus of TniQ and the C terminus of the Cas12k and found to still be active by PCR5 (Panels E and F of FIG. 21).

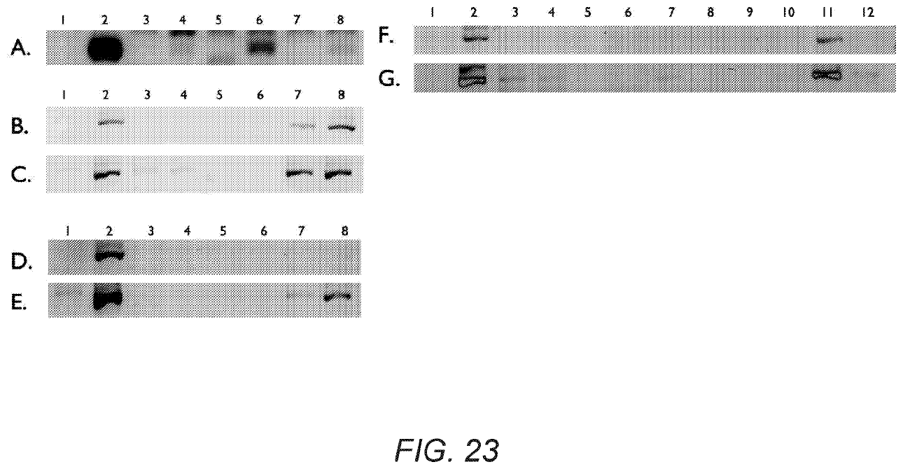

Two other linkers were employed to fuse the effector and TniQ genes. P2A, a self-stopping translation sequence was active in a Cas-NLS-P2A-NLS-TniQ construct (Panels G and H of FIG. 21, lane 6), and an MCV Internal Ribosome Entry Sequence (IRES) mRNA-based linker allowed for independent translation of the two components in cells (Panels F and G of FIG. 23).

### Example 25—Intracellular Expression Coupled In Vitro Transposition Testing

To test the functionality of the NLS constructs in a physiologically relevant environment, constructs cloned with active NLS-tagged CAST components were integrated into K562 cells using lentiviral transduction. Briefly, constructs cloned into lentiviral transfer plasmids were transfected into 293T cells with envelope and packaging plasmids, and virus containing supernatant was harvested from the media after 72 hr incubation. Media containing virus was then incubated with K562 cell lines with 8 μg/mL of polybrene for 72 hrs, and transfected cells were then selected for integration in bulk using Puromycin at 1 μg/mL for 4 days. Cell lines undergoing selection were harvested at the end of 4 days, and differentially lysed for nuclear and cytoplasmic fractions. Subsequent fractions were then tested for transposition capability with a complementary set of in vitro expressed components.

Both NLS-TnsB and TnsB-NLS were tested by cell fractionation and in vitro transposition, and transposition was detected across both cytoplasmic and nuclear fractions, and NLS-TniQ had detectable activity in the cytoplasm (Panels A and B of FIG. 22). NLS-HA-TnsC and NLS-FLAG-TnsC were both active in both cytoplasmic and nuclear fractions when expressed (Panel D of FIG. 22), however PCR4 is formed in the nuclear fraction of both TnsC constructs. (Panel C of FIG. 22).

When both NLS-TnsB or TnsB-NLS were linked with NLS-FLAG-TnsC by using an IRES, NLS-TnsB-IRES-NLS-FLAG-TnsC was largely active in the nuclear fraction while TnsB-NLS-IRES-NLS-FLAG-TnsC was active in both cytoplasmic and nuclear fractions. This is indicative that NLS-TnsB has a higher capacity of trafficking to the nucleus (Panels E and F of FIG. 21).

Cas12k fusions in the cell were similarly fractionated and tested for transposition. Cas-NLS Cas-NLS-P2A-NLS-TniQ were transduced into cells, fractionated, and tested in vitro for subcellular activity. Cas-NLS-P2A-NLS-TniQ was able to transpose in the cytoplasm with the addition of single guide to the reaction (Panel A of FIG. 23). By supplementing holo Cas protein (+sgRNA) or additional TniQ with sgRNA, the Cas-NLS-P2A-NLS-TniQ construct in the nuclear fraction was complemented. This indicates that both Cas-NLS and NLS-TniQ are making it into the nucleus (Panels B and C of FIG. 23). NLS-TniQ-Cas-NLS fusion protein had similar results, but needed more supplementation with TniQ (Panels D and E of FIG. 23), and Cas-NLS-IRES-NLS-TniQ needed supplementation from just the holo Cas-NLS (Panels F and G of FIG. 23) As a whole this indicates that all the components of the CAST have been able to be delivered to the nuclear fraction of the cell.

### Example 26—Transposon End Verification Via Gel Shift

In order to verify the activity of TnsB on the predicted transposon end sequence, the LE of MG64-1 was amplified using FAM labeled oligos. MG64-1 TnsB protein was expressed using a cell free transcription/translation system and incubated with the LE FAM labeled product. After incubation for 30 minutes, binding was observed on a native 5% TBE gel (FIG. 24). Multiple bands of fluorescent product within the co-incubated lane (FIG. 24, lane 3) indicated a minimum of 2 TnsB binding sites.

Systems of the present disclosure may be used for various applications, such as, for example, nucleic acid editing (e.g., gene editing) or binding to a nucleic acid molecule (e.g., sequence-specific binding). Such systems may be used, for example, for remediating (e.g., removing or replacing) a genetically inherited mutation that may cause a disease in a subject; inactivating a gene in order to ascertain its function in a cell; as a diagnostic tool to detect disease-causing genetic elements (e.g., via cleavage of reverse-transcribed viral RNA or an amplified DNA sequence encoding a disease-causing mutation); as deactivated enzymes in combination with a probe to target and detect a specific nucleotide sequence (e.g., sequence encoding antibiotic resistance int bacteria); to render viruses inactive or incapable of infecting host cells by targeting viral genomes; to add genes or amend metabolic pathways to engineer organisms to produce valuable small molecules, macromolecules, or secondary metabolites; to establish a gene drive element for evolutionary selection, and/or to detect cell perturbations by foreign small molecules and nucleotides as a biosensor.

### Example 27—Class 2 Cas12k CAST System Prediction

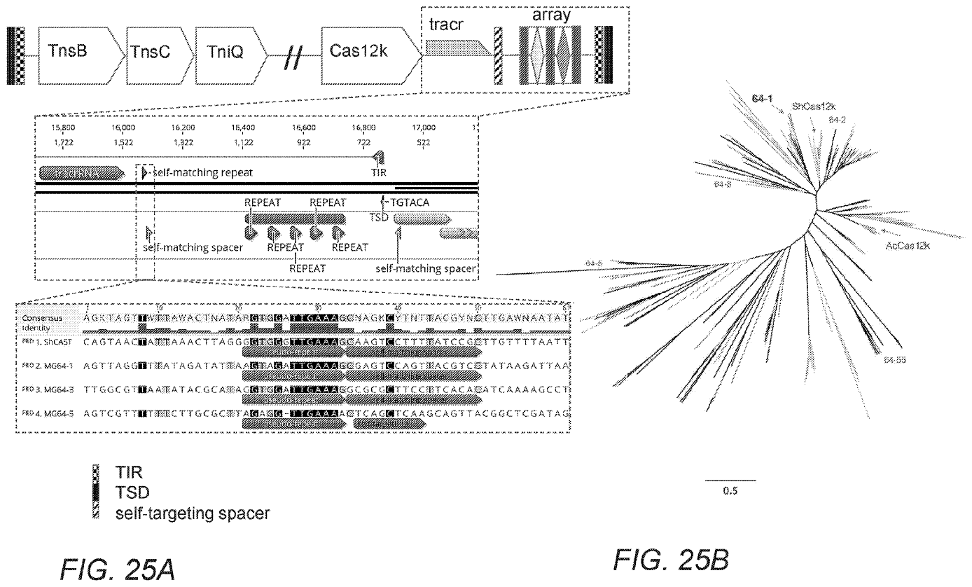

Cas12k CAST systems encode a nuclease-defective CRISPR Cas12k effector, a CRISPR array, a tracrRNA, and Tn5053-like transposition proteins (FIG. 25A). Cas12k effectors are phylogenetically diverse and features that establish their association with CASTs have been confirmed (FIGS. 25A-25B). For example, the transposon left end was identified downstream from many Cas12k effectors and their CRISPR locus, as shown by terminal inverted repeats and self-matching spacer sequences (FIGS. 25A-25B).

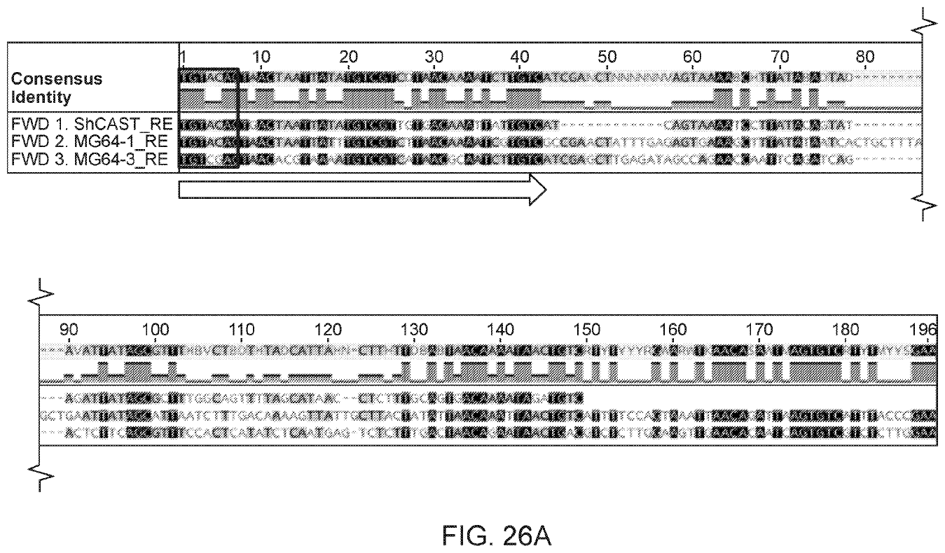

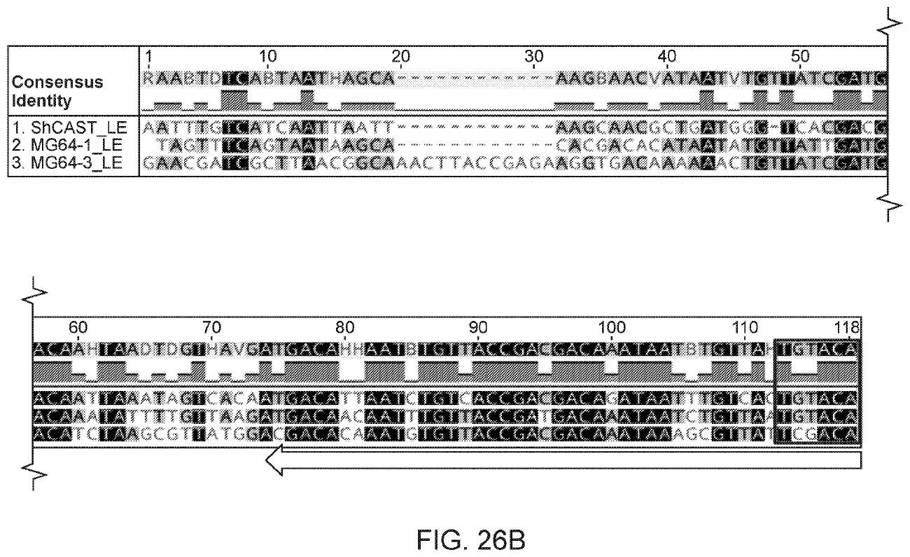

Transposon ends of Cas12k CAST systems were determined from intergenic regions flanking the CRISPR locus and the transposon machinery. For example, the intergenic region located directly upstream from TnsB and directly downstream from the CRISPR locus, were predicted as containing the transposon left and right ends (LE and RE). These intergenic regions were aligned among several homologs and regions of conservation were used to predict the transposon ends boundaries (FIG. 26).

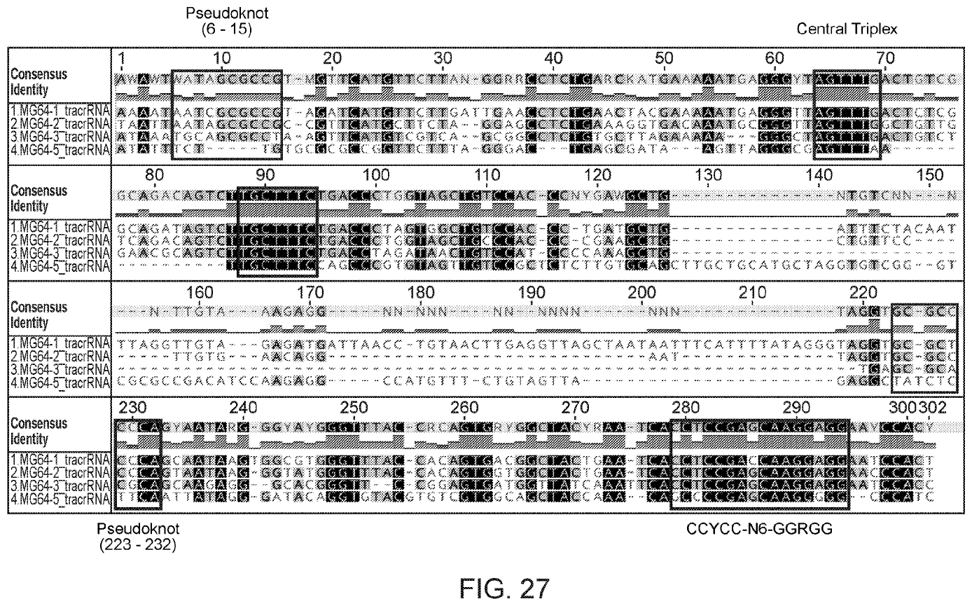

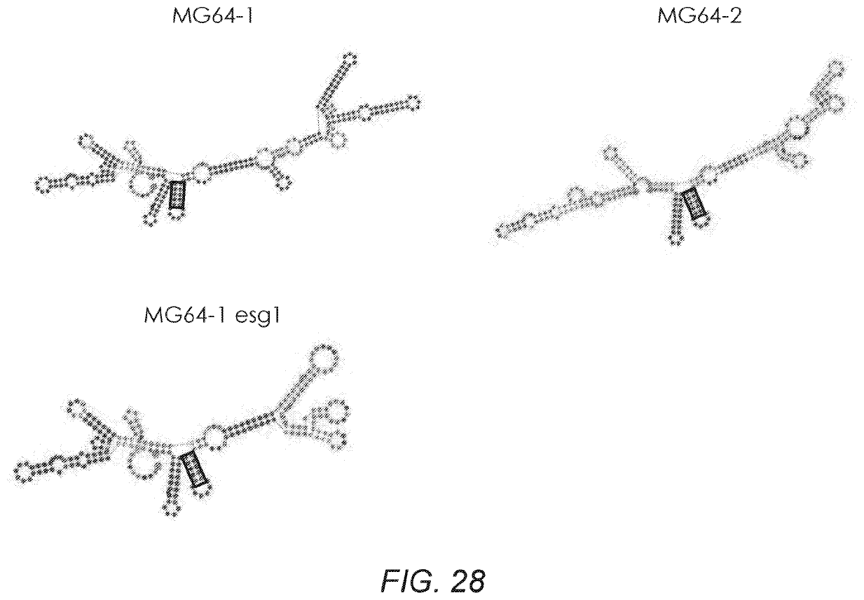

The 3′ end of Cas12k CAST CRISPR repeats (crRNA) contain a conserved motif 5′-GNNGGNNTGAAAG-3′ when aligned among homologs, and they are predicted to bind to different regions of the tracrRNA to form secondary and tertiary guide RNA structures (FIG. 27 and FIG. 28). Self-matching spacers within the CAST transposon are often found next to a pseudo CRISPR repeat in the vicinity of the CRISPR arrays (FIG. 25A, bottom alignment).

Analysis of the intergenic regions surrounding the Cas effector and CRISPR array identified a potential anti-repeat sequence and a conserved “CCYCC(n6)GGRGG” (SEQ ID NO: 1258) stem-loop structure neighboring the antirepeat, corresponding to the duplexing sequence of the tracrRNA (FIG. 27). Good quality tracrRNAs were used to build covariance models and searched on all Cas12k CAST genomic fragments identified in this study.

For single guide RNA (sgRNA) design, tracrRNA and crRNA repeat were folded and trimmed, adding a tetraloop sequence of GAAA to maintain the stem loop region of the crRNA-tracrRNA complementary sequence (FIG. 28). Generally, sgRNAs share conserved structural features despite sharing less than 70% pairwise nucleotide identity (FIG. 27).

### Example 28—In Vitro Characterization of Cas12k CAST Systems

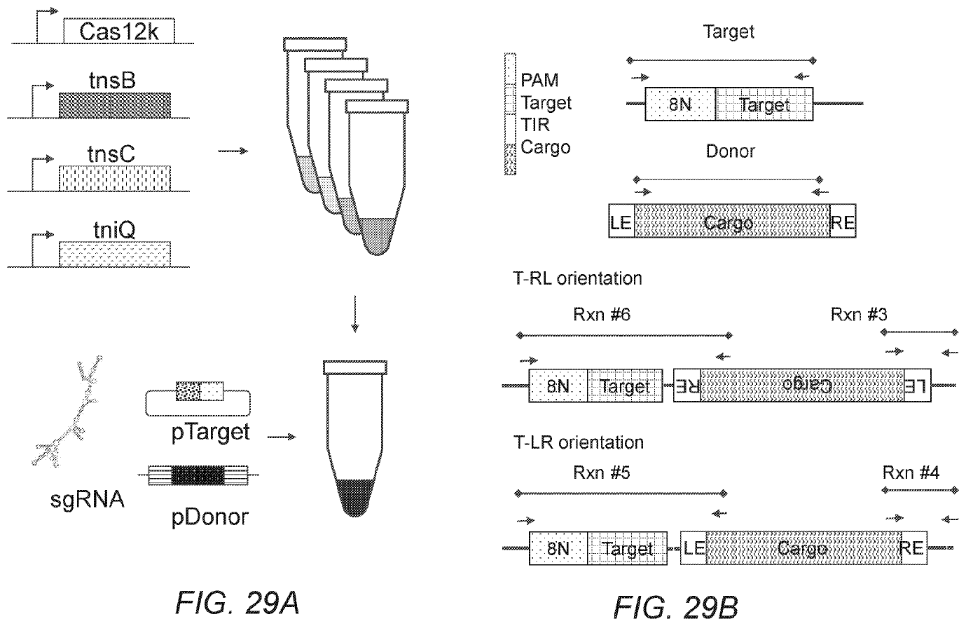

In order to test the function of Cas12k CAST systems and elucidate the potential PAM, a transposition reaction was assembled using synthesized Cas12k effectors and Tn5053-like proteins under the control of a T7 promoter. Each open reading frame was expressed in vitro with an in vitro expression system and assembled in a transposition reaction with a transposition buffer, a donor PCR fragment, and a plasmid based target with an 8N target library (Panel A of FIG. 29). When CAST systems are active and can transpose the donor fragment into the library of target plasmids, the transposition reaction can be PCR amplified to recover each donor-target junction of the two potential products of transposition (Panel B of FIG. 29).

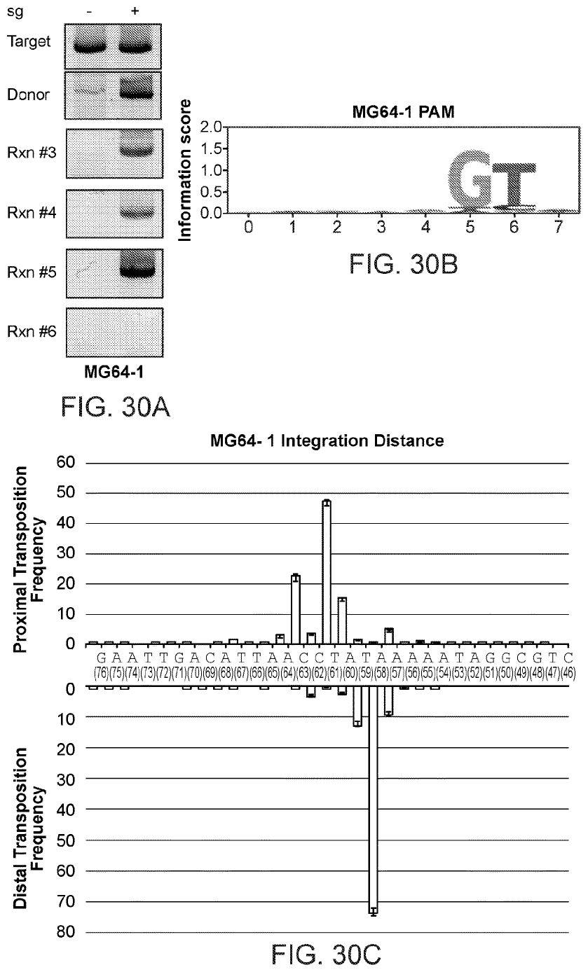

Of the discovered Cas12k CAST systems, three systems were prioritized for their novelty and completeness and tested for transposition potential in vitro. MG64-1 CAST was able to transpose the cargo to the donor plasmid in an sgRNA-dependent manner (FIG. 30A). For each of the potential junction PCR amplifications, it was predicted that all four junctions would be observed if both orientations of integration were complete, or only two of the junctions if integration only occurred in a single orientation (Panel B of FIG. 29). Surprisingly, robust transposition was observed in three of the four junction PCR reactions. The observed reactions represent both potential Left End junction products, in both Target-LE-Cargo-RE (T-LR) and Target-RE-Cargo-LE (T-RL) orientations (PCR5 and PCR3 respectively), and the T-LR oriented right end product (PCR4) (FIG. 30A)

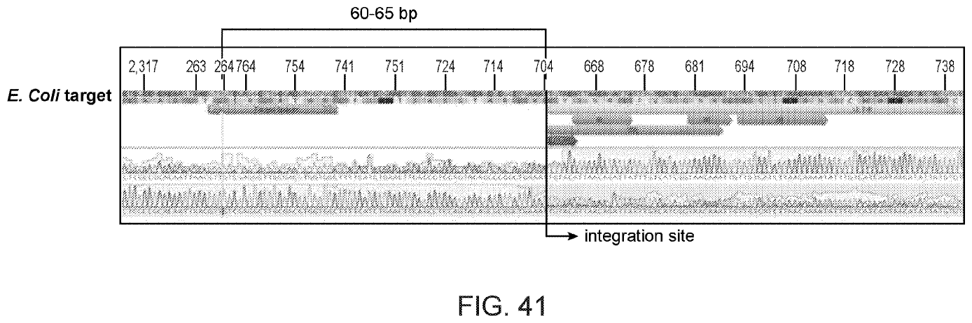

Sanger-based sequencing of the PCR transposition fragments accurately aligned to the target sequence and the sequencing signal quickly degraded at the target-donor junction (FIG. 41). Signal degradation indicates a population of integration products and sequencing from the donor end of the transposition products verified both the LE and RE prediction for the MG64-1 system.

To elucidate the PAM preference of the active CAST systems, successful transposition events from the population of the 8N randomized library that received the donor sequence were sequenced via NGS. Sequencing reads identified a GTN-5′ PAM for MG64-1 FIG. 30B).

In addition to identifying the PAM, it is also beneficial to determine the integration window size: the respective distance from the PAM where integration occurs. Cas12k CASTs integrate cargo at a specific window 60 bp offset from the PAM motif. When NGS reads of the transposition junction for MG64-1 were quantified, it was determined that 99% of the integration events occurred between 57 and 67 base pairs away from the PAM (FIG. 30C). In addition, the offset of distal distances indicates the presence of a target site duplication (TSD). For MG64-1, the TSD window ranges between 3-5 bp, which is consistent with the 4 bp TSD observed in the metagenomic assembly (FIG. 25A).

### Example 29—Single Guide RNA Engineering

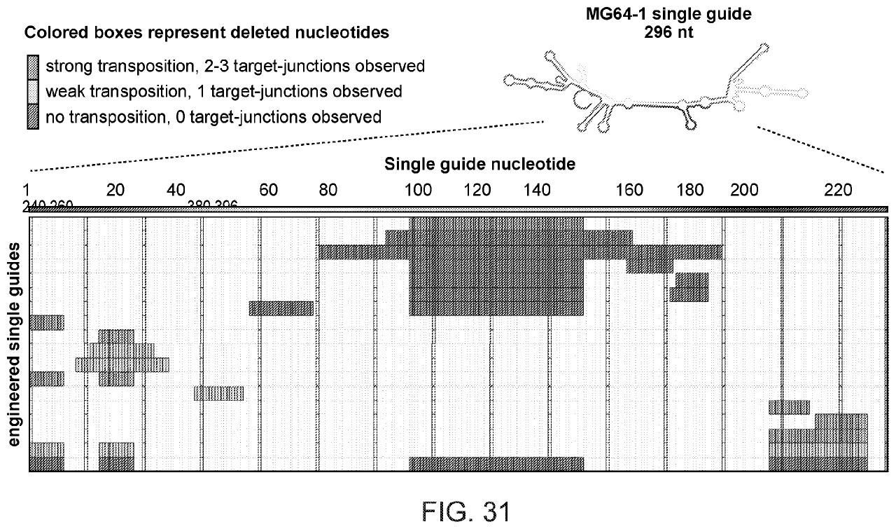

While the nuclease dead effector of the CASTs are usually smaller than 700 aa, the tracrRNA is large. A multiple sequence alignment of the predicted tracrRNAs indicated that the MG64-1 tracrRNA contains a large 58 bp insertion (FIG. 27). When folded, the excess sequence formed the halves of two well predicted hairpins near the GCGC pseudoknot motif (FIG. 28 and FIG. 31). It was hypothesized that while these structures were well predicted to form a hairpin loop, the conservation of the surrounding sequences would help to stabilize this region if this extra sequence was deleted. Therefore, a series of truncations were made to the backbone comprising this region of the single guide, including larger truncations of the GCGC pseudoknot (FIG. 31). A single guide RNA comprising the most conservative deletion was the only functional version of the single guide (FIG. 31 and FIG. 28, esg1). Subsequent studies on deletions at both the 5′ and 3′ terminus of the single guide indicated that crRNA::tracrRNA repeat anti-repeat hairpin is also not necessary for function, while using the base nucleotide pairing is (FIG. 31). Combining the documented functional deletions largely ablates activity. Thus, the smallest functional engineered 64-1 guide is 270 nt (FIG. 27. esg1 4a).

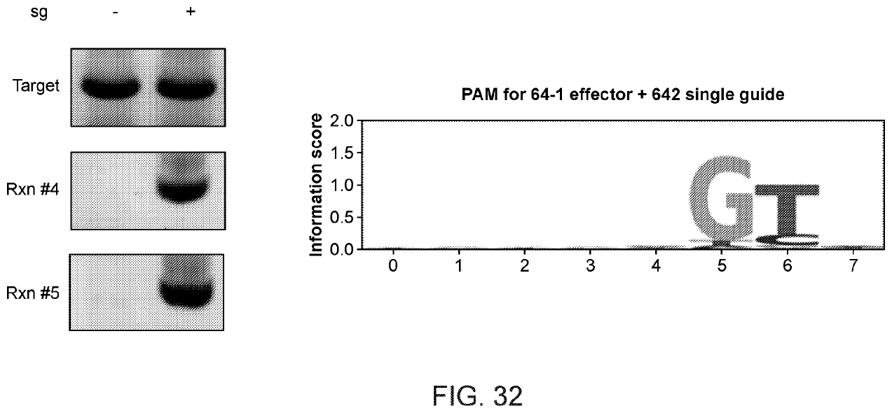

In a parallel effort to minimize the guide, the cross-functional potential of the smallest single guides predicted for MG64-2, MG64-3, MG64-5 was tested with the MG64-1 CAST system. It was observed that the MG64-2 single guide is active for the MG64-1 CAST system, reducing the functional size of the sgRNA down to 251 (FIG. 32). The PAM requirement of the MG64-1 CAST with the 64-2 single guide was not different from the effector with the native single guide.

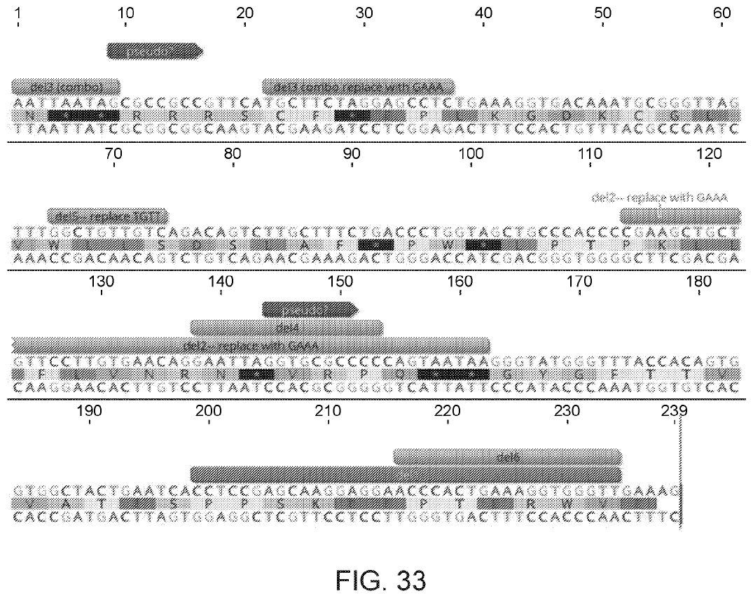

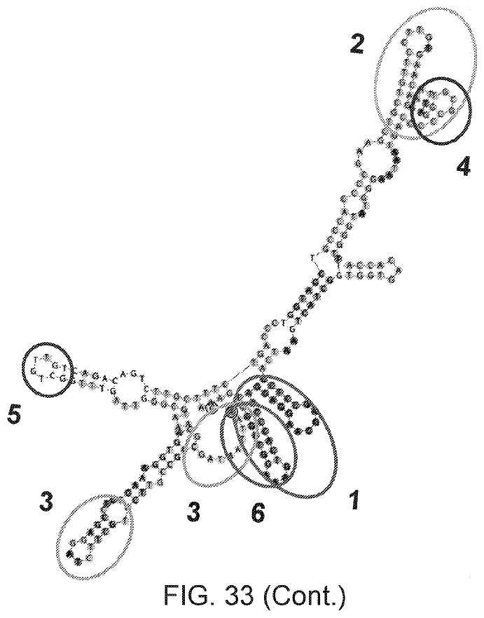

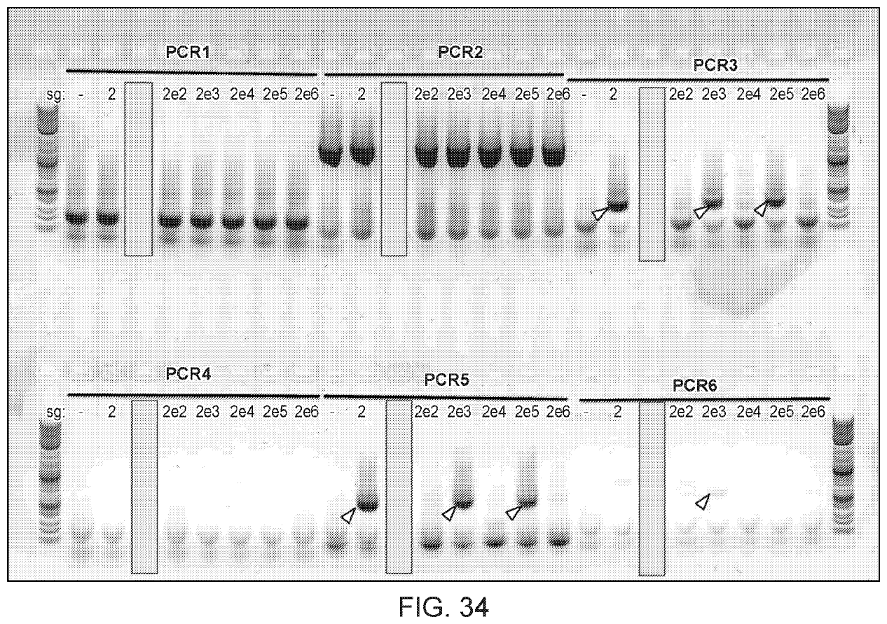

In efforts to further reduce the size of the MG64-2 sgRNA, a series of truncations were designed and used in a test to determine whether the 64-1 CAST could continue to utilize the cross functional single guide for targeted integration (FIG. 33). Six deletions were tested across the MG64-2 single guide, encompassing combinations of truncations of the 5′ pseudoknot, internal deletions at terminal hairpins, and deletions of the CRISPR repeat anti-repeat hairpin. Two engineered sgRNAs that included deletion of the 5′ pseudoknot and a shorter length of hairpin 3 maintained the ability to transpose with MG64-1 (FIG. 34).

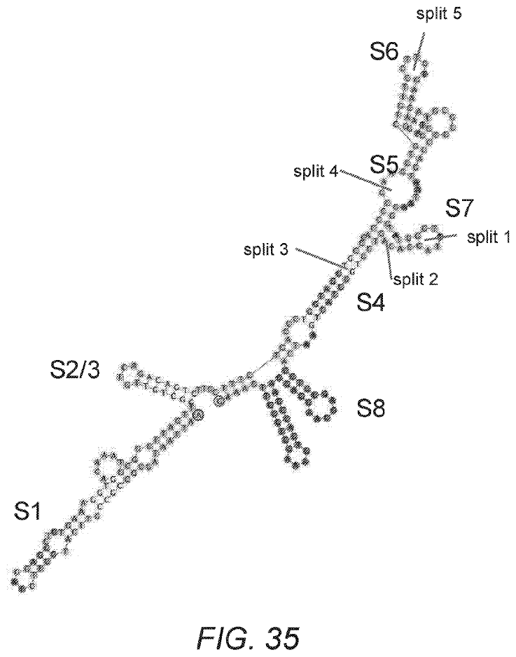

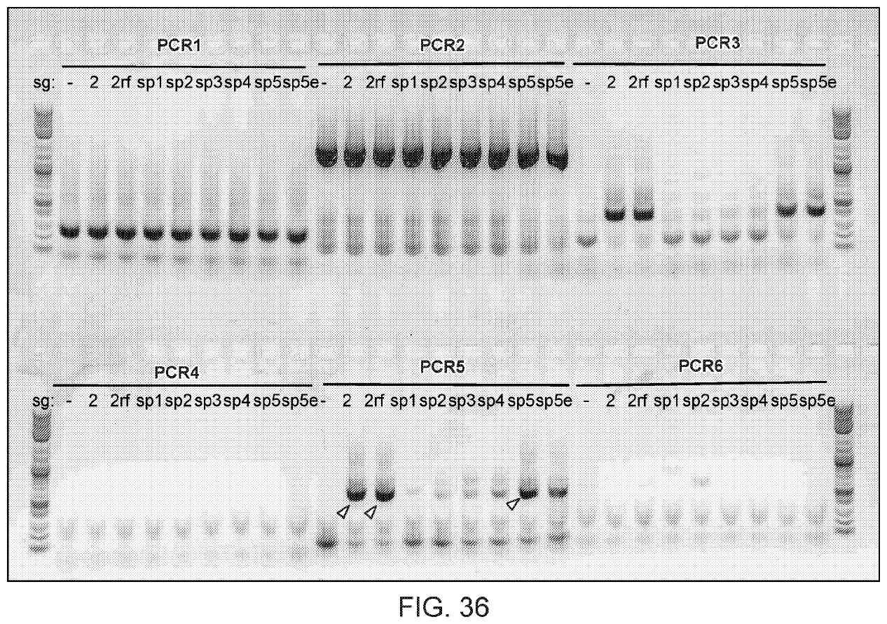

In order to simplify synthesis of a large RNA molecule, the sgRNAs were split into two fragments that can be complexed together during transposition reactions. This approach takes advantage of a structural hairpin loop to complex the two RNA molecules together in order to form a similar structure to the active sgRNA. Using the MG64-2 single guide, 5 split points were designed among the predicted backbone helix, unstructured regions, and highly predicted hairpin loops (FIG. 35). In addition, one split included an extension that improved base pairing of the split hairpin loop. Of the designs tested, hairpin loop representing and the extended hairpin dual guides were able to complex with the Cas12k of the MG64-1 system and direct transposition (FIG. 36).

### Example 30—Transposon LE and RE Engineering

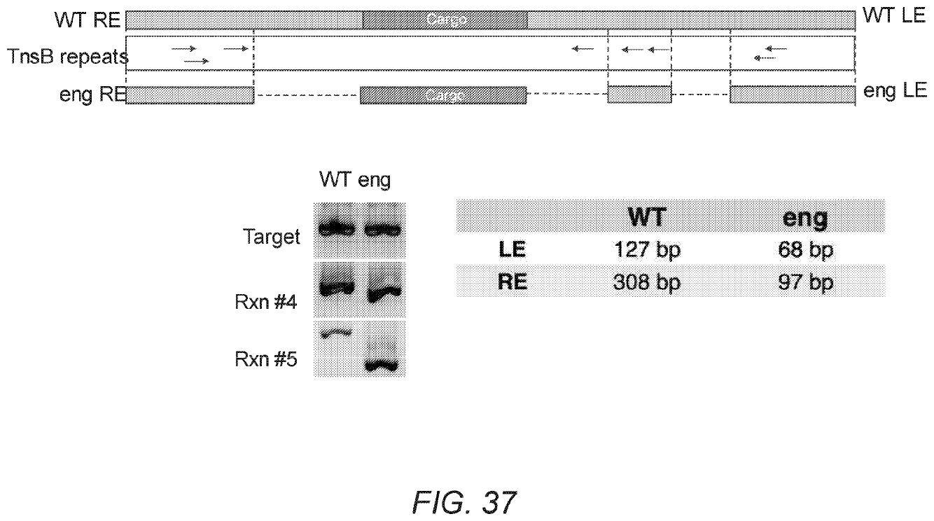

Intergenic regions flanking Cas12k CAST transposon genes TnsB, TniB, TniQ, and Cas12k contain terminal inverted repeats (TDR) of ~12-20 bp, which are predicted to encode transposon binding sites. Because TIRs on the LE and RE are predicted to be TnsB binding sites, a series of deletions of the wild type LE and RE ends was generated to determine essential binding sites. While the LE and RE of MG64-1 was predicted to contain 3 and 5 repeats, respectively, deletions from the cargo end of the TIRs remained active down to a fragment as small as 68 bp. The approach to the RE deletion series included an internal deletion of 81 bp that might allow for a reduction of size of the RE to include 4 repeats down to 97 bp. LE and RE minimization maintained the transposition activity of the system (FIG. 37).

### Example 31—E. coli Integration Activity with MG64-1

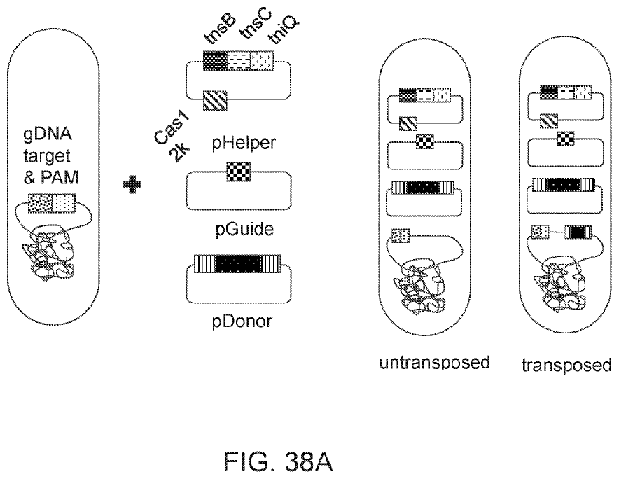

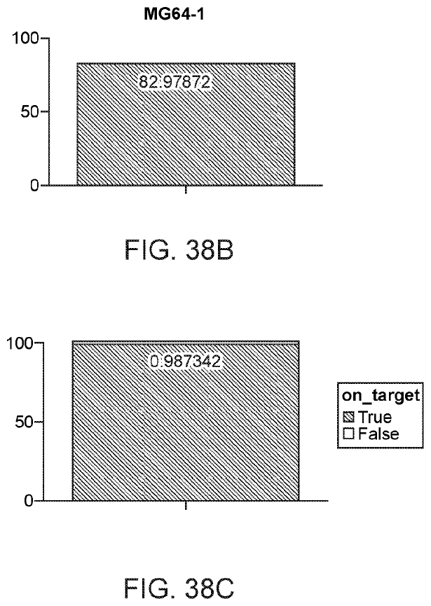

To test the transposition efficiency in a cellular context, a strain of E. coli BL21(DE3) was engineered to include the spacer sequence confirmed for activity in vitro. A plasmid containing the polycistronic Tn5053-like genes and the effector under the T7 promoter was used to express the CAST proteins, and a separate plasmid was co-transformed to introduce the guide under the control of the J23119 promoter (FIG. 38A). The pDonor plasmids contained an antibiotic resistance cargo flanked by the confirmed WT LE and RE and the minimized LE and RE for MG64-1.

An NGS based method was developed to assess transposition efficiency for MG64-1. NGS reads indicate over 80% editing efficiency (FIG. 38B) and enabled determination of the off-target profile associated with each CAST. The off-target editing rate was determined as a single read that mapped to the LE or RE with an additional 14 bases mapping elsewhere in the E. coli genome. Off-target integration greater than 1% of all the summed transposition events was not detected (FIG. 38C).

### Example 32—Endogenous Locus Targeting

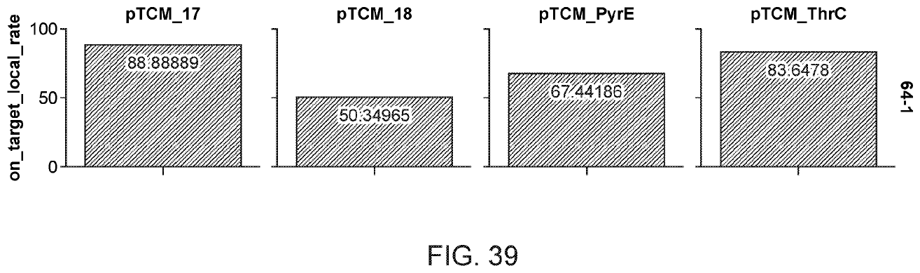

In order to test the programmability of these systems to integrate into the E. coli genome, three target sites with GTN-5′ PAMs were chosen to integrate into. From WGS data, MG64-1 CAST was able to integrate at multiple loci with efficiencies ranging between 50-90% (FIG. 39). Together with the low off-target rate, these data demonstrate that this Cas12k system is capable of achieving high rates of genomic integration with a programmable RNA guide.

### Example 33—Multi Locus Targeting

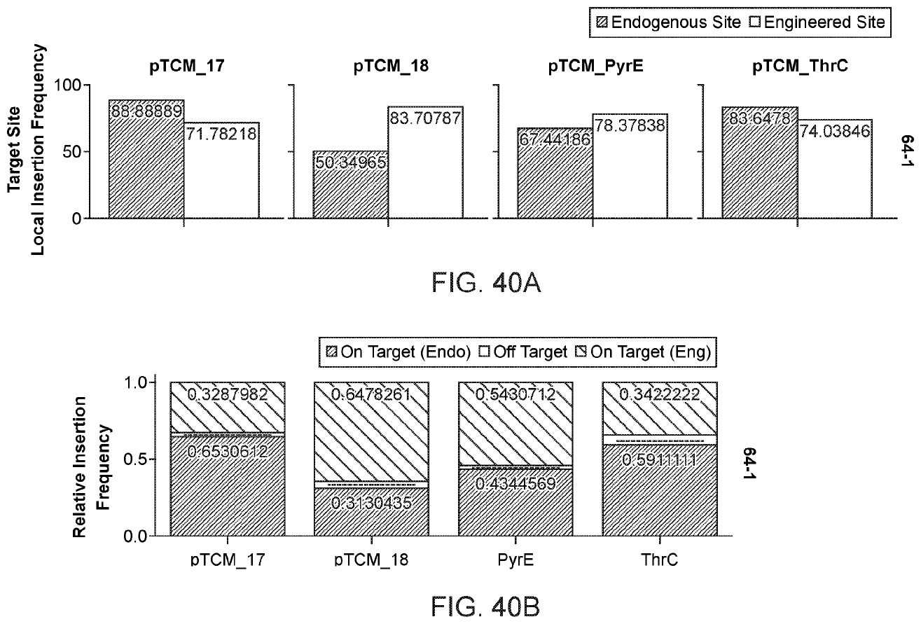

With the high level of integration on endogenous loci and engineered loci, the ability of the CAST to integrate in multicopy in a single reaction was subsequently tested. By introducing both single guides into a single E. coli strain along with the donor plasmid, both loci displayed integration with greater than 50% efficiency (FIG. 40). In all instances, integration at both loci combined accounted for greater than 95% of all integrations that occurred on the genome (FIG. 40A).

### Example 34—Exploration of Target DNA Dilutions in In Vitro Transposition Assays In Vitro Targeted Integrase Activity Experiments

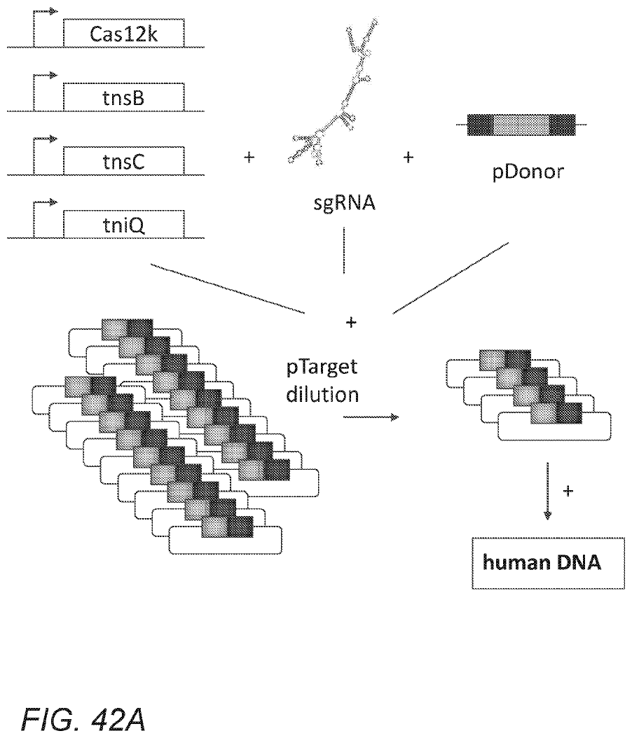

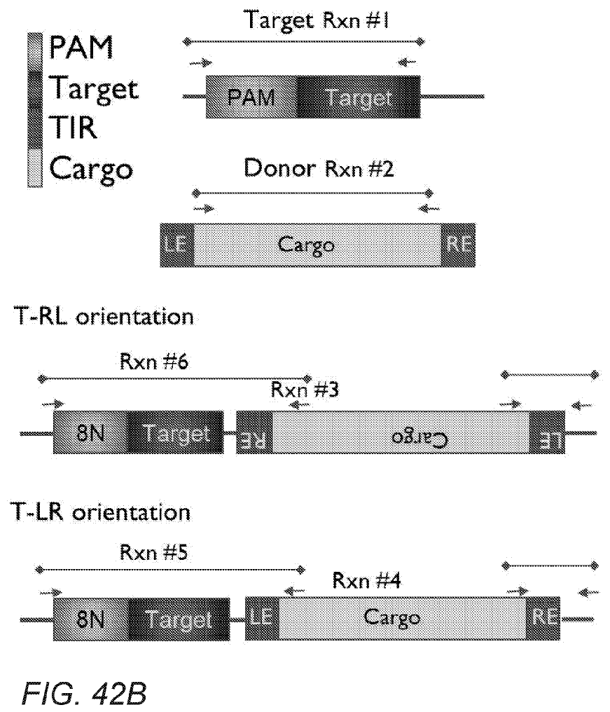

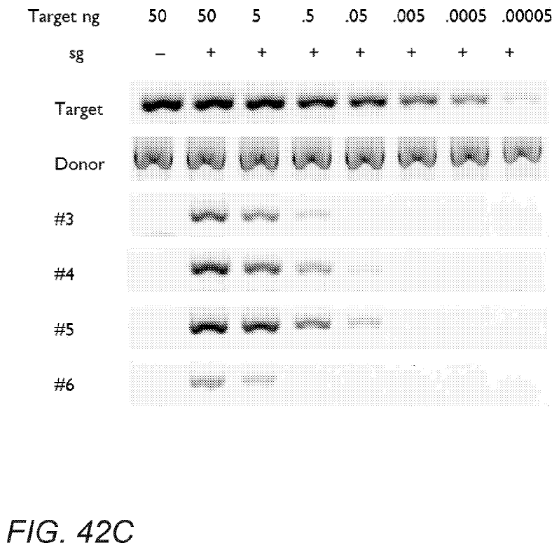

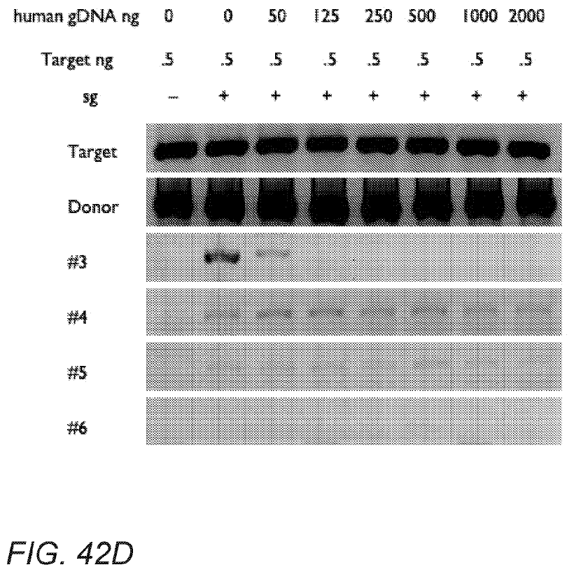

Integrase activity was assayed with a target plasmid containing the PAM adjacent to the protospacer sequence (pTarget) (FIG. 42A). T7 promoter leading gene sequences were introduced by PCR amplification of all transposase, single guide RNA (sgRNA) and effector components, and expressed independently in an in vitro transcription/translation system (FIG. 42A). Purified in vitro transcribed single guide RNA were refolded in duplex buffer (10 mM Tris pH 7.0, 150 mM NaCl, 1 mM MgCl₂) and normalized to 1 μM. Donor fragments were PCR amplified from plasmid pDonor, which contained a kanamycin or tetracycline resistance marker flanked by MG64-1 left end (LE) and right end (RE) transposon motifs, and normalized to 50 ng/μL.

After expression, 1 μL of Cas12k in vitro expression reaction was added to 0.5 picomoles of sgRNA and incubated for 20 minutes at 25° C. Individually expressed transposase proteins were then added volumetrically at 1 μL per expression. Target DNA and 50 ng donor DNA were then added to the transposition reaction in a reaction buffer, with final concentrations of 26 mM HEPES pH 7.5, 4.2 mM Tris pH 8, 50 μg/mL BSA, 2 mM ATP, 2.1 mM TCEP, 0.05 mM EDTA, 0.2 mM MgCl₂, 28 mM NaCl, 21 mM KCl, 1.35% glycerol, (final pH 7.5) and 15 mM Mg(OAc)₂. In vitro transposition reactions were performed at 37° C. for 2 hours, transposition reactions were diluted tenfold in water, and used subsequently as a template for junction PCR analysis.

### Junction PCR Analysis

Junction PCR reactions were performed with Q5 polymerase and amplified with primers flanking: Rxn #1 (Target), Rxn #2 (Donor), Rxn #3 (Reverse LE), Rxn #4 (Forward RE), Rxn #5 (Forward LE), and Rxn #6 (Reverse RE) (FIG. 42B). PCR fragments were run on a 2% agarose gel in 1×TAE and analyzed for size discrimination. Appropriately sized bands of each PCR junction were gel-excised, and the PCR fragments were recovered through purification and sanger sequenced using both amplification primers. Resulting Sanger sequencing was mapped to the donor and target sequences to confirm integration approximately 60 bp away from the PAM.

Transposition Reactions with Target Plasmid DNA Dilutions

Because of the known, low availability of single copy target sites in the human genome (for example, Moreb et al., 2020), it was predicted that low availability targets in in vitro experiments could mimic the search space in human genomic DNA by the MG64-1 CAST. To determine the minimum amount of target plasmid (pTarget) necessary to detect targeting by MG64-1 in vitro, the CAST was used in serial dilutions of total target DNA in the transposition reaction. Target plasmid amounts were serially diluted by 10 fold from 50 ng of DNA per reaction (moles) to 0.00005 ng (moles) and then added to transposition reactions containing the MG64-1 suite, sgRNA, and donor plasmid (FIG. 42A). PCR amplification of target-donor junctions across transposition products were then analyzed by gel electrophoresis (FIG. 42B).

### Results: Serial Dilution of Target Plasmid DNA

In vitro transposition experiments with dilutions of target plasmid indicated that single guide RNA (sgRNA) was necessary for targeted integration of donor onto the plasmid at 50 ng of target DNA (FIG. 42C, lanes 1 and 2). As the target was serially diluted, target DNA was still detectable at 0.00005 ng/reaction but with lower intensity (FIG. 42C, “Target” band on lane 8). Reverse LE integration product (Rxn #3) and reverse RE product (Rxn #6) both decreased with the tenfold dilutions, resulting in the detectable presence of Rxn #3 down to 0.5 ng of target DNA (FIG. 42C). Transposition reactions Rxn #4 and #5 were both equally robust and were detectable down to 0.05 ng of target DNA.

Based on the presence of three of four expected transposition reaction products, the 0.5 ng of target plasmid condition was selected to test whether transposition was detected when increasing the complexity of DNA search space. Increasing amounts of exogenous human genomic DNA (gDNA) were added to the reaction with fixed 0.5 ng of target plasmid, MG64-1 CAST, and sgRNA (FIG. 42A and FIG. 42D). When no gDNA was added, transposition experiments confirmed that sgRNA was necessary for targeted integration of donor onto the target plasmid at 0.5 ng of DNA (FIG. 42D, lanes 1 and 2). When gDNA was added to the reaction at increasing amounts (0-2000 ng of DNA per reaction), transposition products were also diluted, as given by the faint bands compared with the no gDNA control (FIG. 42D). For example, the reverse LE integration product (Rxn #3) was visible when adding at most 50 ng of gDNA. In addition, robust transposition products for the forward RE (Rxn #4) and forward LE (Rxn #5) products at 125 to 1000 ng of gDNA with a fixed amount of 0.5 ng of target plasmid (FIG. 42D, lanes 4-8) was detected.

### Example 35—In Vitro Transposition to Human gDNA with MG64-1 CAST In Vitro Targeting to High Copy Elements Across the Human Genome

This Example assesses a dilution series of natural targets in the genome for transposition as a function of target frequency.

### Results: Target Site Identification in High Copy Regions of the Human Genome

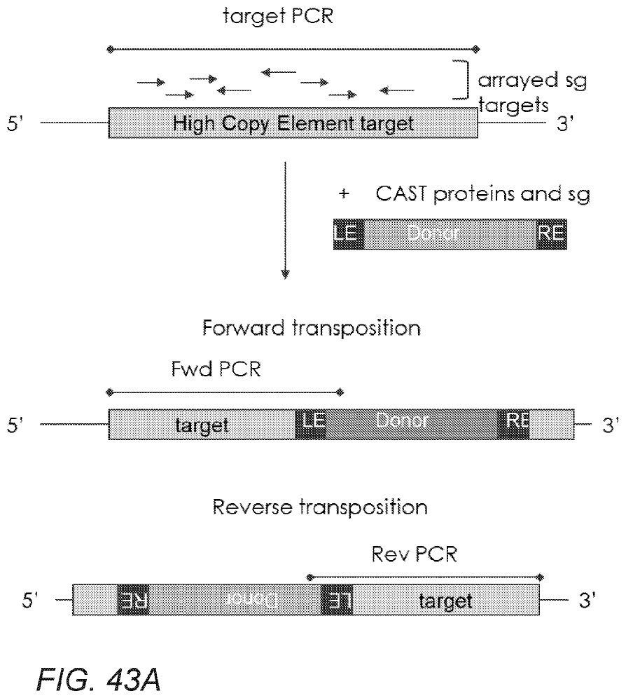

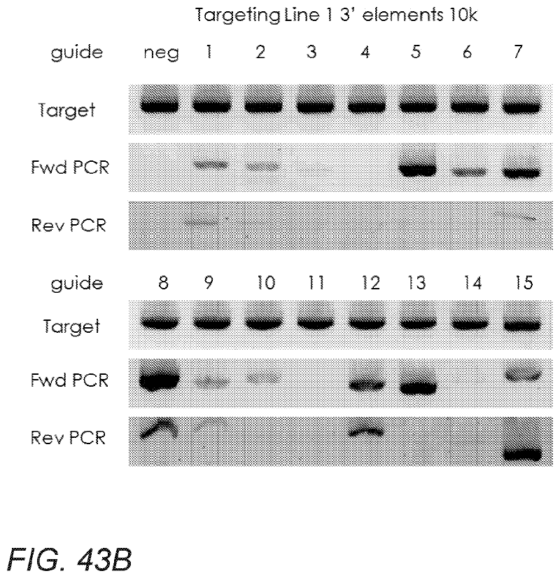

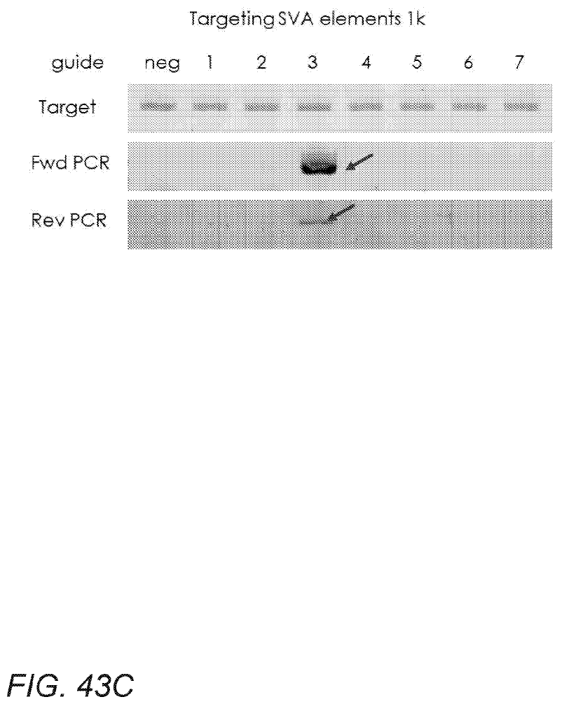

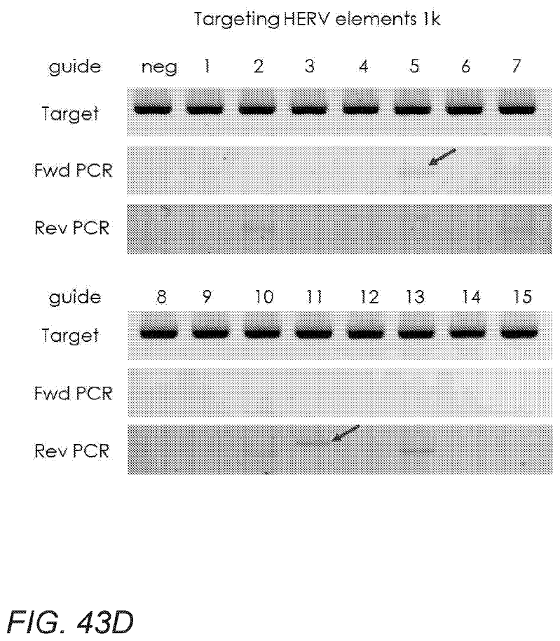

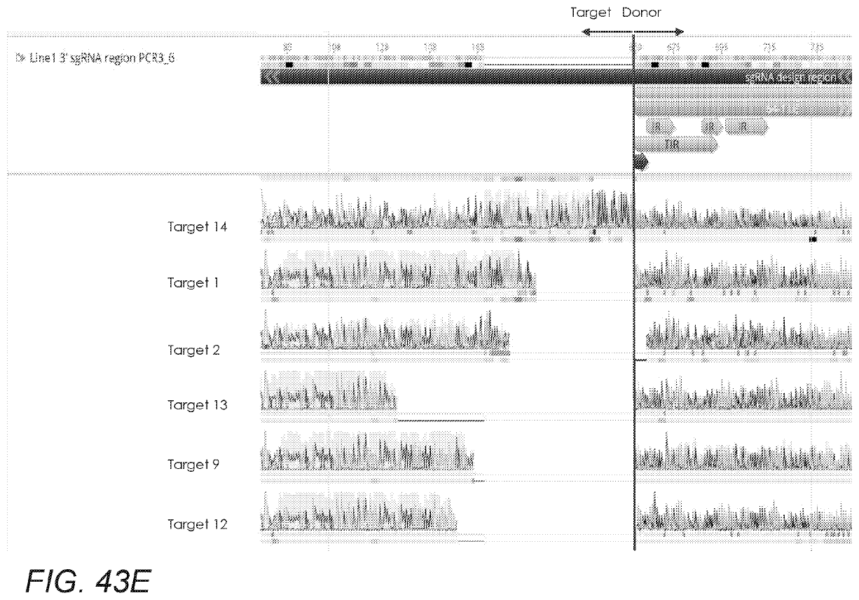

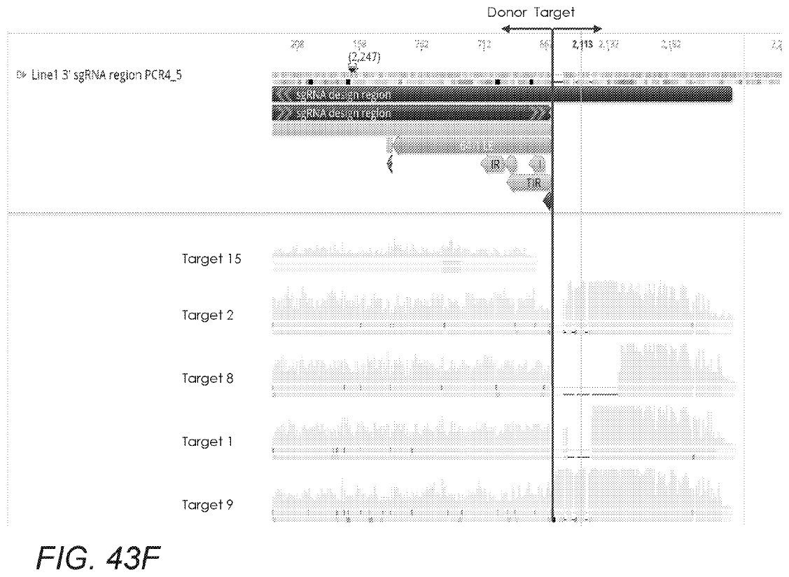

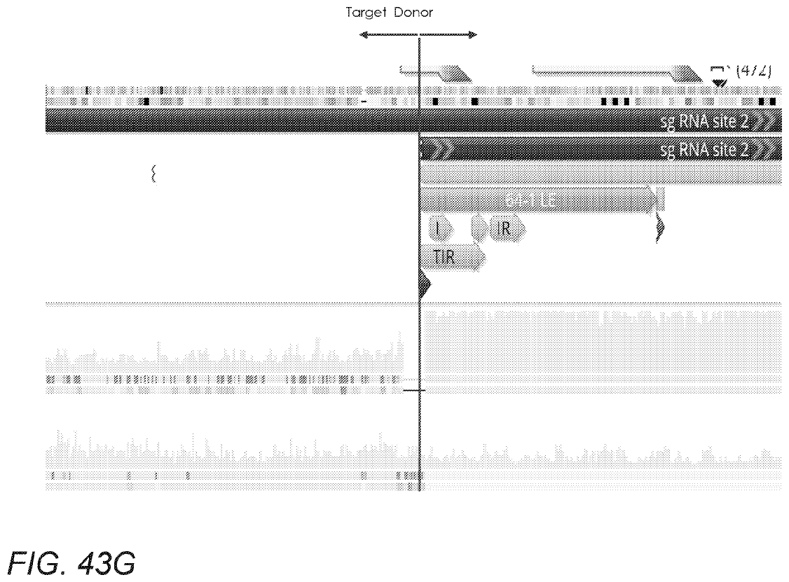

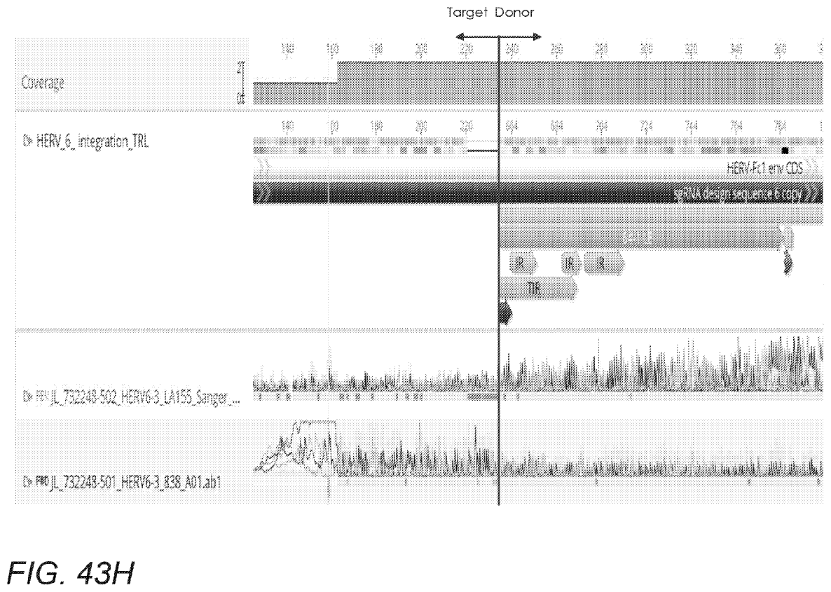

High copy targets were identified in the human genome using a Cas off-target finder. Using a 200-300 bp target sequence in the most conserved spaces of each replicated element, 15 targets sites for each LINE1 3′ and HERV were identified, and 7 target sites were designed for SVA elements with varying GC content, orientations, and permutations of the MG64-1 rGTN PAM (FIG. 43A). Target (spacer) sequences were synthesized as oligos and PCR amplified onto the MG64-1 sgRNA template with a T7 promoter upstream of the single guide backbone. PCR reactions of the MG64-1 sgRNA were then purified and in vitro transcribed. Using NLS-tagged MG64-1 protein components, an in vitro transposition reaction as described above was assembled, with purified HEK293T gDNA at 1 μg/reaction as target DNA.

### Results: In Vitro Targeted Transposition to High Copy Regions of the Human Genome

Successful transposition by MG64-1 was evaluated by the resulting Fwd PCR and Rev PCR junction products at each of the 15 target sites in high copy elements (FIG. 43B). MG64-1 promoted transposition in the forward orientation to LINE1 targets 3, 5, 6, 7, 10, and 13, while targets 1, 2, 8, 9, 12, 14, and 15 were reactive for transposition in both forward and reverse orientations (FIG. 43B). In addition, active transposition into SVA target 3 and HERV target 5 was specific for both forward and reverse orientations (FIGS. 43C-43D). Sanger sequencing of transposition reaction products for LINE1, SVA, and HERV confirmed that in vitro transposition was specific and RNA-guided (FIG. 43E-43H).

### Example 36—NLS-Functional Domain Fusions with MG64-1 CAST are Targetable to High Copy Elements

Functional Domains Fused with CAST Components for Targeted Transposition In Vitro to High Copy Elements in the Human Genome

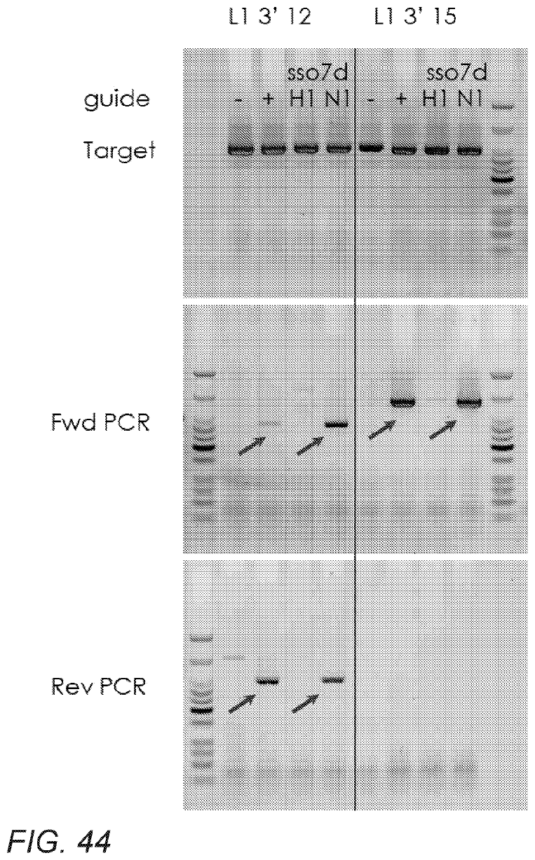

With experiments indicating that high copy elements are efficient targets for integration in vitro, functional domains were tested for their effects on CAST targeting at these sites. Functional domain fusions of Cas12k-sso7d were challenged with H1core-TniQ, or Cas12k-sso7d with HMGN1-TniQ, to transpose donor DNA when in reaction with NLS-TnsB and NLS-TnsC fusions at high copy elements LINE1 target 12 and target 15. Transposition reactions were assembled with either Cas12k alone or with fusion Cas12k-sso7d where indicated, with a no sgRNA (−sg) condition as negative control for transposition, and with sgRNA for target 12 and target 15 of LINE1 3′ elements where indicated (FIG. 44). In addition, transposition reactions were supplemented with translated NLS-TniQ, NLS-H1core-TniQ or NLS-HMGN1-TniQ, NLS-TnsB, NLS-TnsC, pDonor, buffer, and human gDNA for targeting.

### Results

In vitro transposition assays with fusion domains targeting LINE1 target 12 and target 15 indicated successful integration by the MG64-1 no-fusion, positive control (FIG. 44. lanes 3 and 7), as well as with fusions Cas12k-sso7d with HMGN1-TniQ (FIG. 44, lanes 5 and 9). No targeting occurred when no sgRNA was added to the reaction (FIG. 4, lanes 2 and 6).

### Example 37—NLS Fusion to S15 for Targeted Transposition

NLS Fusion with Small Ribosomal Protein Subunit S15 is Necessary for Correct Orientation of Tag

Recently, the small prokaryotic ribosomal protein subunit S15 was deemed necessary for targeted transposition by Cas12k CAST in vitro (Schmidt et al., 2022; Park et al., 2022). Therefore, the need for S15 with and without NLS tags in transposition experiments with MG64-1 was evaluated. Because the in vitro expression reagent already contains S15 (S15 is part of the prokaryotic ribosomal complex needed for protein expression), it is likely that in vitro transposition experiments with CAST proteins that had been expressed with PURExpress had S15 carried over from the expression step and was subsequently recruited for CAST targeting.

### Results: S15 Increases Transposition Efficiency in In Vitro Experiments

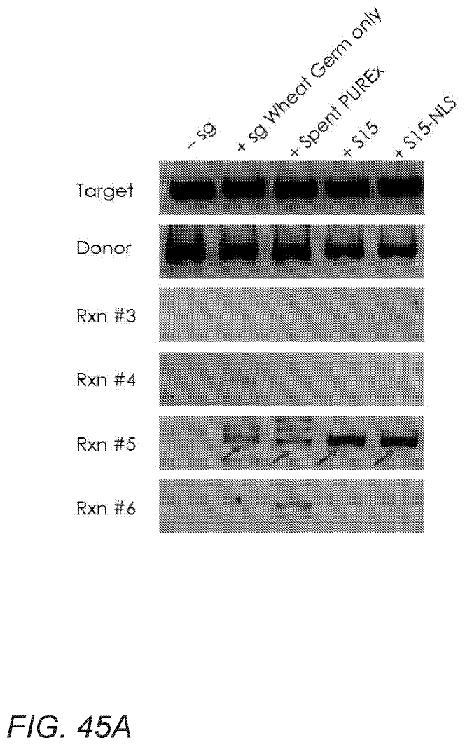

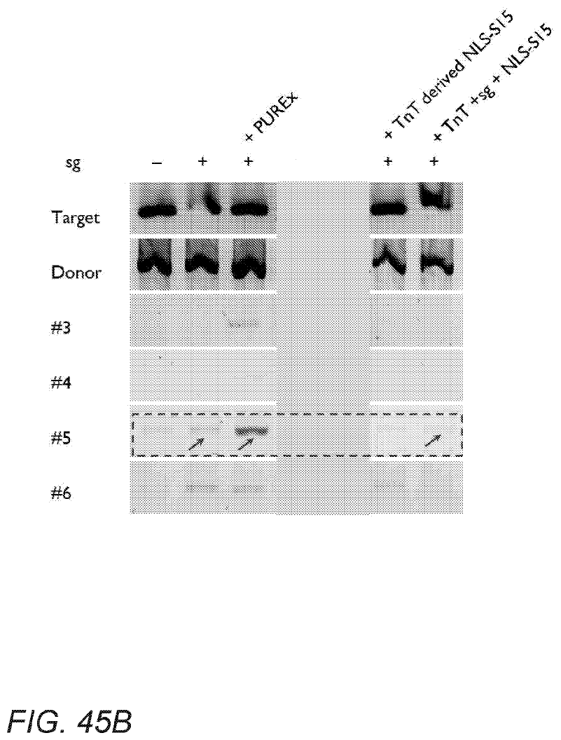

Wheat Germ Extract was used in a Eukaryotic transcription/translation system, which does not contain S15, to express MG64-1 CAST components. CAST templates were amplified to contain a T7 promoter and a 40 bp Poly A tail for transcriptional stability of mRNA templates. Proteins were expressed from the dsDNA template via transcription/translation reactions, which were then used in an in vitro transposition reaction, as described above. Results indicate that S15 addition increased targeted transposition efficiency, as shown by the intensity of the bands from junction PCR products (Rxn #5) (FIG. 45A, lanes 4-5).

### Results: The S15-NLS Fusion is the Preferred Orientation for In Vitro Transposition

In eukaryotic conditions, translation of proteins is exclusively performed in the cytoplasm, while transposition reactions mediated by CAST would most likely occur in the nucleus. The necessity of an NLS tag for S15 nuclear localization was evaluated. NLS tags were fused to both N- and C-termini of S15 and tested in the Eukaryotic in vitro transcription/translation reactions and in vitro transposition experiments (FIG. 45A, lane 5, and FIG. 45B, lanes 4 and 5). The results indicate that the S15-NLS was more efficient for transposition than other tested conditions (FIG. 45A, lane 5).

### Example 38—S15 is Necessary for in Cell Translation of CAST

### Design of CAST Vectors

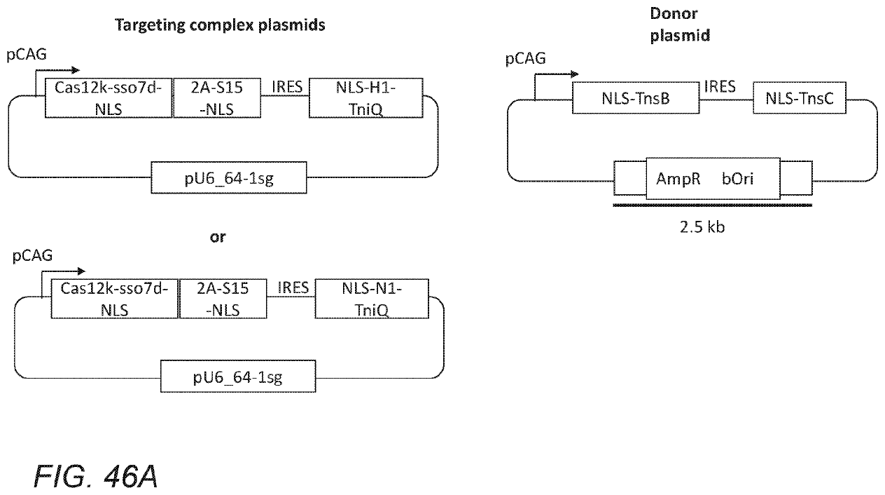

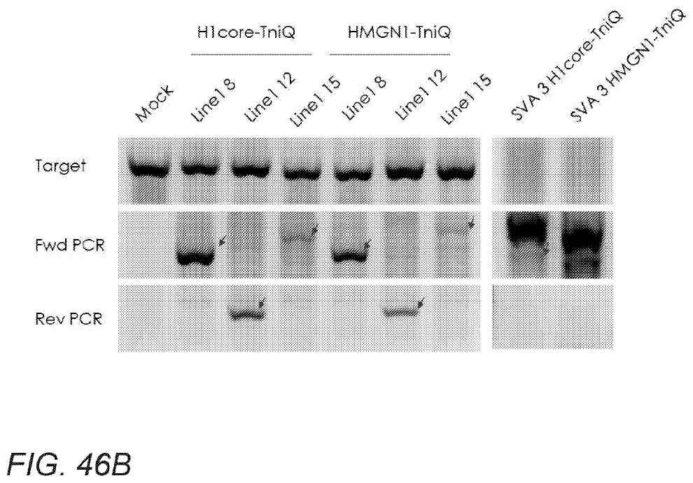

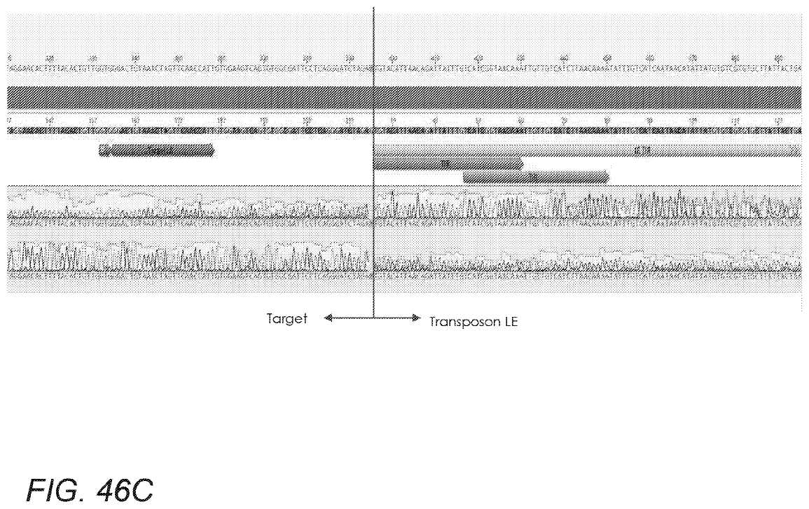

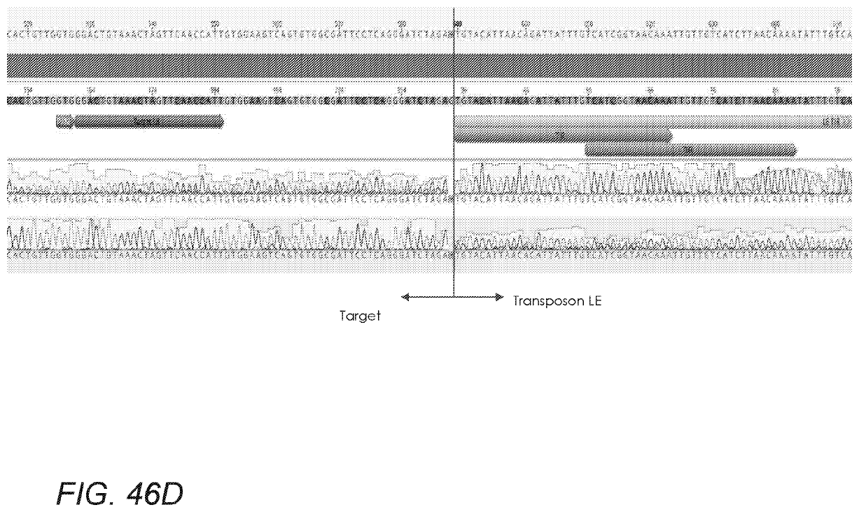

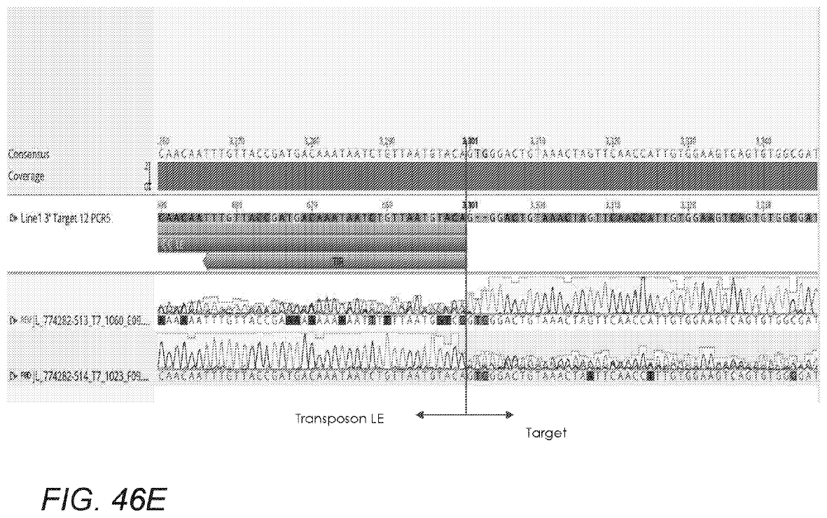

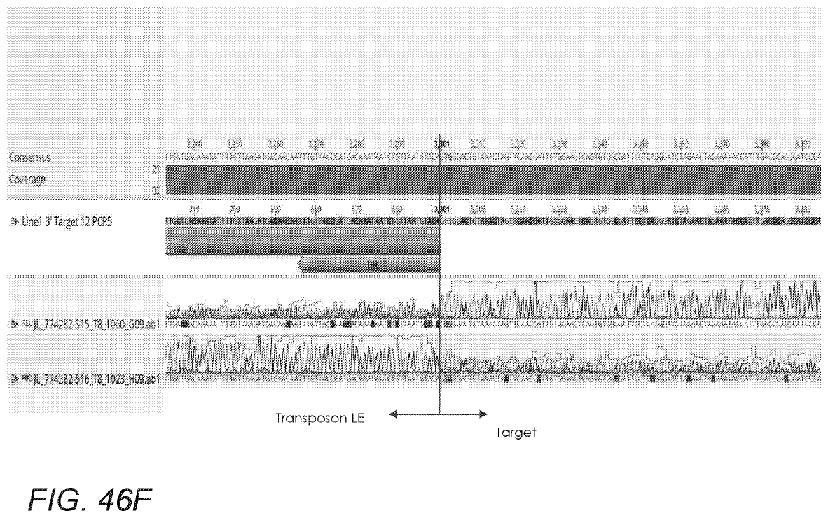

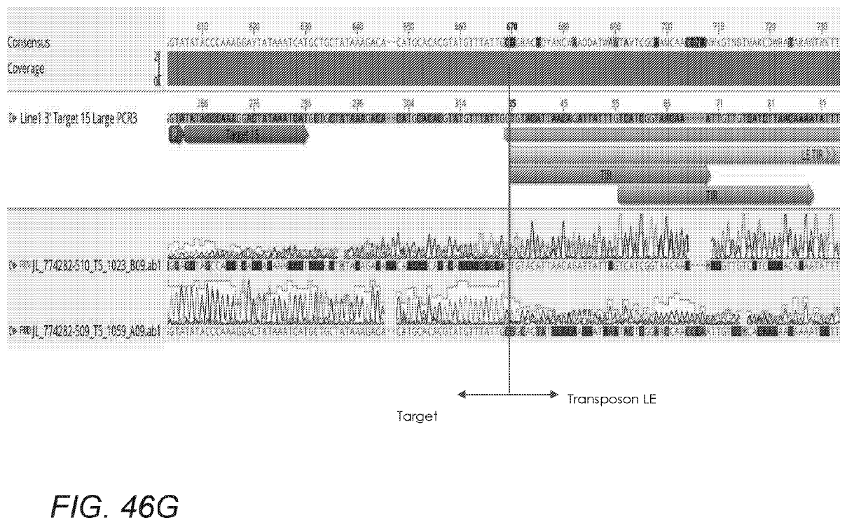

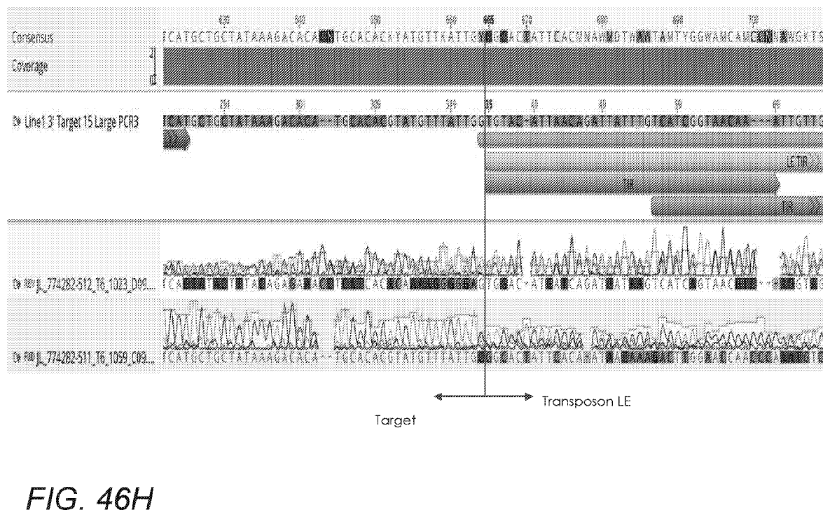

MG64-1 CAST proteins were expressed on two high expression plasmids for transposition experiments in human cells. One plasmid expresses the protein targeting complex under control of a pCAG promoter. Two versions of the protein targeting complex were designed. One version contains a Cas12k-sso7d functional domain fusion, with a 2A peptide fused to S15-NLS, IRES, and NLS-H1core-TniQ (FIG. 46A, top left). A second version contains Cas12k-sso7d-2A-S15-NLS with an NLS-HMGN1-TniQ fusion (FIG. 46A, bottom left). The targeting plasmid also contained a pU6 PolIII promoter driving transcription of a humanized MG64-1 sgRNA for targeting one of LINE1 targets 8, 12, and 15, and SVA target 3. The second plasmid transfected into cells was the donor plasmid containing NLS-TnsB and NLS-TnsC, separated with an IRES under expression of pCAG promoter. On this plasmid, 2.5 kb of DNA cargo was contained between the LE and RE terminal inverted repeats (FIG. 46A, right).

### HEK293T Lipofection

2.5 million HEK293T cells were seeded 24 hours before lipid-based transfection of the two plasmids system in 9 μg: 9 μg of targeting: donor plasmid. Cells were incubated for 72 hours at 37° C., then harvested by resuspension in 4 mL 1×PBS pH 7.2, 2 mL of resuspended cells were harvested for gDNA and eluted in 200 μL of elution buffer. 5 μL extracted gDNA were assayed for transposition in 100 μL Q5 PCR reactions with primers specific for the high copy element targets: transposition in the forward direction was determined by amplification with primers specific for Fwd PCR, and reverse transposition was determined by amplification with primers specific for Rev PCR. Amplified PCR reactions were visualized on a 2% agarose gel. Transpositions were predicted to transpose at 60 nt away from the PAM as observed in in vitro transposition experiments, and were determined to be active by the presence of a single band for junction PCR amplification at the predicted size. PCR amplicons were Sanger sequenced and NGS sequenced for transposition profile analysis.

### Results

Cells transfected with both versions of targeting complex plasmids, with Hlcore-TniQ or HMGN1-TniQ, were analyzed for transposition (FIG. 46B). Both versions of the targeting complex plasmid promoted transposition at all four target sites (FIG. 46B, arrows). LINE1 targets 8 and 15 were only detectable in the LE to 5′ target orientation, while LINE1 target 12 was only detectable in the LE to 3′ orientation (FIG. 46B). Results indicate that targeted integration in human cells has a strong preference for directionality from the PAM.

Sanger sequencing of PCR junctions confirmed integration at LINE1 targets 8, 12, and 15, at 59, 62, and 60 nt away from the PAM, respectively (FIGS. 46C-46H). Sequencing signal degradation was observed at the transposition junction, which was due to a mixture of population events. In order to determine single molecule profiles of each integration event. PCR amplicons were sequenced via NGS. Reads resulting from NGS sequencing confirmed targeted integration at LINE1 targets 8, 12, and 15, and SVA target 3. Variation in the target regions indicates the natural diversity of LINE1 and SVA repeated elements in the human genome.

Overall, MG64-1 was a successful system for targeted integration in human cells, with strong directionality preference relative to the PAM.

### Example 39—NLS-Immunofluorescence

### Fixed Cell Staining Shows NLS-CAST Components are all Capable of Translocation to the Nucleus

While functional NLS-CAST fusions are able to be tested using in vitro transposition assays, the inherent ability of the NLS tags to shuttle proteins into the nucleus must be tested in the cellular context. By exposing the CAST-NLS proteins in cells, whether through genomic expression or in the form of mRNA, the cells can be fixed and a stain performed for the introduced epitope tag using fluorescently conjugated antibodies. By visualizing the fluorescence relative to DAPI nuclear staining, the likelihood of the protein to be translocated into the nucleus for activity can be determined.

### Immunofluorescence in HEK293T Cells

HEK293T cells were plated on a collagen-coated coverslip at 50,000 cells per 24-well plate. Cell cultures were left to adhere to the cover slip overnight. For each of the active MG64-1 NLS fusions proteins, the template was in vitro transcribed with a poly-A tail, and the mRNA of these constructs were transfected in HEK293T at 500 ng/well. After 48 hours of expression, cells were fixed using 4% formaldehyde, cell membranes were permeabilized with Triton X-100, then washed with 2% BSA and probed overnight with anti-HA antibody. Cells were then washed with 2% BSA in PBS and then subsequently stained with FITC-conjugated goat anti-Mouse secondary antibody. Following secondary antibody exposure, cells were washed with PBS, mounted on DAPI mounting epoxy, and cured overnight. Visualization of cells was performed on a cell imaging microscope for fluorescence, and nuclear localization was determined by FITC co-localization with DAPI staining.

### Results

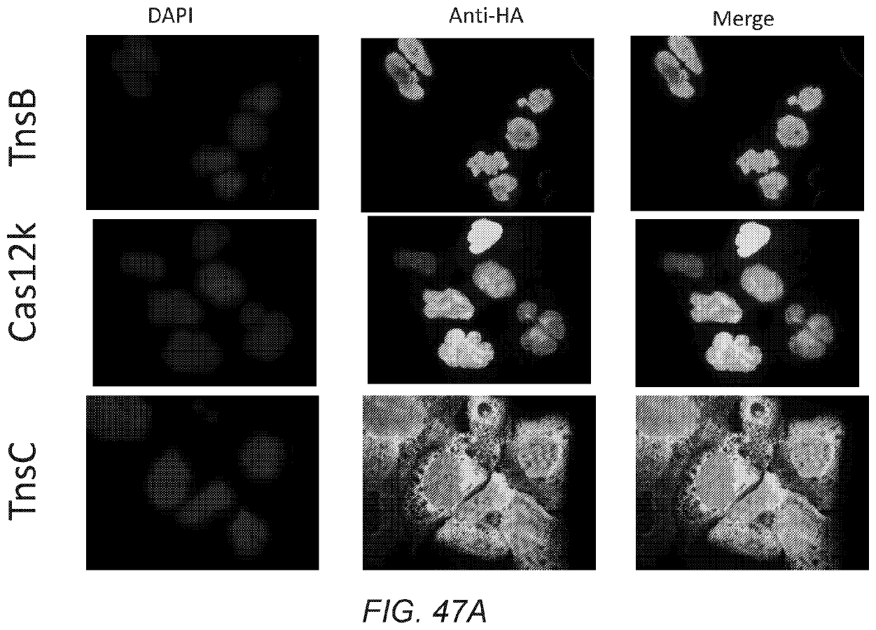

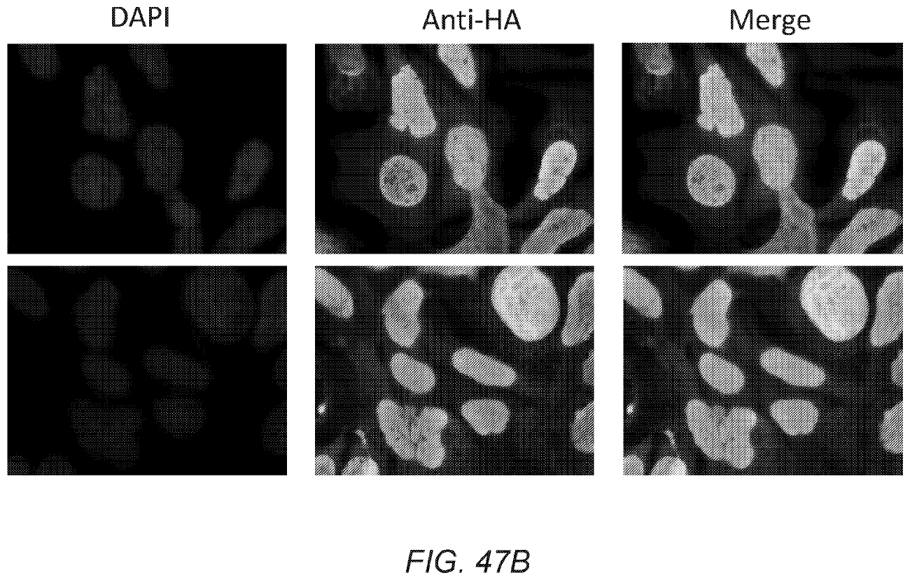

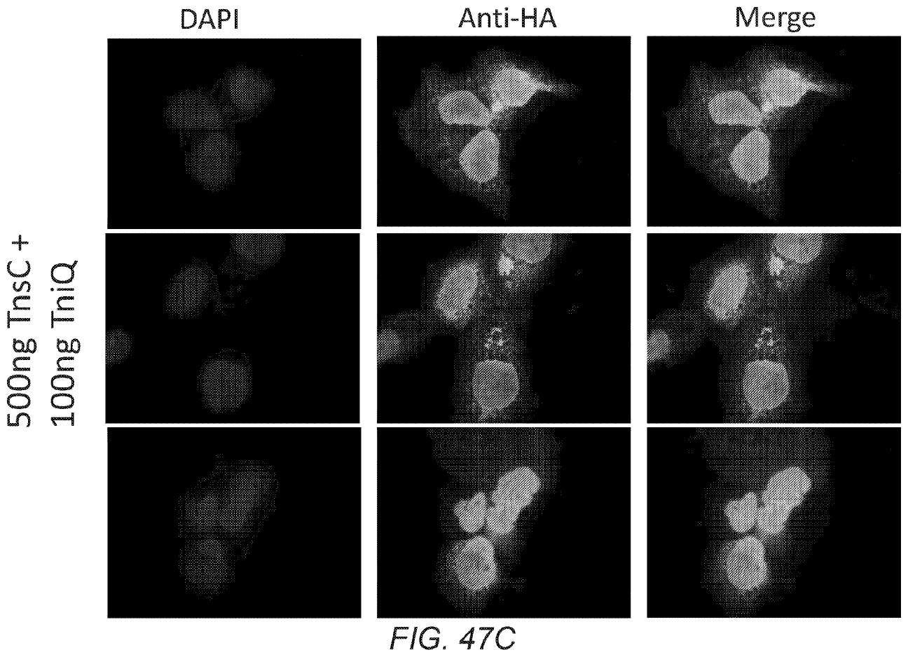

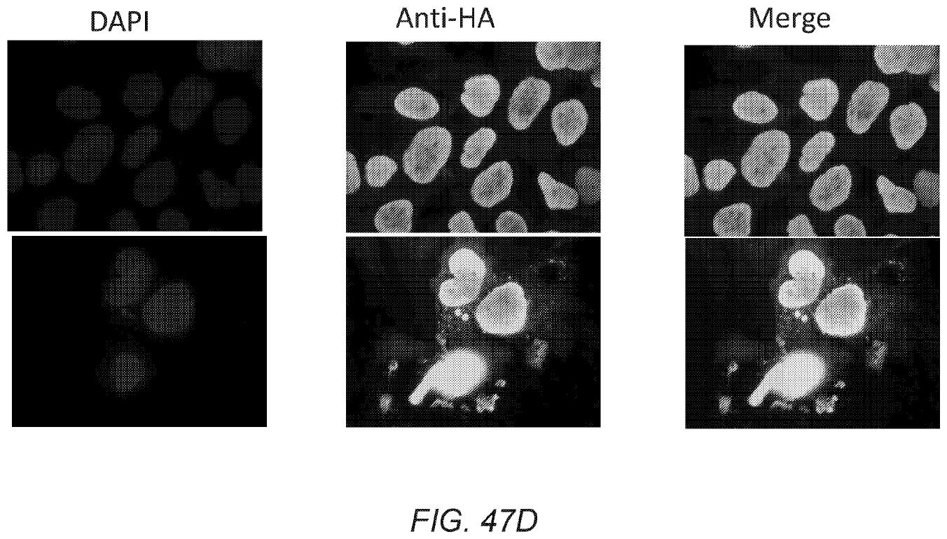

Cas12k-NLS, NLS-TnsB, and NLS-TniQ of the MG64-1 CAST complex were capable of localizing to the nucleoplasm (FIG. 47A, top and mid-panels, and FIG. 47B), while NLS-TnsC localized to the cytoplasm in filamentous aggregates (FIG. 47A, bottom panel). When subsequent studies of immunofluorescence were performed on NLS-TnsC co-expression with NLS-TniQ, nuclear localization of NLS-TnsC was rescued (FIG. 47C). Furthermore, when all MG64-1 Cas12k CAST components were expressed in HEK293T cells via mRNA delivery, all components localized in the nucleus (FIG. 47D). Results indicated that the filamentous aggregation of NLS-TnsC in cells may be abrogated by the presence of TniQ in the cell.

### Example 40—In Vitro Transposition with Purified MG64-1 Targeting Complex Construct Design and E. coli Strain Production

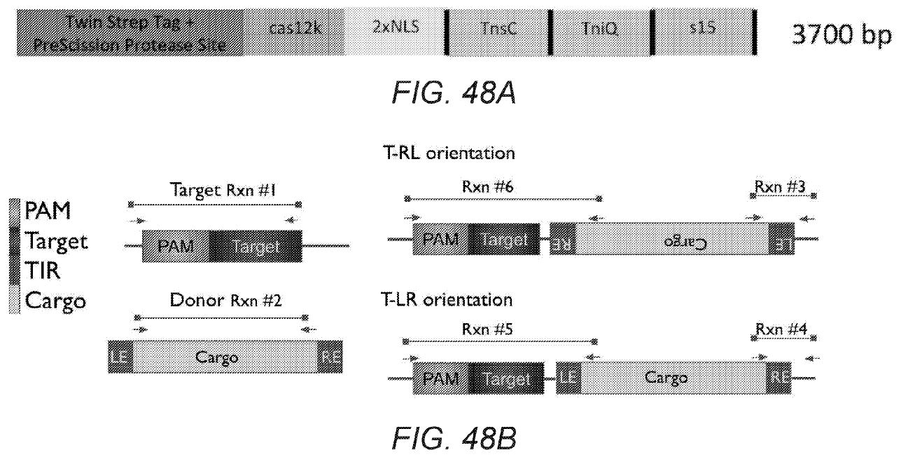

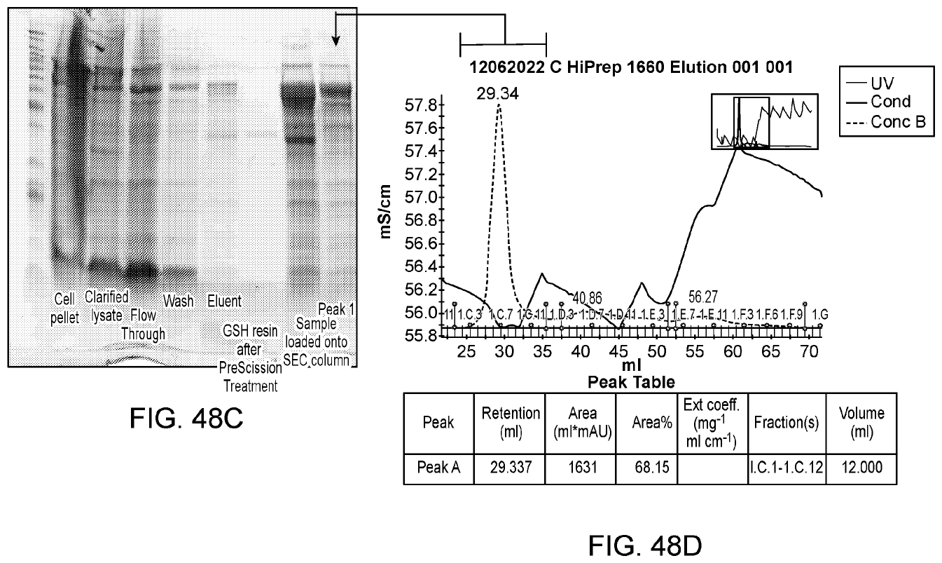

MG64-1 targeting complex was cloned into the BamHI-XhoI sites of the pET-21(+) E. coli expression vector under control of a T7 promoter. Cas12k was expressed with an N-terminal Twin Strep tag and an HRV3C protease site (FIG. 48A). The construct also contained a C-terminal 2×NLS tag on Cas12k, which was expressed in a polycistronic ORF with TniQ, TnsC, and S15 downstream of the Cas12k coding sequence. BL21(DE3) E. coli were transformed with the polycistronic plasmid and co-expressed with an sgRNA containing plasmid under the control of the J23119 constitutively active promoter.

### Purification of Cas12k Targeting Complex

2×1 L cultures of TB were inoculated with 10 mL overnight cultures of the expression construct and guide plasmid grown in LB. The TB medium was supplemented with 50 mM MgCl₂, 1 mL trace elements solution, 100 μg/mL ampicillin, and 15 μg/mL of chloramphenicol per liter. Cultures were grown to OD=0.6, and then induced with addition of IPTG to a final concentration of 0.5 mM. The culture was grown for 3 hours at 37° C. and then harvested by centrifugation.

50 nt oligos were synthesized and annealed in a final concentration of 100 μM. 5 mL of Streptactin resin was loaded onto a gravity flow column and allowed to drain of storage buffer. The resin was then washed with 20 mL wash buffer (50 mM Tris pH 7.4, 750 mM NaCl. 5% glycerol, 0.5 mM TCEP, 1 mM EDTA, 10 mM MgCl₂). Harvested E. coli pellets were lysed using a sonicator in 30 mL Lysis buffer using 12 cycles of (15 sec on 45 sec off) at 75% amplitude. Lysate was cleared by centrifugation at 30,000×g for 25 minutes at 4° C. Clarified lysate was applied to the column and allowed to flow through. The column was then washed with 25 mL of wash buffer. The holo complex was then eluted with 15 mL Elution Buffer (wash buffer with 2.5 mM desthiobiotin). The eluted protein was quantified using Bradford reagent. 50 μL of 100 μM Annealed target oligo and 200 μL of PreScission were added to eluate. Protease reaction was incubated in a rotary shaker at 4° C. overnight. To remove the PreScission protease, 5 mL GSH resin was washed with three cycles of centrifugation at 500×g for 5 minutes and resuspension in 30 mL wash buffer. The protease-treated sample was applied to this resin and incubated for 30 minutes at RT with gentle agitation. The resin was sedimented by centrifugation and the supernatant saved. The GSH resin was washed twice more with 30 mL wash buffer, and the supernatant collected. The protease-treated complex was then added to a prepared SEC column that had been pre-washed with distilled water followed by wash buffer. Samples were loaded onto the column and run at 0.5 mL/min in two column volumes of wash buffer. Protein-containing eluent fractions were pooled, concentrated, and assayed for concentration using Bradford reagent. They were diluted 1:1 into storage buffer (50 mM Tris pH 7.4, 750 mM NaCl, 40% glycerol, 1 mM EDTA, 10 mM MgCl₂, 0.5 mM TCEP) such that the final concentration of glycerol was 20% in the stored, concentrated proteins. Select samples from different stages of purification were run on a denaturing SDS PAGE gel (FIGS. 48C-48D).

### Eukaryotic Transcription and Translation (Tn7) Reactions

Wheat Germ Extract-based in vitro protein expression reactions were used for expression of CAST proteins from templates amplified to contain a T7 promoter and a 40 bp Poly A tail for transcriptional stability of mRNA templates. PCR-amplified templates were normalized to 200 ng/L and loaded into in vitro transcription/translation reactions at a final concentration of 20 ng/L and run for 90 min at 30° C. Crude expressions were then assayed for function by in vitro transposition and used to supplement purified protein fractions.

### In Vitro Transposition

After expression, 1 μL of purified complex (fractions) was added to 0.5 picomoles of sgRNA and incubated for 20 minutes at 25° C. Individually expressed transposase proteins were then added volumetrically at 1 μL per expression with dropouts as noted to test active proteins purified with the complex pulldown. 50 ng of target DNA and 50 ng donor DNA were then added to the transposition reaction in a reaction buffer, with final concentrations of 26 mM HEPES pH 7.5, 4.2 mM TRIS pH 8, 50 μg/mL BSA, 2 mM ATP, 2.1 mM TCEP, 0.05 mM EDTA, 0.2 mM MgCl₂, 28 mM NaCl, 21 mM KCl, 1.35% glycerol (final pH 7.5), and 15 mM Mg(OAc)₂. In vitro transposition reactions were performed at 37° C. for 2 hours, transposition reactions were diluted tenfold in water, and used subsequently as a template for junction PCR analysis (FIG. 48B).

Results: Mass Spectrometry Detection of Cas12k, TnsC, s15, and TniQ in Complex Pulldowns

Proteins were purified from crude lysates according to the protocol described above as holo targeting components (complexed with sgRNA) and fractionated through size exclusion chromatography as described above. Eluted samples of the purified protein complex were submitted for analysis by mass spectrometry. Resulting detection of proteolytic treated protein fragments confirmed that Cas12k, TnsC, TniQ, and s15 were detected (Table 2).

TABLE 2
Identification of components of the MG64-1
holocomplex by mass spectrometry

| Rank | Protein Query | emPAI |
|---|---|---|
| 1 | 64-1_Effector_AA | 23.16 |
| 2 | 64-1_TnsB_AA | 1.33 |
| 3 | 64-1_TnsC_AA | 2.43 |
| 4 | 64-1_TniQ_AA | 1.61 |
| 5 | PreScission_Protease | 0.18 |
| 6 | Nucleoplasmin_NLS | 4.29 |
| 7 | s15_AA | 0.46 |

Tryptic peptides from SEC-eluted protein were searched against a proprietary database of MG64-1 protein sequences. The database also contained reference sequences for PreScission_Protease, NLS and ribosomal protein S15. Multiple components of the MG64-1 transpososome were detected. PreScission_Protease is an expected residual impurity used to cleave the N-terminal strep II tag. emPAI is a relative measure of abundance.

Examples of unique peptide sequences identified by mass spectrometry mapping to the expected sequences of MG64-1 components are presented in Table 3.

TABLE 3
Representative unique peptide sequences identified by mass spectrometry
of tryptic digests from the purified MG64-1 holocomplex

| | Observed m/z | e-value | Peptide sequence | SEQ ID NO: |
|---|---|---|---|---|
| Cas12k | 3624.1189 | 1.30E−18 | K.ATEILQSYEGTEQLFNTLFQAYNSEEDILTR.T | 1259 |
| TnsB | 2402.2166 | 1.90E−11 | R.AGQDLSVEYSNHVWQCDHTR.A | 1260 |
| TnsC | 2033.2468 | 5.60E−05 | K.TVVTLSHVEALHNWLEGK.R | 1261 |
| TniQ | 2627.4727 | 4.90E−20 | R.FIPIPTEEELTALSEVVQVEVER.L | 1262 |

Significant e-values are included. Lower scores indicate more significant results
and a more precise match.

### Results: In Vitro Testing of Purified CAST Targeting Complex Confirms Functional Cas12k, TniQ, TnsC, and s15

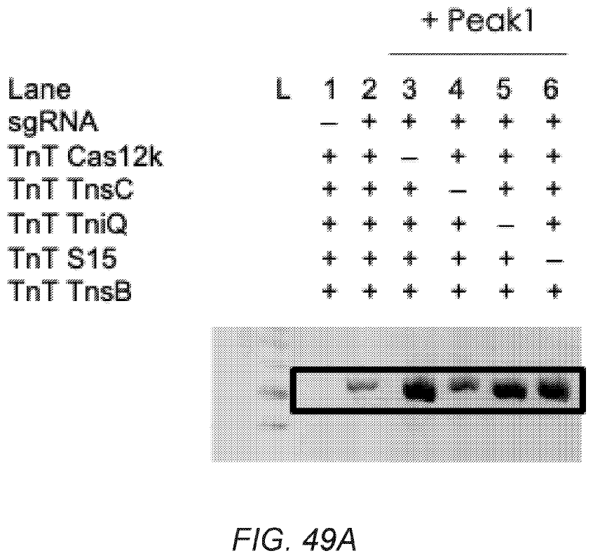

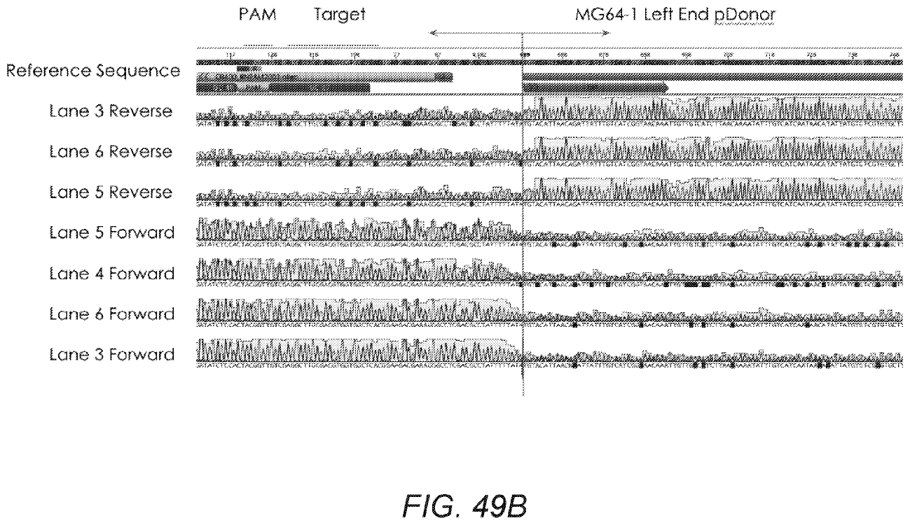

Protein components were purified as a holo targeting complex (with sgRNA) and fractionated through size exclusion chromatography as previously described. Purified complex from Peak 1 was active for transposition without the need for additional Cas12k, TnsC, TniQ nor S15 expressed with Eukaryotic TnT in the reaction (FIG. 49A, lane 3-6). Positive transposition bands for the LE to Target junction were sequenced using Sanger sequencing from both donor and target specific primers, and sequencing results confirmed integration of the LE into the target DNA, as evidenced by the signal degradation at the integration site (FIG. 49B).

### Example 41—Ribosomal Protein S15 Homologs for Targeted Integration

### Results: Bioinformatic Identification of S15 From Cyanobacteria

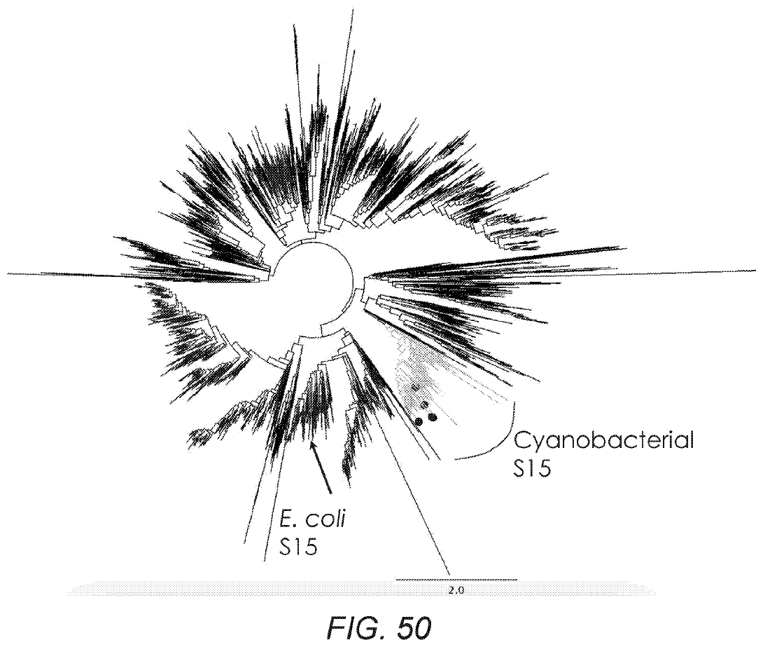

Recently, the small prokaryotic ribosomal protein subunit S15 was deemed necessary for targeted transposition by Cas12k CAST in vitro (Schmidt et al., 2022; Park et al., 2022). Ribosomal protein S15 distant homologs were identified from Pfam PF00312 domain searches with significant e-value of 1e⁻⁵. Of >1 million S15 protein hits, nearly 3,500 full-length, unique S15 sequences were identified in metagenomic assemblies in which Cas12k CAST effectors were also identified. Clustering at 99% average amino acid identity enabled classification of nearly 2,700 S15 cluster members by taxonomic affiliation, of which 11 (SEQ ID NOs: 209-219) were derived from Cyanobacteria (FIG. 50). Five ribosomal protein S15 candidate sequences (MG190-178 through MG190-182, SEQ ID NOs: 209-213) were identified in the same sample in which the MG64-1 CAST was identified (FIG. 50) and are likely associated with this CAST system.

### Example 42—NLS Fusion with S15 of the MG190 Family is Necessary for Transposition (Prophetic)

The need for S15 with and without NLS tags in transposition experiments with MG64-1 or a Cas12k CAST of the MG64 family is evaluated. NLS tags are fused to the N- and/or C-termini of S15 and tested in in vitro transposition experiments. Wheat Germ Extract is used in a Eukaryotic transcription/translation system, which does not contain S15, to express MG64-1 CAST components and NLS-S15 constructs. CAST templates are amplified to contain a T7 promoter and a 40 bp Poly A tail for transcriptional stability of mRNA templates. Proteins are expressed from the dsDNA template via transcription/translation reactions, which are then used in an in vitro transposition reaction as described previously.

### Example 43—in Cell Transposition with CAST and S15 of the MG190 Family (Prophetic)

NLS-tagged CAST proteins are expressed on high expression plasmids for transposition experiments in human cells. A targeting plasmid expresses the protein targeting complex, including S15, under control of a pCAG promoter. The targeting plasmid also contains a pU6 PolIII promoter driving transcription of a humanized sgRNA for in-cell targeted integration. A second donor plasmid containing DNA cargo flanked by the LE and RE terminal inverted repeats is transfected into cells. Cells are seeded 24 hours before lipid-based transfection of the two plasmid system in 9 μg: 9 μg of targeting: donor plasmid. Cells are incubated for 72 hours at 37° C., then harvested by resuspension in 4 mL 1×PBS pH 7.2, 2 mL of resuspended cells are harvested for gDNA extraction and eluted in 200 μL of elution buffer. 5 μL extracted gDNA is assayed for transposition in 100 μL Q5 PCR reactions with primers specific for the target site. Amplified PCR reactions are visualized on a 2% agarose gel. Transpositions are predicted to transpose at 60-65 bp away from the PAM and are determined to be active by the presence of a single band for junction PCR amplification at the predicted size. PCR amplicons are Sanger sequenced and NGS sequenced for transposition profile analysis.

### Example 44—Transposition Off a Single Transcript Plasmid System

Previously, a two-plasmid system encoding the MG64-1 CAST components and donor was constructed for transposition experiments in human cells and were used here as negative controls (FIG. 51A). A single helper plasmid (pHelper) containing all the protein components of the MG64-1 system was constructed, separated with IRES elements. The construct encodes a Cas12k-sso7d-NLS-2A-EcS15-NLS, NLS-H1core-TniQ or NLS-HMGN1-TniQ, NLS-TnsB, and NLS-TnsC (FIG. 51B). This single transcript is expressed from under control of a pCMV-BetaGlobin promoter. A donor plasmid (pDonor) containing a pCMV-BetaGlobin promoter drives expression of an mNeon fluorescent protein flanked by both LE and RE TIR (SEQ ID NO: 223) (FIG. 51B).

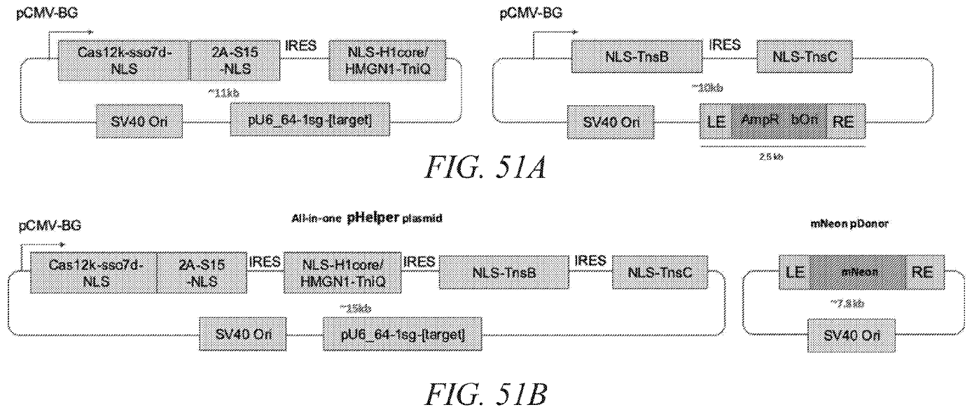

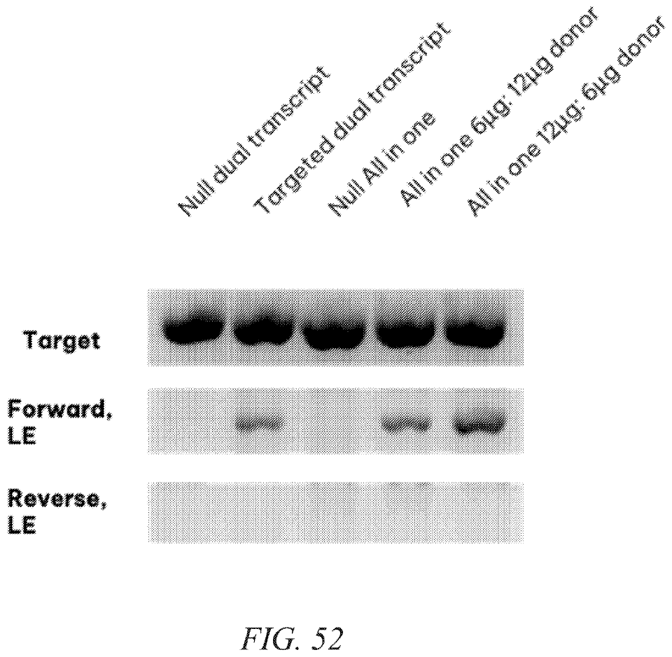

Co-transfection of the pHelper plasmid with the pDonor plasmid was sufficient for transposition of the donor to occur into the Line 1 3′ target 8 of HEK293T cells (FIG. 52). Junction PCRs were performed using primers designed for Line 1 3′ (SEQ ID NOs: 224-225) and an internal donor primer assaying LE transposition (SEQ ID NO: 226). Changes of the pHelper to pDonor concentration ratios helped to identify the optimal transposition frequency at 12:6 μg of pHelper to pDonor.

### Example 45—Transposition with a Non-Replicative Donor

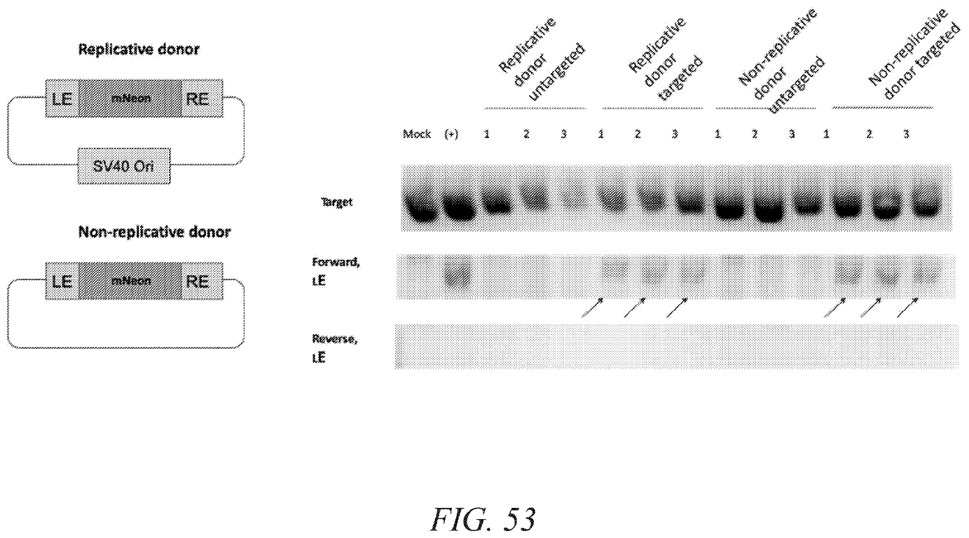

In an engineered HEK293T cell line, plasmids containing the SV40 origin of replication are capable of high retention and replication in cells. This allows for the high prevalence of donor fragments for transposition. Primary cells, however, would not have the ability to self-replicate plasmids, and would need to be tested in conditions without a self-propagating plasmid. In order to simulate these conditions, the SV40 promoter and origin (SEQ ID NO: 227) were deleted from the plasmid resulting in a non-replicative pDonor (pDonor-ND, FIG. 53, diagram).

At the exposed 12:6 μg ratio of pHelper to pDonor, transposition of both the replicative and non-replicative donor into the Line 1 3′ Target 8 site was observed (FIG. 53, gels. Experiments were done in triplicate). Resulting junction PCRs indicated comparable efficiencies of transposition with both replicative and non-replicative donors.

### Example 46—ClpX Enhancement of Transposition

ClpX has been previously shown to enhance the dissociation of MuA (a TnsB paralogue) in the Mu phage genome to help resolve the integration reaction of the Mu Phage. In addition, Cascade CAST (Type I) Tn7 systems are hypothesized to be resolved with the addition of ClpX. ClpX is not present in the human genome, so the E. coli ClpX was delivered into the human genome by synthesizing both NLS versions of ClpX, ClpX-NLS and NLS-ClpX (SEQ ID NOs: 228-230) under control of a CMV-BetaGlobin promoter.

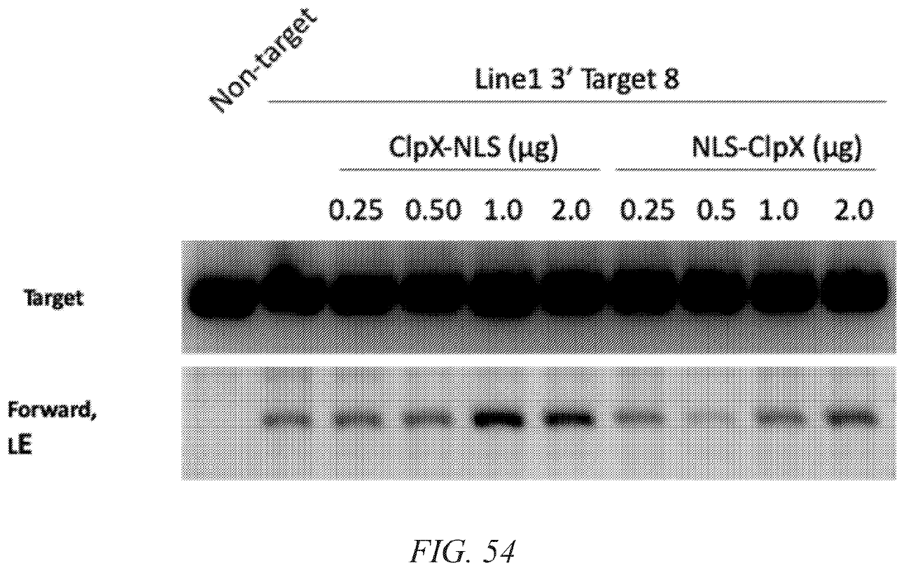

Versions of NLS-tagged ClpX were tested in transposition experiments at various concentrations to determine the active ratio of ClpX with the pHelper and pDonor. Amounts of 0.25, 0.5, 1, and 2 μg of ClpX plasmid were added to cell transfections. After 72 hours, cells were harvested for gDNA and screened for activity at the L1 3′ Target 8 locus. 1 μg of ClpX was observed as the most active, enhancing transposition of the pDonor to the target locus (FIG. 54).

### Example 47—Targeted Transposition to a Single Copy Locus in the Haploid Genome with MG64-1 Cas12k CAST

Cas12k CASTs have only been functional for integration into plasmid targets in human cell lines. It has been previously demonstrated that MG64-1 is capable of targeted transposition into high copy targets of the genome of human cells. Here, with the help of the activity of a bacterial ClpX chaperone, the efficiency of transposition of MG64-1 into low copy number human genomic targets was increased, detectable integration at single copy targets was demonstrated. One of these targets is the safe harbor locus AAVS1, which is a relatively innocuous locus for integration of exogenous genes for mammalian genome expression.

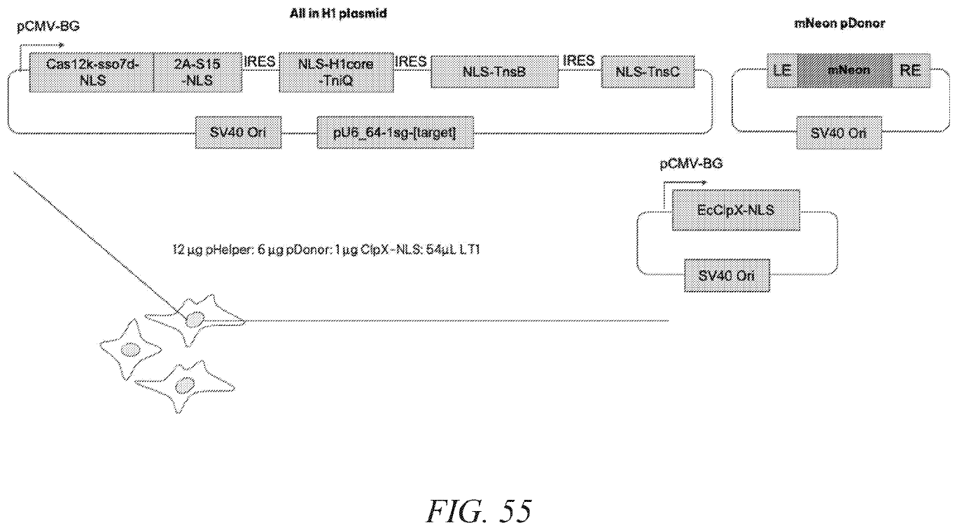

An all-in-one pHelper design that includes a single promoter driving all five protein coding components, along with a cloned single guide RNA target tailored to the AAVS1 locus, were employed for this study (FIG. 55). A second plasmid contained the LE and RE (TIR) flanking a donor mNeon reporter gene. A third replicative plasmid contained the ClpX-NLS (FIG. 55). The three-plasmid system was delivered via lipofectamine in the ratio of 12 μg pHelper:6 μg pDonor:1 μg pClpX:54 μL of lipofectamine reagent LT1 to 24 hour seeded 2.5×10⁶ HEK293T cells. Once transfected, cells were recovered for 72 hours at 37° C., gDNA was extracted from the cells using a Midi Blood L kit, and donor-target junctions were PCR amplified with primers (SEQ ID NOs: 226, 231, and 232) and visualized on a 2% agarose gel.

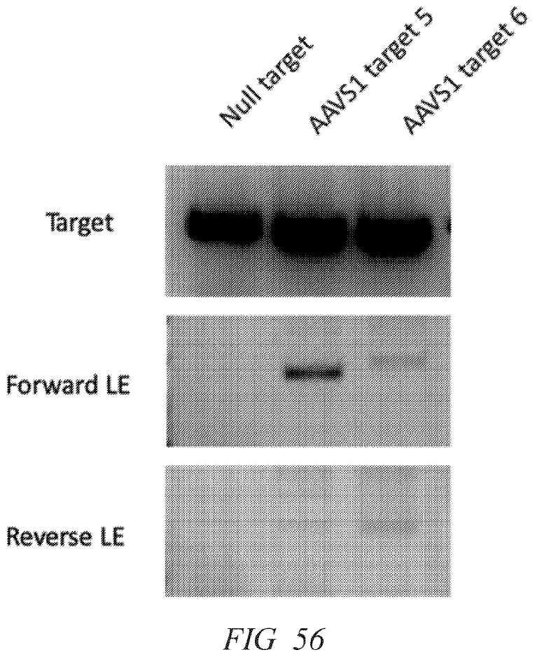

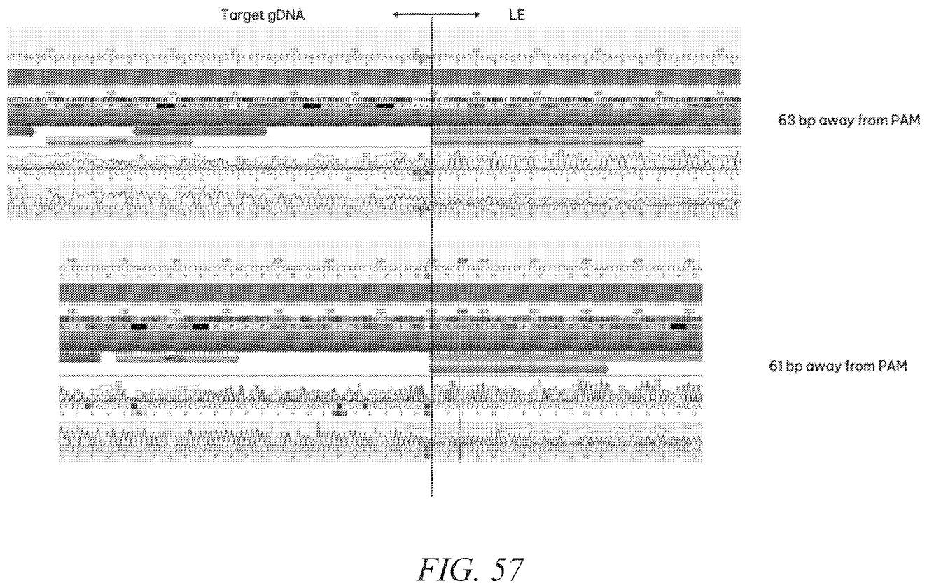

Co-transfection of the pHelper, pDonor, and pClpX plasmids was sufficient for transposition of the donor into the AAVS1 targets 5 and 6 of the AAVS1 safe harbor locus in the Forward LE direction, as evidenced by the presence of a band vs. the null control (FIG. 56). Sequencing of the PCR amplified products confirmed transposition at the AAVS1 target 5 (SEQ ID NO: 233), occurring 63 bp away from the PAM, and at AAVS1 target 6 (SEQ ID NO: 234), which occurs 61 bp away from the PAM (FIG. 57).

### Example 48—Bioinformatic Identification of ClpX Accessory Proteins

Metagenomics databases were mined for proteins with homology to the E. coli ClpP and ClpX. Hits were retained if they had a bit score >50, partial proteins were removed, and proteins were retained if they had a length between 400-500 aa and if ClpX was adjacent to a ClpP homolog. In total, >10,000 ClpX homologs clustered at 80% amino acid identity were identified. Selected ClpX homologs (SEQ ID NOs: 235-249) may improve transposition efficiency of MG64-1.

### Example 49—NGS Reads of AAVS1 Target 5 and 6

Cas12k CASTs have only been functional for integration into plasmid targets in human cell lines. It has been demonstrated that MG64-1 is capable of targeted transposition into high copy targets of the genome of human cells. Here, with the help of the activity of a bacterial ClpX chaperone, the efficiency of transposition of MG64-1 into low copy number human genomic targets has been increased, detectable integration at single copy targets has been demonstrated. One of these targets is the safe harbor locus AAVS1, which is a relatively innocuous locus for integration of exogenous genes for mammalian genome expression.

An all-in-one pHelper design that includes a single promoter driving all five protein coding components was used along with a cloned single guide RNA target tailored to the AAVS1 locus (FIG. 55). A second plasmid contained the LE and RE (TIR) flanking a donor mNeon reporter gene. A third replicative plasmid contained the ClpX-NLS (FIG. 55). The three-plasmid system was delivered via lipofectamine in the ratio of 12 μg pHelper:6 μg pDonor:1 μg pClpX:54 μL of lipofectamine reagent LT1 to 2.5×10⁶ HEK293T cells seeded for 24 hours. Once transfected, cells were recovered for 72 hours at 37° C., and then the gDNA was extracted from the cells using a Midi Blood L kit. Donor-target junctions were PCR amplified with primers (SEQ ID NOs: 250-251) and sequenced by NGS.

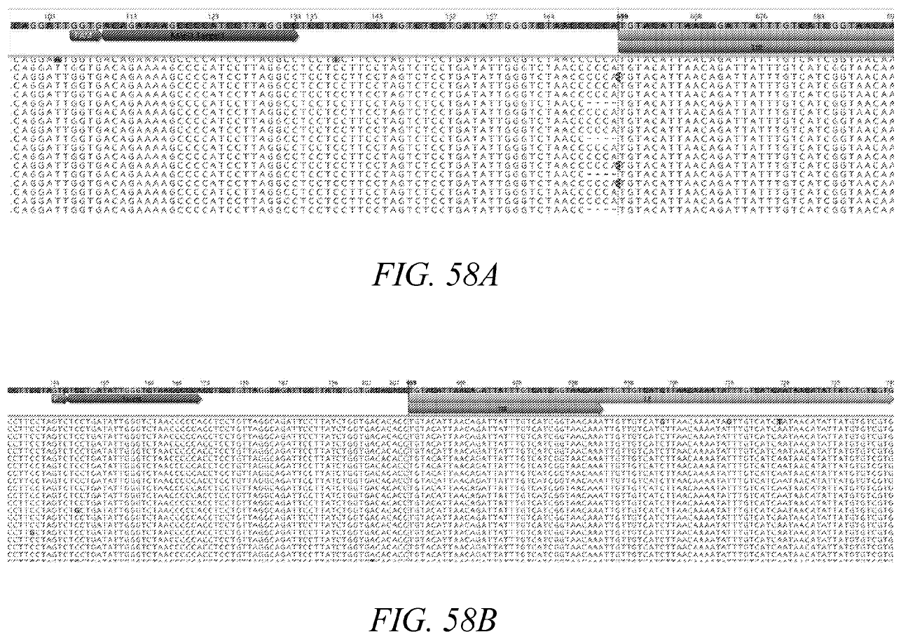

Single molecule allele of target to LE transposition products were visualized at the intended locus. AAVS1 target 5 has a primary transposition product of 63 bp away from PAM and is a heterogenous mix of other transposition products in the window of 10 bp (FIG. 58A), and AAVS1 target 6 has a primary transposition product 61 bp away from PAM (FIG. 58B).

### Example 50—Efficiency Determination of Single Copy Site AAVS1 Target 5 by NGS

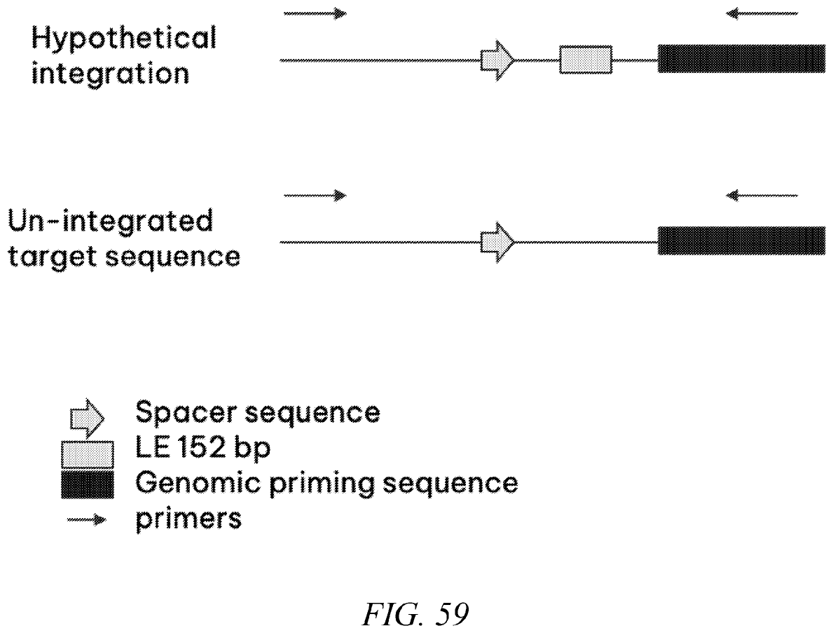

When utilizing a donor for NGS efficiency measurements, a gDNA fragment is incorporated into the pDonor for the specific locus targeted. By modeling the primary transposition sequence with the major transposition outcome as determined by sanger sequencing, genomic sequences with a 57.9° C. priming Tm were determined and placed on the pDonor in the same spacing from the primary transposition product, 152 bp away from the 5′ LE (FIG. 59). This genomic fragment allows for the simultaneous amplification of the AAVS1 transposition product and the genomic sequence (SEQ ID NO: 252) with the same PCR primer set (SEQ ID NOs: 250 and 253).

Genomic and transposed LE forward sequences were amplified using 100 μL PCR reactions of Q5 polymerase for 25 cycles using oJL1109 and oJL1125 primers (SEQ ID NOs: 250 and 253). Primers included an adaptor sequence and a 5 bp diversity stub. Sequencing adapters were then amplified for 10 cycles onto the PCR product library after 1×SPRI cleanup and sequenced with a 2×300 cycle V3 kit on MiSeq. Resulting NGS reads were analyzed, using the 63 bp target 1 transposition sequence (SEQ ID NO: 254) as the reference amplicon, the genomic fragment as the HDR amplicon (SEQ ID NO: 255), and the Left End 5′ sequence as the spacer within a 20 bp window (SEQ ID NO: 256). Resulting alignments were then filtered. Unmodified reference sequence, NHEJ sequences, HDR sequences, and modified HDR sequences were then pulled from the editing profile. Transposition frequencies were calculated by summing reference aligned sequences and NHEJ sequences over the total amount of reads.

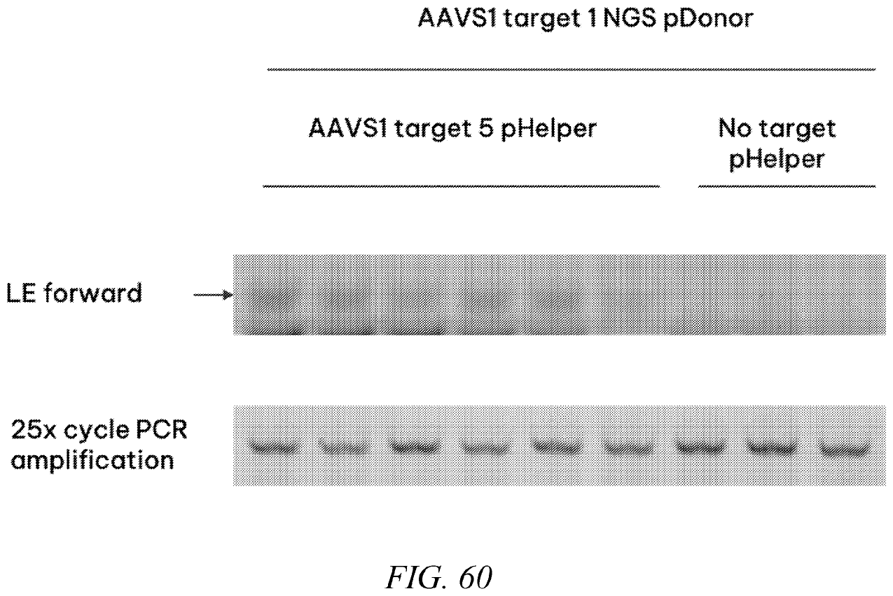

Co-transfection of the pHelper, pDonor, and pClpX in a 12:6:1 μg ratio of plasmids was sufficient for transposition of the donor into the AAVS1 target 5 of the AAVS1 safe harbor locus in the Forward LE direction, as evidenced by the presence of a band vs. the null control (FIG. 60, top panel). Genomic amplification was performed with no target controls at 25 cycles (FIG. 60, lower panel) and visualized on a 2% gel. NGS sequencing was performed with an indexing PCR with 10 cycles and sequenced by 2×300 cycles.

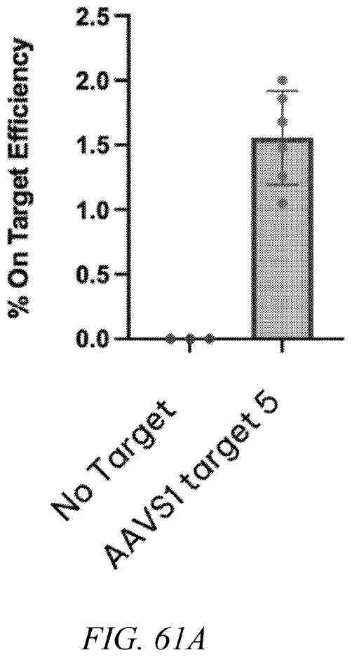

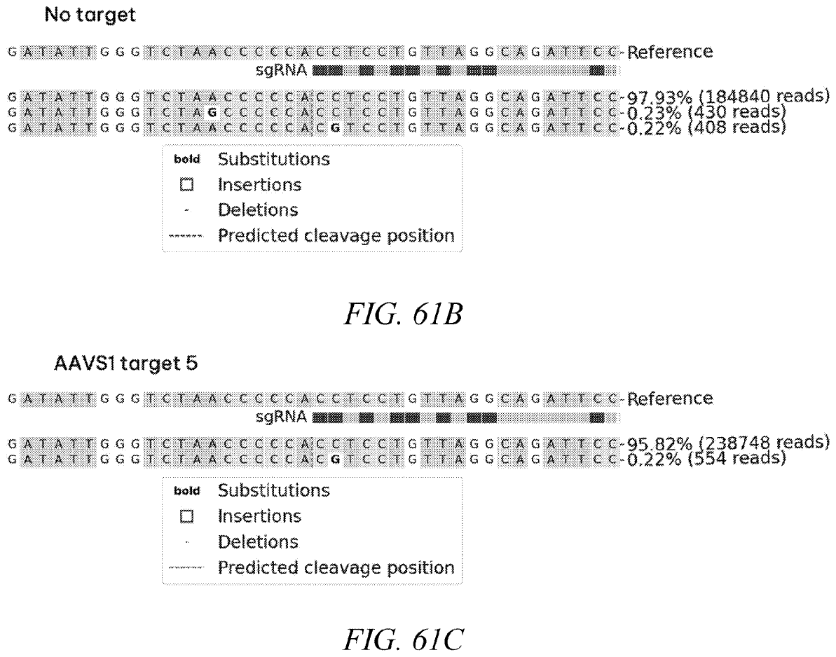

Resulting reads were analyzed with reference genomic sequence (SEQ ID NO: 254), HDR sequence (SEQ ID NO: 255), and spacer sequence (SEQ ID NO: 256). Summed Unmodified reference sequence and NHEJ sequences were summed over total reads aligned to reference, NHEJ, HDR, and imperfect HDR as a percentage of total reads. AAVS5 achieved a mean of 1.55±0.35% transposition (N=6) (FIG. 61A). No detectable transposition reads were found from NGS sequencing of the no-target control allele frequency normalized to AAVS1 target 1 targeted allele frequency (FIGS. 61B and 61C). No edited reads were found in the genomic reads as a result of CAST expression.

### Example 51—Delivery of Linear pDonor in HEK293T

Cas12k CASTs have only been functional for integration into plasmid targets in human cell lines. We have demonstrated that MG64-1 is capable of targeted transposition into high copy and single copy targets of the genome of human cells. While conventional DNA donor introduction in cells uses a plasmid for donor integration, the backbone of the DNA donor may potentially be integrated into the genome. To resolve this problem, a linear donor was tested and found to be sufficient for integration.

### Construct Design and HEK293T Cell Transfection

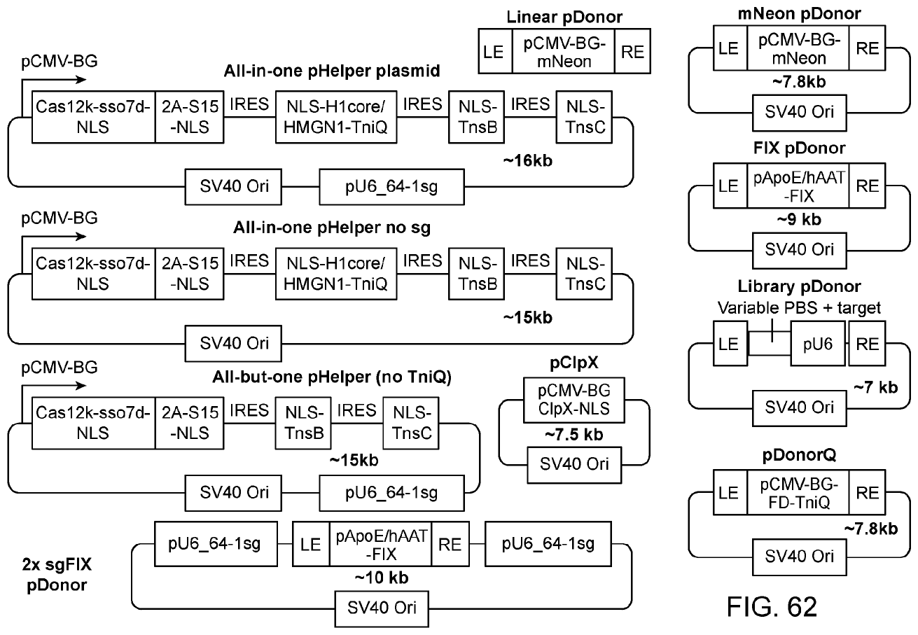

To generate a linear donor fragment, the non-replicative donor plasmid containing the AAVS1 target 5 Primer binding sequence (PBS) (SEQ ID NO: 368) was cut with the enzyme, SphI-HF. Cut donor molecules were then concentrated and purified using a DNA clean-up kit, such as DNA Clean and Concentrator Kit-100. The experiment utilized an all-in-one Helper plasmid, featuring a single promoter driving all five protein coding components, along with a cloned single guide RNA target tailored to the AAVS1 locus (FIG. 62) and an additional plasmid containing the host factor ClpX-NLS. The two-plasmid system with a linear donor was delivered via lipofection in the ratio of 12 μg pHelper:6 μg linear donor:1 μg pClpX:54 μL of lipofectamine reagent LT1 to 24 hour seeded 2.5×10⁶ HEK293T cells. After a 72-hour transfection incubation at 37° C., genomic DNA was extracted from the cells using a Midi Blood L kit, and the left end (LE) donor-target junctions were assayed by NGS and ddPCR.

### NGS Sequencing and Analysis

Extracted gDNA was amplified with target specific primers for AAVS1 (SEQ ID NOs: 366 and 367). Amplicons were then indexed with TruSeq dual unique indices for NGS. The resulting reads were analyzed with CRISPResso2 with a reference genomic sequence (SEQ ID NO: 369), a HDR sequence (SEQ ID NO: 370), and a spacer sequence (SEQ ID NO: 371) with settings of window centered at 0, window of 10, and alignment filtering “-amas 95”. The summed unmodified reference sequence and NHEJ sequences were summed over total reads aligned to reference, NHEJ, HDR, and imperfect HDR as a percentage of total reads.

ddPCR Analysis

Prior to Droplet Digital PCR (ddPCR), the gDNA was digested with EcoRI-HF). A total reaction mix containing digested gDNA, 2×ddPCR Supermix for Probes (no dUTP), primers (SEQ ID NOs: 360 and 361), and the probe (SEQ ID NO: 362) designed for each predicted integration site from LE was loaded for droplet generation and PCR amplification. The data was then generated and analyzed using the QX200 droplet reader and software to quantify edited alleles in each sample. The data was normalized by using a housekeeping gene, RPL13A (Biorad ddPCR Copy Number Assav:RPL13A, Human. Assay ID: dHsaCNS189783948).

### Results

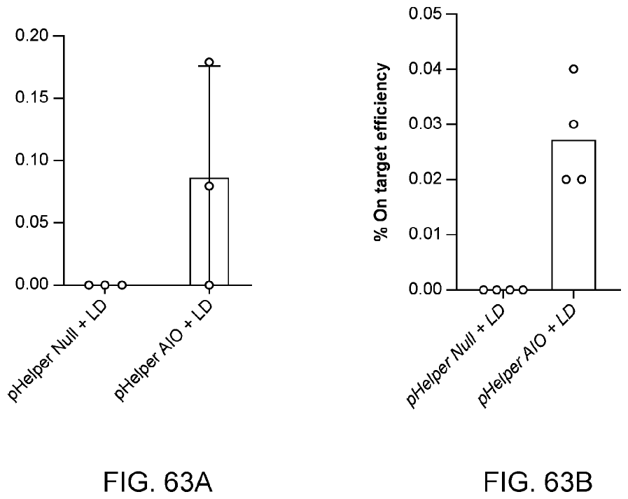

Co-transfection of the pHelper, pClpX, and linear plasmid in HEK293T cells was sufficient for transposition of the donor into the AAVS1 target 5 of the AAVS1 safe harbor locus in the forward LE direction, as evidenced by the presence of the target-donor junction at 0.03% efficiency by ddPCR and 0.15% by NGS (FIG. 63A and FIG. 63B).

### Example 52—Targeted Transposition to Single Copy Locus in K562 and Hep3B Cells

While the HEK293T cell line is a well-established chassis for plasmid replication and protein production, the application of CASTs into a therapeutic would require translation of the system into a broader range of cells that are more closely related to the organ type intended for the final therapeutic. As an alternative, K562 cells were selected because they are frequently used in immunology research as an immortalized immune cell type. These cell types may not typically be amenable to the same plasmid transfection process as determined by HEK293T cells, thus novel dosing of plasmids needed to be determined and introduced using nucleofection.

### Construct Design and K562 Cell Transfection

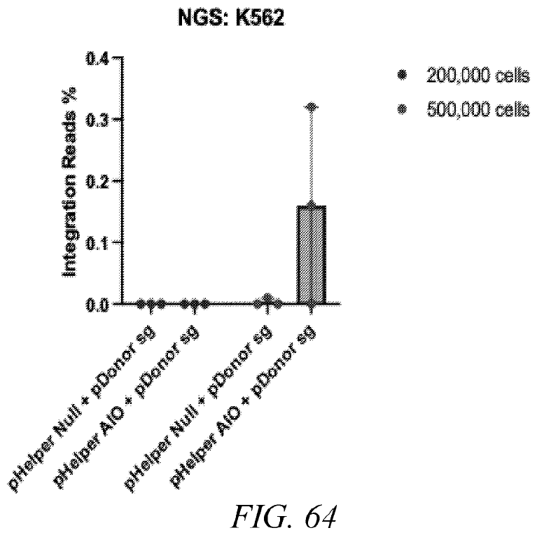

An all-in-one pHelper design that included a single promoter driving all five protein coding components, along with a cloned single guide RNA target tailored to the AAVS1 target 5 locus was employed for this study (FIG. 62). A second plasmid contained the LE and right end (RE) Toll/interleukin-1 receptor (TIR) domain, flanking a donor cargo of single guide encoding the spacer for AAVS1 target 5 locus. A third replicative plasmid contained the ClpX-NLS (FIG. 62). The three-plasmid system was delivered by nucleofection using a ratio of 1.3 μg pHelper:3.2 μg pDonor:0.1 μg pClpX, along with 20 μL of SF Nucleofector Solution with Supplement, in a Nucleocuvette Strip containing either 2×10⁵ or 5×10⁵ K562 cells, using the FF-120 program on a 4D nucleofector. Nucleofected cells were incubated for 72 hours at 37° C. in 24-well plates, followed by genomic DNA extraction using 100 μL of QuickExtract, and quantification was performed by NGS using LE primers (SEQ ID NOs: 366 and 367). Co-transfection of the pHelper with guides targeting AAVS1-5, pDonor with a single guide targeting AAVS1-5, and pClpX plasmids was sufficient for transposition of the donor into the AAVS1 targets 5 of K562 when nucleofected with 5×10⁵ cells (FIG. 64).

### Construct Design and Hep3B Cell Transfection

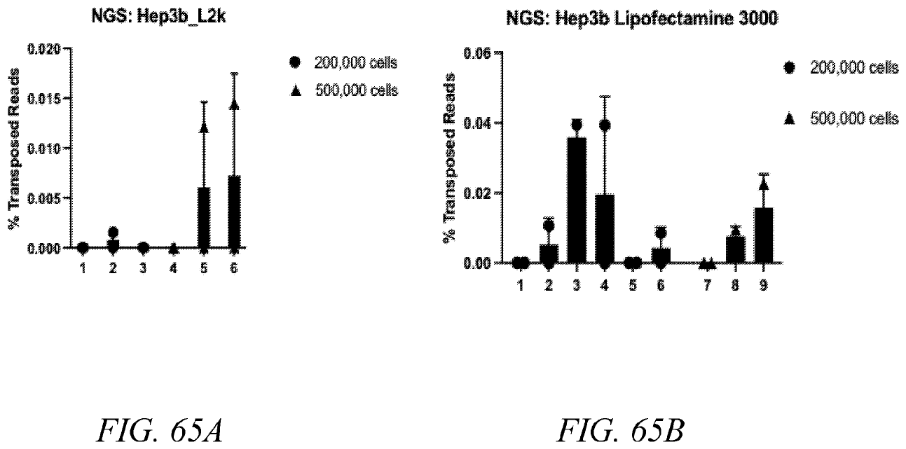

An all-in-one pHelper design that includes a single promoter driving all five protein coding components, along with a cloned single guide RNA target tailored to the AAVS1 target 5 locus was used in this experiment (FIG. 62). A second plasmid contained the LE and RE (TIR) flanking a donor cargo of single guide encoding the spacer for AAVS1 target 5 locus and AAVS1 target 5 PBS (SEQ ID NO: 368). A third replicative plasmid contains the ClpX-NLS (FIG. 62). The three-plasmid system was transfected using either Lipofectamine 2000 (L2K) using 3-5 μL for 200,000 cells or 6-12 μL for 500,000 cells or Lipofectamine 3000 (L3K), between 1.5-3.75 μL, for 200,000 cells or 3.75-8.25 μL for 500,000 cells. pHelper:pDonor:pClpX dosing varied between 0.3-1.3 μg pHelper, 0.051-0.1 μg pDonor, and 0.8-3.2 μg pClpX in a 12-well for 200,000 cells or 6-well plate 500,000 cells. Transfected cells were incubated for 72 hours at 37° C., and genomic DNA was extracted at post-72 hours transfection using the Kingfisher MagMax 2.0 Extraction Kit, then quantified by NGS using LE primers (SEQ ID NOs: 366 and 367). Co-transfection of the pHelper, pDonor, and pClpX plasmids was sufficient for transposition of the donor into the AAVS1 targets 5 of Hep3B in 24 well plates (FIGS. 65A and 65B). FIG. 65A depicts conditions tested for (1) 0.33 μg pHelper:0.051 μg pDonor:0.8 μg ClpX with 3 μl L2k, 12-well with 2e5 cells, (2) 0.33 μg pHelper:0.051 μg pDonor:0.8 μg ClpX with 4 μl L2k, 12-well with 2e5 cells, (3) 0.33 μg pHelper:0.051 μg pDonor:0.8 μg ClpX with 5 μl L2k, 12-well with 2e5 cells, (4) 1.3 μg pHelper:0.1 μg pDonor:3.2 μg ClpX with 6 μl L2k, 6-well with 5e5 cells, (5) 1.3 μg pHelper:0.1 μg pDonor:3.2 μg ClpX with 9 μl L2k, 6-well with 5e5 cells, (6) 1.3 μg pHelper:0.1 μg pDonor:3.2 μg ClpX with 12 μl L2k, 6-well with 5e5 cells. FIG. 65B shows conditions tested for (1) 0.33 μg pHelper:0.051 μg pDonor:0.8 μg ClpX with 1.5 μl L3k, 12-well with 2e5 cells, (2) 0.66 μg pHelper:0.051 μg pDonor:1.6 μg ClpX with 1.5 μl L3k, 12-well with 2e5 cells, (3) 0.33 μg pHelper:0.051 μg pDonor:0.8 μg ClpX with 3 μl L3k, 12-well with 2e5 cells, (4) 0.66 μg pHelper:0.051 μg pDonor:1.6 μg ClpX with 3 μl L3k, 12-well with 2e5 cells, (5) 0.33 μg pHelper:0.051 μg pDonor:0.8 μg ClpX with 3.75 μl L3k, 12-well with 2e5 cells, (6) 0.66 μg pHelper:0.051 μg pDonor:1.6 μg ClpX with 3.75 μl L3k, 12-well with 2e5 cells, (7) 1.3 μg pHelper:0.1 μg pDonor:3.2 μg ClpX with 3.75 μl L3k, 6-well with 5e5 cells. (8) 1.3 g pHelper:0.1 μg pDonor:3.2 μg ClpX with 7.5 μl L3k, 6-well with 5e5 cells, (9) 1.3 μg pHelper:0.1 μg pDonor:3.2 μg ClpX with 8.25 μl L3k, 6-well with 5e5 cells. Transposition of CAST at AAVS1 in Hep3B was maintained with either transfection reagent when 5e5 cells were transfected (FIGS. 65A and 65B), Lipofectamine 3000 was able to achieve robust transfection of cells seeded at 2e5 (FIG. 65B).

### Example 53—Single Guide Target Screening to hALB

Transposition into an intronic region of a dysfunctional gene would allow for the correction of all single nucleotide polymorphisms, truncations, deletions or other allelic heterogeneities downstream of the targeted region. By introducing cargoes in frame with the upstream exons, protein fusions could also be achieved to allow for exogenous gene expression. ALB1, the gene encoding albumin, is a highly expressed gene in hepatocytes and its genomic region is frequently used as a model for protein expression in cell biology. We evaluated CAST transposition potential into the ALB1 locus.

### Construct and Library Design

All potential single guides for human ALB (hALB) intron 1 targets were predicted (SEQ ID NOs: 754-815) and constructed in a pDonor construct with LE and RE flanking a U6 promoter driving a CAST single guide to a single target. A primer binding sequence (PBS) specific for each target site that mimics the genomic locus that allows for NGS profiling was cloned into each pDonor (SEQ ID NOs: 945-1006). Arrayed pDonors were cloned, amplified purified by miniprep, and then normalized to 100 ng/μL. Two targets (SEQ ID NOs: 816 and 817) for AAVS1 were cloned in a similar matter for positive controls, with cognate PBS sequences (SEQ ID NOs: 1007 and 1008).

### HEK293T Cell Transfection

An all-in-one pHelper design that includes a single promoter driving all five protein coding components was used in this experiment. To this, a second replicative ClpX-NLS plasmid was pooled with the arrayed pDonor guide plasmid. The three-plasmid system was delivered via Lipofectamine in 1e5 HEK293T cells pre-seeded for 24 hours in 24 well plates at the ratio of 0.66 μg pHelper:0.33 μg pDonor:0.051 μg pClpX. Transfected cells recovered for 72 hours at 37° C. in 24 well plates, gDNA was then extracted from the cells using Kingfisher MagMax 2.0 Extraction Kit, and percent editing was quantified by NGS using primers designed for each potential integration site (SEQ ID NOs: 372-435 and 563-626).

### Results

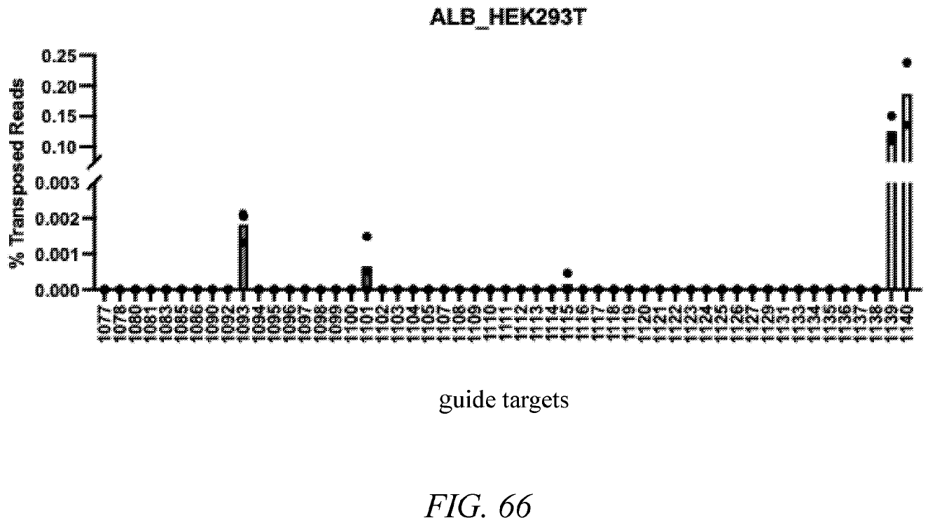

Co-transfection of the pHelper, pClpX, and arrayed pDonor plasmids is sufficient for transposition of the donor into intron 1 of the human ALB gene. Of the arrayed pDonors, guides for targets 1093, 1101, and 1115 had detectable transposition over background (FIG. 66). NGS reads of targets 1139 and 1140 show a positive control integration at AAVS1 using the same integration setup.

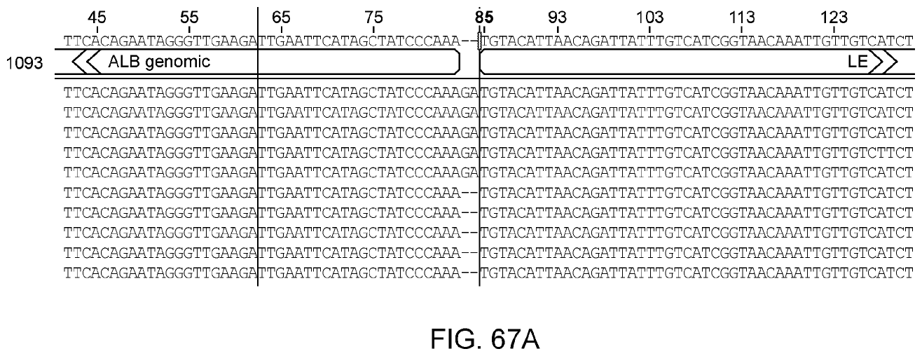

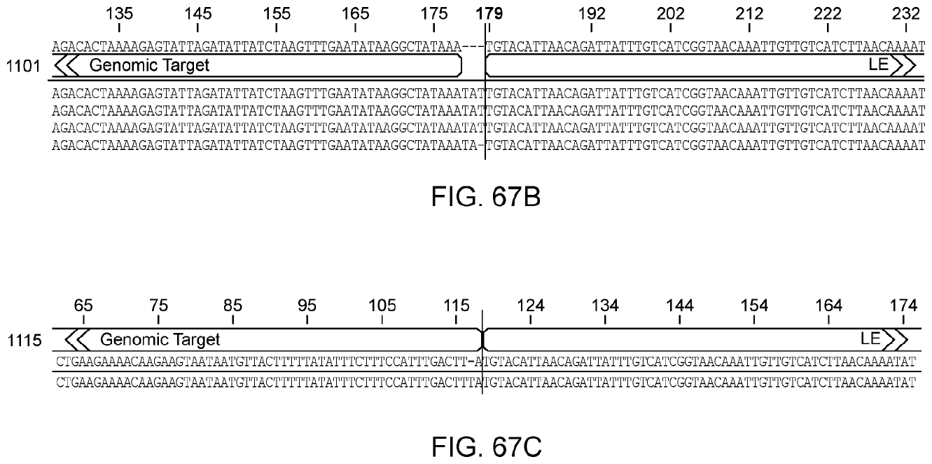

NGS reads for targets 1093 (FIG. 67A), 1101 (FIG. 67B) and 1115 (FIG. 67C) were aligned to modeled reference reads at an estimated 60 bp away from the PAM. These integration reads indicated that efficiencies detected at human ALB intron 1 were bonafide integrations with clear alignment to genomic targets with LE TIR integrations. Preferential integration was indicated across each target, and was preferential for 60/62 bp, 62/63 bp, and 61 bp away from PAM for targets 1093, 1101, and 1115, respectively.

### Example 54—Single Guide Target Screening to mALB1 and mROSA26

Transposition into an intronic region of a dysfunctional gene would allow for the correction of all single nucleotide polymorphisms, truncations, deletions or other allelic heterogeneities downstream of the targeted region. By introducing cargoes in frame with the upstream exons, protein fusions could also be achieved to allow for exogenous gene expression. In mouse cells, mALB1 is the gene encoding albumin, and is a highly expressed gene in mouse hepatocytes, and mROSA26 is a safe harbor locus that is frequently targeted for exogenous DNA integration. Both genomic regions are frequently used as an expression model for protein expression and genomic editing.

### Construct and Library Design

All potential single guides for mouse ALB1 (mALB1) and mouse ROSA26 (mRosa26) were constructed in a pDonor construct with LE and RE flanking a U6 promoter driving a CAST single guide to single target (SEQ ID NOs: 818-944). A primer binding sequence (PBS) specific for each target site that mimics the genomic locus that allows for NGS profiling was cloned into each pDonor (SEQ ID NOs: 1009-1135). Arrayed pDonors were cloned, amplified, and purified by miniprep and normalized to 50 ng/μL.

### Hepa1-6 Cell Transfection

An all-in-one pHelper design that includes a single promoter driving all five protein coding components was used in this experiment. To this, a second replicative ClpX-NLS plasmid was pooled with the arrayed pDonor guide plasmid. The three-plasmid system was delivered via Lipofectamine in 1×10⁵ Hepa1-6 cells pre-seeded for 24 hours in 24 well plates at the ratio of 0.66 μg pHelper:0.33 μg pDonor:0.051 μg pClpX. Transfected cells were recovered for 72 hours at 37° C. in 24 well plates, gDNA was extracted from the cells using Kingfisher MagMax 2.0 Extraction Kit, and percent editing was quantified by NGS using primers designed for each potential integration site (SEQ ID NOs: 436-562 and 627-753).

### Results

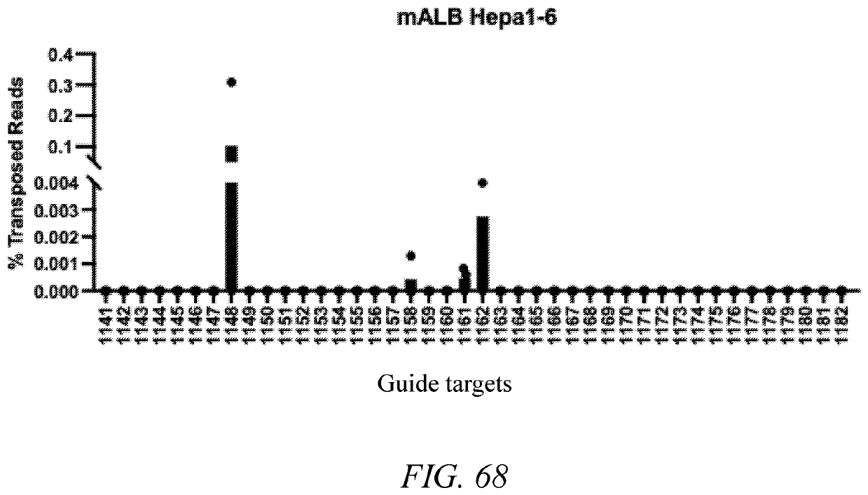

Editing in Hepa1-6 was achieved at multiple donors across both mRosa26 and mAlb in Hepa1-6 cells. Integration into intron 1 of mouse Alb resulted in integration across 4 targets, targets 1148, 1158, 1161 and 1162 (FIG. 68).

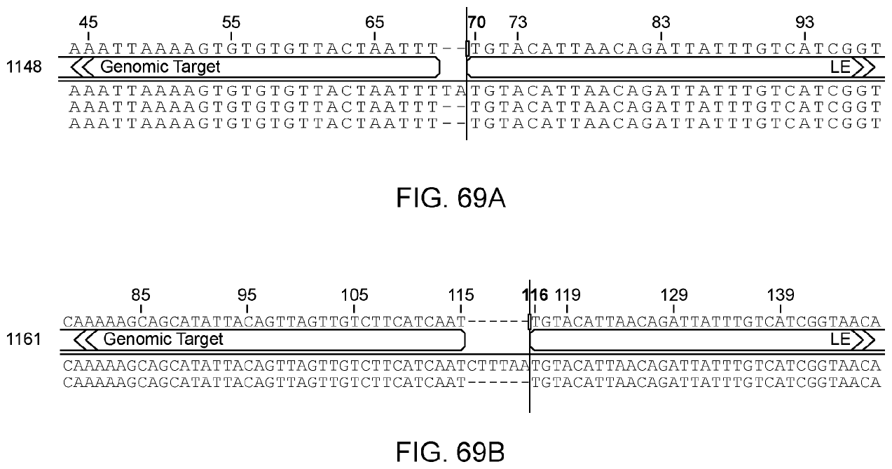

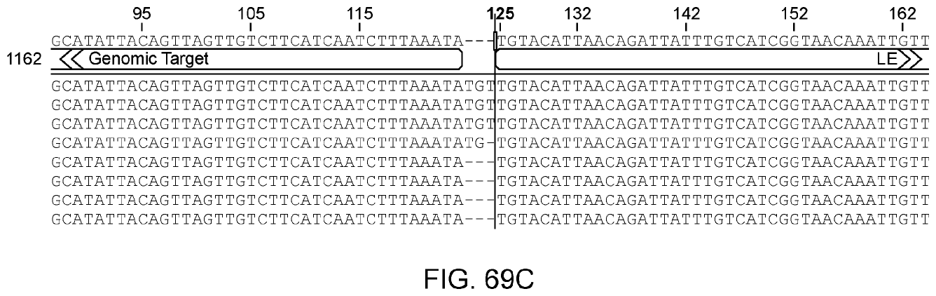

NGS reads for targets 1148 (FIG. 69A), 1161 (FIG. 69B) and 1162 (FIG. 69C) were aligned to modeled reference reads at an estimated 60 bp away from the PAM. These integration reads indicated that efficiencies detected at mouse ALB intron 1 were bonafide integrations with clear alignment to genomic targets with LE TIR integrations. Preferential integration was indicated across each target, and was preferential for 60/62 bp, 60/65 bp, and 60-63 bp away from PAM for targets 1148, 1161 and 1162, respectively.

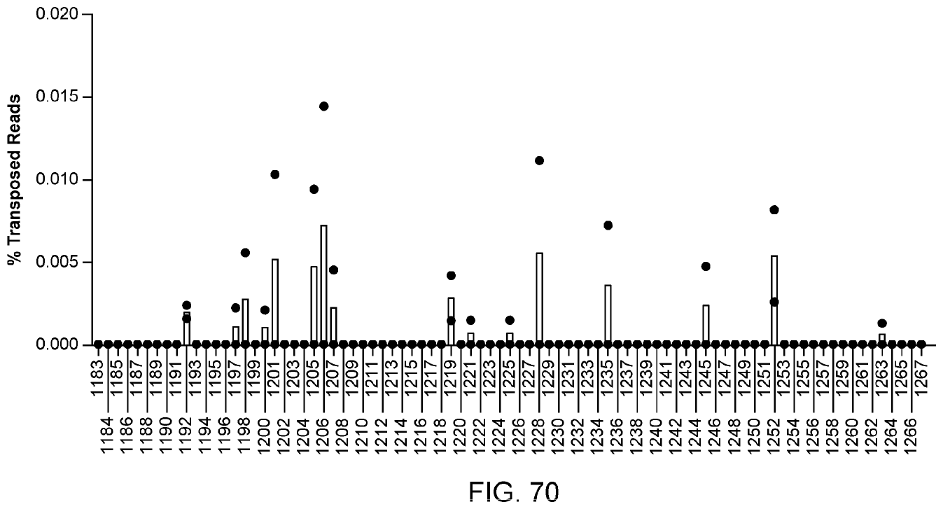

Integrations determined by NGS reads of transposition from mRosa26 locus determined multiple sites of integration including targets 1192, 1197, 1198, 1200, 1201, 1205, 1206, 1207, 1219, 1221, 1225, 1228, 1235, 1245, 1252, and 1263 (FIG. 70).

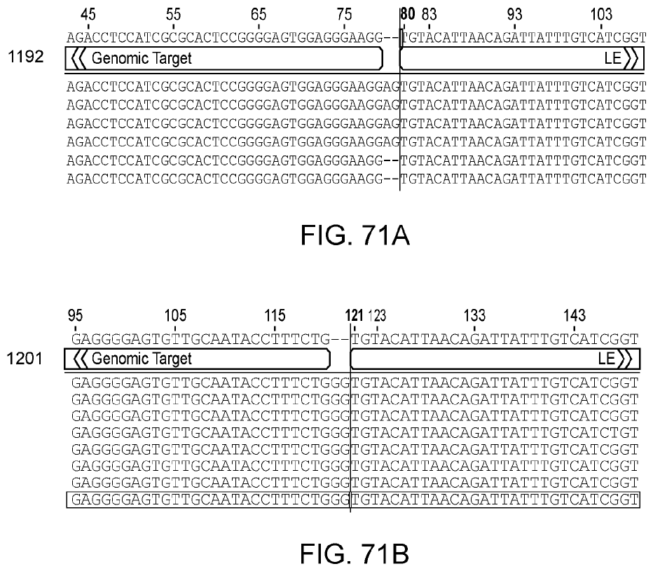

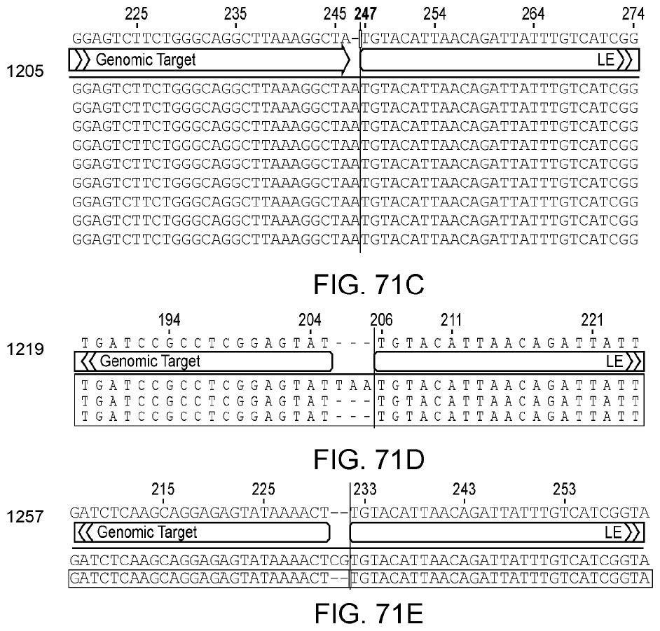

NGS reads for targets 1192 (FIG. 71A), 1201 (FIG. 71B), 1205 (FIG. 71C), 1219 (FIG. 71D) and 1252 (FIG. 71E) were aligned to modeled reference reads at an estimated 60 bp away from the PAM. These integration reads indicated that efficiencies detected at mouse Rosa26 locus were bonafide integrations with clear alignment to genomic targets with LE TIR integrations. Preferential integration was indicated across each target, and is preferential for 60/62 bp. 62 bp, 61 bp, 60/63 bp, 60/62 bp away from PAM for targets 1192, 1201, 1205, 1219, and 1252, respectively.

### Example 55—Single Guide Dosage Increases Integration Efficiency in 293T Cells

Single guides for CAST transposition may be limiting in cells due to limited expression from plasmid replication and transcription.

### Construct Design and HEK293T Cell Transfection

An all-in-one pHelper plasmid with a single promoter driving all five protein coding components was used across all conditions in this experiment. In some instances, the pHelper also contained a cloned single guide RNA targeting the AAVS1 target 5 locus. A second plasmid containing the LE and RE (TIR) flanking a donor cargo was used and, where indicated, the pDonor plasmid contained two additional pU6 driven single guide cassettes targeting AAVS1 target 5. A third replicative plasmid contains the ClpX-NLS. The three-plasmid system was delivered via lipofection in the ratio of 0.66 μg pHelper:0.33 μg pDonor:0.051 μg pClpX by lipofectamine 2000 on 100,000 HEK293T cells in a well of a 24-well plate. Transfected cells were recovered for 72 hours at 37° C., genomic DNA was then extracted from the cells using Kingfisher MagMax 2.0 genomic DNA extraction kit at 72-hour post-transfection, and edited alleles of each sample were quantified by NGS using LE primers (SEQ ID NOs: 366 and 367).

### Results

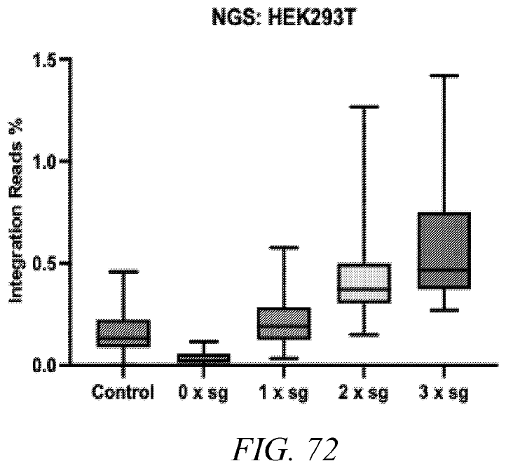

By introducing increasing amounts of pU6-sg cassettes for expression in the cell, the efficiency of transposition directly correlated with measurements by NGS. Through combinations of a single guide on pHelper and two single guides on pDonor, 3-fold increases were able to be achieved over single cassette control for integration at the AAVS1 locus (FIG. 72).

### Example 56—Integration of Factor IX with CAST is a Complete Integration

CAST integration of DNA cargos is enzymatic in nature where the 3′ ends of the terminal inverted repeats provide the substrate for nucleophilic attack at the target locus. If only one enzymatic reaction was preferred or had a significant lag in transposition, exonucleases may prevent full integration of the cargo by excising the ends of the terminal inverted repeats. By being able to detect the rates of integration for both LE and RE genomic junctions and comparing the relative efficiency by ddPCR, this would show the rate of complete cargo integration.

Construct Design, HEK293T Cell Transfection, and ddPtCR Quantification

An all-in-one pHelper plasmid with a single promoter driving all five protein coding components was used across all conditions in this experiment. In the null pHelper condition, the single guide cassette encodes a non-targeting spacer. In the conditions with AAVS1 targeting, pHelper contained a single guide RNA targeting the AAVS1 target 5 locus. A second plasmid containing the LE and RE (TIR) flanking a donor cargo that includes a chimeric apolipoprotein E human al anti-trypsin (ApoE-hAAT) promoter (SEQ ID NO: 1136) driving full Factor IX (SEQ ID NO: 1137). A third replicative plasmid contains the ClpX-NLS. The three-plasmid system was delivered by LT1 transfection reagent in a 12 μg pHelper:6 μg pDonor:1 μg pClpX on 2,500,000 HEK293T cells in a 10 cm petri dish. Transfected cells were recovered for 72 hours at 37° C., genomic DNA (gDNA) was extracted from the cells at 72 hours post-transfection using the Macherey Nagel L Blood Kit. Integrations for LE were determined by ddPCR using LE specific primers (SEQ ID NOs: 360 and 361) and LE specific probes (SEQ ID NO: 362) and compared with ddPCR of RE specific primers (SEQ ID NOs: 363 and 364) and RE specific probe (SEQ ID NO: 365).

### Results

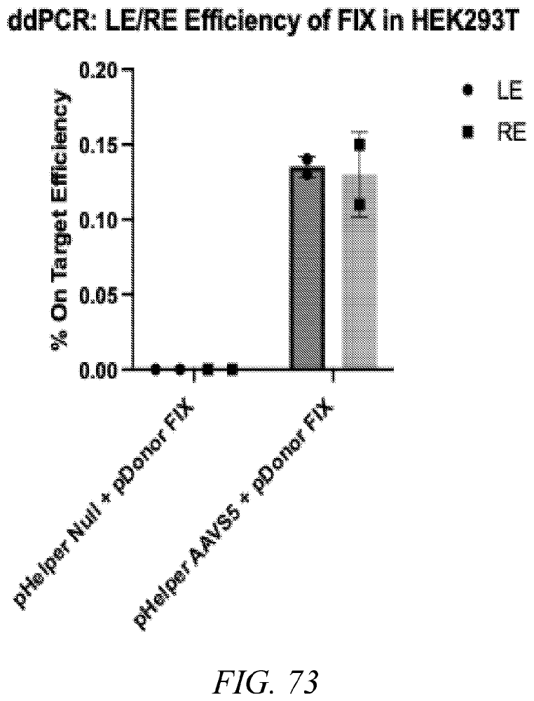

Detection of the LE junction was performed and showed no integration without a single guide to give programmable transposition. In this case, both LE and RE junctions did not detect any quantifiable integration events. When AAVS1-5 target was added to the pHelper, detection of the LE and the RE were both detected in a 1:1 ratio to each other. This indicates that cargo integration of the full Factor IX cargo by CAST delivery was complete where both ends were quantifiable and equal (FIG. 73).

### Example 57—Novel Functional Domain Discovery

Low or lack of activity of some effectors may be explained by their inability to access genomic target sites, for example, due to tight chromatin structure. Fusions of functional domains (FD) to these effectors can significantly improve enzymatic activity. For example, fusions of Taq polymerase to the sso7d dsDNA binding protein improved processivity of the enzyme, requiring much less enzyme yields and a much shorter extension time compared to wild type Taq polymerase (Wang, et al., 2004). Similarly, dsDNA nuclease editing efficiency of CjCas9 in K562 cells improved when fused with a variety of functional domains (Ding, et al., 2019).

Functional domains (FD), including DNA Binding domains (DBD) and chromatin modulating domains (CMD), are small proteins capable of facilitating protein-protein interactions, as well as interactions of proteins with nucleic acids. Some FD bind “loosely” to DNA in a non-sequence-specific manner and help with search function along genomic DNA (Dyson, 2012). Previously, it was found that Cas12k CAST protein component engineering for expression and nuclear localization in human cells may not be sufficient to unlock CAST integration activity despite all components properly localizing in the nuclear fraction. Fusions of the targeting effector Cas12k to the archaeal protein sso7d (residues 1-64), and of the transposon component TniQ to the human histone 1 central globular domain (H1 core, residues 22-10l) or a High-Mobility Group domain (HMGN1, residues 1-100) activated CAST integration into the genome of human cells. Here, functional domains (FD) discovered from large metagenomics databases may unlock additional potential of CRISPR systems for programmable gene editing technology development.

### Bioinformatic Discovery of Functional Domains Front Metagenomics

Distant FD homologs (SEQ ID NOs: 257-307 and 1138-1242) were discovered by mining a large assembly-driven metagenomic database of microbial, viral, and eukaryotic genomes. Hits to the Pfam domains PF02294 (7kD DNA-binding domain) and PF01101 (HMGN1) with e-value lower than le⁻⁵ were identified and dereplicated with mmseqs easy-cluster at 100% and 95% average amino acid identity (AAI), respectively, over 80% alignment coverage (Steinegger and Soeding, 2017). Sequences for both FD types were aligned using MAFFT (Katoh and Standley, 2013) with local parameters (L-INS-i) and phylogenetic trees were built using FastTree (Price, et al., 2010).

### MG161 Family of FD are Divergent Sso7d Homologs

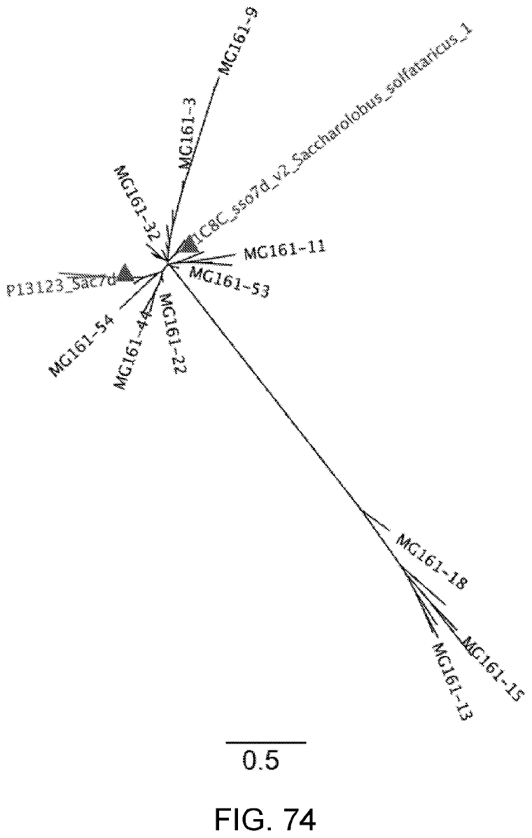

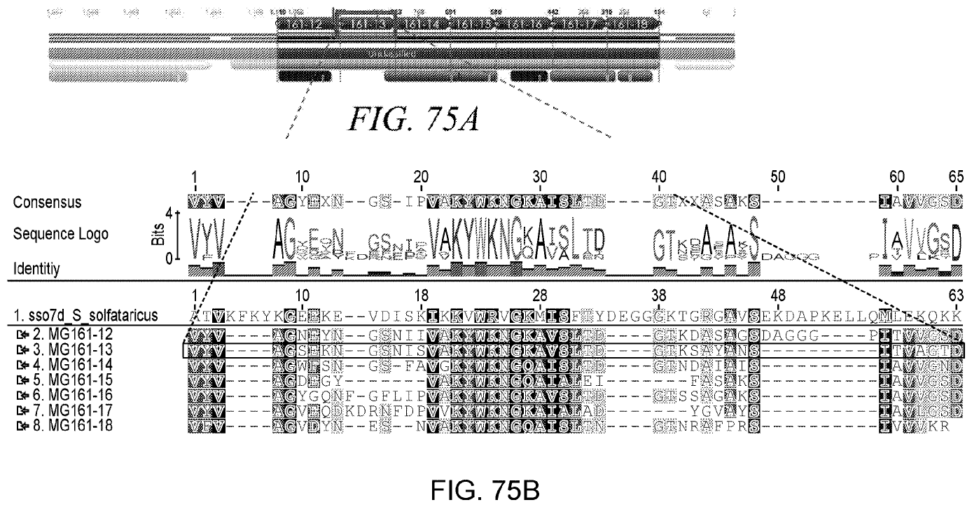

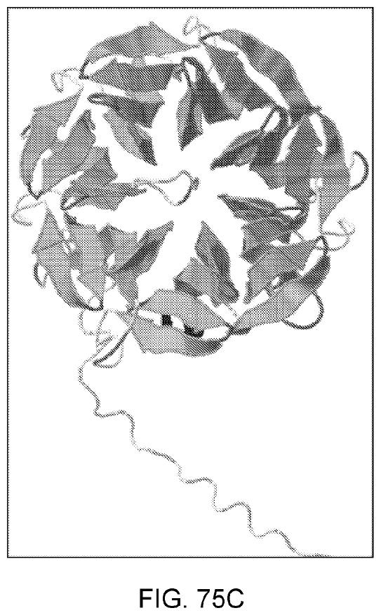

Sso7d from the archaeon Sulfolobus solfataricus belongs to a family of small 7 kDa, non-sequence specific DNA binding proteins found primarily in hyperthermophilic archaea, which play a role in stabilizing genomic DNA (Lemmens, et al., 2022). Phylogenetic analysis of sso7d homologs suggests that members of the MG161 family are divergent from the reference sso7d sequences, as observed by long branches separating homologs (FIG. 74). Some MG161 functional domains (FD) are encoded as short, single domain proteins in thermophilic bacterial genomes (SEQ ID NOs: 257-278, 1148-1160, 1202-1229), while others are encoded as perfect or imperfect tandem direct repeats within long predicted open reading frames (ORF) recovered from viral genomes (FIGS. 75A-75B; SEQ ID NOs: 279-282, 1141-1201, and 1243-1249). 3D structure prediction of these long tandem repeat-encoding ORFs indicates that some of these repeats form well defined structures may be important for function (FIG. 75C). The novel MG161 tandem repeat FD identified here share conserved residues with the reference sso7d sequences but shared on average less than 40% average amino acid identity.

### MG162 are Divergent HMGN1 Homologs

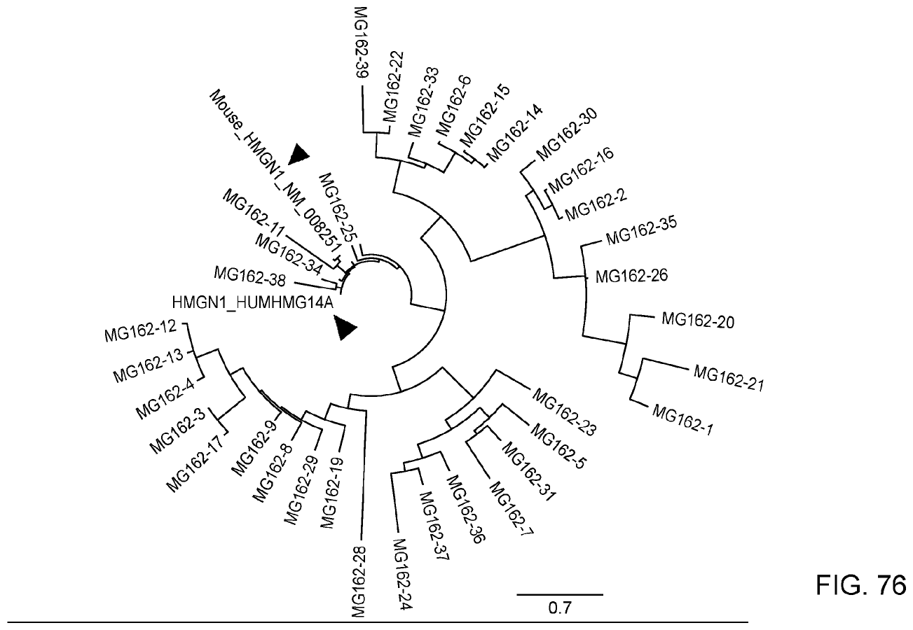

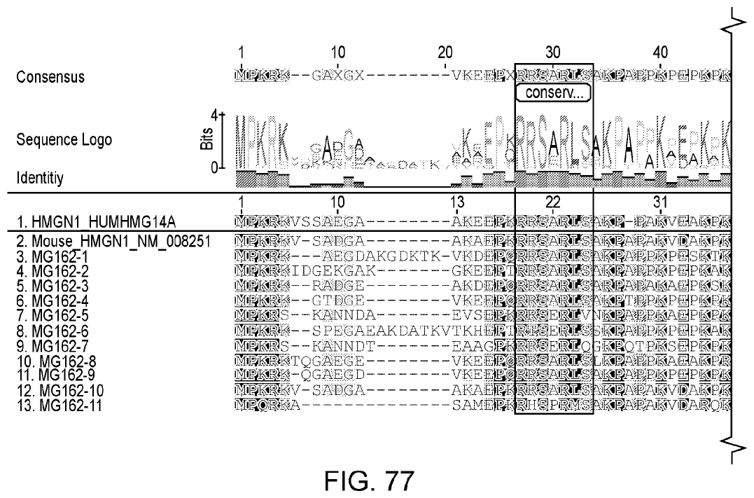

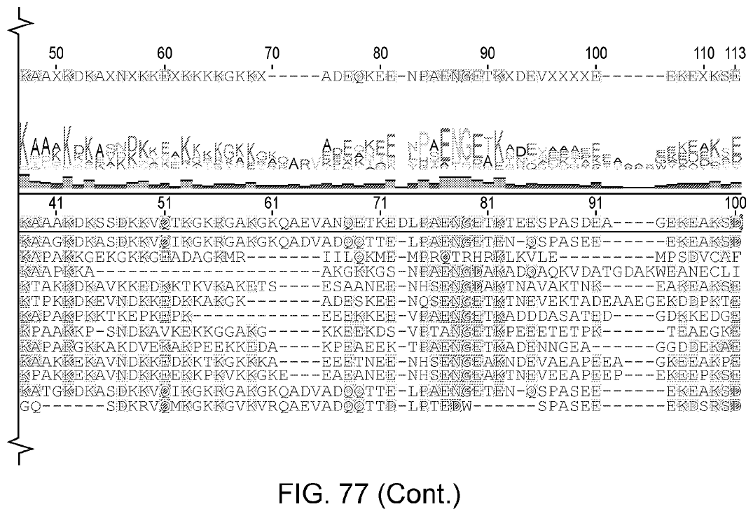

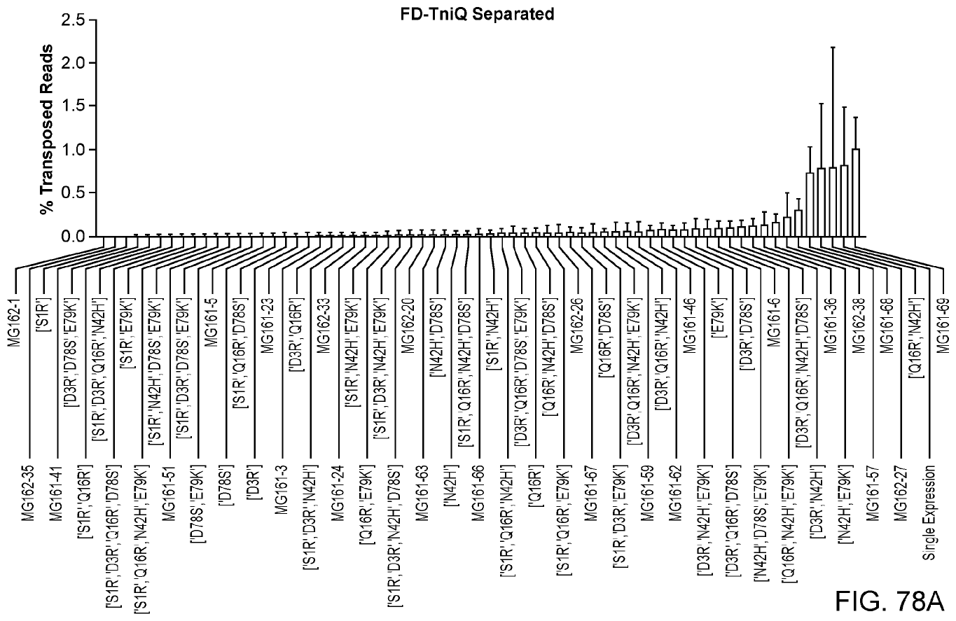

High-Mobility Group proteins bind to nucleosomes and induce chromatin structural changes (Postnikov and Bustin, 2015). HMGN1 is an example of a high-mobility group protein. Distant homologs of HMGN1 were identified in Eukaryotic genomes recovered from metagenomics data (FIG. 76), exhibit 40% average pairwise amino acid identity (AAI) with the HMGN1 reference sequences, and most homologs contain a conserved RXSXRL amino acid motif (SEQ ID NO: 1263) (FIG. 77; SEQ ID NOs: 283-307 and 1230-1242), which is known to play a role in protein-DNA binding interaction (Postnikov and Bustin, 2010).

### Example 58—Novel Functional Domain Fused to TniQ Enhance Integration Rates in Expression Systems where Engineered TniQ is Expressed from a Separate Construct and in Single Expression Constructs

Novel FD-TniQ fusions and engineered variants of known FD may enhance the integration efficiency of the CAST complex. Many instances of the FD-TniQ constructs were evaluated, including instances where the FD-TniQ fusion was expressed along with the other CAST coding sequences from a single construct and instances where the FD-TniQ fusion was expressed from a second construct independently from the other coding sequences. The FDs that have been tested come from 3 sources, (1) metagenomic discovery of novel sso7d-like functional domains; (2) metagenomic discovery of novel HMGN1-like functional domains; and (3) the human histone H1 globular (H1core) domain with rationally designed, site-specific amino acid substitutions.

In addition, target amino acid substitutions on the Hlcore domain were designed according to three criteria: (1) amino acid sites with high mutational entropy and high-frequency substitutions, as determined by deep multiple-sequence alignment of natural homologous of H1core; (2) sites with high solvent accessible surface areas were identified and prioritized; and (3) high-frequency substitutions identified from natural homologs that introduce positive charges or remove negative charges were then selected. According to these criteria, 6 amino acid sites were selected for targeted substitutions and a combinatorial mutagenesis library containing all 63 possible combinations of these substitutions was generated and tested.

### Construct Design and HEK293T Cell Transfection

Two separate experimental designs were used to test the FD-TniQ candidates. In the first design, a pHelper plasmid was generated that contained all CAST protein coding sequences except for TniQ. A second plasmid was created to provide a single FD-TniQ candidate (SEQ ID NOs: 257-359) along with the donor molecule. These plasmids, along with a replicative pClpX plasmid, provided all necessary components for editing. Experiments testing this design were carried out in 24 well plates containing with 50,000 HEK293T cells wherein a combination of 0.66 μg of pHelper, 0.33 μg pDonor:FD-TniQ, and 0.051 μg ClpX-NLS was delivered to cells with LT1 transfection reagent. Cells were recovered for 72 hours at 37° C., gDNA was then extracted from the cells using King Fisher MagMax 2.0 kit, and editing of alleles was calculated for each FD-TniQ fusion by NGS.

In the second design, a single all-in-one pHelper plasmid was generated for each screened FD-TniQ candidate. Each tested construct expressed from a single promoter all required CAST protein coding sequences, including a single FD-TniQ fusion. A pDonor plasmid containing the LE and RE (TIR) flanking a donor cargo that includes a human chimeric apolipoprotein E human al anti-trypsin (ApoE-hAAT) promoter driving a full Factor VIX coding sequence, with a total cargo size of approximately 4,000 bp. A third replicative plasmid expressed ClpX-NLS. These three plasmids were combined at ratios of 0.33 μg pHelper, 0.165 μg of pDonor, and 0.025 μg of pClpX and delivered to 25,000 HEK293T cells in a 96 well plate with Lipofectamine-2000 transfection reagent. Transfected cells were recovered for 72 hours at 37° C., gDNA was then extracted from the cells using King Fisher MagMax 2.0 kit, and editing of alleles was calculated for each FD-TniQ fusion by NGS.

### Results

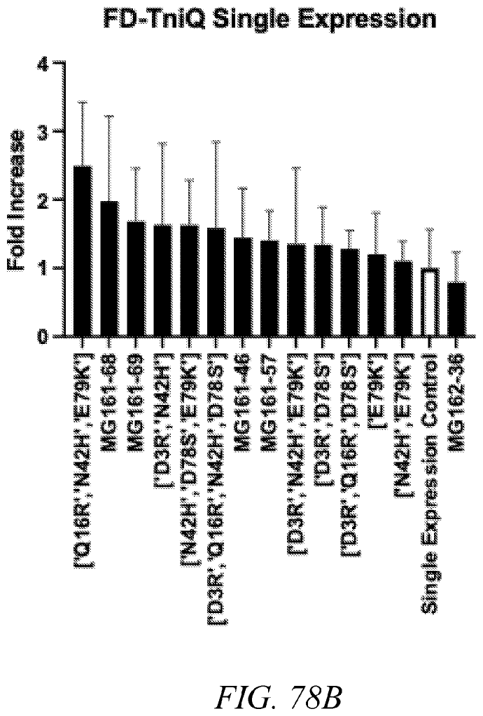

Percent editing was increased in both experimental designs, relative to the Hlcore-TniQ FD fusion. In the split-plasmid expression system, where FD-TniQ candidates were expressed from an independent pDonor plasmid, positive percent editing was detected by NGS from 31 of the 63 FD-TniQ candidate fusions by NGS using LE primers (SEQ ID NOs: 366 and 367). The wild type Hlcore-TniQ FD fusion did not have detectable editing when expressed in the two-plasmid expression platform, suggesting that any FD fusion with detectable editing is an improved variant relative to H1core FD (FIG. 78A). The 15 FD-TniQ fusions with the highest detected editing from the two-plasmid expression approach were assembled into the single plasmid expression system and retested. When expressed from a single plasmid system, 14 of the 15 candidate FD-TniQ fusions had a fold improvement greater than 1 relative to the Hlcore-TniQ fusion (FIG. 78B). Results indicate that novel and engineered functional domains are promising tools to improve effectors activity in cells.

### REFERENCES

Ding X, Seebeck T, Feng Y, Jiang Y, Davis G D, Chen F. Improving CRISPR-Cas9 Genome Editing Efficiency by Fusion with Chromatin-Modulating Peptides. CRISPR J. 2019 February; 2:51-63. doi: 10.1089/crispr.2018.0036. PMID: 31021236.
Katoh K, Standley D M. MAFFT multiple sequence alignment software version 7: improvements in performance and usability. Mol Biol Evol. 2013, 30(4):772-780.
Lemmens L, Wang K, Ruykens E, Nguyen V T, Lindas A C, Willaert R, Couturier M. Peeters E. DNA-Binding Properties of a Novel Crenarchaeal Chromatin-Organizing Protein in Sulfolobus acidocaldarius. Biomolecules 2022 Mar. 30; 12(4):524. doi: 10.3390/biom12040524. PMID: 35454113; PMCID: PMC9025068.

Postnikov Y V, Bustin M. Functional interplay between histone H1 and HMG proteins in chromatin. Biochim Biophys Acta. 2016 March; 1859(3):462-7. doi: 10.1016/j.bbagrm.2015.10.006. Epub 2015 Oct. 8. PMID: 26455954; PMCID: PMC4852864.

Postnikov Y, Bustin M. Regulation of chromatin structure and function by HMGN proteins. Biochim Biophys Acta. 2010 January-February; 1799(1-2):62-8.

Price, M. N., Dehal, P. S., and Arkin, A. P. FastTree 2—Approximately Maximum—Likelihood Trees for Large Alignments. PLoS ONE, 2010, 5(3):e9490.

Steinegger M and Soeding J. MMseqs2 enables sensitive protein sequence searching for the analysis of massive data sets. Nature Biotechnology, 2017, doi: 10.1038/nbt.3988.

Wang Y, Prosen D E, Mei L, Sullivan J C, Finney M, Vander Horn P B. A novel strategy to engineer DNA polymerases for enhanced processivity and improved performance in vitro. Nucleic Acids Res. 2004 Feb. 18; 32(3):1197-207. doi: 10.1093/nar/gkh271. PMID: 14973201; PMCID: PMC373405.

While preferred embodiments of the present disclosure have been shown and described herein, it will be obvious to those skilled in the art that such embodiments are provided by way of example only. It is not intended that the disclosure be limited by the specific examples provided within the specification. While the disclosure has been described with reference to the aforementioned specification, the descriptions and illustrations of the embodiments herein are not meant to be construed in a limiting sense. Numerous variations, changes, and substitutions will now occur to those skilled in the art without departing from the disclosure. Furthermore, it shall be understood that all aspects of the disclosure are not limited to the specific depictions, configurations or relative proportions set forth herein which depend upon a variety of conditions and variables. It should be understood that various alternatives to the embodiments of the disclosure described herein may be employed in practicing the disclosure. It is therefore contemplated that the disclosure shall also cover any such alternatives, modifications, variations, or equivalents. It is intended that the following claims define the scope of the disclosure and that methods and structures within the scope of these claims and their equivalents be covered thereby.

1. A system for transposing a cargo nucleotide sequence into a target nucleic acid site in a target nucleic acid comprising:
a) a Cas effector complex comprising a class 2, type V Cas effector, a small prokaryotic ribosomal protein subunit S15, and an engineered guide polynucleotide that hybridizes to the target nucleic acid site;
b) a Tn7 type transposase complex that binds the Cas effector complex and comprises a TnsB, TnsC, and TniQ component and an accessory protein comprising a sequence having at least 70% sequence identity to any one of SEQ ID NOs: 228-230 and 235-249; and
c) a double-stranded nucleic acid that interacts with the Tn7 type transposase complex and comprises the cargo nucleotide sequence.

2. The system of claim 1, wherein the Cas effector complex binds non-covalently to the Tn7 type transposase complex.

3. The system of claim 1, wherein the Cas effector complex is covalently linked to the Tn7 type transposase complex.

4. The system of claim 1, wherein the Cas effector complex is fused to the Tn7 type transposase complex.

5. The system of claim 1, wherein the cargo nucleotide sequence is flanked by a left-hand transposase recognition sequence and a right-hand transposase recognition sequence recognized by the Tn7 type transposase complex.

6. The system of claim 5, wherein the left-hand transposase recognition sequence comprises a sequence having at least 80% identity to any one of SEQ ID NOs: 9, 11, 36-38, 76, and 78.

7. The system of claim 5, wherein the right-hand transposase recognition sequence comprises a sequence having at least 80% identity to any one of SEQ ID NOs: 8, 10, 39-44, 77, 79, and 93.

8. The system of claim 1, wherein the target nucleic acid comprises a PAM sequence compatible with the Cas effector complex.

9. The system of claim 8, wherein the PAM sequence comprises SEQ ID NO: 31.

10. The system of claim 8, wherein the PAM sequence is located about 50 to about 70 base pairs from the target nucleic acid site.

11. The system of claim 10, wherein the PAM sequence is located 3′ of the target nucleic acid site.

12. The system of claim 10, wherein the PAM sequence is located 5′ of the target nucleic acid site.

13. The system of claim 1, wherein the class 2, type V Cas effector is a Cas12k effector.

14.-28. (canceled)

29. A system for transposing a cargo nucleotide sequence into a target nucleic acid site in a target nucleic acid comprising:
a. a Cas effector complex comprising a class 2, type V Cas effector, a small prokaryotic ribosomal protein subunit S15, and an engineered guide polynucleotide that hybridizes to the target nucleic acid site;
b. a Tn7 type transposase complex that binds the Cas effector complex and comprises a functional domain (FD)-TniQ fusion, and an accessory protein; and
c. a double-stranded nucleic acid that interacts with the Tn7 type transposase complex and comprises the cargo nucleotide sequence.

30.-51. (canceled)

52. A system for transposing a cargo nucleotide sequence into a target nucleic acid site in a target nucleic acid comprising:
a. a Cas effector complex comprising a class 2, type V Cas effector and an engineered guide polynucleotide that hybridizes to the target nucleic acid site, wherein the Cas effector complex comprises a polypeptide comprising a sequence having at least 80% sequence identity to any one of SEQ ID NOs: 1, 12, 16, 20-30, 64, 80-85, and 220;
b. a Tn7 type transposase complex that binds the Cas effector complex and comprises a TnsB, TnsC, and TniQ component, the TnsB, TnsC, or TniQ component comprising a sequence having at least 80% sequence identity to any one of SEQ ID NOs: 2-4, 13-15, 17-19, 65-67, and 109-111, and an accessory protein comprising a sequence having at least 70% sequence identity to any one of SEQ ID NOs: 228-230 and 235-249; and
c. a double-stranded nucleic acid that interacts with the Tn7 type transposase complex and comprises the cargo nucleotide sequence.

53.-74. (canceled)

75. A system for transposing a cargo nucleotide sequence into a target nucleic acid site in a target nucleic acid comprising:
a. a Cas effector complex that hybridizes to the target nucleic acid site and comprising:
i) a class 2, type V Cas effector comprising a sequence having at least 80% sequence identity to any one of SEQ ID NOs: 1, 81, 82, 83, and 85; and
ii) an engineered guide polynucleotide having at least 80% identity to any one of SEQ ID NOs: 5, 6, 45-63, 68-75, 96-103, 123-140, and 754-944;
b. a Tn7 type transposase complex that binds the Cas effector complex and comprises a TnsB, TnsC, and TniQ component, the TnsB, TnsC, or TniQ component comprising a sequence having at least 80% identity to any one of SEQ ID NOs: 2-4, and an accessory protein comprising a sequence having at least 70% sequence identity to any one of SEQ ID NOs: 228-230 and 235-249; and
c. a double-stranded nucleic acid that interacts with the Tn7 type transposase complex and comprising in 5′ to 3′ order:
i) a left-hand transposase recognition sequence comprising a sequence having at least 80% sequence identity to any one of SEQ ID NOs: 9, 11, 36, 37, and 38;
ii) the cargo nucleotide sequence; and
ii) a right-hand transposase recognition sequence comprising a sequence having at least 80% identity to any one of SEQ ID NOs: 8, 39-44, and 93.

76. A system for transposing a cargo nucleotide sequence into a target nucleic acid site in a target nucleic acid comprising:
a. a Cas effector complex that hybridizes to the target nucleic acid site and comprising:
i) a class 2, type V Cas effector comprising a sequence having at least 80% sequence identity to SEQ ID NOs: 12; and
iii) an engineered guide polynucleotide having at least 80% identity to any one of SEQ ID NOs: 32, 102, 104, and 107;
b. a Tn7 type transposase complex that binds the Cas effector complex and comprising a TnsB, TnsC, and TniQ component, the TnsB, TnsC, or TniQ component comprising a sequence having at least 80% identity to any one of SEQ ID NOs: 13-15, and an accessory protein comprising a sequence having at least 70% sequence identity to any one of SEQ ID NOs: 228-230 and 235-249; and
c. a double-stranded nucleic acid that interacts with the Tn7 type transposase complex and comprising in 5′ to 3′ order:
i) a left-hand transposase recognition sequence comprising a sequence having at least 80% sequence identity to SEQ ID NO: 76;
ii) the cargo nucleotide sequence; and
iii) a right-hand transposase recognition sequence comprising a sequence having at least 80% identity to SEQ ID NO: 77.

77. A system for transposing a cargo nucleotide sequence into a target nucleic acid site in a target nucleic acid comprising:
a. a Cas effector complex that hybridizes to the target nucleic acid site and comprising:
i) a class 2, type V Cas effector comprising a sequence having at least 80% sequence identity to SEQ ID NOs: 16; and
ii) an engineered guide polynucleotide having at least 80% identity to any one of SEQ ID NOs: 33, 103, 105, and 108;
b. a Tn7 type transposase complex that binds the Cas effector complex and comprises a TnsB, TnsC, and TniQ component, the TnsB, TnsC, or TniQ component comprising a sequence having at least 80% identity to any one of SEQ ID NOs: 17-19, an accessory protein comprising a sequence having at least 70% sequence identity to any one of SEQ ID NOs: 228-230 and 235-249; and
c. a double-stranded nucleic acid that interacts with the Tn7 type transposase complex and comprising in 5′ to 3′ order:
i) a left-hand transposase recognition sequence comprising a sequence having at least 80% sequence identity to SEQ ID NO: 78;
ii) the cargo nucleotide sequence; and
iii) a right-hand transposase recognition sequence comprising a sequence having at least 80% identity to SEQ ID NO: 79.

78.-85. (canceled)

86. An engineered nuclease system comprising:
a) an endonuclease comprising a RuvC domain, the endonuclease being derived from an uncultivated microorganism and is a Class 2, type V-K Cas effector comprising at least 80% identity to any one of SEQ ID NOs: 1, 12, 16, 20-30, 64, 80-85, and 220; and
b) an engineered guide RNA that forms a complex with the endonuclease and comprising a spacer sequence that hybridizes to a target nucleic acid sequence wherein the engineered guide polynucleotide comprises a sequence comprising at least 80% identity to any one of SEQ ID NOs: 754-944.

87. A method for transposing a cargo nucleotide sequence into a target nucleic acid site comprising introducing the system of claim 1 to a cell.

88.-99. (canceled)
